(19) United States
(12) Patent Application Publication
(10) Pub. No.: US 2021/0361774 A1
(43) Pub. Date: Nov. 25, 2021

(54) DEGRADERS OF WILD-TYPE AND MUTANT FORMS OF LRRK2

(71) Applicant: DANA-FARBER CANCER INSTITUTE, INC., Boston, MA (US)

(72) Inventors: Nathanael S. Gray, Boston, MA (US); John Hatcher, Boston, MA (US)

(73) Assignee: DANA-FARBER CANCER INSTITUTE, INC., Boston, MA (US)

(21) Appl. No.: 17/284,250

(22) PCT Filed: Oct. 16, 2019
(86) PCT No.: PCT/US19/56537

§ 371 Date: Apr. 9, 2021

Related U.S. Application Data
(60) Provisional application No. 62/884,410, filed on Aug. 8, 2019.
(60) Provisional application No. 62/746,283, filed on Oct. 16, 2018.

(51) Int. Cl. A61K 47/54; C07D 401/14; C07D 417/14

(52) U.S. Cl. CPC A61K 47/545; C07D 417/14; C07D 401/14; A61K 47/555

(57) ABSTRACT

Disclosed are bifunctional compounds (degraders) that target LRKK2 for degradation. Also disclosed are pharmaceutical compositions containing the degraders and methods of using the degraders to treat neurodegenerative diseases and disorders such as Parkinson's disease and brain cancer (e.g., gliomas and glioblastomas).

### RELATED APPLICATIONS

This application claims the benefit of priority under 35 U.S.C. § 119(e) to U.S. Provisional Application No. 62/746,283, filed on Oct. 16, 2018 and to U.S. Provisional Application No. 62/884,410, filed on Aug. 8, 2019, each of which is incorporated herein by reference in its entirety.

### BACKGROUND OF THE INVENTION

Parkinson's disease (PD) is a movement disorder resulting from progressive loss of dopamine producing neurons. It is the second most common neurodegenerative disease in the world, and affects over 1 million Americans. More than 60 000 patients are newly diagnosed each year (Gandhi et al., J. Neurosci. Res. 87:1283-1295 (2009), Daniëls et al., Neurosignals 19:1-15 (2011)). Symptoms associated with Parkinson's disease include motor impairment, tremor, bradykinesia, instability, and other movement related disorders. There are also non-motor symptoms such as cognitive dysfunction, autonomic dysfunction, and sleep disruption. These symptoms greatly reduce the quality of life of those suffering from Parkinson's disease.

Insofar as the genes associated with PD are concerned, leucine-rich repeat kinase 2 (LRRK2) having a missense mutation, G2019S, has been frequently found in both familial and sporadic PD cases (Healy et al., Lancet Neurol. 7:583-590 (2008), Dächsel et al., Neurol. 67:542-547 (2010), Lee et al., Trends Pharmacol. Sci. 33(7):365-373 (2012), Liu et al., Hum. Mol. Genet. 20:3933-3942 (2011)). The G2019S mutation has been shown to increase kinase activity, which resulted in activation of the neuronal death signal pathway (Greggio et al., ASN Neuro 1(1):e00002 (2009), Kumar et al., Expert Rev. Mol. Med. 13:e20 (2011)). Transgenic G2019S LRRK2 mice aged to 12-16 months have been shown to display progressive degeneration of the substantia nigra pars compacta (SNpc) dopaminergic neurons and Parkinson's phenotypes of motor dysfunction (Chen et al., Cell Death Differ. 19(10):1623-33 (2012)).

### SUMMARY OF THE INVENTION

A first aspect of the present invention is directed to a bifunctional compound (also referred to herein as a “degrader” or “PROTAC”), which has a structure represented by formula (I):

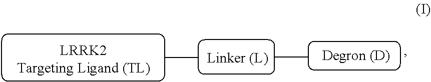

wherein the targeting ligand represents an aminopyrimidine or indazole that binds leucine-rich repeat kinase 2 (LRRK2), the degron represents a ligand that binds an E3 ubiquitin ligase, and the linker represents a moiety that connects covalently the degron and the targeting ligand, or a pharmaceutically acceptable salt or stereoisomer thereof.

A second aspect of the present invention is directed to a pharmaceutical composition containing a therapeutically effective amount of a compound of formula I, or a pharmaceutically acceptable salt or stereoisomer thereof, and pharmaceutically acceptable carrier.

A further aspect of the invention is directed to a method of treating a disease or disorder mediated by aberrant (e.g., dysregulated or dysfunctional) LRRK2 activity, that includes administering a therapeutically effective amount of a bifunctional compound of formula (I) or a pharmaceutically acceptable salt or stereoisomer thereof, to a subject in need thereof. In some embodiments, the inventive compounds are used to treat a neurodegenerative disease such as Parkinson's disease and brain cancer (e.g., gliomas and glioblastomas).

Further aspects of the present invention are directed to methods of making the bifunctional compounds.

Without intending to be bound by any particular theory of operation, the bifunctional compounds of formula (I) are believed to degrade LRRK2 that is involved in the genesis and/or progression of disease via the cell's ubiquitin/proteasome system, whose function is to routinely identify and remove damaged proteins. The degron functional moiety recruits the E3 ubiquitin ligase to tag LRRK2 (which is bound by the targeting ligand functionality) for ubiquitination and degradation through the proteasome, which is a large endogenous complex that degrades the ubiquitinated protein into small peptide fragments. After destruction of a LRRK2 molecule, the degrader is released and continues to be active. Thus, by engaging and exploiting the body's own natural protein disposal system, the bifunctional compounds of formula (I) may represent a potential improvement over traditional small molecule inhibitors of LRRK2 in the treatment of diseases or disorders that have proven or may prove to be difficult to treat.

LRRK2 degraders may offer several additional advantages over existing LRRK2 inhibitors. For example, in view of data suggesting that degraders act in a catalytic fashion (i.e., a single degrader molecule can induce degradation of multiple target proteins), effective intracellular concentrations of degraders may be significantly lower than for conventional kinase antagonists. Also, because degraders cause complete elimination of the protein by the proteasome, pharmacodynamic effects of the degraders are dictated by protein resynthesis rates similar to what is observed for covalent inhibitors. Further, kinase degradation addresses TKI (tyrosine kinase inhibitor) resistance imparted by intrinsic ‘scaffolding’ functions of kinases. Even further, de novo resistance mutations to selective degraders of LRRK2 are less likely to emerge, given that efficient degradation can be achieved even with lower affinity warheads. Thus, bifunctional compounds of formula (I) may have the potential to represent a major advancement over the existing LRRK2-targeted small molecule inhibitors and overcome some of their most significant limitations.

### BRIEF DESCRIPTION OF THE DRAWINGS

FIG. 1 is a Western blot that shows the cellular degradation of LRRK2 (C-terminus) and LRRK2 (N-terminus), and the inhibition of phosphorylation of S935 and Rab10 in a time course experiment with 0 nM-1000 nM inventive compound 1.

FIG. 2 is a Western blot that shows the cellular degradation of LRRK2 (C-terminus) and LRRK2 (N-terminus), and the inhibition of phosphorylation of S935 and Rab10 in a time course experiment with 0 nM-1000 nM inventive compound 2.

FIG. 3A is a Western blot that shows the cellular degradation of LRRK2 (C-terminus) and LRRK2 (N-terminus), and the inhibition of phosphorylation of S935 and Rab10 in a time course experiment with 0 nM-1000 nM inventive compound 3.

FIG. 3B is a Western blot that shows the degradation of LRRK2(C-terminus) and LRRK2 (N-terminus), and the inhibition of phosphorylation of S935 and Rab10 in a time course experiment with 0 nM-1000 nM inventive compound 3 in RC1441C homozygous cells.

FIG. 4 is a Western blot that shows the degradation of LRRK2 (C-terminus) and LRRK2 (N-terminus), and the inhibition of phosphorylation of S935 and Rab10 in a time course experiment with 0 nM-1000 nM inventive compound 4.

FIG. 5 is a Western blot that shows the degradation of LRRK2 (C-terminus) and LRRK2 (N-terminus), and the inhibition of phosphorylation of S935 and Rab10 in a time course experiment with 0 nM-1000 nM inventive compound 5.

FIG. 6 is a Western blot that shows the degradation of LRRK2 (C-terminus) and LRRK2 (N-terminus), and the inhibition of phosphorylation of S935 and Rab10 in a time course experiment with 0 nM-1000 nM inventive compound 6.

FIG. 7 is a Western blot that shows the degradation of LRRK2 (C-terminus) and LRRK2 (N-terminus), and the inhibition of phosphorylation of S935 and Rab10 in a time course experiment with 0 nM-1000 nM inventive compound 7.

FIG. 8 is a graph that shows the intracellular CRBN binding of lenalidomide, pomalidomide, and MLi-2 based inventive compounds at different concentrations (M).

FIG. 9A is a Western blot that shows the degradation of LRRK2 total and the inhibition of phosphorylation of S935 in a time course experiment with 0 nM-1000 nM MLi-2 analog 5-(1-methylcyclopropyl)oxy-3-[6-(4-methylpiperazin-1-yl)pyrimidin-4-yl]-1H-indazole.

FIG. 9B is a graph that shows the inhibition of LRRK2 in a time course experiment with 0 nM-1000 nM MLi-2 analog 5-(1-methylcyclopropyl)oxy-3-[6-(4-methylpiperazin-1-yl)pyrimidin-4-yl]-1H-indazole.

FIG. 9C is a graph that shows the inhibition of LRRK2 pS935 in a time course experiment with 0 nM-1000 nM MLi-2 analog 5-(1-methylcyclopropyl)oxy-3-[6-(4-methylpiperazin-1-yl)pyrimidin-4-yl]-1H-indazole.

FIG. 10A is a Western blot that shows the degradation of LRRK2 total and the inhibition of phosphorylation of S935 in a time course experiment with 0 nM-1000 nM inventive compound 8.

FIG. 10B is a graph that shows the degradation of LRRK2 in a time course experiment with 0 nM-1000 nM inventive compound 8.

FIG. 10C is a graph that shows the inhibition of phosphorylation of S935 in a time course experiment with 0 nM-1000 nM inventive compound 8.

FIG. 11A is a Western blot that shows the degradation of LRRK2 total and the inhibition of phosphorylation of S935 in a time course experiment with 0 nM-1000 nM inventive compound 9.

FIG. 11B is a graph that shows the degradation of LRRK2 in a time course experiment with 0 nM-1000 nM inventive compound 9.

FIG. 11C is a graph that shows the inhibition of phosphorylation of S935 in a time course experiment with 0 nM-1000 nM inventive compound 9.

FIG. 12A is a Western blot that shows the degradation of LRRK2 total and the inhibition of phosphorylation of S935 in a time course experiment with 0 nM-1000 nM inventive compound 10.

FIG. 12B is a graph that shows the degradation of LRRK2 in a time course experiment with 0 nM-1000 nM inventive compound 10.

FIG. 12C is a graph that shows the inhibition of phosphorylation of S935 in a time course experiment with 0 nM-1000 nM inventive compound 10.

FIG. 13A is a Western blot that shows the degradation of LRRK2 total and the inhibition of phosphorylation of S935 in a time course experiment with 0 nM-1000 nM inventive compound 11.

FIG. 13B is a graph that shows the degradation of LRRK2 in a time course experiment with 0 nM-1000 nM inventive compound 11.

FIG. 13C is a graph that shows the inhibition of phosphorylation of S935 in a time course experiment with 0 nM-1000 nM inventive compound 11.

FIG. 14A is a Western blot that shows the degradation of LRRK2 total and the inhibition of phosphorylation of S935 in a time course experiment with 0 nM-1000 nM inventive compound 12.

FIG. 14B is a graph that shows the degradation of LRRK2 in a time course experiment with 0 nM-1000 nM inventive compound 12.

FIG. 14C is a graph that shows the inhibition of phosphorylation of S935 in a time course experiment with 0 nM-1000 nM inventive compound 12.

FIG. 15A is a Western blot that shows the degradation of LRRK2 total and the inhibition of phosphorylation of S935 in a time course experiment with 0 nM-1000 nM inventive compound 13.

FIG. 15B is a graph that shows the degradation of LRRK2 in a time course experiment with 0 nM-1000 nM inventive compound 13.

FIG. 15C is a graph that shows the inhibition of phosphorylation of S935 in a time course experiment with 0 nM-1000 nM inventive compound 13.

FIG. 16A is a Western blot that shows the degradation of LRRK2 total and the inhibition of phosphorylation of S935 and RAB(E8261) in a time course experiment with 0 nM-1000 nM inventive compound 14.

FIG. 16B is a graph that shows the degradation of LRRK2 in a time course experiment with 0 nM-1000 nM inventive compound 14.

FIG. 16C is a graph that shows the inhibition of phosphorylation of S935 in a time course experiment with 0 nM-1000 nM inventive compound 14.

FIG. 16D is a graph that shows the inhibition of phosphorylation of Rab (E8261) in a time course experiment with 0 nM-1000 nM inventive compound 14.

FIG. 17A is a Western blot that shows the degradation of LRRK2 total and the inhibition of phosphorylation of S935 and RAB (E8261) in a time course experiment with 0 nM-1000 nM inventive compound 15.

FIG. 17B is a graph that shows the degradation of LRRK2 in a time course experiment with 0 nM-1000 nM inventive compound 15.

FIG. 17C is a graph that shows the inhibition of phosphorylation of S935 in a time course experiment with 0 nM-1000 nM inventive compound 15.

FIG. 17D is a graph that shows the degradation of phospho-Rab (E8261) in a time course experiment with 0 nM-1000 nM inventive compound 15.

FIG. 18A is a set of Western blots that show the degradation of LRRK2 total and the inhibition of phosphorylation of S935 and Rab10 (E8261) after 48 hours with a known MLi-2 analog 5-(1-methylcyclopropyl)oxy-3-[6-(4-methylpiperazin-1-yl)pyrimidin-4-yl]-1H-indazole, inventive compound 8, inventive compound 16 (negative control), and negative control DMSO.

FIG. 18B is a graph that shows the degradation of LRRK2 (UDD3) after 48 hours with MLi-2 analog 5-(1-methylcyclopropyl)oxy-3-[6-(4-methylpiperazin-1-yl)pyrimidin-4-yl]-1H-indazole, inventive compound 8, negative control 16, and negative control DMSO.

FIG. 18C is a graph that shows the inhibition of phosphorylation of S935 after 48 hours with MLi-2 analog 5-(1-methylcyclopropyl)oxy-3-[6-(4-methylpiperazin-1-yl)pyrimidin-4-yl]-1H-indazole, inventive compound 8, negative control 16, and negative control DMSO.

FIG. 18D is a graph that shows the inhibition of phosphorylation of Rab10 after 48 hours with MLi-2 analog 5-(1-methylcyclopropyl)oxy-3-[6-(4-methylpiperazin-1-yl)pyrimidin-4-yl]-1H-indazole, inventive compound 8, negative control 16, and negative control DMSO.

### DETAILED DESCRIPTION OF THE INVENTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present invention.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present invention, and to the extent the following terms are used herein to further describe them, the following definitions apply.

As used herein, the term “alkyl” refers to a saturated linear or branched-chain monovalent hydrocarbon radical. In one embodiment, the alkyl radical is a C₁-C₁₈ group. In other embodiments, the alkyl radical is a C₀-C₆, C₀-C₅, C₀-C₃, C₁-C₁₂, C₁-C₈, C₁-C₆, C₁-C₅, C₁-C₄ or C₁-C₃ group (wherein C₀ alkyl refers to a bond). Examples of alkyl groups include methyl, ethyl, 1-propyl, 2-propyl, i-propyl, 1-butyl, 2-methyl-1-propyl, 2-butyl, 2-methyl-2-propyl, 1-pentyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, heptyl, octyl, nonyl, decyl, undecyl and dodecyl. In some embodiments, an alkyl group is a C₁-C₃ alkyl group. In some embodiments, an alkyl group is a C₁-C₂ alkyl group.

As used herein, the term “alkylene” refers to a straight or branched divalent hydrocarbon chain linking the rest of the molecule to a radical group, consisting solely of carbon and hydrogen, containing no unsaturation and having from one to 12 carbon atoms, for example, methylene, ethylene, propylene, n-butylene, and the like. The alkylene chain may be attached to the rest of the molecule through a single bond and to the radical group through a single bond. In some embodiments, the alkylene group contains one to 8 carbon atoms (C₁-C₈ alkylene). In other embodiments, an alkylene group contains one to 5 carbon atoms (C₁-C₅ alkylene). In other embodiments, an alkylene group contains one to 4 carbon atoms (C₁-C₄ alkylene). In other embodiments, an alkylene contains one to three carbon atoms (C₁-C₃ alkylene). In other embodiments, an alkylene group contains one to two carbon atoms (C₁-C₂ alkylene). In other embodiments, an alkylene group contains one carbon atom (C₁ alkylene).

As used herein, the term “haloalkyl” refers to an alkyl group as defined herein that is substituted with one or more (e.g., 1, 2, 3, or 4) halo groups.

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In one example, the alkenyl radical is a C₂-C₁₈ group. In other embodiments, the alkenyl radical is a C₂-C₁₂, C₂-C₁₀, C₂-C₈, C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

As used herein, the term “alkynyl” refers to a linear or branched monovalent hydrocarbon radical with at least one carbon-carbon triple bond. In one example, the alkynyl radical is a C₂-C₁₈ group. In other examples, the alkynyl radical is C₂-C₁₂, C₂-C₁₀, C₂-C₈, C₂-C₆ or C₂-C₃. Examples include ethynyl prop-1-ynyl, prop-2-ynyl, but-1-ynyl, but-2-ynyl and but-3-ynyl.

As used herein, the term “aldehyde” is represented by the formula-C(O)H. The terms “C(O)” and C═O are used interchangeably herein.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “halogen” (or “halo” or “halide”) refers to fluorine, chlorine, bromine, or iodine.

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, a cyclic group can contain one or more carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. In one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂.

Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocycyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

Thus, the term carbocyclic also embraces carbocyclylalkyl groups which as used herein refer to a group of the formula —R^{c}-carbocyclyl where R^{c} is an alkylene chain. The term carbocyclic also embraces carbocyclylalkoxy groups which as used herein refer to a group bonded through an oxygen atom of the formula —O—R^{c}-carbocyclyl where R^{c} is an alkylene chain.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro-ring systems, and combinations thereof. In some embodiments, a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and Spiro ring systems, wherein the ring atoms are carbon, and one to 5 ring atoms is a heteroatom such as nitrogen, sulfur or oxygen. In some embodiments, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl. The pyridine N-oxides and pyridazine N-oxides and the pyridyl, pyrimid-2-yl, pyrimid-4-yl, pyridazinyl and the 1,3,4-triazin-2-yl groups, are yet other examples of heterocyclyl groups. In some embodiments, a heterocyclic group includes a heterocyclic ring fused to one or more (e.g., 1, 2 or 3) different cyclic groups (e.g., carbocyclic rings or heterocyclic rings), where the radical or point of attachment is on the heterocyclic ring, and in some embodiments wherein the point of attachment is a heteroatom contained in the heterocyclic ring.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. Representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. Representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, aryl includes groups having 6-18 carbon atoms. In another embodiment, aryl includes groups having 6-10 carbon atoms.

Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-1H-indenyl, naphthyridinyl, and the like, which may be substituted or independently substituted by one or more substituents described herein. A particular aryl is phenyl. In some embodiments, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R^{c}-aryl where R^{c} is an alkylene chain such as methylene or ethylene. In some embodiments, the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, heteroaryl includes 4-6 membered monocyclic aromatic groups where one or more ring atoms is nitrogen, sulfur or oxygen that is independently optionally substituted. In another embodiment, heteroaryl includes 5-6 membered monocyclic aromatic groups where one or more ring atoms is nitrogen, sulfur or oxygen. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

Thus, the term heteroaryl embraces N-heteroaryl groups which as used herein refer to a heteroaryl group as defined above containing at least one nitrogen and where the point of attachment of the heteroaryl group to the rest of the molecule is through a nitrogen atom in the heteroaryl group. The term heteroaryl also embraces C-heteroaryl groups which as used herein refer to a heteroaryl group as defined above and where the point of attachment of the heteroaryl group to the rest of the molecule is through a carbon atom in the heteroaryl group. The term heteroaryl also embraces heteroarylalkyl groups which as disclosed above refer to a group of the formula —R^{c}-heteroaryl, where R^{c} is an alkylene chain as defined above. The term heteroaryl also embraces heteroaralkoxy (or heteroarylalkoxy) groups which as used herein refer to a group bonded through an oxygen atom of the formula —O—R^{c}-heteroaryl, where R^{c} is an alkylene group as defined above.

Any of the groups described herein may be substituted or unsubstituted. As used herein, the term “substituted” broadly refers to all permissible substituents with the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

Representative substituents include halogens, hydroxyl groups, and any other organic groupings containing any number of carbon atoms, e.g., 1-14 carbon atoms, and which may include one or more (e.g., 1 2 3, or 4) heteroatoms such as oxygen, sulfur, and nitrogen grouped in a linear, branched, or cyclic structural format.

Representative examples of substituents may thus include alkyl, substituted alkyl, alkoxy, substituted alkoxy, alkenyl, substituted alkenyl, alkynyl, substituted alkynyl, cyclic, substituted cyclic, carbocyclic, substituted carbocyclic, heterocyclic, substituted heterocyclic, aryl (e.g., benzyl and phenyl), substituted aryl (e.g., substituted benzyl or phenyl), heteroaryl, substituted heteroaryl, aralkyl, substituted aralkyl, halo, hydroxyl, aryloxy, substituted aryloxy, alkylthio, substituted alkylthio, arylthio, substituted arylthio, cyano, carbonyl, substituted carbonyl, carboxyl, substituted carboxyl, amino, substituted amino, amido, substituted amido, sulfonyl, substituted sulfonyl, amino acid, and peptide groups.

The term “binding” as it relates to interaction between the targeting ligand and LRRK2 refers to an inter-molecular interaction that is sufficient to achieve recruitment of LRRK2 to close proximity of the E3 ligase and subsequent degradation of LRRK2. The binding may also be substantially selective in that binding of the targeting ligand with other proteinaceous entities present in the cell is functionally insignificant.

The term “binding” as it relates to interaction between the degron and the E3 ubiquitin ligase, typically refers to an inter-molecular interaction that may or may not exhibit an affinity level that equals or exceeds that affinity between the targeting ligand and the target protein, but nonetheless wherein the affinity is sufficient to achieve recruitment of the ligase to the targeted degradation and the selective degradation of the targeted protein.

Broadly, the bifunctional compounds of the present invention have a structure represented by formula (I):

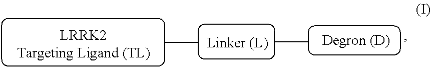

wherein the targeting ligand represents an aminopyrimidine or indazole that binds leucine-rich repeat kinase 2 (LRRK2), the degron represents a ligand that binds an E3 ubiquitin ligase, and the linker represents a moiety that connects covalently the degron and the targeting ligand, or a pharmaceutically acceptable salt or stereoisomer thereof.

In some embodiments, the targeting ligand is an aminopyrimidine and has a structure represented by any one of the following formulae:

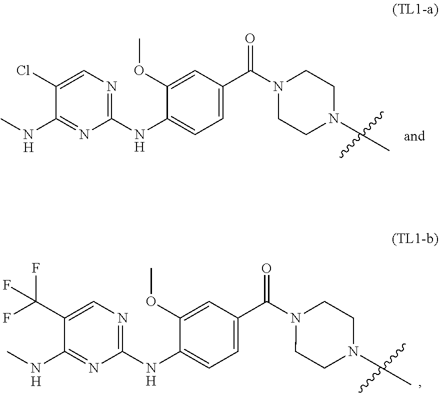

wherein the squiggle represents the point of attachment to the linker.

Other aminopyrimidine analogs thereof that may be useful as targeting ligands in the present bifunctional compounds are described in U.S. Pat. No. 8,802,647.

Thus, in some embodiments, the compounds of the present invention have structures represented by formula (I-1a) or (I-1b):

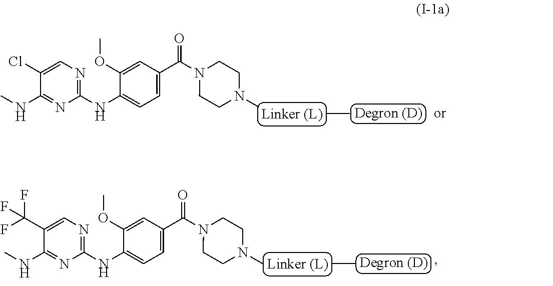

or a pharmaceutically acceptable salt or stereoisomer thereof.

In some embodiments, the targeting is an indazole and has a structure represented by formula TL2-a:

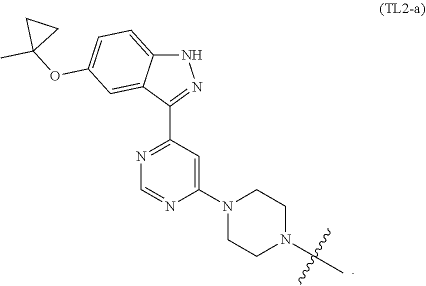 [SMILES: *N1CCN(c2cc(-c3n[nH]c4ccc(OC5(C)CC5)cc34)ncn2)CC1]

Other indazoles that may be useful as targeting ligands in the present bifunctional compounds are described in U.S. Patent Application Publication No. 2016/0009689 A1.

Thus, in some embodiments, the compounds of the present invention have a structure as represented by formula I-2a:

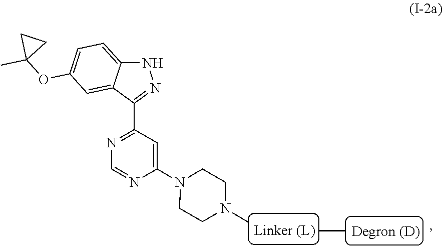

or a pharmaceutically acceptable salt or stereoisomer thereof.

In some embodiments, the targeting ligand has a structure represented by formula TL2-b:

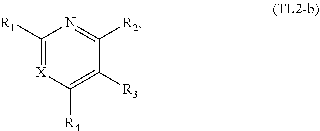

wherein:
X represents N, CR₅, or CR₆; wherein R₅ represents

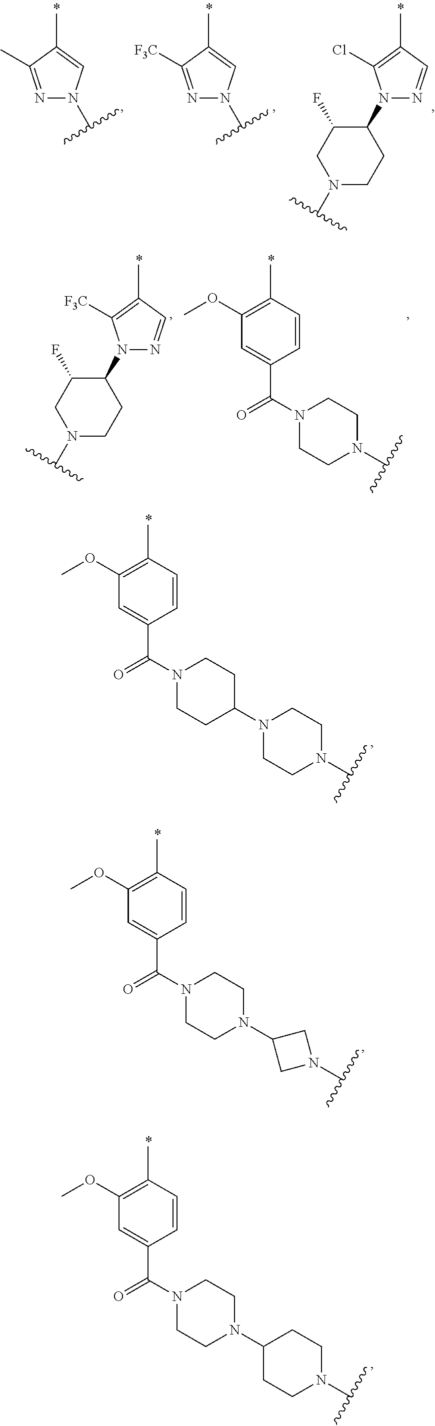

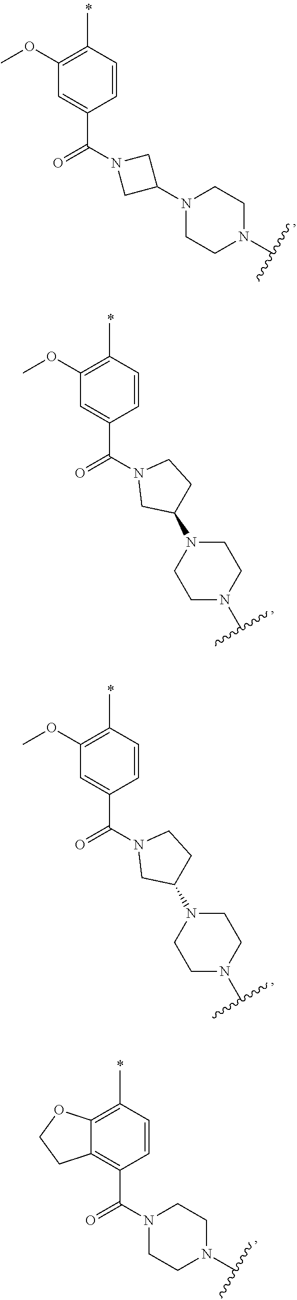

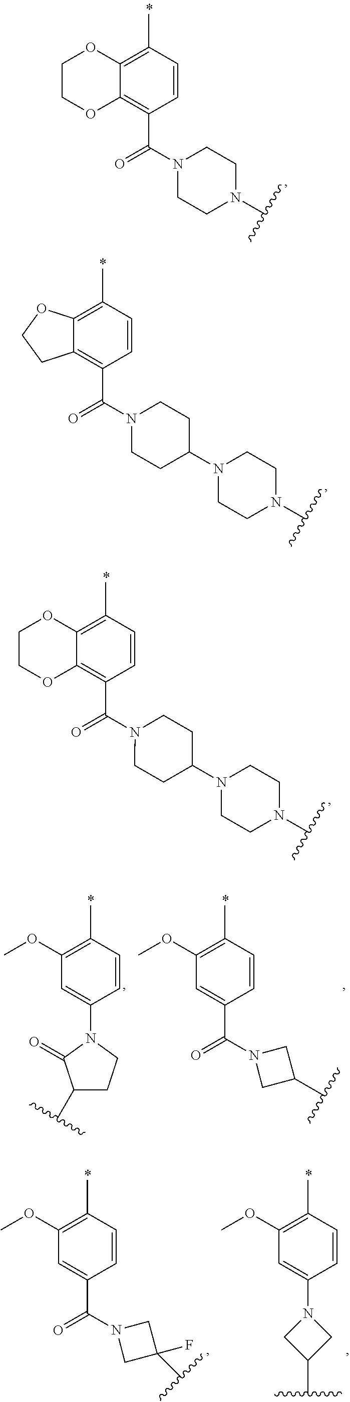

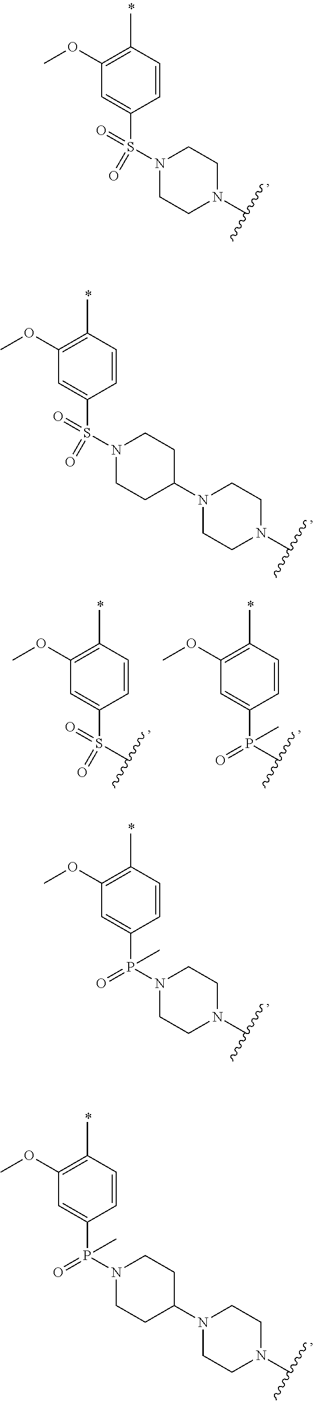

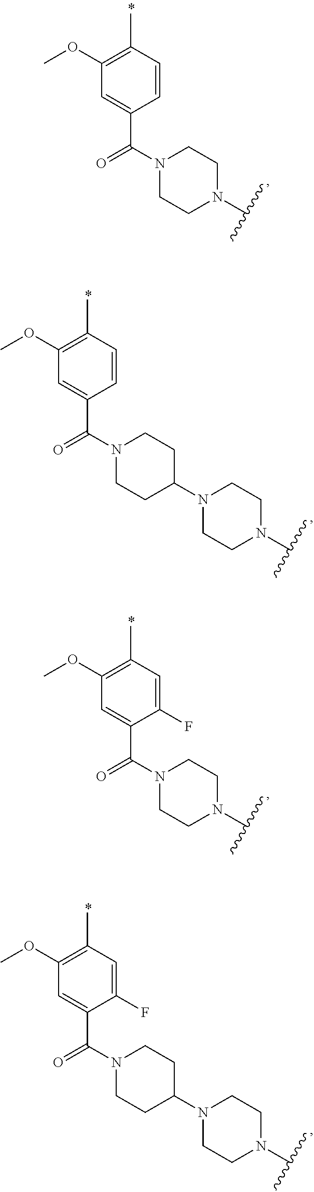

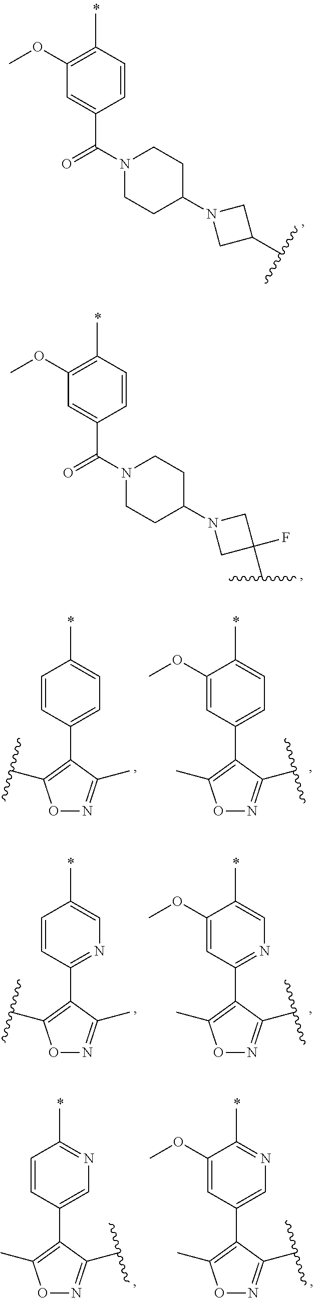

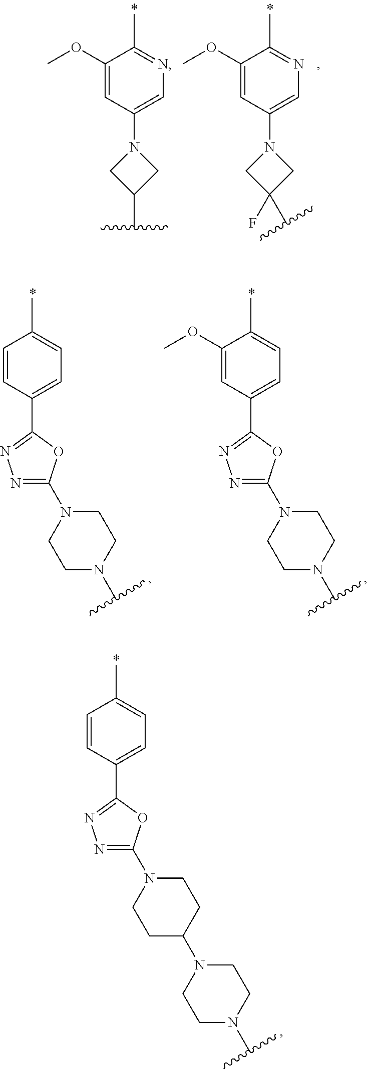

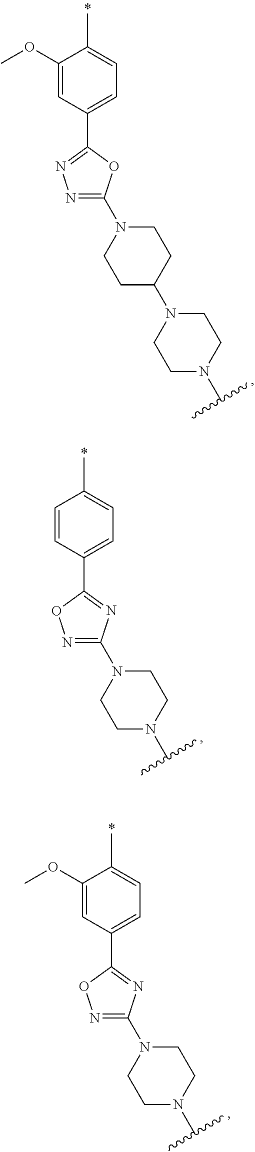 [SMILES: *N1CCN(C2CCN(c3nnc(-c4ccc(C)c(OC)c4)o3)CC2)CC1 ; "," ; *N1CCN(c2noc(-c3ccc(C)cc3)n2)CC1 ; "," ; *N1CCN(c2noc(-c3ccc(C)c(OC)c3)n2)CC1 ; ","]

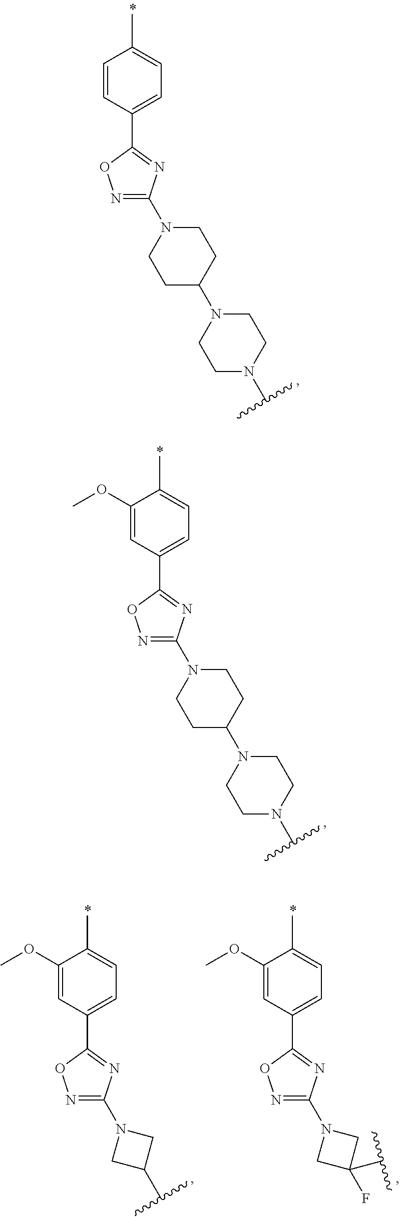

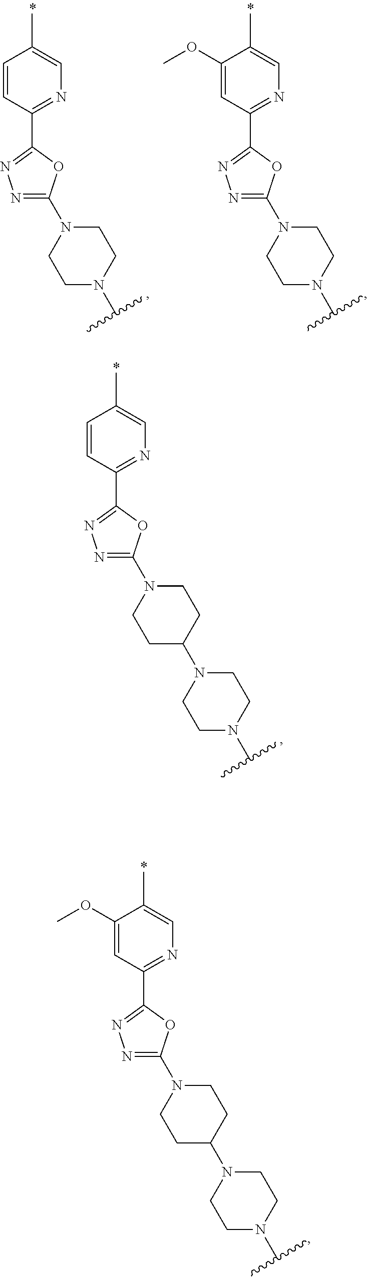

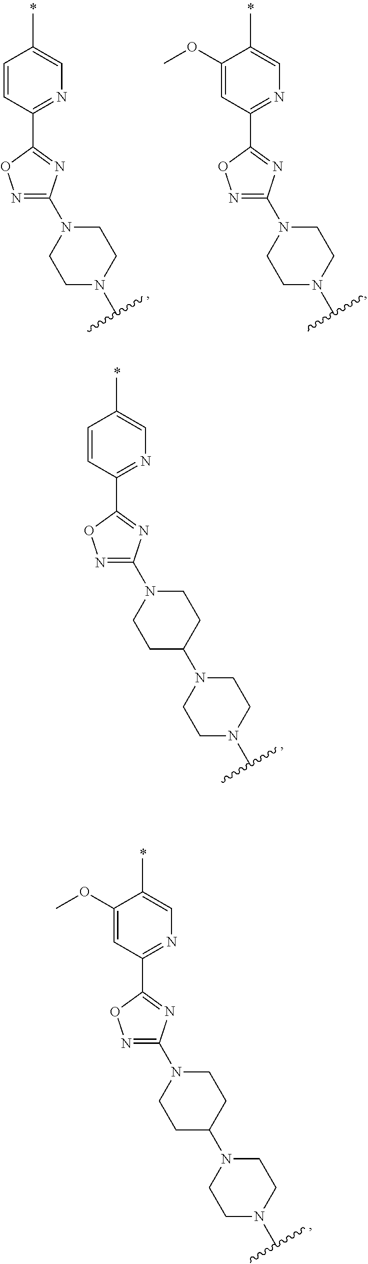

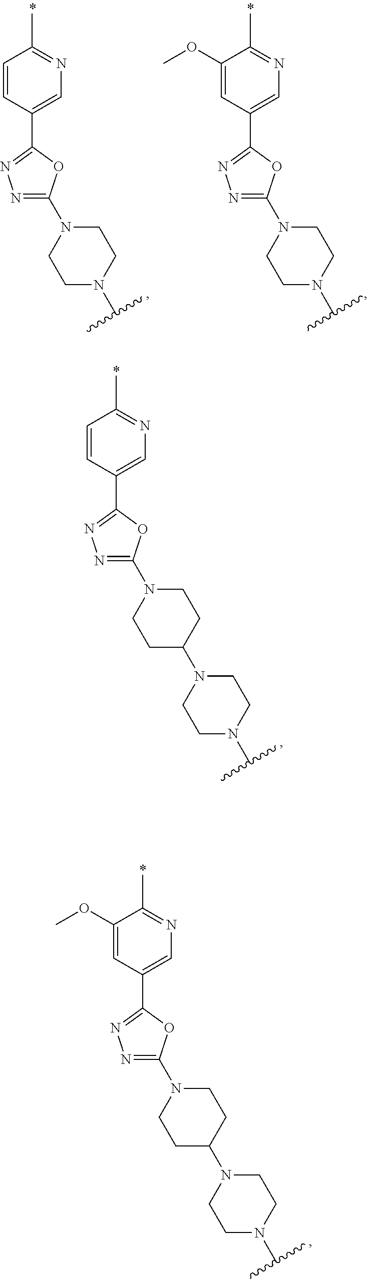

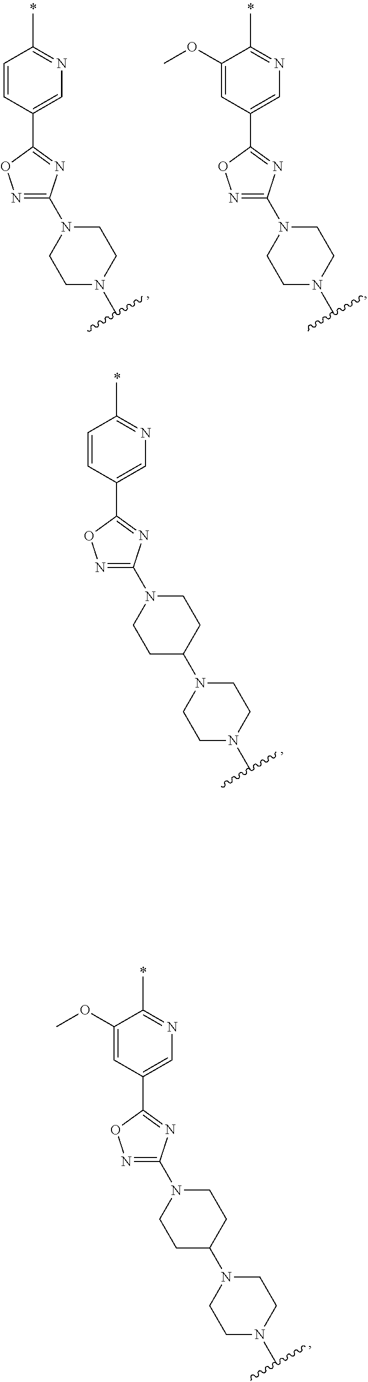

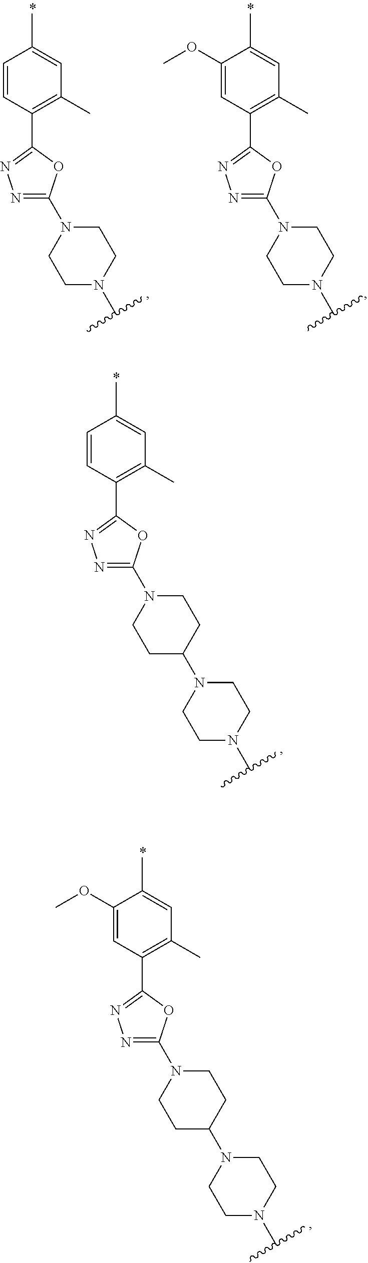

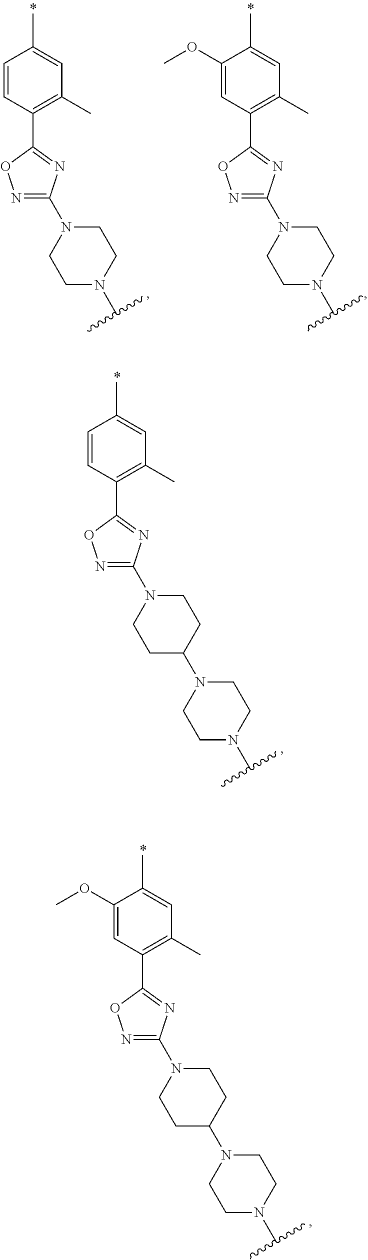

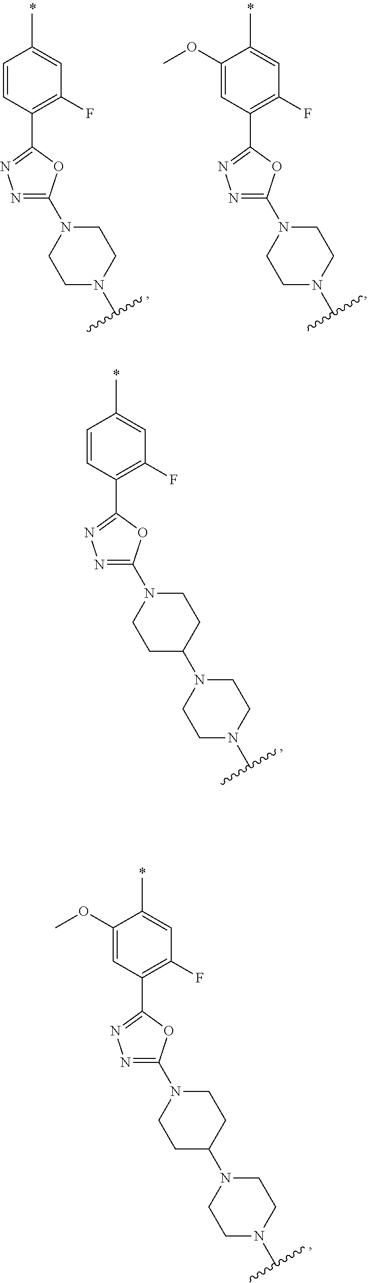

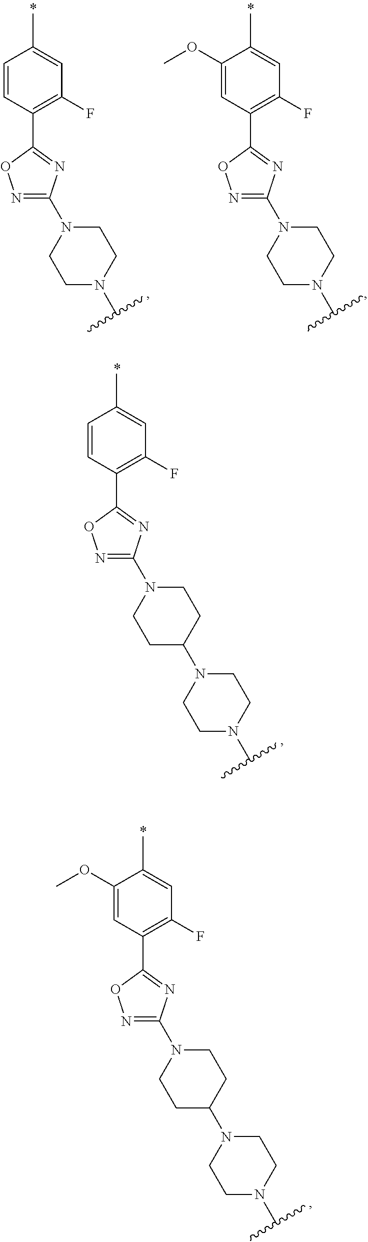

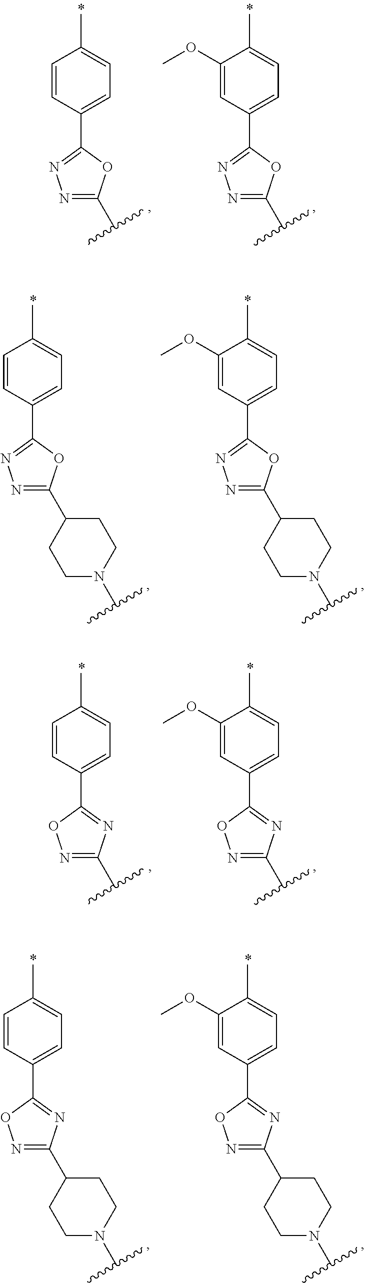

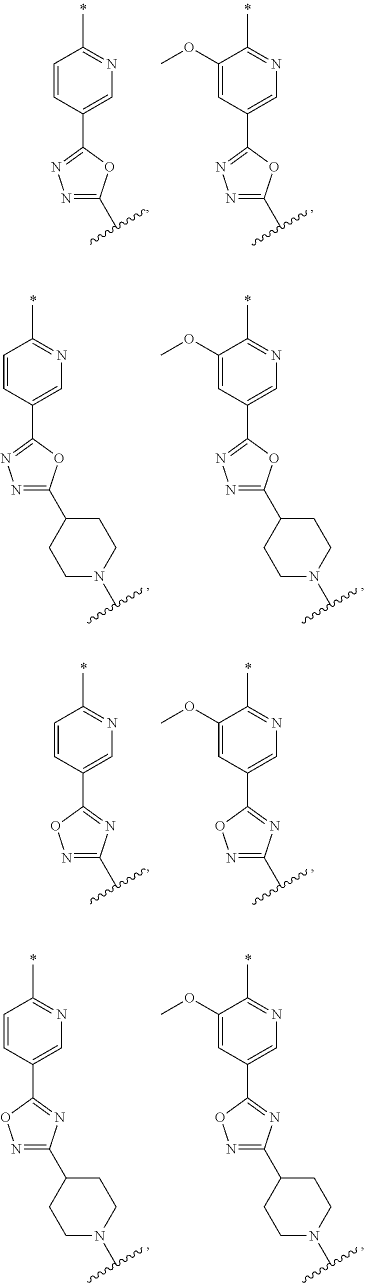

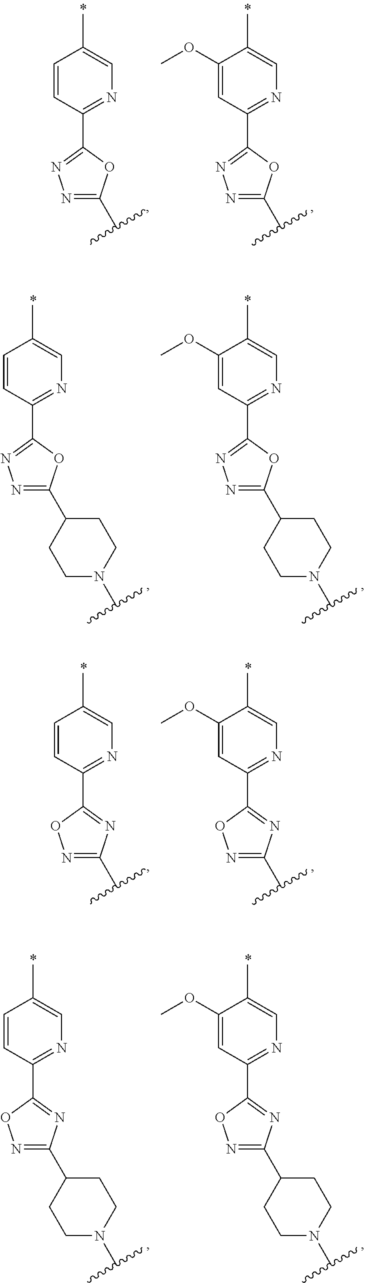

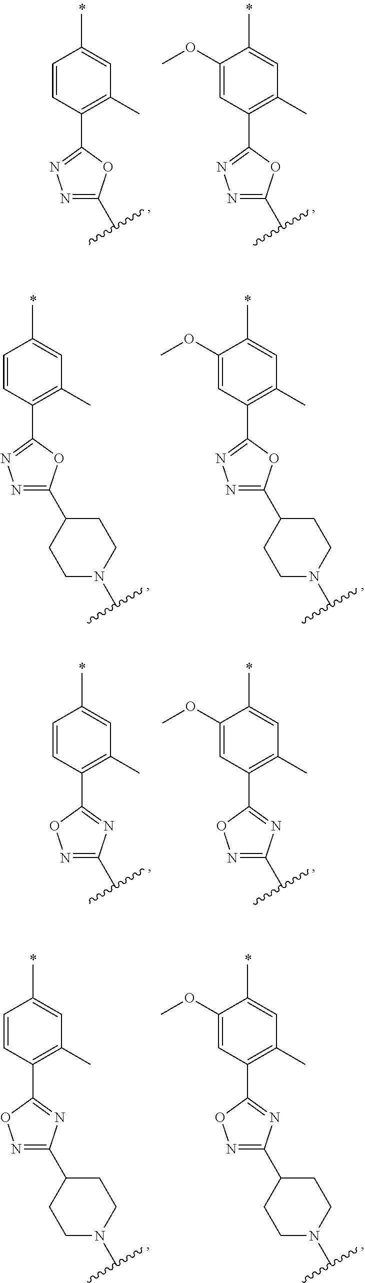

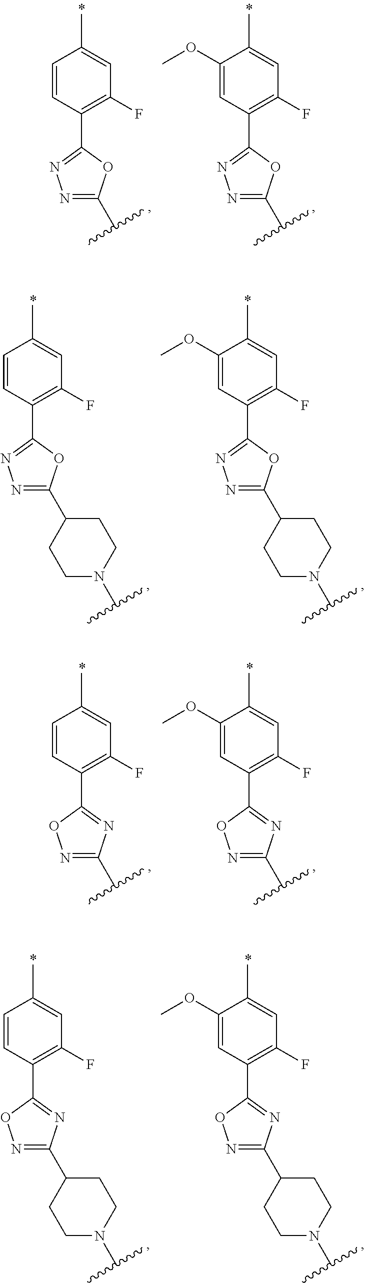

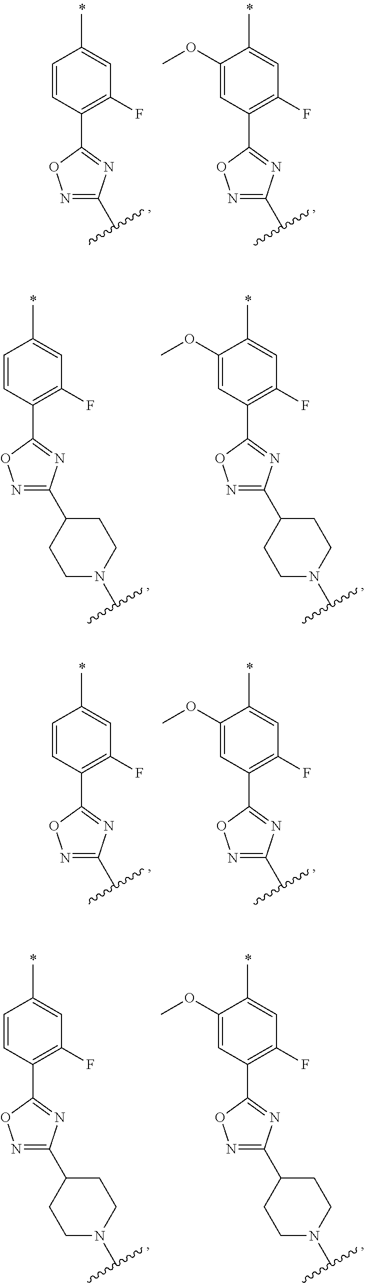

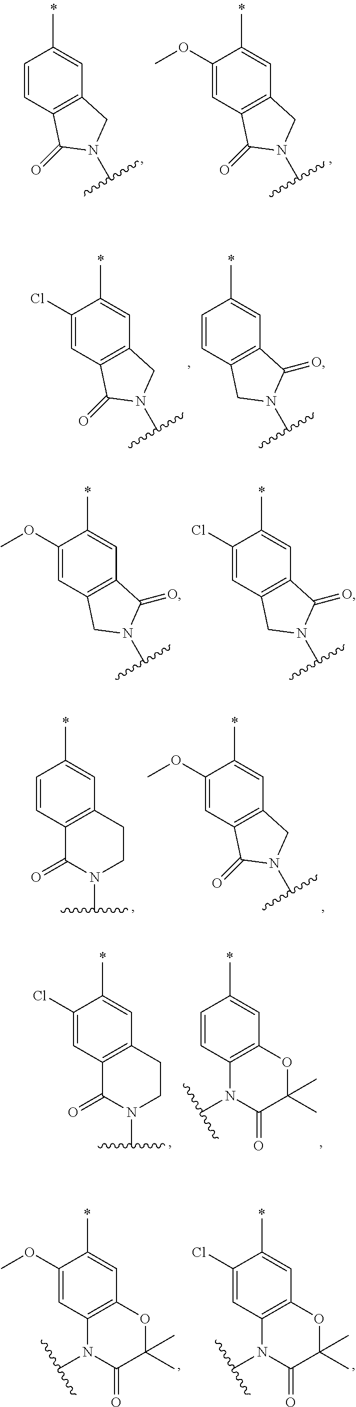

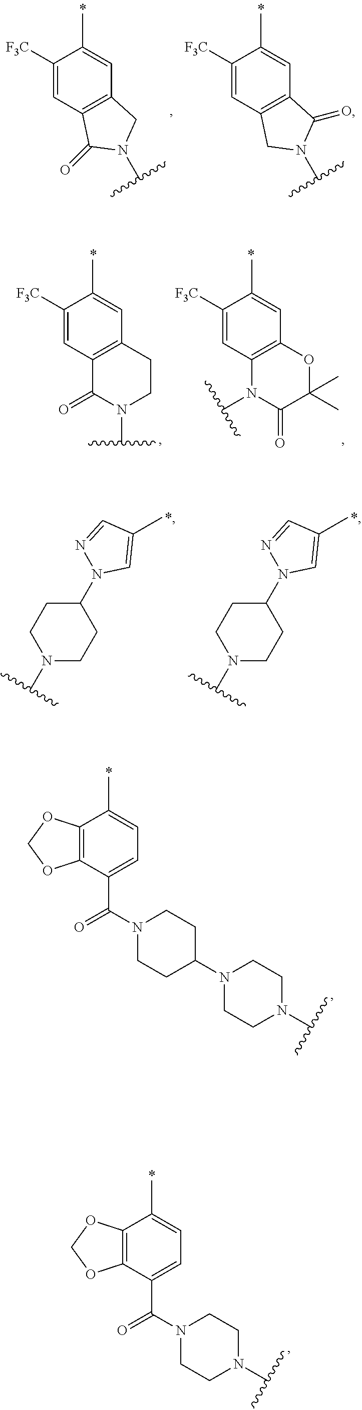

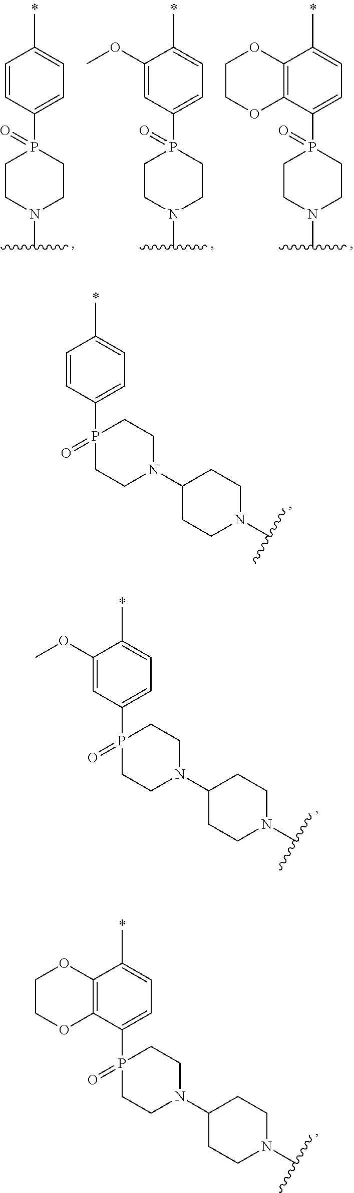

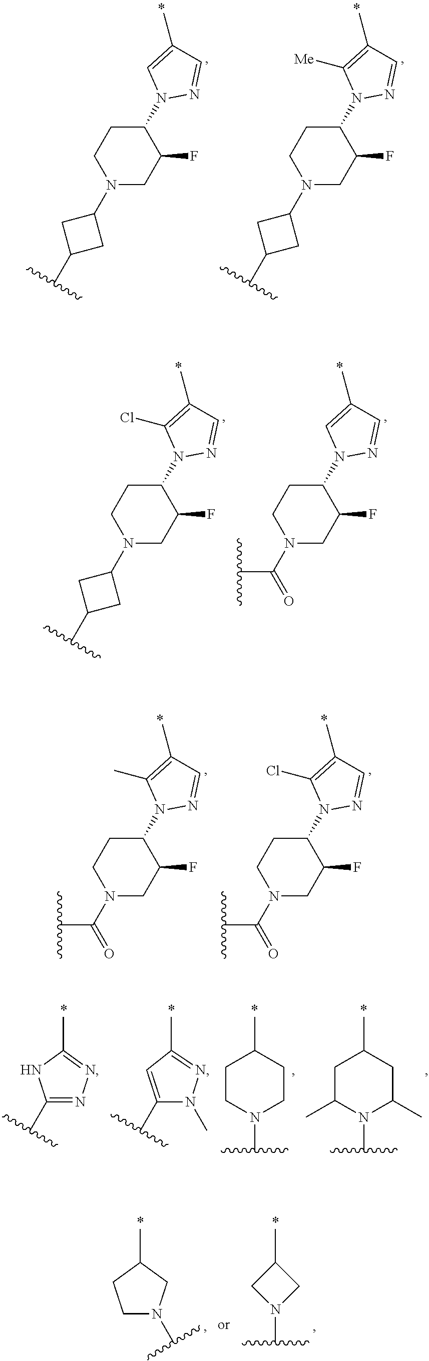

or represents H, wherein the asterisk (*) represents the point of attachment to the heterocyclic ring and the squiggle represents the point of attachment to

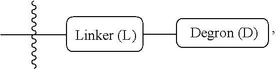

R₆ represents H, halo (e.g., F or Cl) or CF₃;
R₁ represents

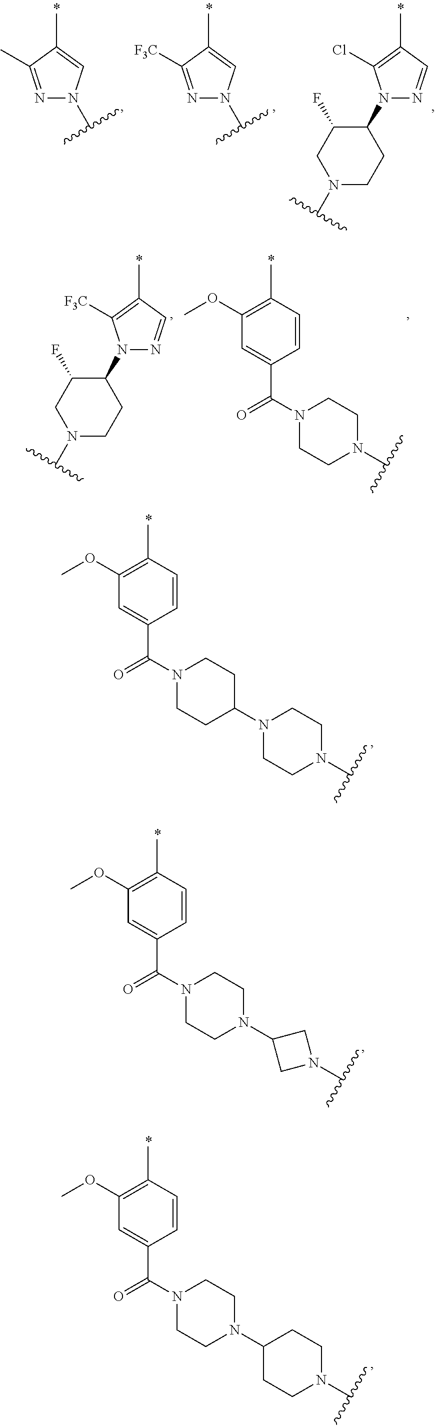

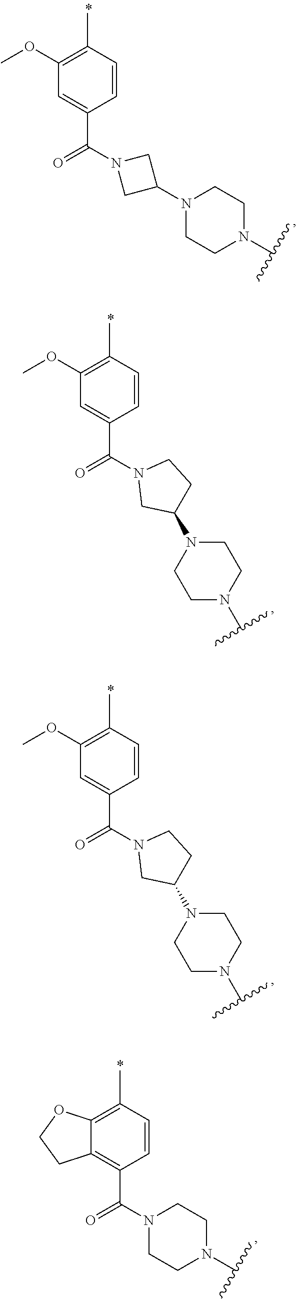

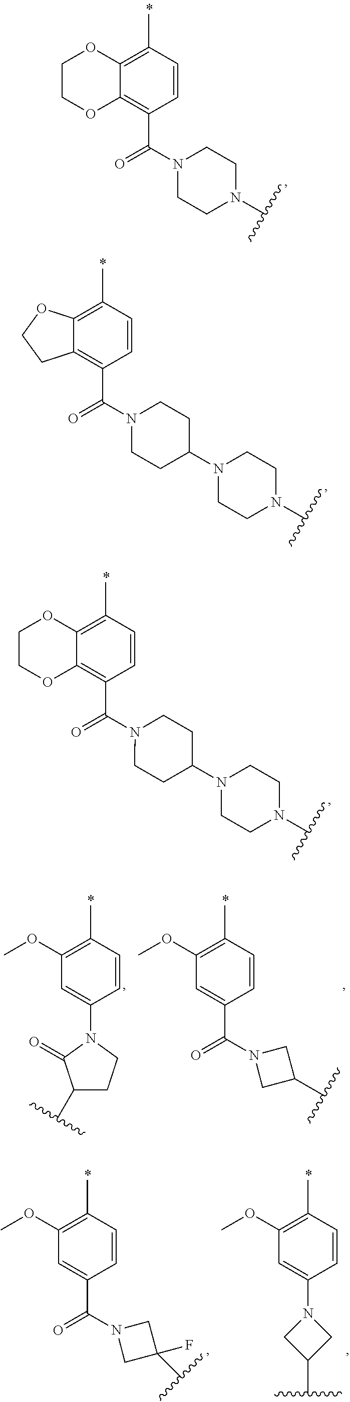

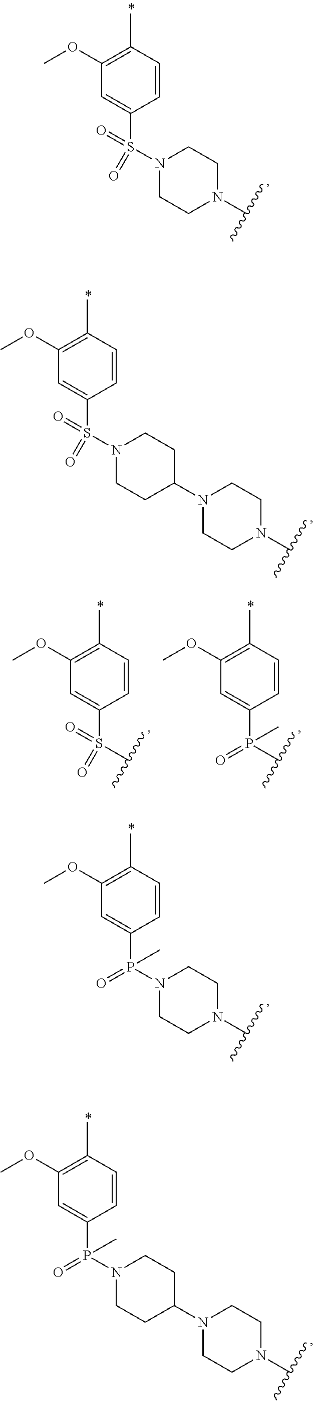

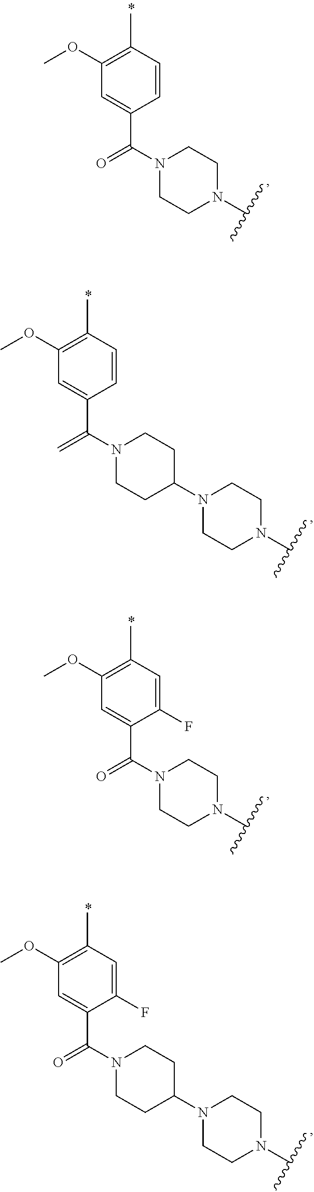

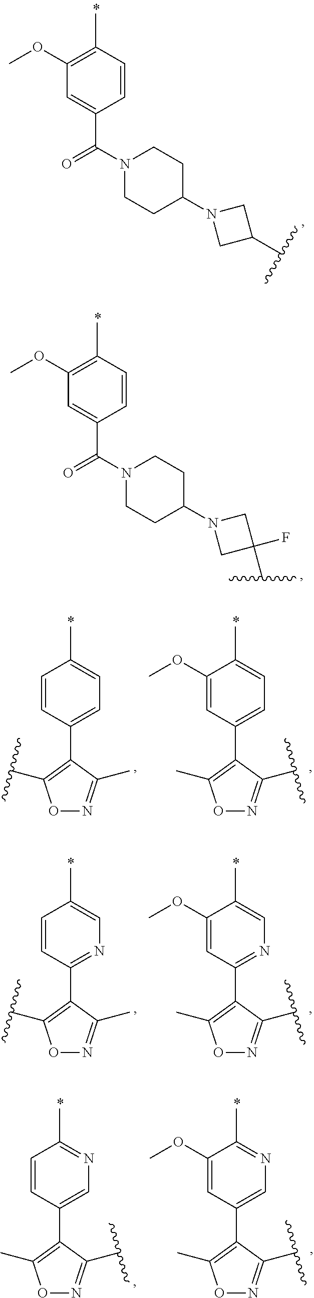

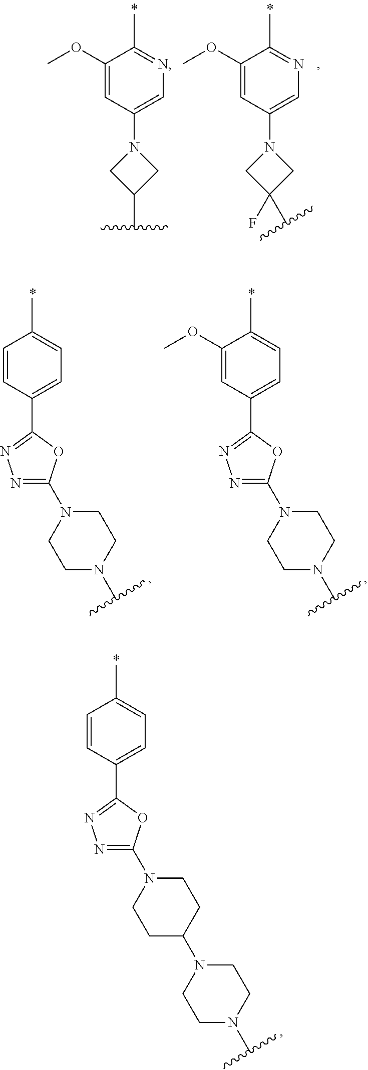

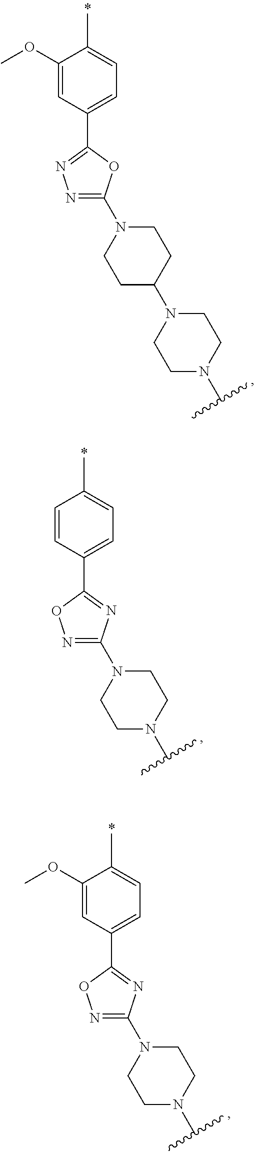 [SMILES: *N1CCN(C2CCN(c3nnc(-c4ccc(C)c(OC)c4)o3)CC2)CC1 ; "," ; *N1CCN(c2noc(-c3ccc(C)cc3)n2)CC1 ; "," ; *N1CCN(c2noc(-c3ccc(C)c(OC)c3)n2)CC1 ; ","]

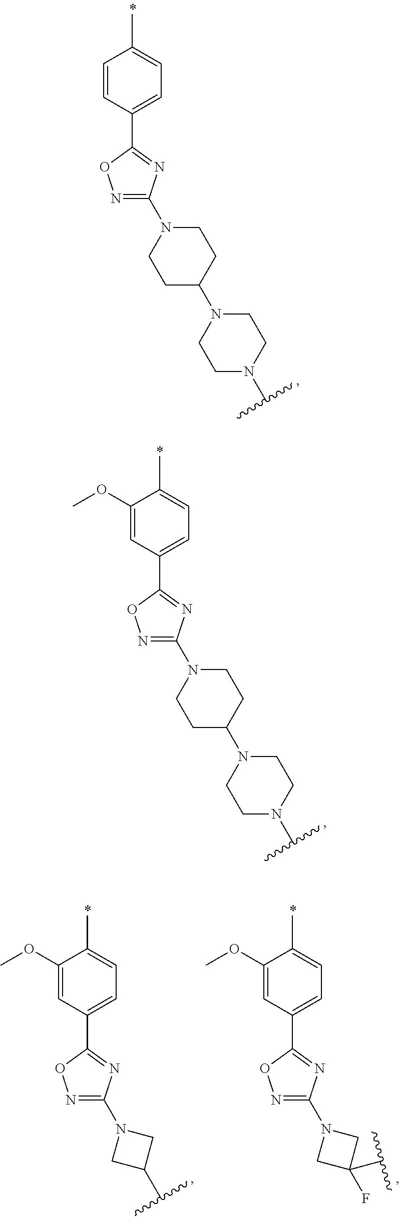

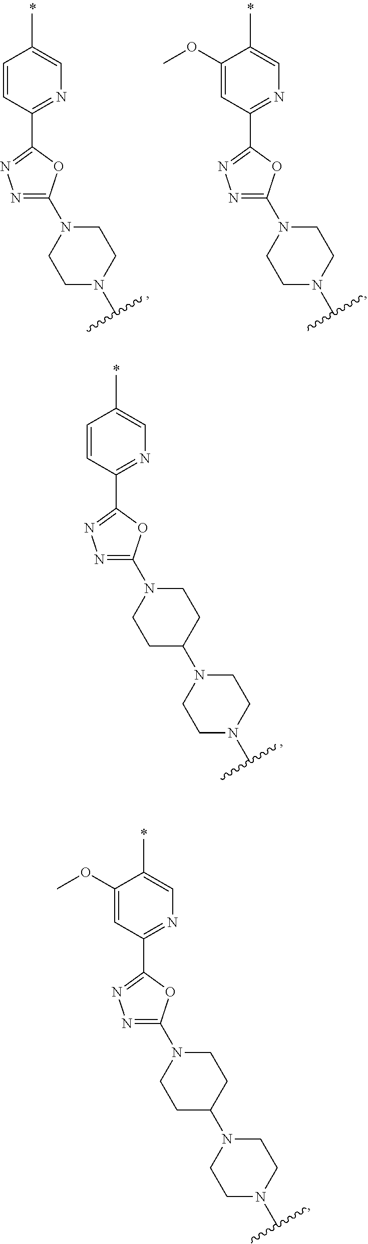

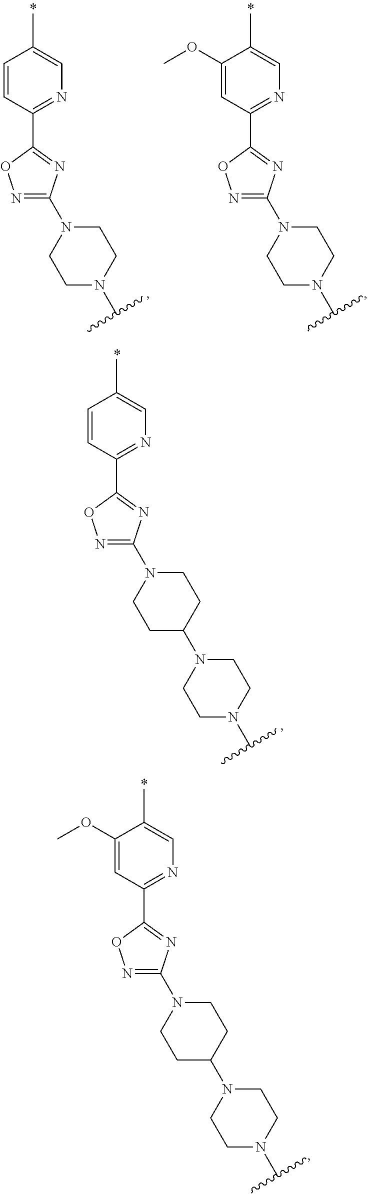

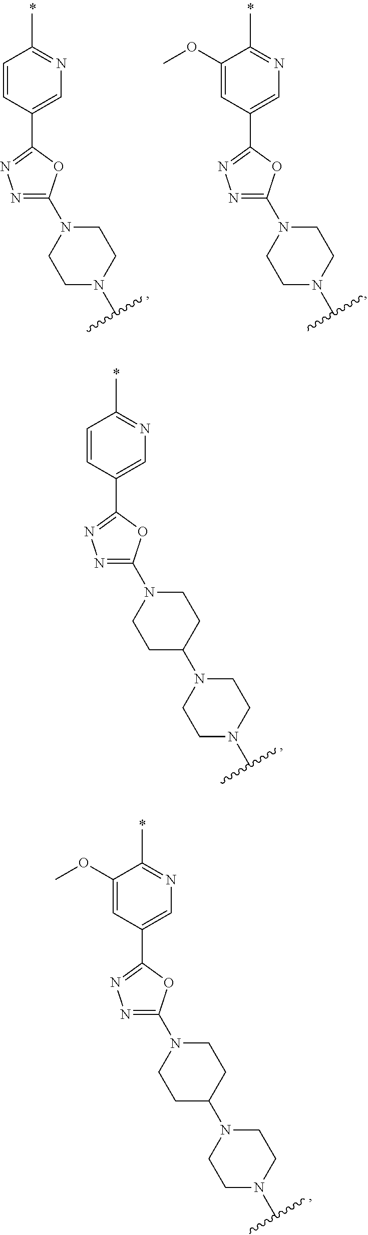

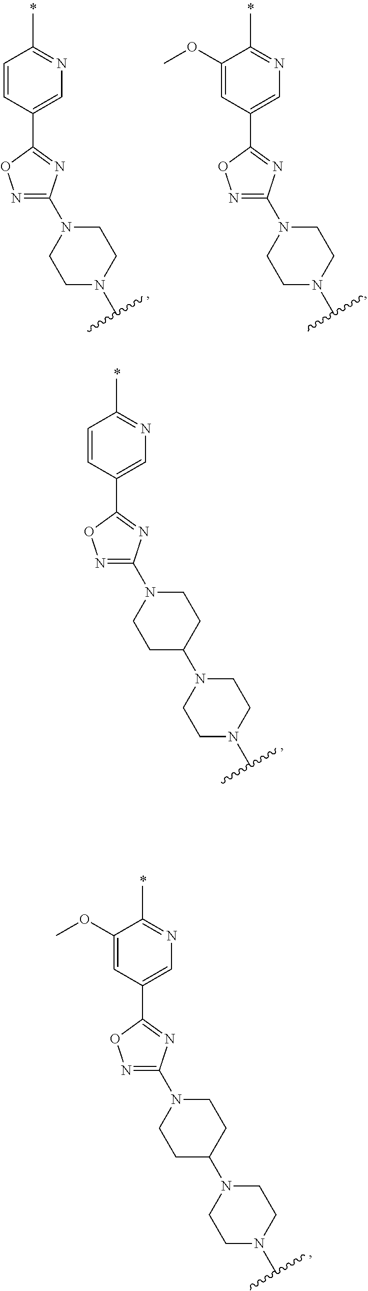

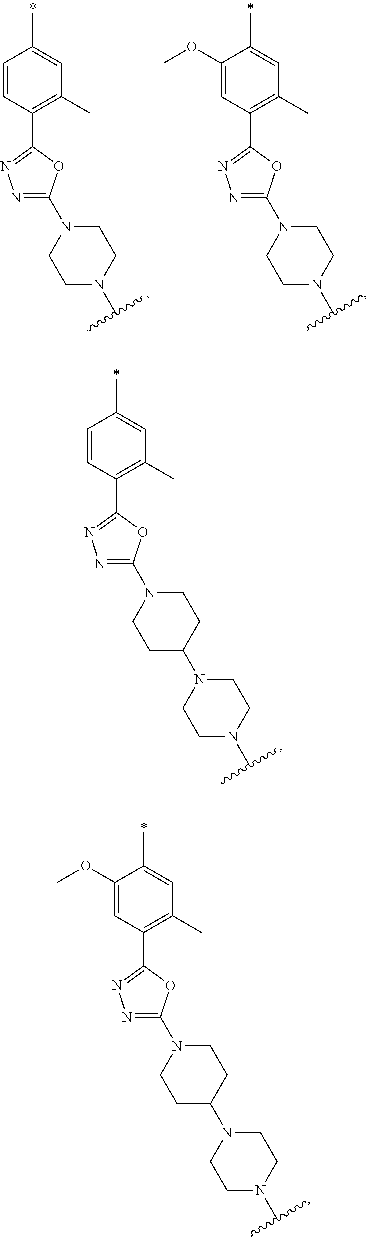

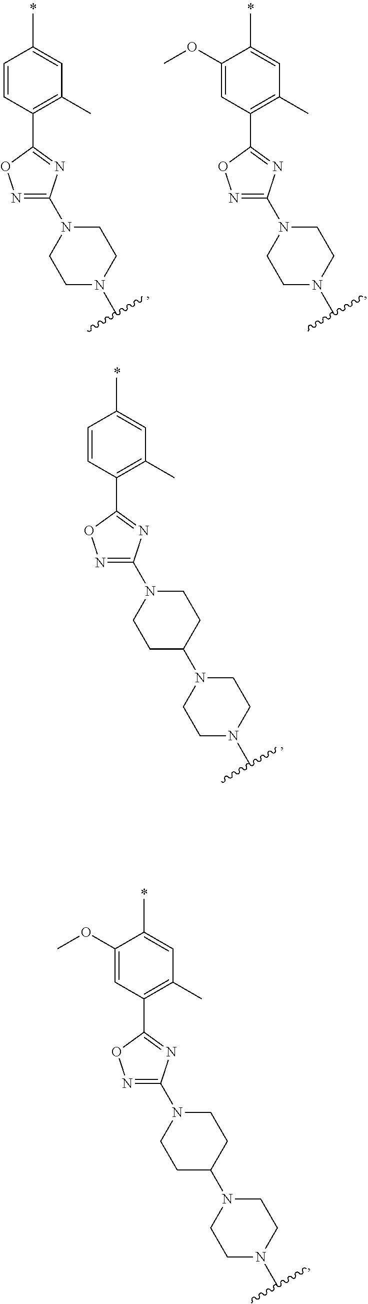

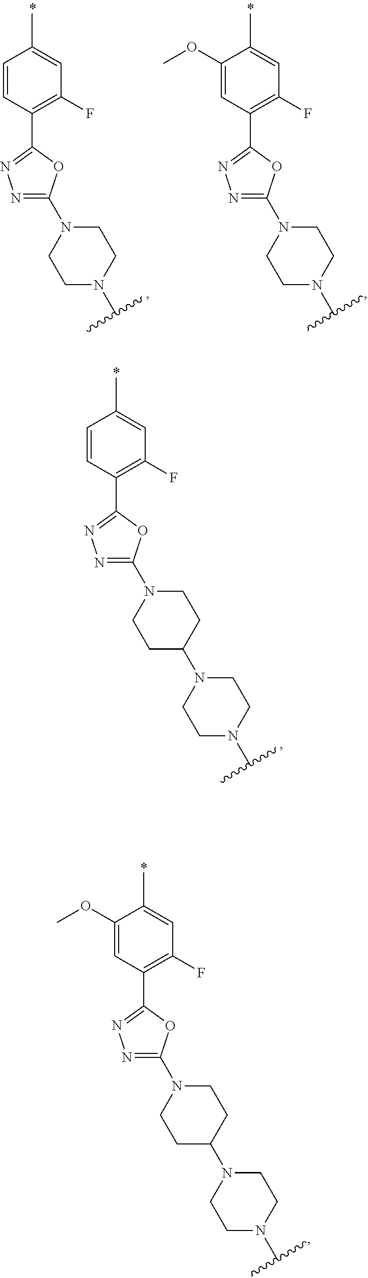

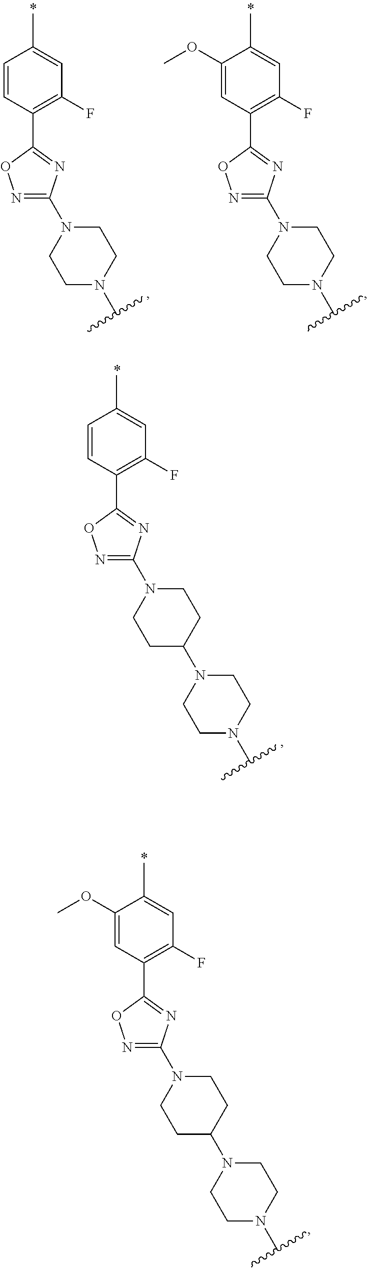

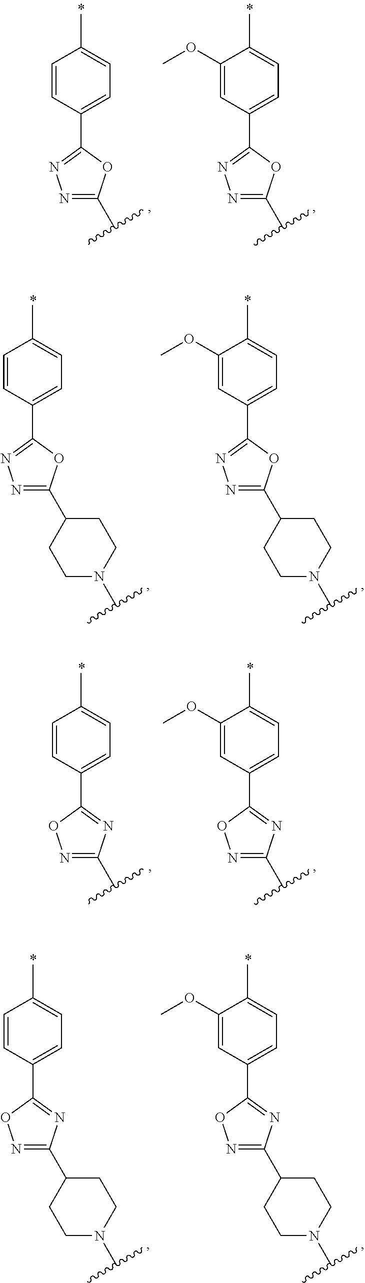

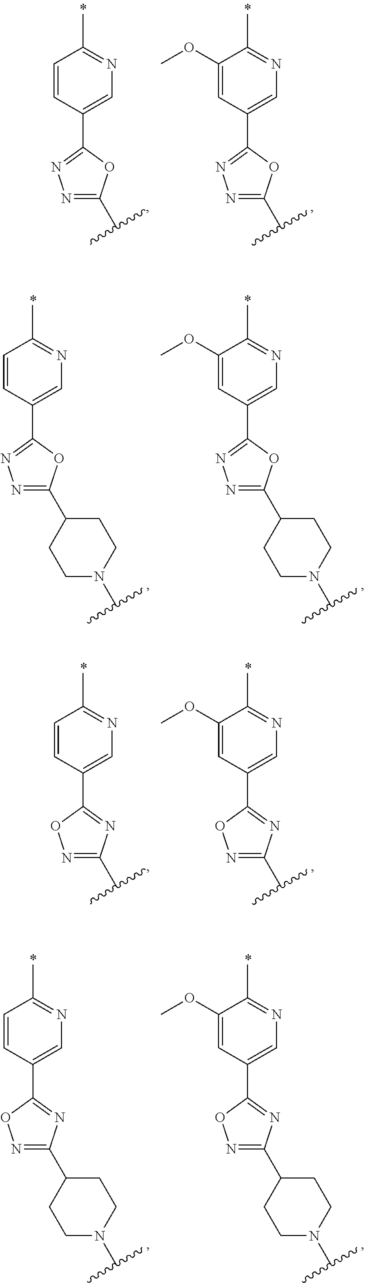

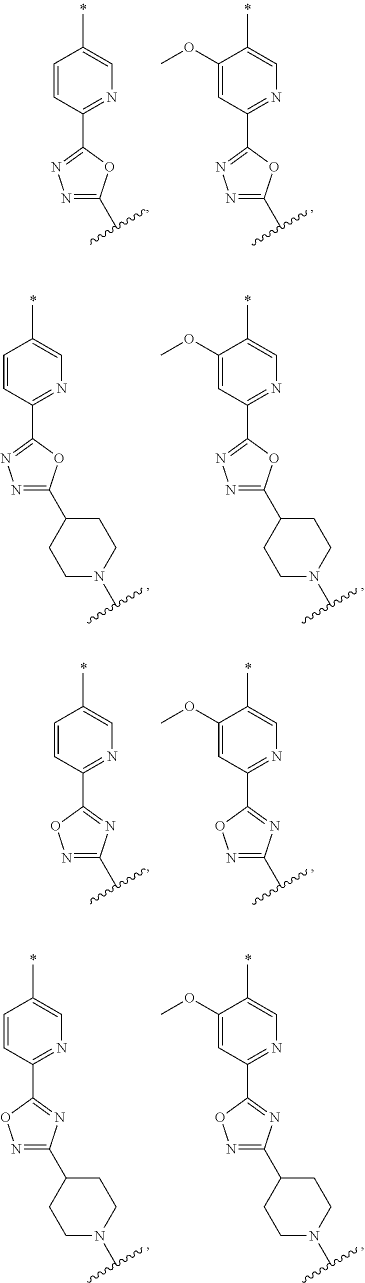

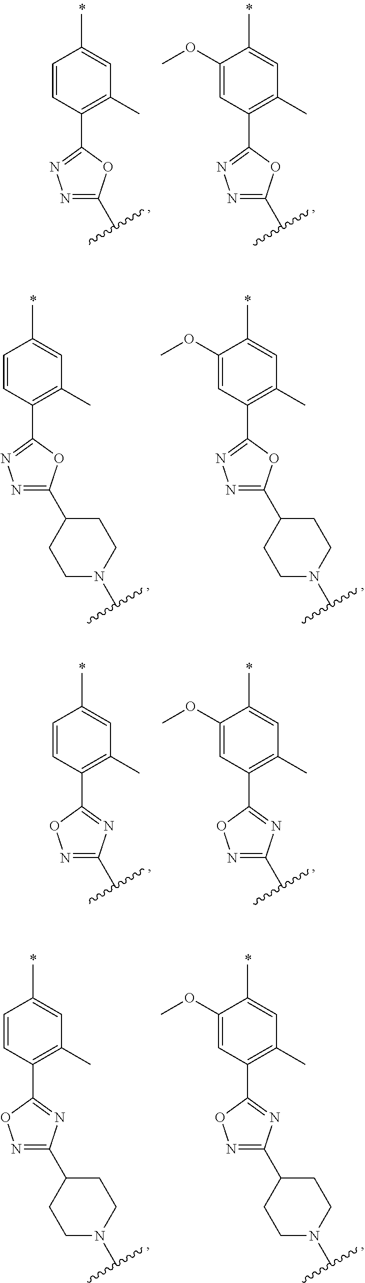

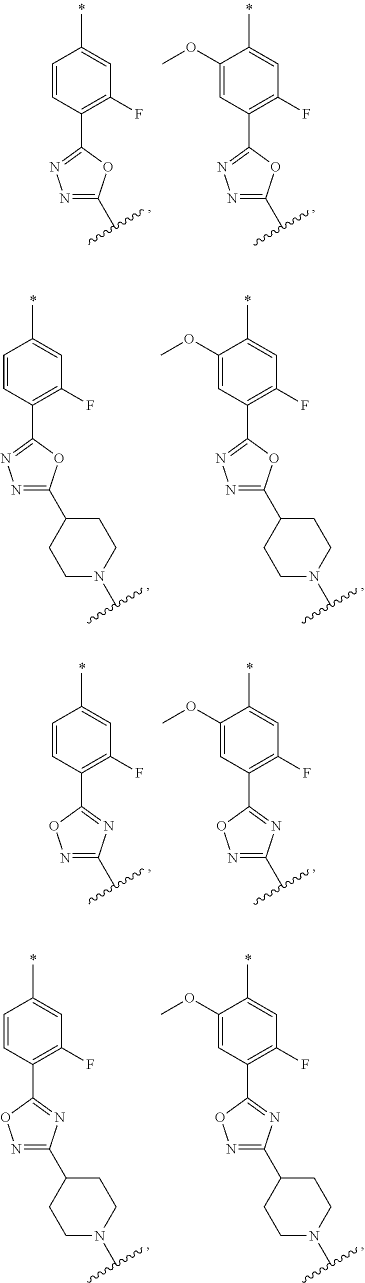

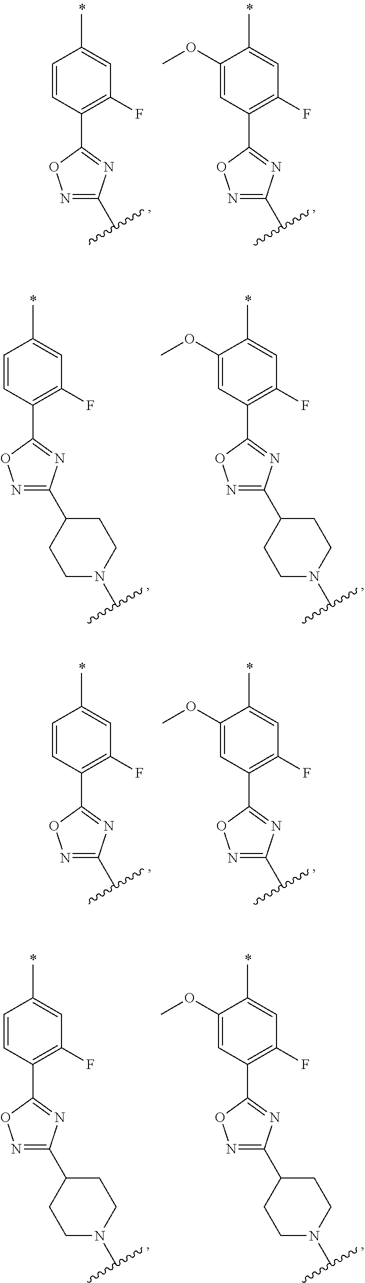

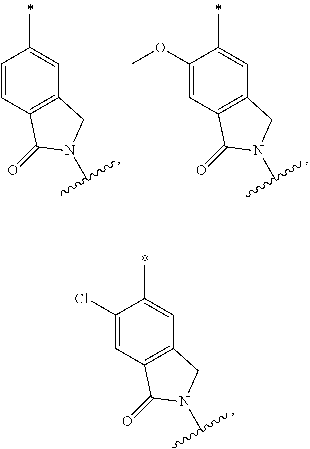 [SMILES: *N1Cc2cc(C)ccc2C1=O ; "," ; *N1Cc2cc(C)c(OC)cc2C1=O ; "," ; *N1Cc2cc(C)c(Cl)cc2C1=O ; ","]

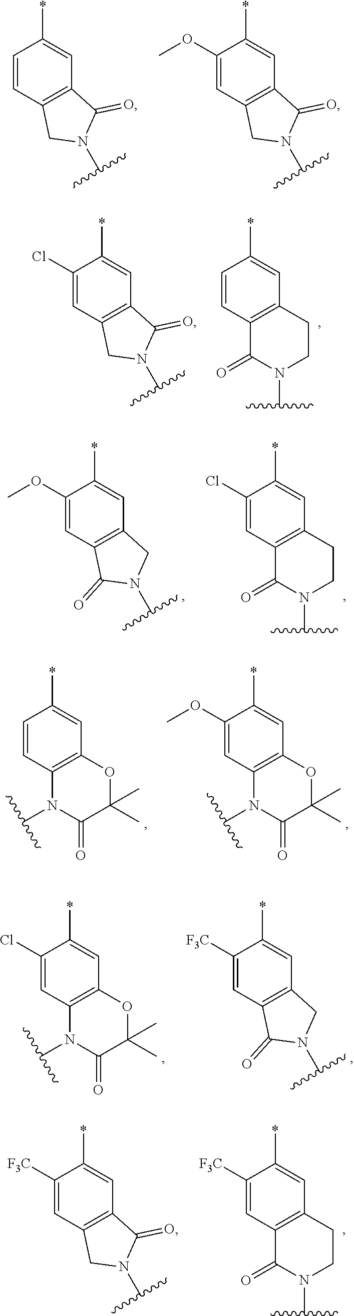

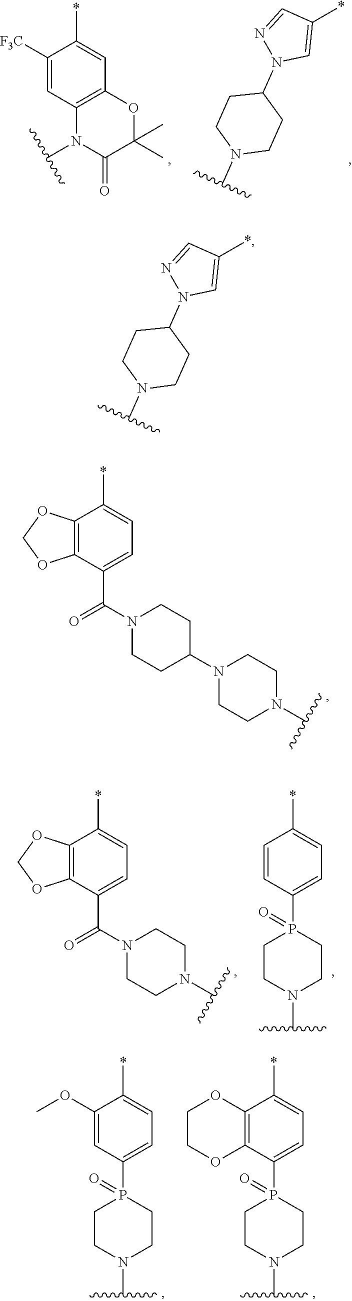

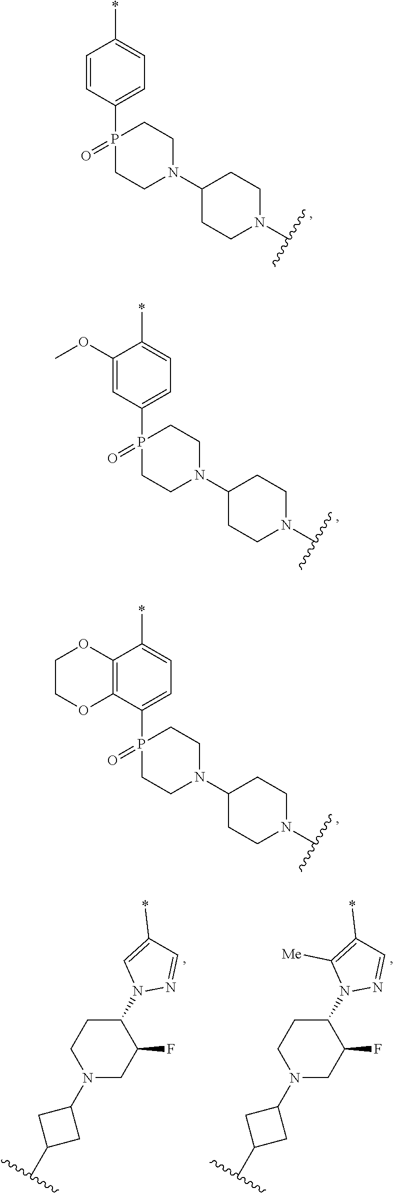 [SMILES: *c1ccc(P2(=O)CCN(C3CCN(*)CC3)CC2)cc1 ; *c1ccc(P2(=O)CCN(C3CCN(*)CC3)CC2)cc1OC ; *c1ccc(P2(=O)CCN(C3CCN(*)CC3)CC2)c2c1OCCO2 ; *c1cnn([C@H]2CCN(C3CC(*)C3)C[C@@H]2F)c1 ; *c1cnn([C@H]2CCN(C3CC(*)C3)C[C@@H]2F)c1C]

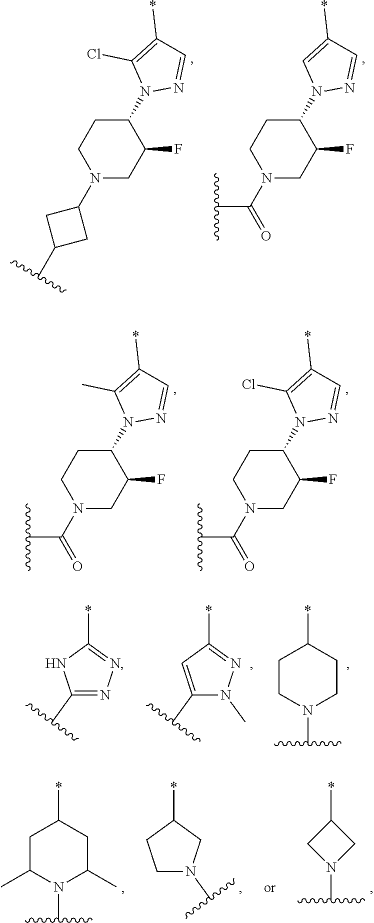

or represents H;
R₂ represents

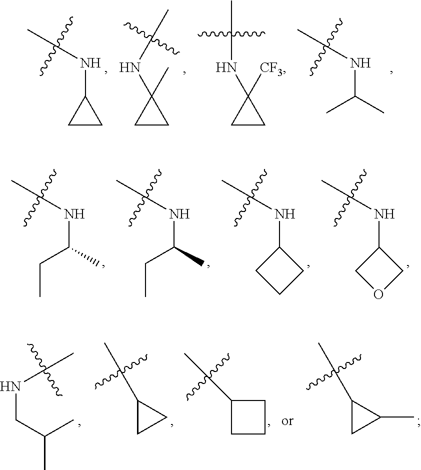

R₃ represents H, halo (e.g., F or Cl), CF₃, or wherein R₃ represents CR₆, R₂ represents NH and together with the atoms to which they are bound form a pyrrolyl group substituted with R₆;
R₄ represents H,

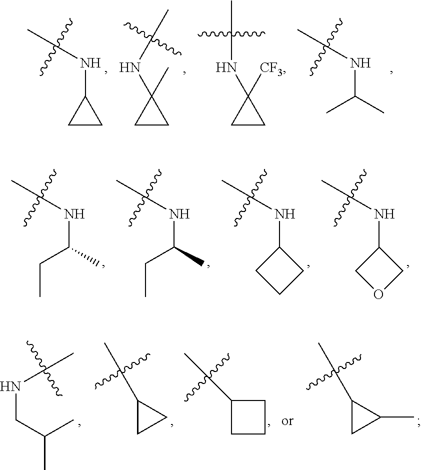

provided that one of R₁ and R₅ provides an attachment point for the

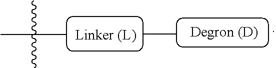

In some embodiments, wherein the X represents N and R₄ is H, the targeting ligand has a structure represented by formula TL2-b1:

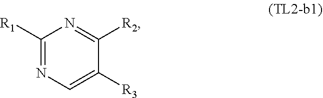

wherein:
R₂ represents

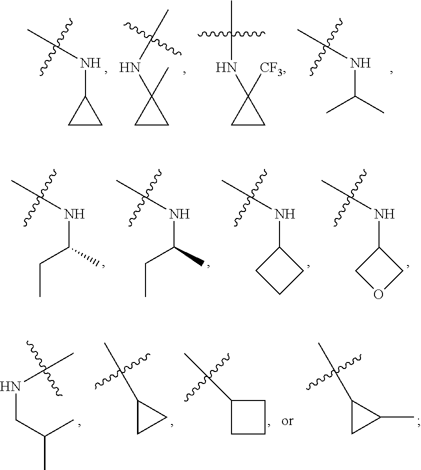

and
R₃ represents H, halo (e.g., F or Cl), or CF₃.

In some embodiments, wherein X represents N and R₂ represents NH, R₃ represents CR₆, and together with the atoms to which they are bound form a pyrrolyl group substituted with R₆, the targeting ligand has a structure represented by formula TL2-b2:

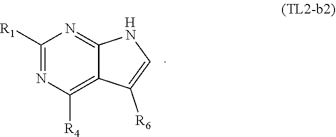

In some embodiments, wherein X represents CR₅, wherein R₅ is Hand R₂ represents NH, R₃ represents CR₆, and together with the atoms to which they are bound form a pyrrolyl group substituted with R₆, the targeting ligand has a structure represented by formula TL2-b3:

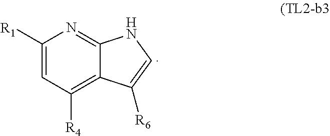

In some embodiments, wherein R₁ is absent (which also means R₁ represents H), X represents CR₅, and R₂ represents NH, R₃ represents CR₆, and together with the atoms to which they are bound form a pyrrolyl group substituted with R₆, the targeting ligand has a structure represented by formula TL2-b4:

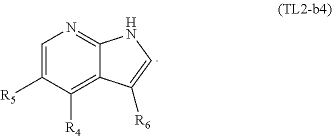

In some embodiments, wherein X represents CR₆, wherein R₆ represents H, halo, or CF₃, R₁ is absent (which also means R₁ represents H), R₂ represents NH, R₃ represents CR₅, and together with the atoms to which they are bound form a pyrrolyl group substituted with R₅, the targeting ligand has a structure represented by formula TL2-b5:

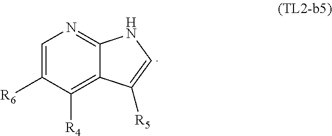

Thus, in some embodiments, the compounds of the present invention are represented by any structures generated by the combination of the targeting ligands TL2-b (including TL2-b1-TL2-b5 and

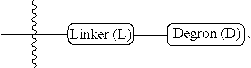

or a pharmaceutically acceptable salt or stereoisomer thereof.

### Linkers

The Linker (“L”) provides a covalent attachment of the LRRK2 targeting ligand to the Degron. The structure of linker may not be critical, provided it does not substantially interfere with the activity of the targeting ligand or the degron.

In some embodiments, the linker may be an alkylene chain or a bivalent alkylene chain, either of which may be interrupted by, and/or terminate (at either or both termini) in at least one of —O—, —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃-C₁₂ carbocyclene, 3- to 12-membered heterocyclene, 5- to 12-membered heteroarylene or any combination thereof, wherein R′ is H or C₁-C₆ alkyl, wherein the interrupting and the one or both terminating groups may be the same or different.

In some embodiments, the linker may be a polyethylene glycol chain which may terminate (at either or both termini) in at least one of —S—, —N(R′)—, —C≡C—, —C(O)—, —C(O)O—, —OC(O)—, —OC(O)O—, —C(NOR′)—, —C(O)N(R′)—, —C(O)N(R′)C(O)—, —C(O)N(R′)C(O)N(R′)—, —N(R′)C(O)—, —N(R′)C(O)N(R′)—, —N(R′)C(O)O—, —OC(O)N(R′)—, —C(NR′)—, —N(R′)C(NR′)—, —C(NR′)N(R′)—, —N(R′)C(NR′)N(R′)—, —OB(Me)O—, —S(O)₂—, —OS(O)—, —S(O)O—, —S(O)—, —OS(O)₂—, —S(O)₂O—, —N(R′)S(O)₂—, —S(O)₂N(R′)—, —N(R′)S(O)—, —S(O)N(R′)—, —N(R′)S(O)₂N(R′)—, —N(R′)S(O)N(R′)—, C₃₋₁₂ carbocyclene, 3- to 12-membered heterocyclene, 5- to 12-membered heteroarylene or any combination thereof, wherein R′ is H or C₁-C₆ alkyl, wherein the one or both terminating groups may be the same or different.

In certain embodiments, the linker is an alkylene chain having 1-10 alkylene units and interrupted by or terminating in

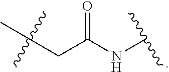 [SMILES: *CC(=O)N*]

In other embodiments, the linker is a polyethylene glycol linker having 2-8 PEG units and terminating in

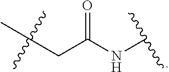 [SMILES: *CC(=O)N*]

“Carbocyclene” refers to a bivalent carbocycle radical, which is optionally substituted.

“Heterocyclene” refers to a bivalent heterocyclyl radical which may be optionally substituted.

“Heteroarylene” refers to a bivalent heteroaryl radical which may be optionally substituted.

Representative examples of linkers that may be suitable for use in the present invention include alkylene chains, e.g.:

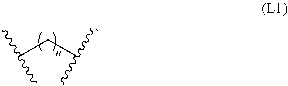

wherein n is an integer of 1-10, inclusive, e.g., 1-9, 1-8, 1-7, 1-6, 1-5, 1-4, 1-3, 1-2, 2-10, 2-9, 2-8, 2-7, 2-6, 2-5, 24, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10 and 1, 2, 3, 4, 5, 6, 7, 8, 9 and 10 examples of which include:

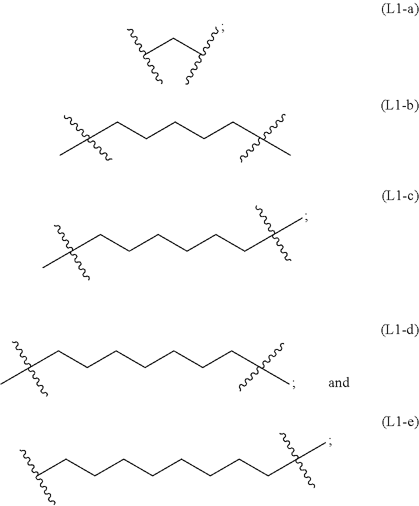

alkylene chains terminating in various functional groups (as described above), examples of which are as follows:

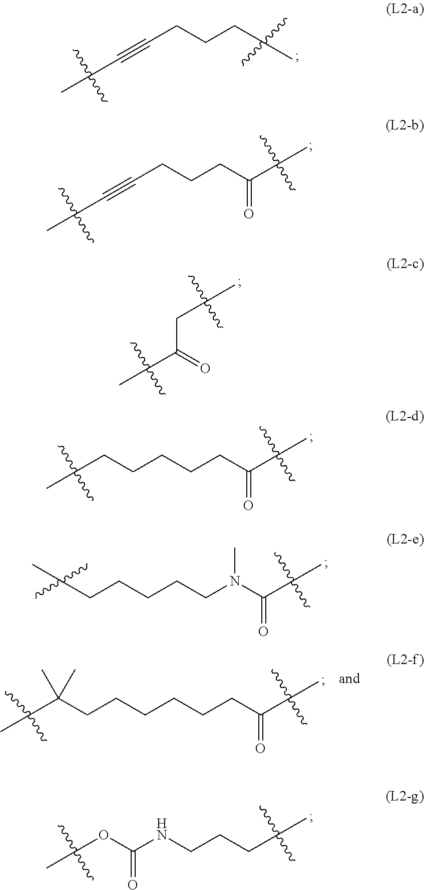

alkylene chains interrupted with various functional groups (as described above), examples of which are as follows:

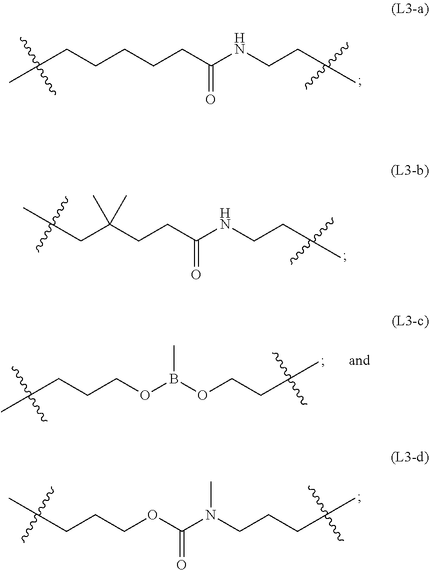 [SMILES: *CCCCCC(=O)NCC* ; *CCNC(=O)CCC(C)(C)C* ; *CCCOB(C)OCC* ; *CCCOC(=O)N(C)CCC*]

alkylene chains interrupted or terminating with heterocyclene groups, e.g.,

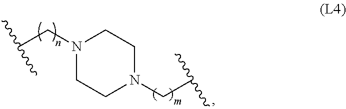

herein m and n are independently integers of 0-10 examples of which include:

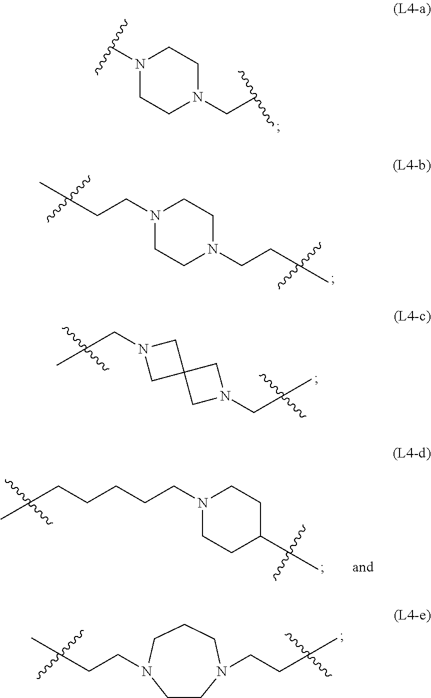 [SMILES: *CN1CCN(*)CC1 ; *CCN1CCN(CC*)CC1 ; *CN1CC2(C1)CN(C*)C2 ; *CCCCCN1CCC(*)CC1 ; *CCN1CCCN(CC*)CC1]

alkylene chains interrupted by amide, heterocyclene and/or aryl groups, examples of which include:

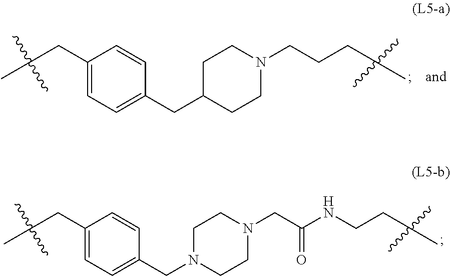 [SMILES: *CCCN1CCC(Cc2ccc(C*)cc2)CC1 ; *CCNC(=O)CN1CCN(Cc2ccc(C*)cc2)CC1]

alkylene chains interrupted by heterocyclene and aryl groups, and a heteroatom, examples of which include:

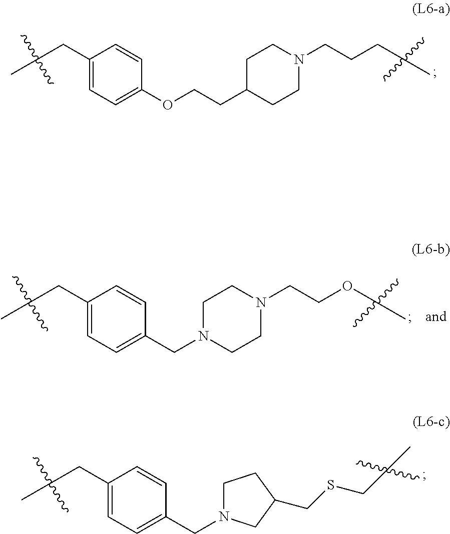 [SMILES: *CCCN1CCC(CCOc2ccc(C*)cc2)CC1 ; *Cc1ccc(CN2CCN(CCO*)CC2)cc1 ; *CSCC1CCN(Cc2ccc(C*)cc2)C1]

and
alkylene chains interrupted by a heteroatom such as N, O or B, e.g.,

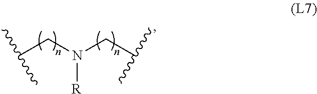

wherein n is an integer of 1-10, e.g., 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, 9-10, and 1, 2, 3, 4, 5, 6, 7, 8, 9 and 10, and R is H, or C1 to C4 alkyl, an example of which is

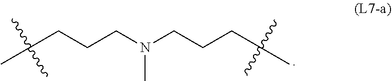 [SMILES: *CCCN(C)CCC*]

In some embodiments, the linker is a polyethylene glycol linker, examples of which include:

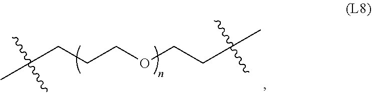

wherein n is an integer of 2-10, examples of which include:

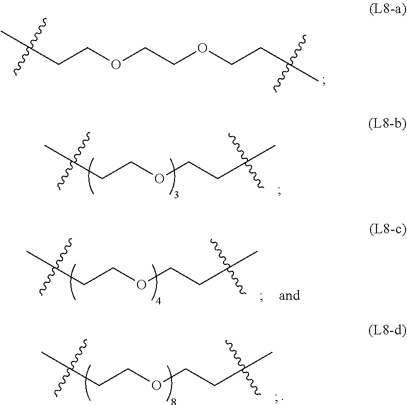

In some embodiments, the polyethylene glycol linker may terminate in a functional group, examples of which are as follows:

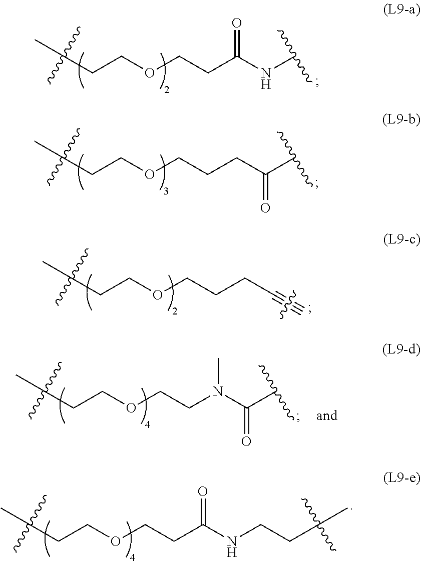

In some embodiments, the bifunctional compound of formula (I) includes a linker that is represented by any one of the following structures:

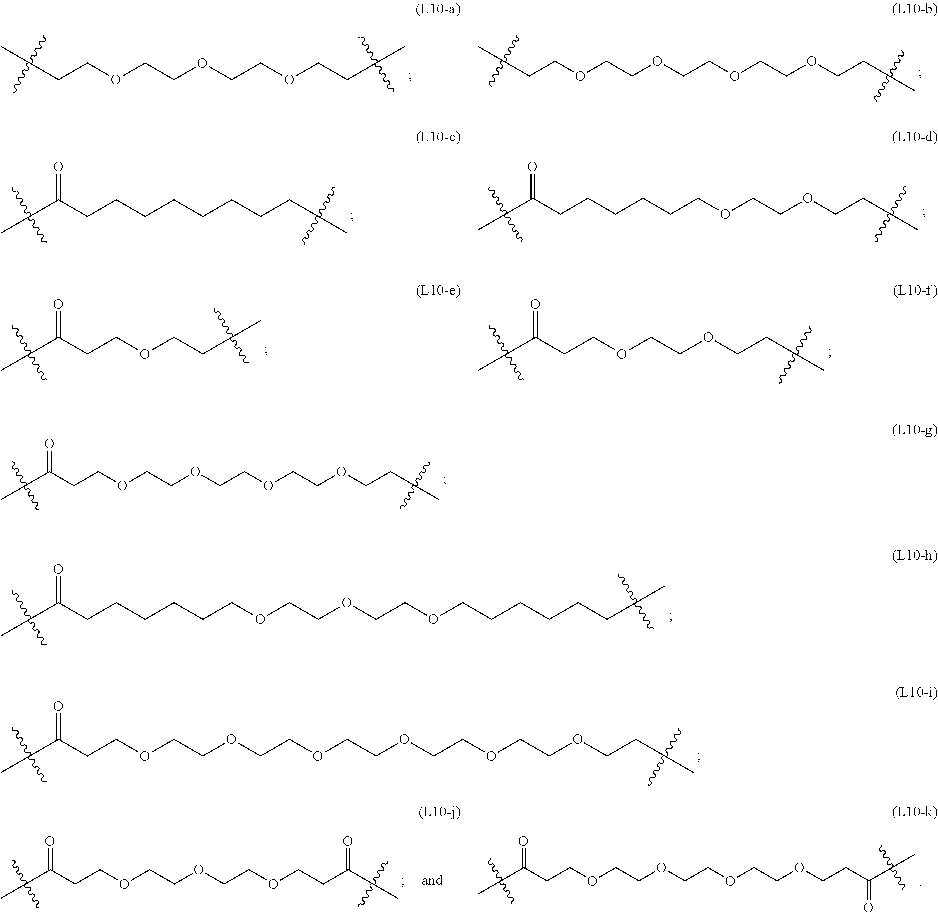

Thus, in some embodiments, the bifunctional compound of the present invention is represented by any of the following structures:

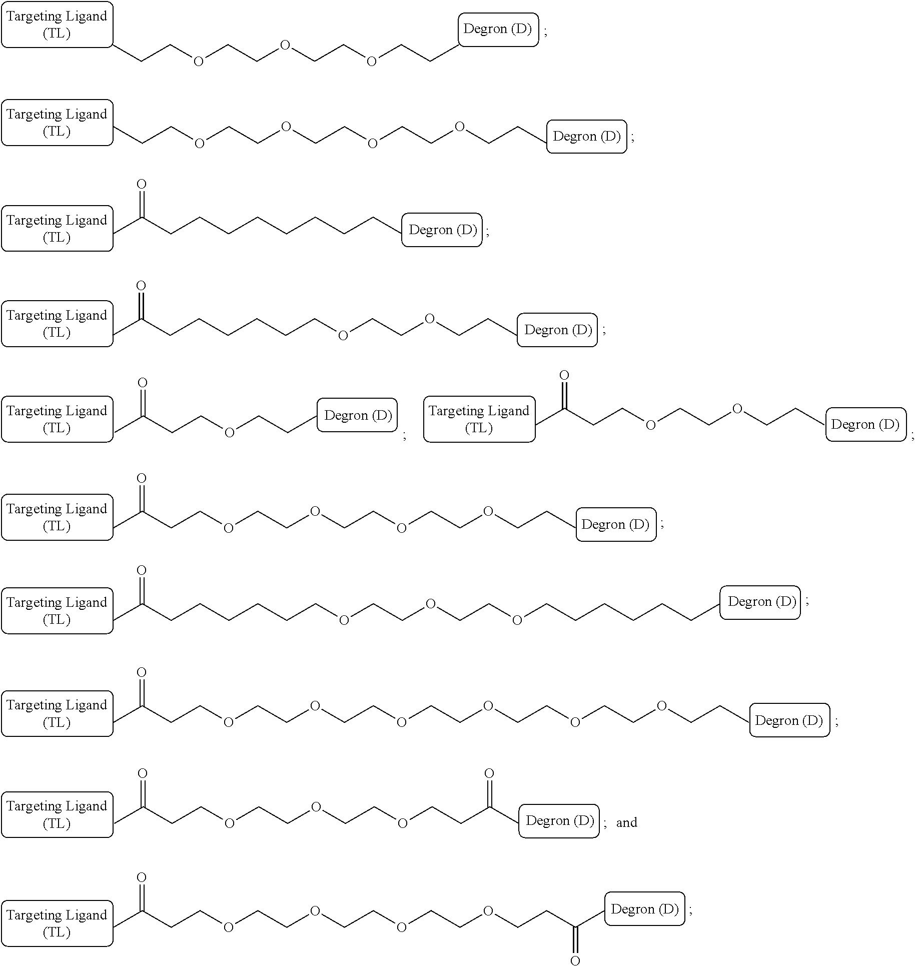

or a pharmaceutically acceptable salt or stereoisomer thereof.

In some embodiments, the bifunctional compound of the present invention is represented by any of the following structures:

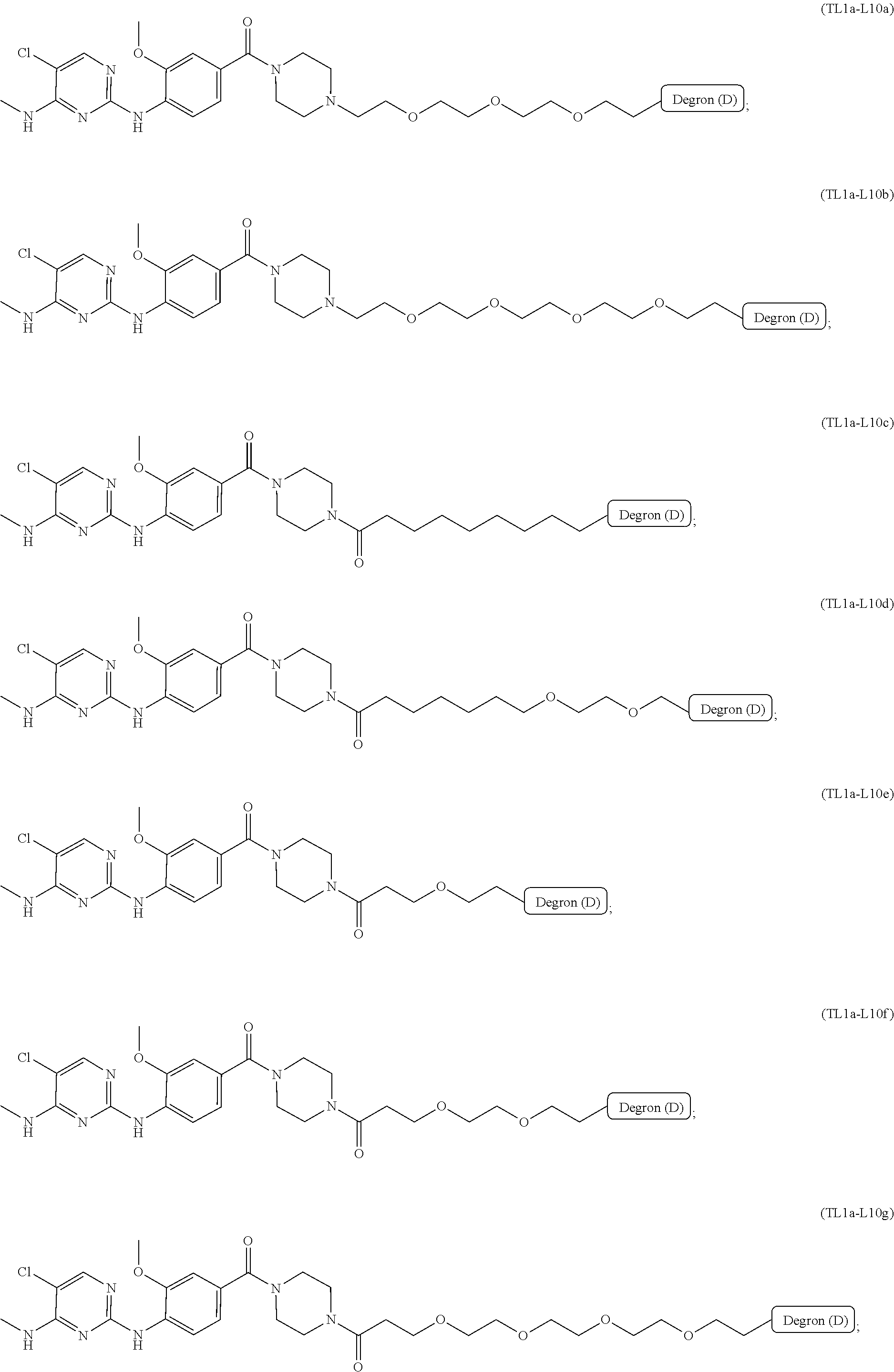

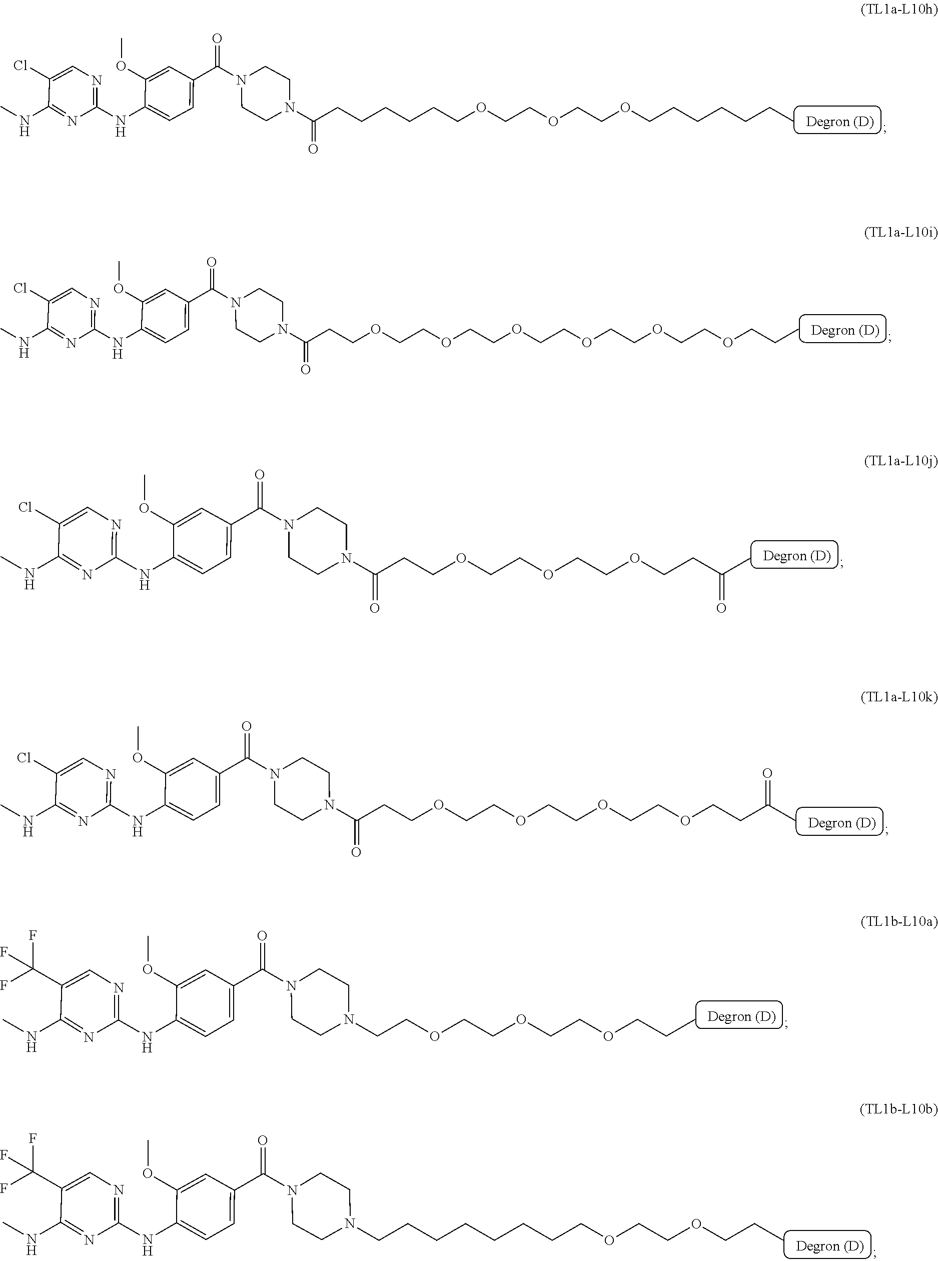

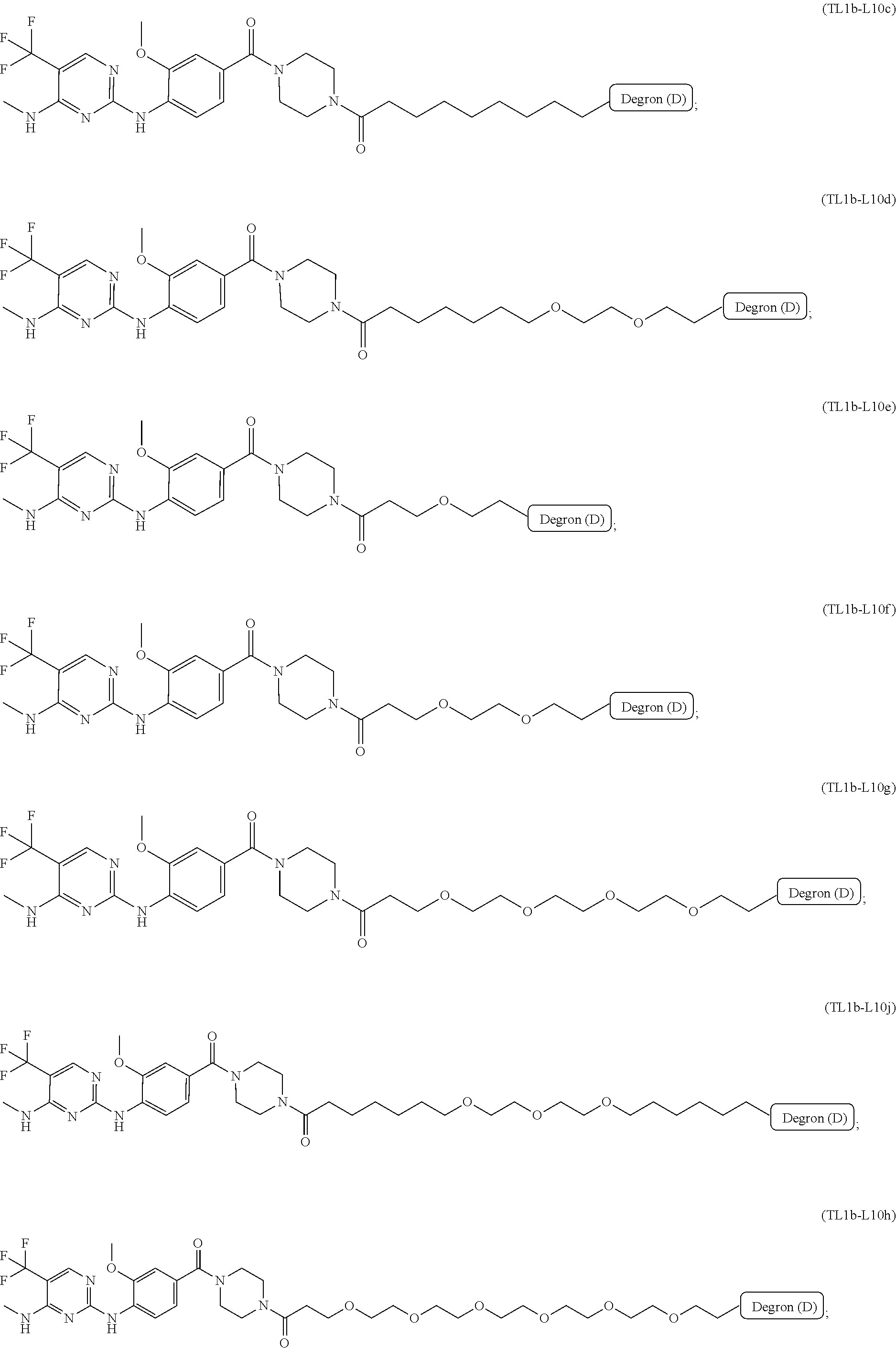

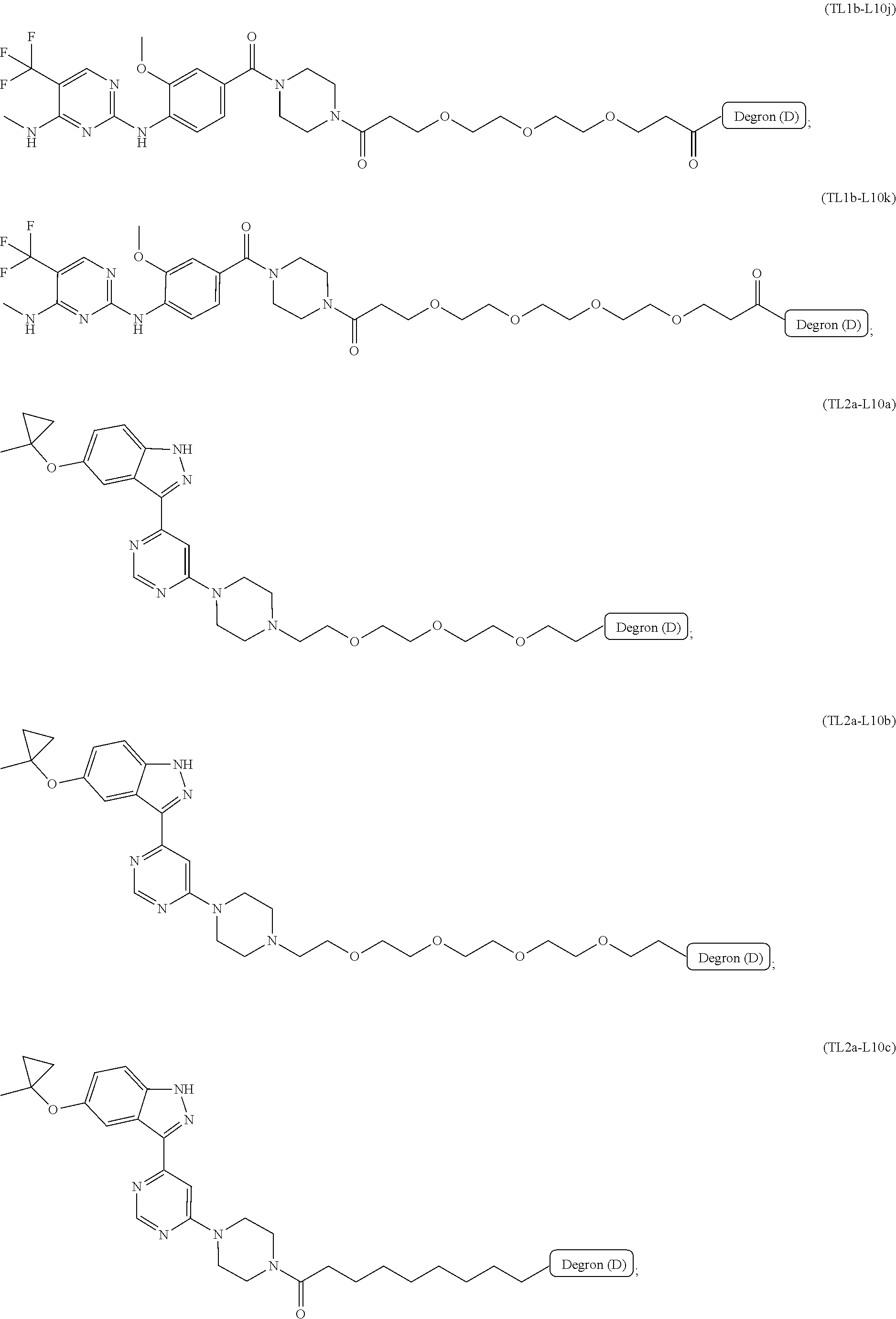

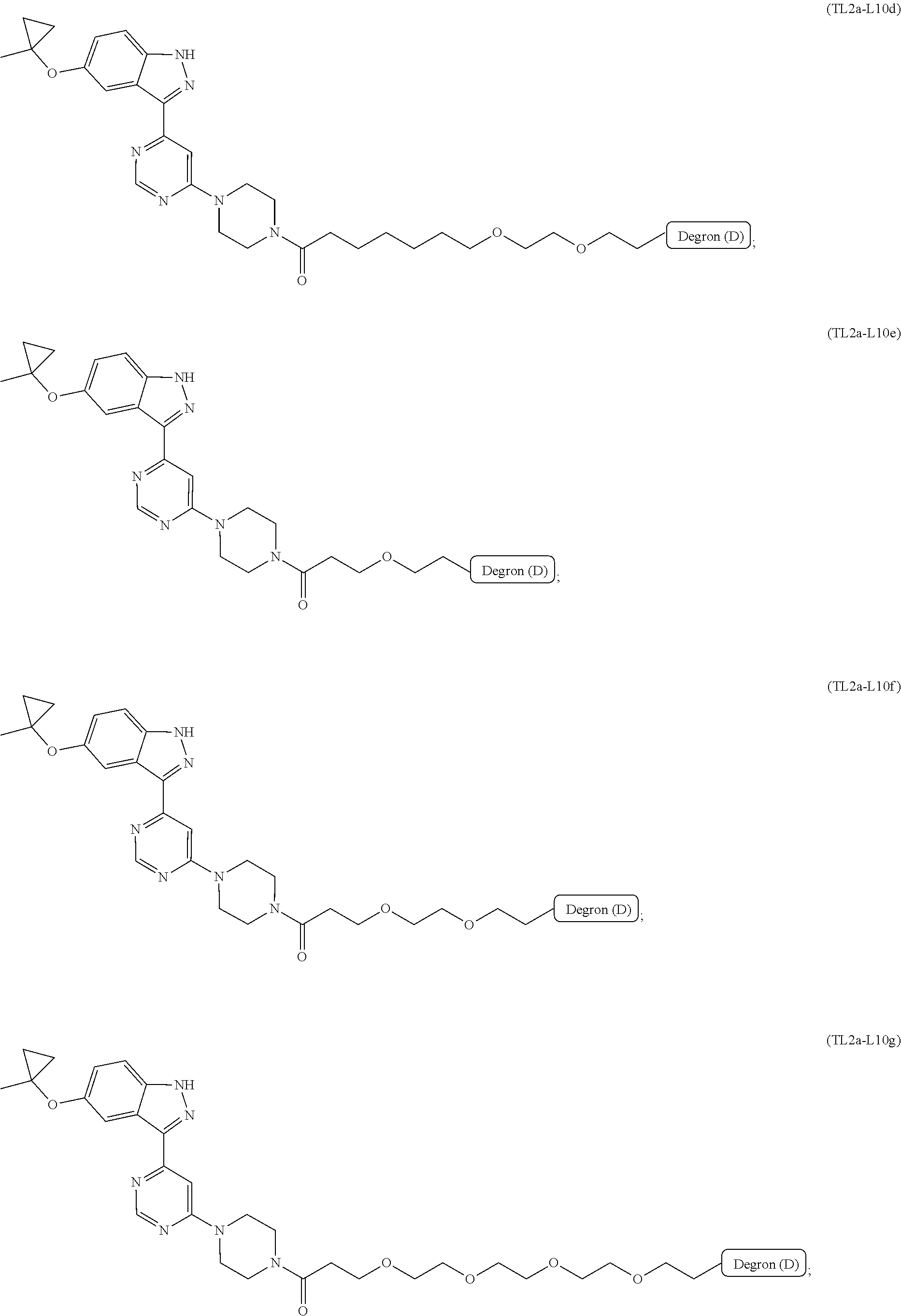

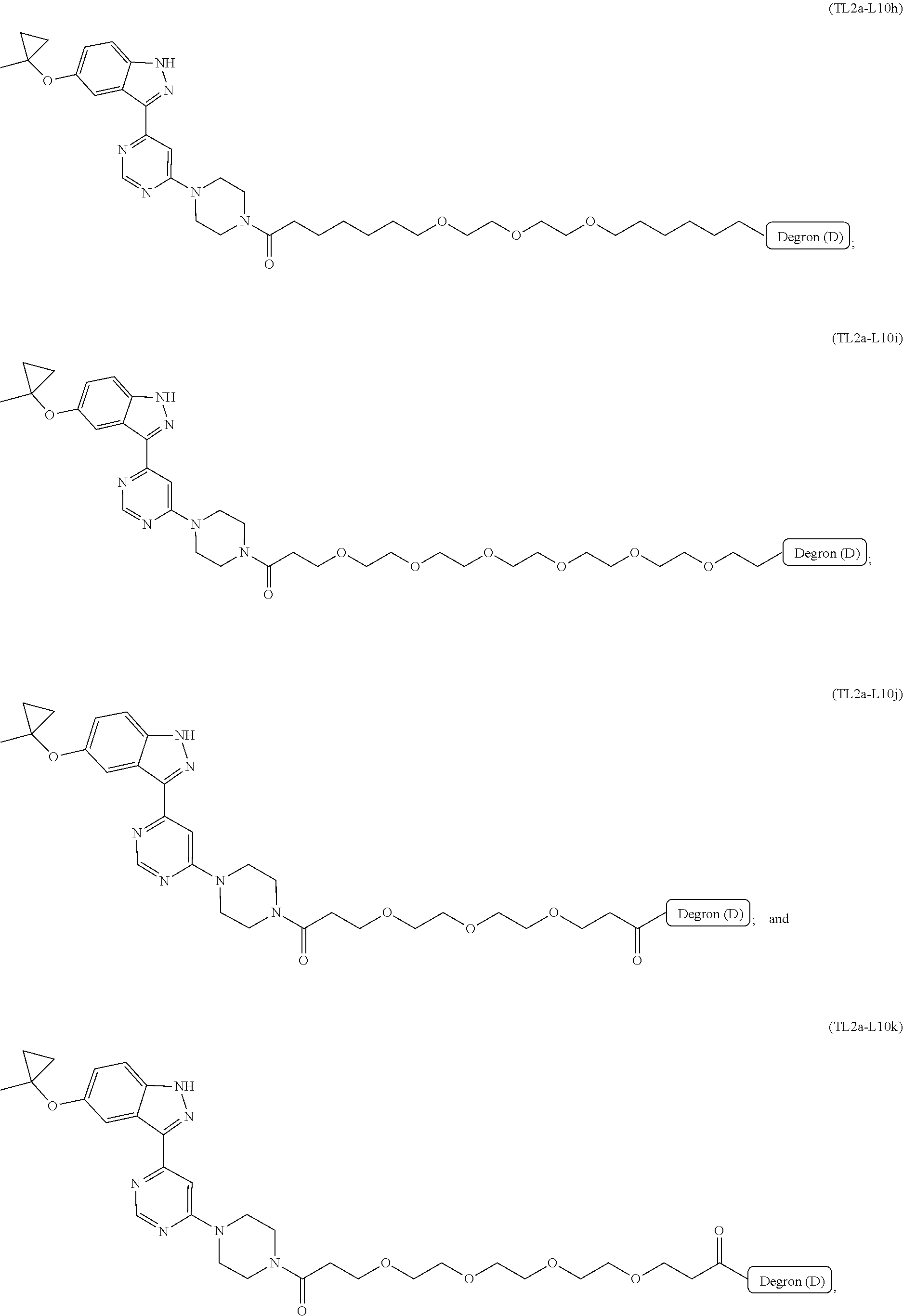

or a pharmaceutically acceptable salt or stereoisomer thereof.

### Degrons

The degron (“D”) is a functional moiety or ligand that binds an E3 ubiquitin ligase.

In some embodiments, the bifunctional compound of formula (I) includes a degron that binds cereblon. Representative examples of degrons that bind cereblon and which may be suitable for use as degrons in the present invention are described in U.S. Patent Application Publication 2018/0015085 (e.g., the indolinones such as isoindolinones and isoindoline-1,3-diones embraced by formulae IA ad IA′ therein, and the bridged cycloalkyl compounds embraced by formulae IB and IB′ therein).

In some embodiments, the bifunctional compound of formula (I) includes a degron that binds cereblon, and is represented by any one of the following structures:

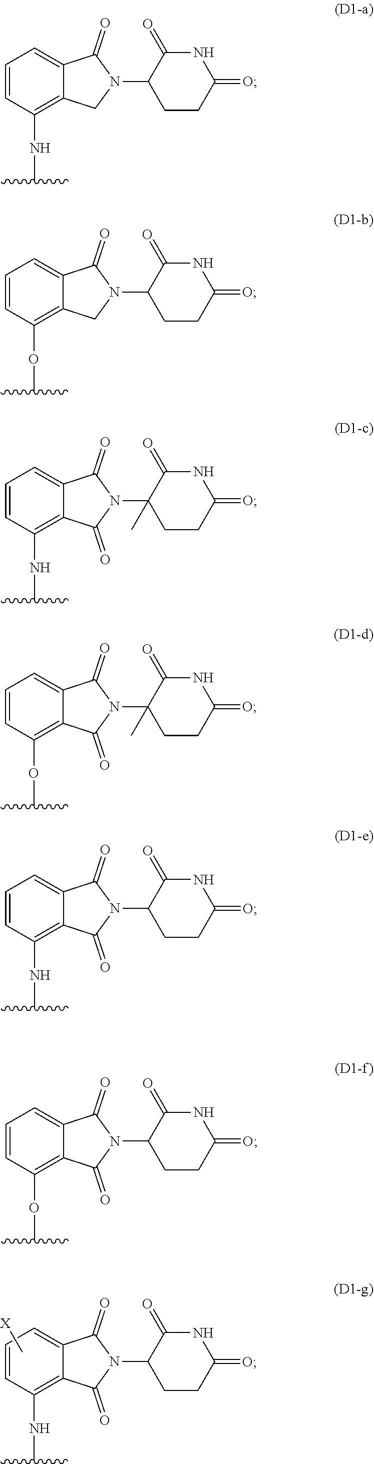

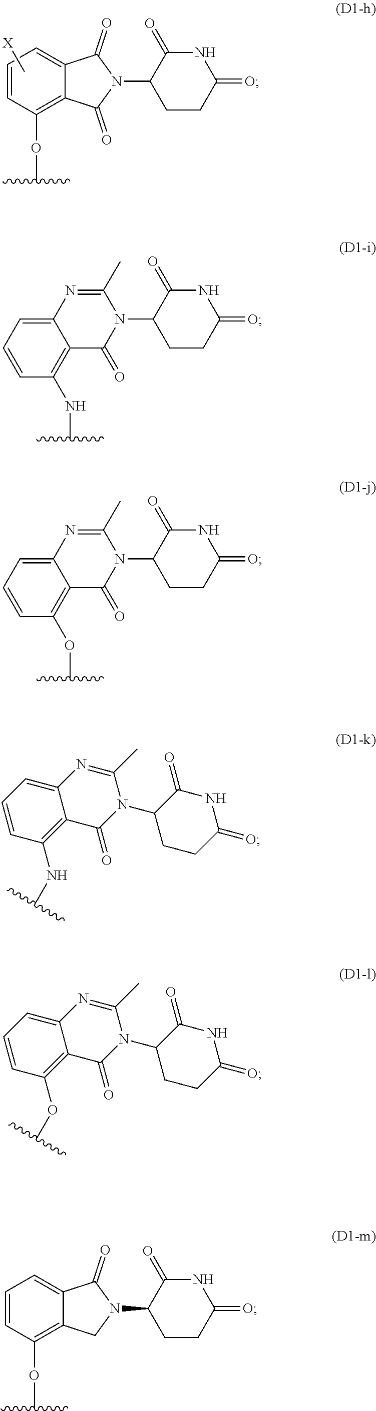

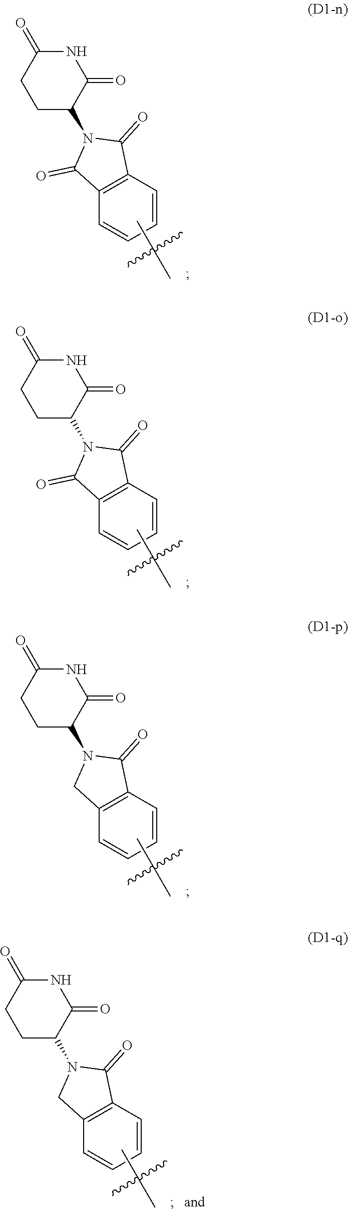

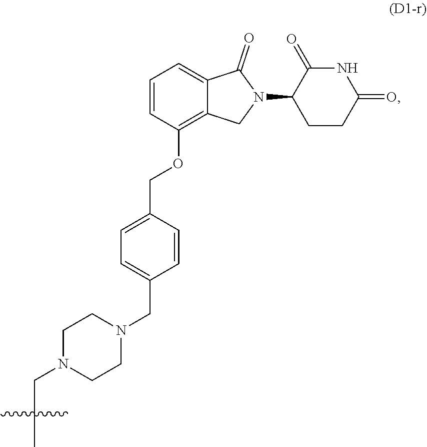

wherein X is alkyl, halo, CN, CF₃, OCHF₂ or OCF₃.

In some embodiments, the degron binds a Von Hippel-Lindau (VHL) tumor suppressor. Representative examples of degrons that bind VHL are as follows:

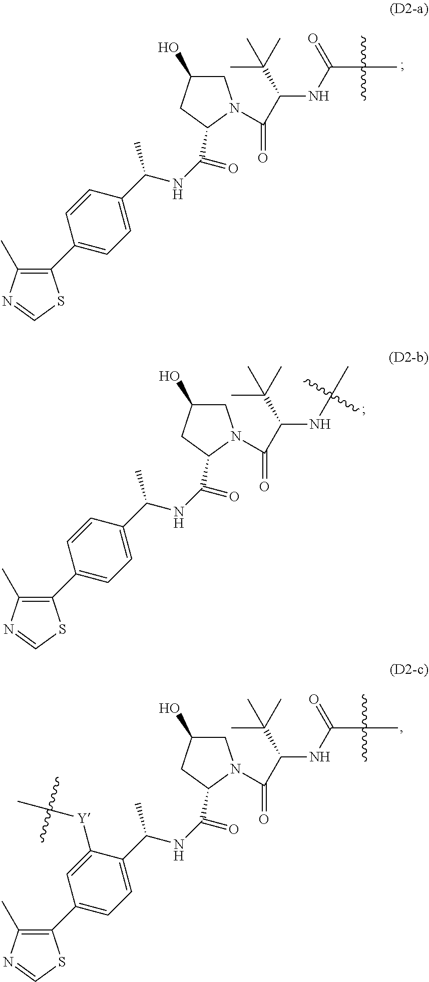

wherein Y′ is a bond, N, O or C;

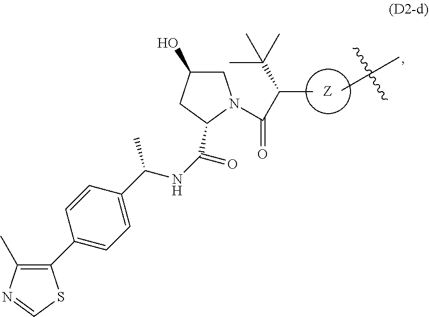

wherein Z is a C₅-C₆ carbocyclic or C₅-C₆ heterocyclic group, and

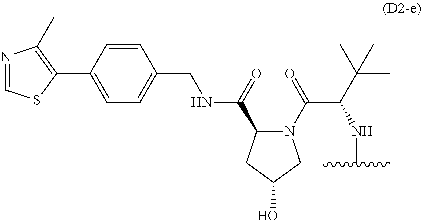 [SMILES: *N[C@H](C(=O)N1C[C@H](O)C[C@H]1C(=O)NCc1ccc(-c2scnc2C)cc1)C(C)(C)C]

Yet other degrons that bind VHL and which may be suitable for use as degrons in the present invention are disclosed in U.S. Patent Application Publication 2017/0121321 A1.

In some embodiments, the degron binds an inhibitor of apoptosis protein (IAP), and is represented by any one of the following structures:

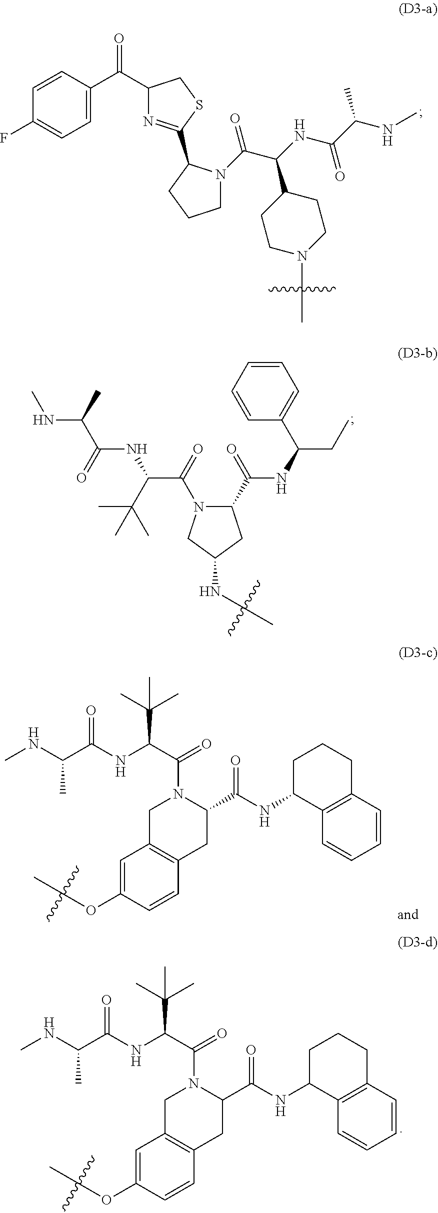

Yet other degrons that bind IAPs and which may be suitable for use as degrons in the present invention are disclosed in International Patent Application Publications WO 2008128171, WO 2008/016893, WO 2014/060768, WO 2014/060767, and WO 15092420. IAPs are known in the art to function as ubiquitin-E3 ligases.

In some embodiments, the bifunctional compound of formula (I) includes a degron that binds murine double minute 2 (MDM2), and is represented by any one of the following structures:

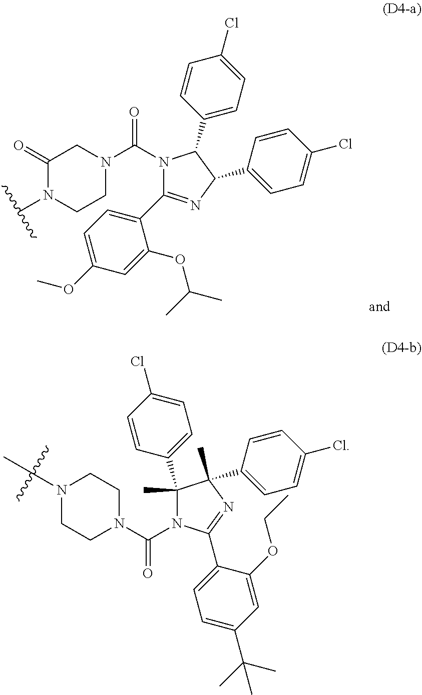 [SMILES: *N1CCN(C(=O)N2C(c3ccc(OC)cc3OC(C)C)=N[C@@H](c3ccc(Cl)cc3)[C@H]2c2ccc(Cl)cc2)CC1=O ; *N1CCN(C(=O)N2C(c3ccc(C(C)(C)C)cc3OCC)=N[C@@](C)(c3ccc(Cl)cc3)[C@@]2(C)c2ccc(Cl)cc2)CC1]

Yet other degrons that bind MDM2 and which may be suitable for use as degrons in the present invention are disclosed in U.S. Pat. No. 9,993,472 B2. MDM2 is known in the art to function as a ubiquitin-E3 ligase.

Thus, in some embodiments, the bifunctional compounds of the present invention are represented by any structures TL1a-L10a to TL2a-L10k, each of which may have as the degron, any of the structures described herein, including D1-a to D1-q, D2-a to D2-e, D3-a to D3-d and D4-a to D4-b, or a pharmaceutically acceptable salt or stereoisomer thereof.

In some embodiments, the bifunctional compound of the present invention is represented by any of the following structures:

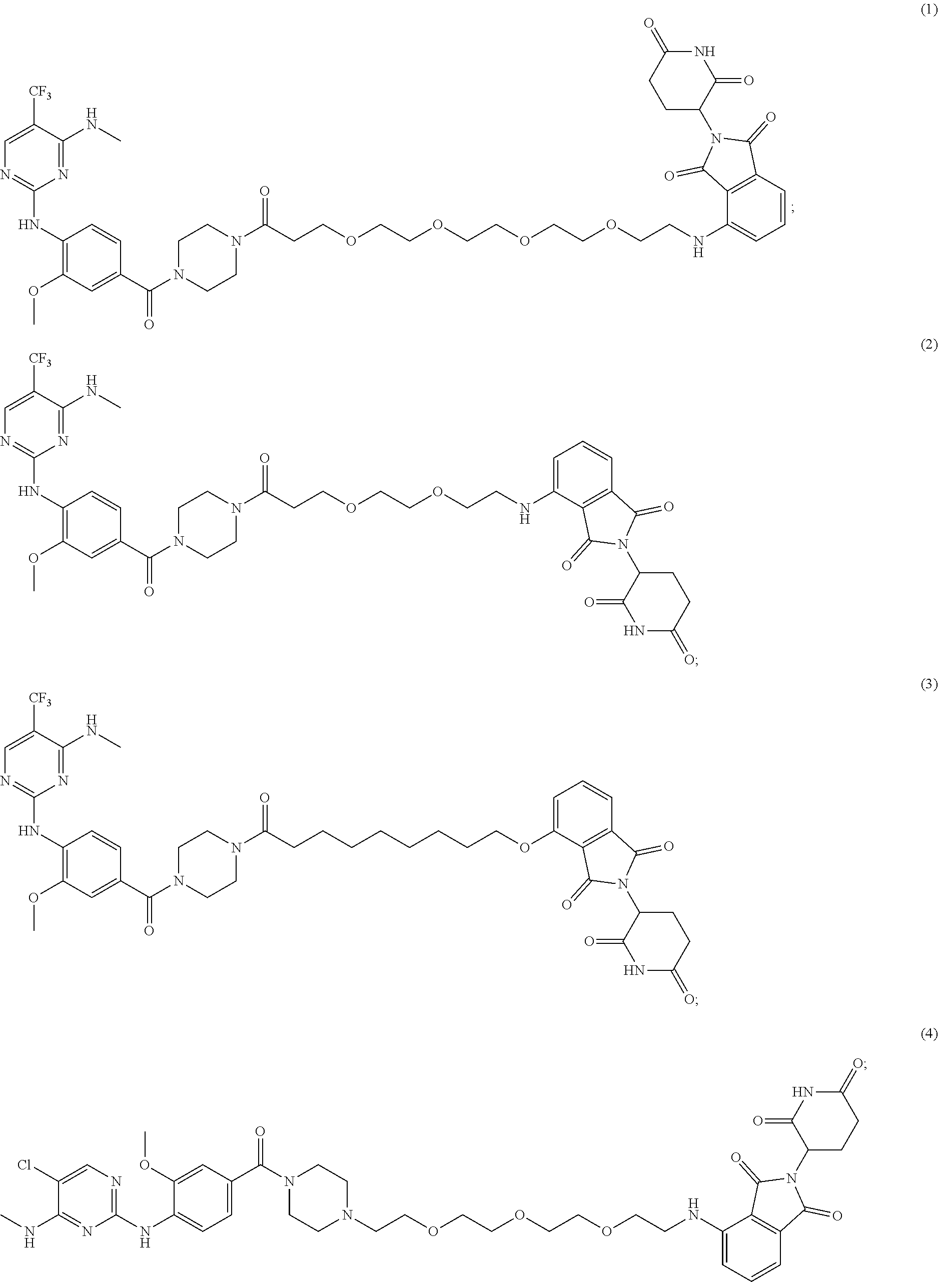

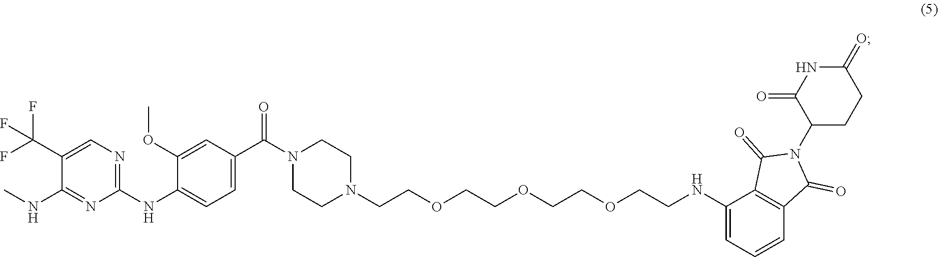 [SMILES: CNc1nc(Nc2ccc(C(=O)N3CCN(CCOCCOCCOCCNc4cccc5c4C(=O)N(C4CCC(=O)NC4=O)C5=O)CC3)cc2OC)ncc1C(F)(F)F]

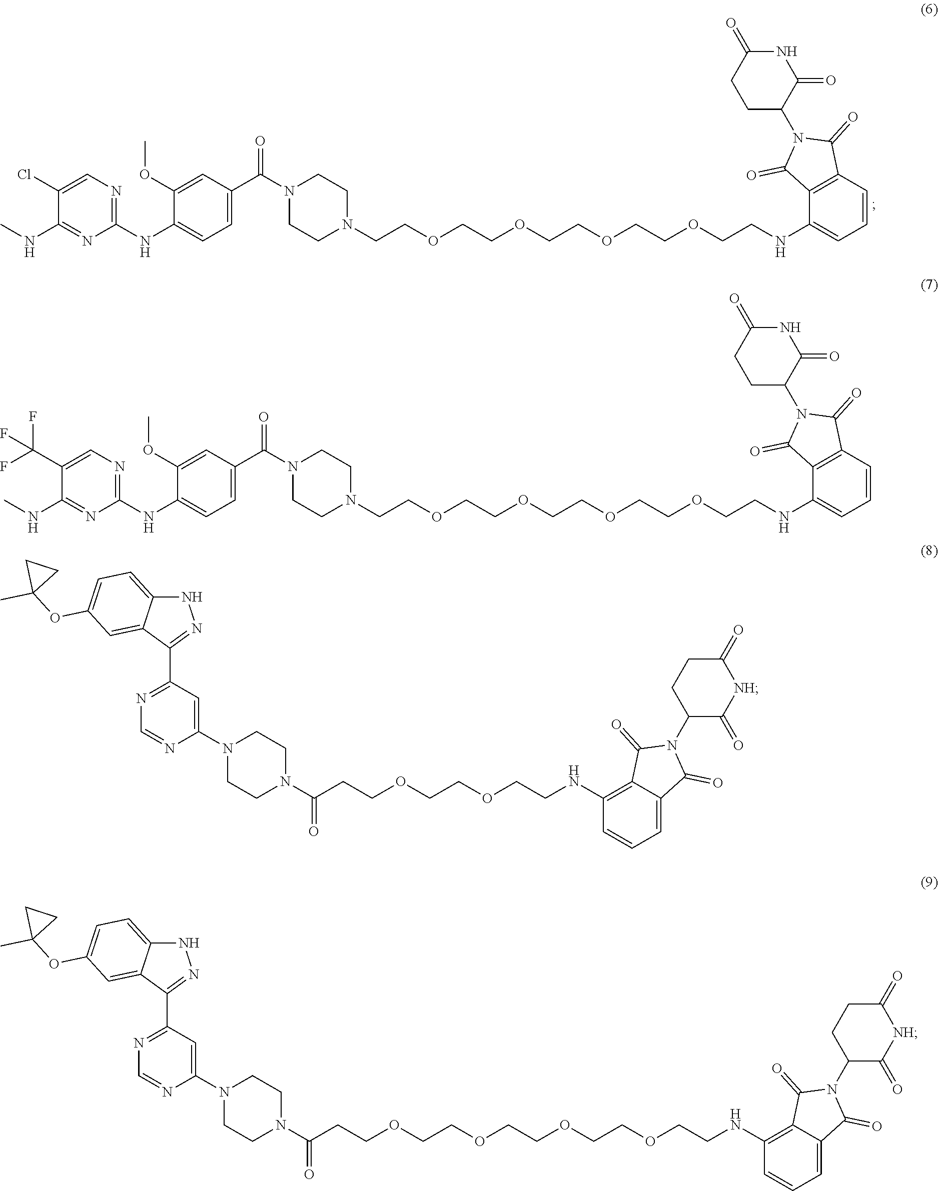

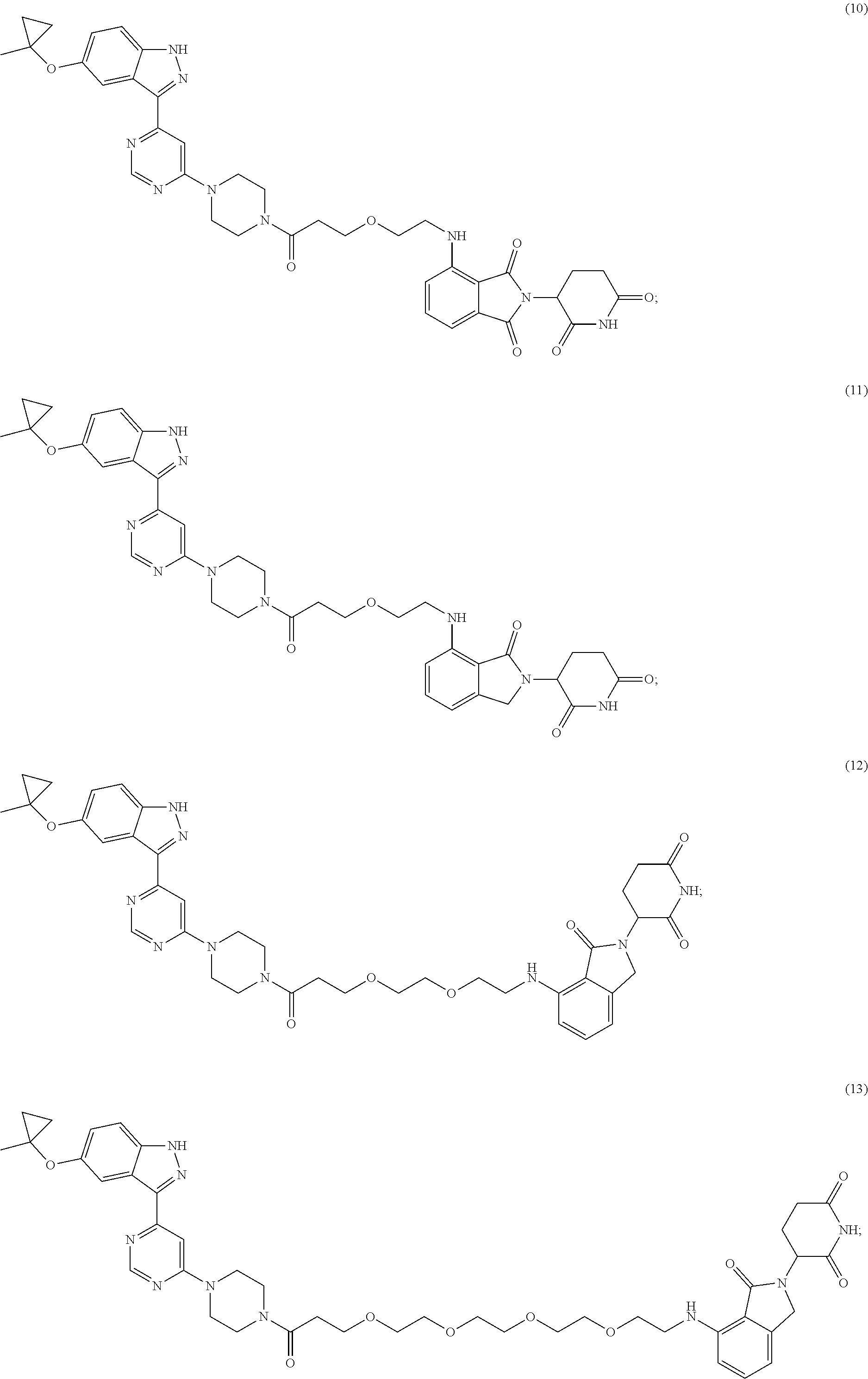 [SMILES: CC1(Oc2ccc3[nH]nc(-c4cc(N5CCN(C(=O)CCOCCNc6cccc7c6C(=O)N(C6CCC(=O)NC6=O)C7=O)CC5)ncn4)c3c2)CC1 ; CC1(Oc2ccc3[nH]nc(-c4cc(N5CCN(C(=O)CCOCCNc6cccc7c6C(=O)N(C6CCC(=O)NC6=O)C7)CC5)ncn4)c3c2)CC1 ; CC1(Oc2ccc3[nH]nc(-c4cc(N5CCN(C(=O)CCOCCOCCNc6cccc7c6C(=O)N(C6CCC(=O)NC6=O)C7)CC5)ncn4)c3c2)CC1 ; CC1(Oc2ccc3[nH]nc(-c4cc(N5CCN(C(=O)CCOCCOCCOCCOCCNc6cccc7c6C(=O)N(C6CCC(=O)NC6=O)C7)CC5)ncn4)c3c2)CC1]

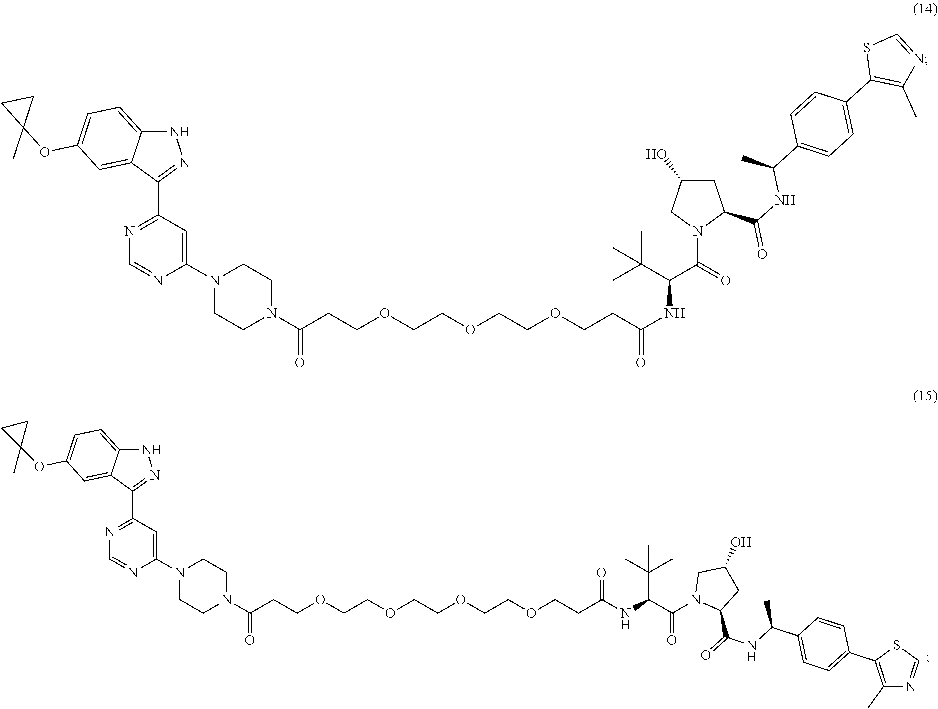 [SMILES: Cc1ncsc1-c1ccc([C@H](C)NC(=O)[C@@H]2C[C@@H](O)CN2C(=O)[C@@H](NC(=O)CCOCCOCCOCCC(=O)N2CCN(c3cc(-c4n[nH]c5ccc(OC6(C)CC6)cc45)ncn3)CC2)C(C)(C)C)cc1 ; Cc1ncsc1-c1ccc([C@H](C)NC(=O)[C@@H]2C[C@@H](O)CN2C(=O)[C@@H](NC(=O)CCOCCOCCOCCOCCC(=O)N2CCN(c3cc(-c4n[nH]c5ccc(OC6(C)CC6)cc45)ncn3)CC2)C(C)(C)C)cc1]

and pharmaceutically acceptable salts and stereoisomers thereof.

Bifunctional compounds of formula (I) may be in the form of a free acid or free base, or a pharmaceutically acceptable salt. As used herein, the term “pharmaceutically acceptable” in the context of a salt refers to a salt of the compound that does not abrogate the biological activity or properties of the compound, and is relatively non-toxic, i.e., the compound in salt form may be administered to a subject without causing undesirable biological effects (such as dizziness or gastric upset) or interacting in a deleterious manner with any of the other components of the composition in which it is contained. The term “pharmaceutically acceptable salt” refers to a product obtained by reaction of the compound of the present invention with a suitable acid or a base. Examples of pharmaceutically acceptable salts of the compounds of this invention include those derived from suitable inorganic bases such as Li, Na, K, Ca, Mg, Fe, Cu, Al, Zn and Mn salts. Examples of pharmaceutically acceptable, nontoxic acid addition salts are salts of an amino group formed with inorganic acids such as hydrochloride, hydrobromide, hydroiodide, nitrate, sulfate, bisulfate, phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, 4-methylbenzenesulfonate or p-toluenesulfonate salts and the like. Certain compounds of the invention can form pharmaceutically acceptable salts with various organic bases such as lysine, arginine, guanidine, diethanolamine or metformin.

In some embodiments, the bifunctional compound of formula (I) is an isotopic derivative in that it has at least one desired isotopic substitution of an atom, at an amount above the natural abundance of the isotope, i.e., enriched. In one embodiment, the compound includes deuterium or multiple deuterium atoms. Substitution with heavier isotopes such as deuterium, i.e. ²H, may afford certain therapeutic advantages resulting from greater metabolic stability, for example, increased in vivo half-life or reduced dosage requirements, and thus may be advantageous in some circumstances.

Bifunctional compounds of formula (I) may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present invention may be made and used in the form of individual isomers and substantially free of other isomers, or in the form of a mixture of various isomers, e.g., racemic mixtures of stereoisomers.

In addition, the bifunctional compounds of formula (I) embrace the use of N-oxides, crystalline forms (also known as polymorphs), active metabolites of the compounds having the same type of activity, tautomers, and unsolvated as well as solvated forms with pharmaceutically acceptable solvents such as water, ethanol, and the like, of the compounds. The solvated forms of the conjugates presented herein are also considered to be disclosed herein.

### Methods of Synthesis

In another aspect, the present invention is directed to a method for making a bifunctional compound of formula (I), or a pharmaceutically acceptable salt or stereoisomer thereof. Broadly, the inventive compounds or pharmaceutically-acceptable salts or stereoisomers thereof, may be prepared by any process known to be applicable to the preparation of chemically related compounds. The compounds of the present invention will be better understood in connection with the synthetic schemes that described in various working examples and which illustrate nonlimiting methods by which the compounds of the invention may be prepared.

### Pharmaceutical Compositions

Another aspect of the present invention is directed to a pharmaceutical composition that includes a therapeutically effective amount of a bifunctional compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present invention to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may include one or more pharmaceutically acceptable excipients.

Broadly, bifunctional compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the bifunctional compounds are formulated for oral or intravenous administration (e.g., systemic intravenous injection).

Accordingly bifunctional compounds of formula (I) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Bifunctional compounds of formula (I) may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, bifunctional compounds of formula (I) may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

In some embodiments, bifunctional compounds of formula (I) may be formulated into tablets that may include excipients such as lactose monohydrate, microcrystalline cellulose, sodium starch glycolate, magnesium tartrate, and hydrophobic colloidal silica.

They may be formulated as solutions for parenteral and oral delivery forms, particularly to the extent that they are water-soluble. Parenteral administration may also be advantageous in that the compound may be administered relatively quickly such as in the case of a single-dose treatment and/or an acute condition.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, the bifunctional compounds of formula (I) may be administered in a local rather than systemic manner, for example, via injection of the conjugate directly into an organ, often in a depot preparation or sustained release formulation. In specific embodiments, long acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the bifunctional compound of formula (I) is delivered in a targeted drug delivery system, for example, in a liposome coated with organ-specific antibody. In such embodiments, the liposomes are targeted to and taken up selectively by the organ.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include excipients such as wetting agents, suspending agents, coloring, sweetening, flavoring, and perfuming agents.

The bifunctional compounds may be formulated for buccal or sublingual administration, examples of which include tablets, lozenges and gels.

The bifunctional compounds of formula (I) may be formulated for administration by inhalation. Various forms suitable for administration by inhalation include aerosols, mists and powders. Pharmaceutical compositions may be delivered in the form of an aerosol spray presentation from pressurized packs or a nebulizer, with the use of a suitable gaseous propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Bifunctional compounds of formula I may be formulated for topical administration which as used herein, refers to administration intradermally by application of the formulation to the epidermis. These types of compositions are typically in the form of ointments, pastes, creams, lotions, gels, solutions and sprays.

Representative examples of carriers useful in formulating compositions for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active bifunctional compound of formula I through the stratum corneum and into the epidermis or dermis, preferably, with little or no systemic absorption. Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the bifunctional compound of formula (I) is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the bifunctional compound of formula (I) may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of the bifunctional compound of formula (I) or a pharmaceutically acceptable salt or a stereoisomer thereof effective in producing the desired therapeutic response in a particular patient suffering from a disease or disorder. The term “therapeutically effective amount” includes the amount of the bifunctional compound of formula (I) or a pharmaceutically acceptable salt or a stereoisomer thereof, that when administered, may induce a positive modification in the disease or disorder to be treated (e.g., to inhibit and/or reduce LRRK2 GTP binding activity and/or LRRK2 protein kinase activity and microglial activation, and to inhibit mutant LRRK2-induced neuronal degeneration), or is sufficient to inhibit or arrest development or progression of the disease or disorder, or otherwise alleviates to some extent, one or more symptoms of the disease or disorder being treated in a subject, or which simply kills or inhibits the growth of diseased cells, or reduces the amount of LRRK2 in diseased cells (e.g. the basal ganglia and the substantia nigra nerve cells).

The total daily dosage of the bifunctional compound of formula (I) and usage thereof may be decided in accordance with standard medical practice, e.g., by the attending physician using sound medical judgment. The specific therapeutically effective dose for any particular subject will depend upon a variety of factors including the disease or disorder being treated and the severity thereof (e.g., its present status); the activity of the specific compound employed; the specific composition employed; the age, body weight, general health, sex and diet of the subject; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, 10th Edition, A. Gilman, J. Hardman and L. Limbird, eds., McGraw-Hill Press, 155-173, 2001).

The bifunctional compound of formula (I) may be effective over a wide dosage range. In some embodiments, the total daily dosage (e.g., for adult humans) may range from about 0.001 to about 1600 mg, from 0.01 to about 1000 mg, from 0.01 to about 500 mg, from about 0.01 to about 100 mg, from about 0.5 to about 100 mg, from 1 to about 100-400 mg per day, from about 1 to about 50 mg per day, from about 5 to about 40 mg per day, and in yet other embodiments from about 10 to about 30 mg per day. Individual dosages may be formulated to contain the desired dosage amount depending upon the number of times the compound is administered per day. By way of example, capsules may be formulated with from about 1 to about 200 mg of compound (e.g., 1, 2, 2.5, 3, 4, 5, 10, 15, 20, 25, 50, 100, 150, and 200 mg). In some embodiments, the bifunctional compound of formula (I) may be administered at a dose in range from about 0.01 mg to about 200 mg/kg of body weight per day. A dose of from 0.1 to 100, e.g., from 1 to 30 mg/kg per day in one or more dosages per day may be effective. By way of example, a suitable dose for oral administration may be in the range of 1-30 mg/kg of body weight per day, and a suitable dose for intravenous administration may be in the range of 1-10 mg/kg of body weight per day.

In some embodiments, the daily dosage of the bifunctional compound of formula (I) is from about 37.5 mg to about 50 mg. To facilitate such dosing, the compounds may be formulated in capsules in dosages of 12.5 mg, 25 mg, and 50 mg.

### Methods of Use

In some aspects, the bifunctional compound of formula (I) may be useful in the treatment of diseases and disorders mediated by aberrant (e.g., dysregulated (e.g., upregulated)) LRRK2 activity. The diseases or disorders may be said to be characterized or mediated by dysfunctional protein activity (e.g., elevated levels of protein relative to a non-pathological state). A “disease” is generally regarded as a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the animal's state of health.

The bifunctional compounds of formula (I) may be useful in the treatment of neurodegenerative diseases and disorders. As used herein, the term “neurodegenerative diseases and disorders” refers to conditions characterized by progressive degeneration or death of nerve cells, or both, including problems with movement (ataxias), or mental functioning (dementias). Representative examples of such diseases and disorders include Alzheimer's disease (AD) and AD-related dementias, Parkinson's disease (PD) and PD-related dementias, Prion disease, Motor neuron diseases (MND), Huntington's disease (HD), Spinocerebellar ataxia (SCA), Spinal muscular atrophy (SMA), Primary Progressive Aphasia (PPA), Amyotrophic Lateral Sclerosis (ALS), Traumatic Brain Injury (TBI), Multiple Sclerosis (MS), and dementias (e.g., vascular dementia (VaD), Lewy body dementia (LBD), Semantic Dementia, and frontotemporal lobar dementia (FTD)).

Other representative examples of such diseases and disorders include brain cancer. Representative examples of brain cancers include, capillary hemangioblastomas, meningiomas, cerebral metastases, gliomas, neuroblastomas, medulloblastomas and ependymomas.

Representative examples of gliomas that may be treatable with the modalities of the present invention include recurrent high-grade gliomas, including glioblastoma, anaplastic astrocytoma and anaplastic oligodendroglioma, and high-grade pediatric gliomas such as DIPG.

Representative examples of glioblastomas that may be treatable with the modalities of the present invention include grade II (low-grade astrocytoma), grade III (anaplastic astrocytoma), and grade IV (glioblastoma) and glioblastoma multiforme (GBM).

The present methods thus include administering a therapeutically effective amount of a bifunctional compound of formula (I) or a pharmaceutically acceptable salt or a stereoisomer thereof to a subject in need thereof. The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats as well as livestock such as cows, horses, sheep, goats, pigs, and other domesticated and wild animals. A subject “suffering from or suspected of suffering from” a specific disease or disorder may have a sufficient number of risk factors or presents with a sufficient number or combination of signs or symptoms such that a medical professional could diagnose or suspect that the subject was suffering from the disease or disorder. Thus, subjects suffering from, and suspected of suffering from, a specific disease or disorder are not necessarily two distinct groups.

The bifunctional compounds formula (I) may be administered to a patient, e.g., a patient suffering from a neurodegenerative disease or disorder, or brain cancer (e.g., gliomas and glioblastomas), as a monotherapy or by way of combination therapy, and as a front-line therapy or a follow-on therapy for patients who are unresponsive to front line therapy. Therapy may “front/first-line”, i.e., as an initial treatment in patients who have undergone no prior anti-neurodegenerative or anti-cancer treatment regimens, either alone or in combination with other treatments; or “second-line”, as a treatment in patients who have undergone a prior anti-neurodegenerative or anti-cancer treatment regimen, either alone or in combination with other treatments; or as “third-line”, “fourth-line”, etc. treatments, either alone or in combination with other treatments. Therapy may also be given to patients who have had previous treatments which have been partially successful but are intolerant to the particular treatment.

The methods of the present invention may entail administration of the bifunctional compound of formula (I) or pharmaceutical compositions containing the compound to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails a 28-day cycle which includes daily administration for 3 weeks (21 days). In other embodiments, the bifunctional compound of formula (I) may be dosed twice a day (BID) over the course of two and a half days (for a total of 5 doses) or once a day (QD) over the course of two days (for a total of 2 doses). In other embodiments, the bifunctional compound of formula (I) may be dosed once a day (QD) over the course of five days.

The bifunctional compounds of the present invention may be administered to a patient, e.g., a patient suffering from a neurodegenerative disease or disorder, or brain cancer (e.g., gliomas and glioblastomas), as a monotherapy or by way of combination therapy. The bifunctional compounds may be administered concurrently with another active agent. Representative examples of active agents known to treat neurodegenerative diseases and disorders include dopaminergic treatments (e.g., Carbidopa-levodopa, pramipexole (Mirapex), ropinirole (Requip) and rotigotine (Neupro, given as a patch)). Apomorphine and monoamine oxidase B (MAO-B) inhibitors (e.g., selegiline (Eldepryl, Zelapar), rasagiline (Azilect) and safinamide (Xadago)) for PD and movement disorders, cholinesterase inhibitors for cognitive disorders (e.g., benztropine (Cogentin) or trihexyphenidyl), antipsychotic drugs for behavioral and psychological symptoms of dementia, as well as agents aimed to slow the development of diseases, such as Riluzole for ALS, cerebellar ataxia and Huntington's disease, non-steroidal anti-inflammatory drugs for Alzheimer's disease, and caffeine A2A receptor antagonists and CERE-120 (adeno-associated virus serotype 2-neurturin) for the neuroprotection of Parkinson's disease. Representative examples of active agents known to treat brain cancer include temozolomide (Temodar), bevacizumab (Avastin), lomustine (CCNU, Ceenu), carmustine wafer (BCNU, Gliadel), and Toca 5 (Tocagen). The term “concurrently” is not limited to the administration of the anti-neurodegenerative or anti-cancer therapeutics at exactly the same time. Rather, it is meant that they are administered to a subject as part of the same course of treatment such as in a sequence and within a time interval such that they can act together (e.g., synergistically) to provide an increased benefit than if they were administered otherwise.

### Pharmaceutical Kits

The present compositions may be assembled into kits or pharmaceutical systems. Kits or pharmaceutical systems according to this aspect of the invention include a carrier or package such as a box, carton, tube or the like, having in close confinement therein one or more containers, such as vials, tubes, ampoules, or bottles, which contain the bifunctional compound of formula (I) of the present invention or a pharmaceutical composition. The kits or pharmaceutical systems of the invention may also include printed instructions for using the compounds and compositions.

### EXAMPLES

### Example 1: Synthesis of 2-(2,6-dioxopiperidin-3-yl)-4-((2-(2-(3-(4-(3-methoxy-4-((4-(methylamino)-5-(trifluoromethyl)pyrimidin-2-yl)amino)benzoylpiperazin-1-yl)-3-oxopropoxy)ethoxy)ethyl)amino)isoindoline-1,3-dione (2)

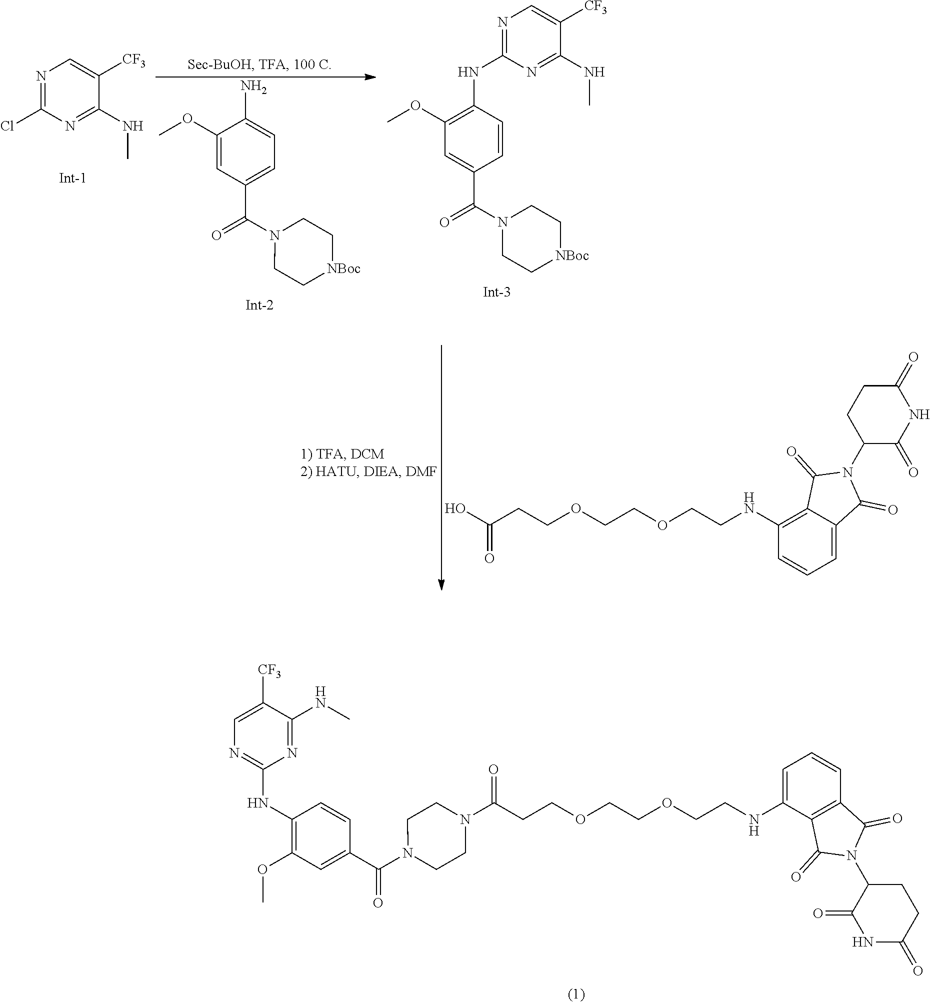

Intermediates Int-1, Int-2, and Int-3 were prepared using the appropriate pyrimidine, aniline and boc-protected piperazine according to the procedures described in Choi et al., ACS Med. Chem. Lett. 3(8):658-662 (2012) and Scott et al., J. Med. Chem. 60(7):2983-2992 (2017).

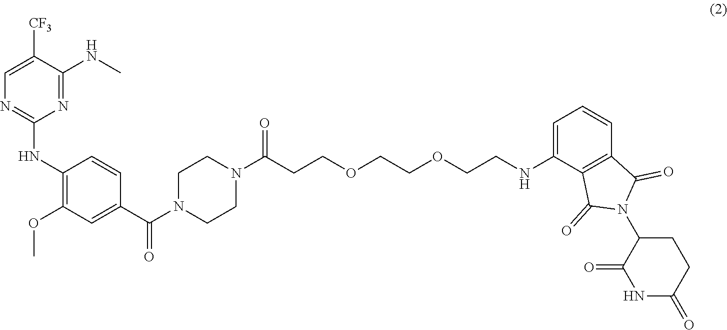 [SMILES: CNc1nc(Nc2ccc(C(=O)N3CCN(C(=O)CCOCCOCCNc4cccc5c4C(=O)N(C4CCC(=O)NC4=O)C5=O)CC3)cc2OC)ncc1C(F)(F)F]

tert-Butyl 4-(3-methoxy-4-((4-(methylamino)-5-(trifluoromethyl)pyrimidin-2-yl)amino)benzoyl)piperazine-1-carboxylate (Int-3) (12 mg, 0.024 mmol) was dissolved in DCM (10 mL). Trifluoroacetic acid TFA (1 mL) was added and the mixture was stirred for 30 minutes. The solvent was removed under reduced pressure. The resulting residue was dissolved in DMF (2 mL) before adding 3-(2-(2-((2-(2,6-dioxopiperidin-3-yl)-1,3-dioxoisoindolin-4-yl)amino)ethoxy)ethoxy)propanoic acid (10 mg, 0.024 mmol) and (1-[Bis(dimethylamino)methylene]-1H-1,2,3-triazolo[4,5-b]pyridinium 3-oxid hexafluorophosphate (HATU) (18 mg, 0.048 mmol), followed by N,N-diisopropylethylamine (DIEA) (20 μL, 0.115 mmol). The mixture was stirred for 30 minutes. The crude product was purified by reverse phase HPLC using a gradient of 1% to 70% MCCN in H₂O to give the desired product as a yellow solid (12 mg, 63% yield).

¹H NMR (500 MHz, DMSO) δ 11.10 (br, 1H), 8.72 (br, 1H), 8.29 (s, 1H), 8.21 (d, J=9 Hz, 1H), 7.79 (br, 1H), 7.57 (m, 1H), 7.13 (m, 2H), 7.03 (m, 2H), 6.59 (br, 1H), 5.05 (dd, J=5 Hz, 6 Hz, 1H), 4.0-3.41 (m, 22H), 2.94 (d, 5 Hz, 3H), 2.87 (m, 1H), 2.62-2.55 (m, 3H), 2.04 (m, 1H).

MS (ESI) m/z: 826.74 (M+H)⁺.

### Example 2: Synthesis of 2-(2,6-dioxopiperidin-3-yl)-4-((15-(4-(3-methoxy-4-((4-(methylamino)-5-(trifluoromethyl)pyrimidin-2-yl)amino)benzoylpiperazin-1-yl)-15-oxo-3,6,9,12-tetraoxapentadecyl)amino)isoindoline-1,3-dione (1)

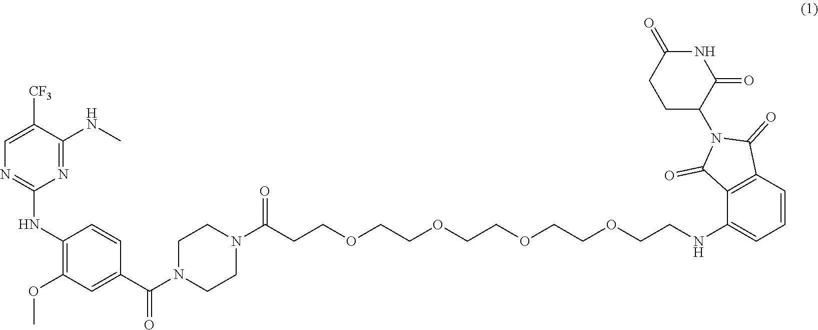 [SMILES: CNc1nc(Nc2ccc(C(=O)N3CCN(C(=O)CCOCCOCCOCCOCCNc4cccc5c4C(=O)N(C4CCC(=O)NC4=O)C5=O)CC3)cc2OC)ncc1C(F)(F)F]

Compound 1 was prepared in an analogous manner to compound 2 in Example 1. The desired product was isolated as a yellow solid (18 mg, 86% yield).

MS (ESI) m/z: 914.39 (M+H)⁺.

### Example 3: Synthesis of 2-(2,6-dioxopiperidin-3-yl)-4-((9-(4-(3-methoxy-4-((4-(methylamino)-5-(trifluoromethyl)pyrimidin-2-yl)amino)benzoyl)piperazin-1-yl)-9-oxononyl)oxy)isoindoline-1,3-dione (3)

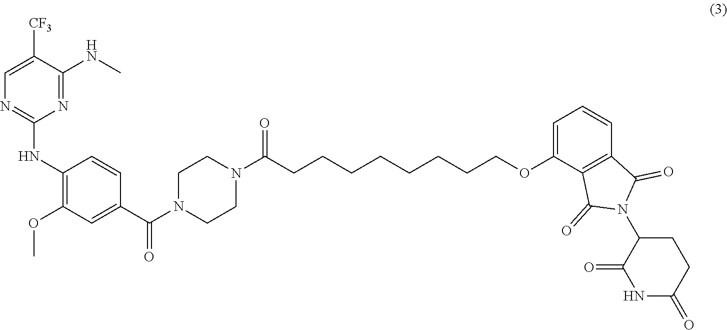 [SMILES: CNc1nc(Nc2ccc(C(=O)N3CCN(C(=O)CCCCCCCCOc4cccc5c4C(=O)N(C4CCC(=O)NC4=O)C5=O)CC3)cc2OC)ncc1C(F)(F)F]

Compound 3 was prepared in an analogous manner to compound 2 in Example 1. The desired product was isolated as a brown solid (10 mg, 53% yield).

MS (ESI) m/z: 823.52 (M+H)⁺.

### Example 4: Synthesis of 2-(2,6-dioxopiperidin-3-yl)-4-((2-(2-(2-(2-(4-(3-methoxy-4-((4-(methylamino)-5-(trifluoromethyl)pyrimidin-2-yl)amino)benzoylpiperazin-1-yl)ethoxy)ethoxy)ethoxy)ethyl)amino)isoindoline-1,3-dione (5)

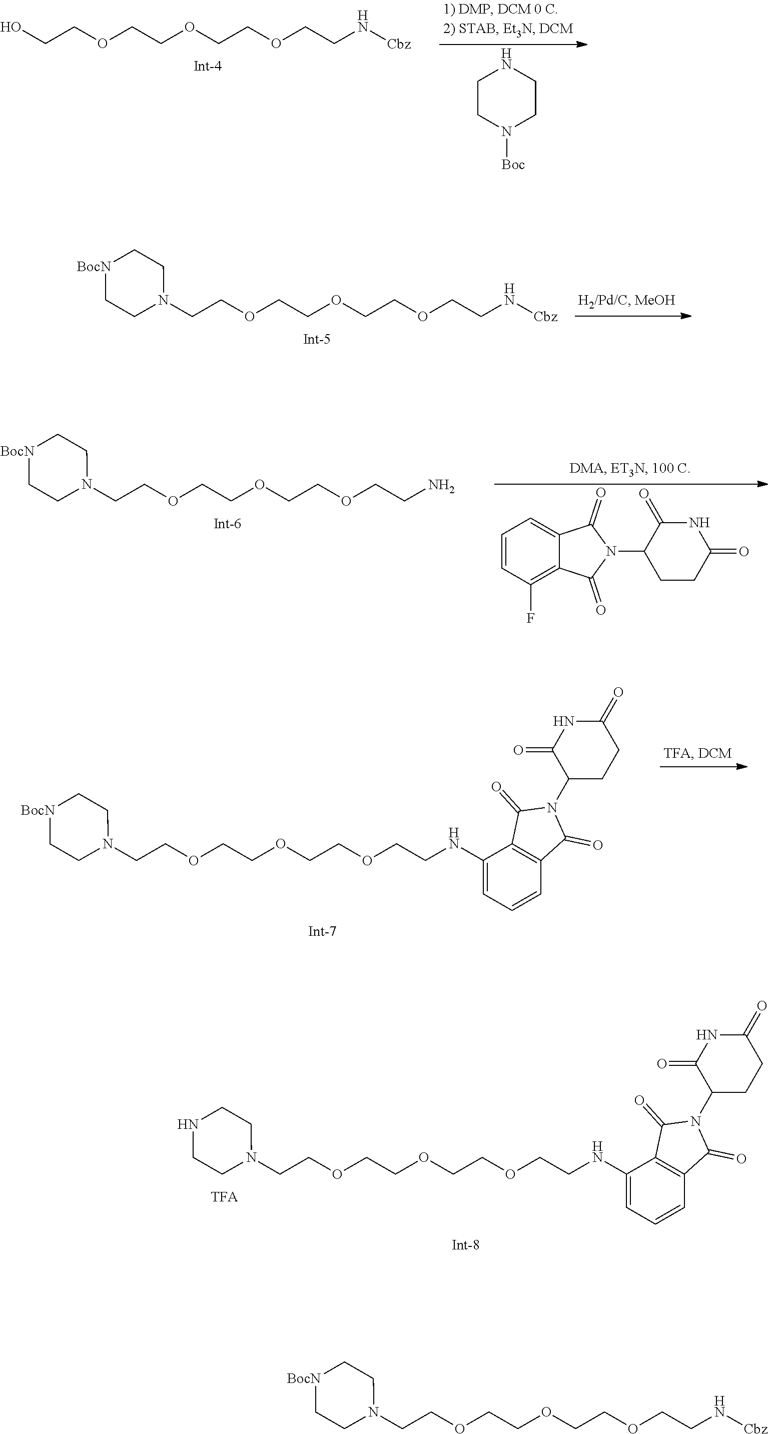

### tert-Butyl 4-(3-oxo-1-phenyl-2,7,10,13-tetraoxa-4-azapentadecan-15-yl)piperazine-1-carboxylate (Int-5)

To a solution of benzyl (2-(2-(2-(2-hydroxyethoxy)ethoxy)ethoxy)ethyl)carbamate (Int-4) (1 g, 3.05 mmol) in DCM (50 mL) was DMP (1.94 g, 4.58 mmol) at 0° C. The mixture was stirred for 1 hour at room temperature (rt). The reaction was quenched with saturated aqueous sodium thiosulfate solution and saturated aqueous NaHCO₃, and extracted with DCM. The combined organic extracts were washed with H₂O, brine, dried over MgSO₄ and condensed in vacuo to give a clear oil. To a solution of the oily product in DCM (50 mL) was added tert-butyl piperazine-1-carboxylate (852 mg, 4.58 mmol), along with Et₃N (2.13 mL, 15.25 mmol), and the mixture was stirred for 30 minutes. Sodium triacetoxyborohydride (STAB) (1.97 g, 9.30 mmol) was added, and the mixture was stirred overnight. The reaction was quenched with saturated aqueous NaHCO₃ and extracted with DCM. The combined organic layers were washed with H₂O, brine, dried over MgSO₄, and condensed under vacuum to give a clear oil that was used without further purification (1.41 g, 93% yield).

MS (ESI) m/z: 496.38 (M+H)⁺.

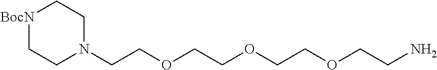 [SMILES: CC(C)(C)OC(=O)N1CCN(CCOCCOCCOCCN)CC1]

### tert-Butyl 4-(2-(2-(2-(2-aminoethoxy)ethoxy)ethoxy)ethyl)piperazine-1-carboxylate (Int-6)

To a solution of tert-butyl 4-(3-oxo-1-phenyl-2,7,10,13-tetraoxa-4-azapentadecan-15-yl)piperazine-1-carboxylate (Int-5) (1.41 g, 2.84 mmol) in MeOH (30 mL) was added Pd/C 10% (301 mg, 0.28 mmol), and the mixture was stirred under an H₂ atmosphere for 3 hours. The reaction was filtered through celite, and the filtrate was condensed under reduced pressure to give the desired product as a light brown oil (965 mg, 94% yield).

MS (ESI) m/z: 362. 57 (M+H)⁺.

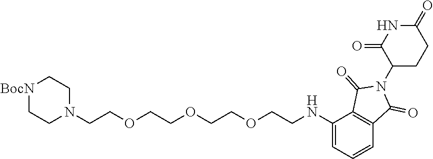 [SMILES: CC(C)(C)OC(=O)N1CCN(CCOCCOCCOCCNc2cccc3c2C(=O)N(C2CCC(=O)NC2=O)C3=O)CC1]

### tert-Butyl 4-(2-(2-(2-(2-((2-(2,6-dioxopiperidin-3-yl)-1,3-dioxoisoindolin-4-yl)amino)ethoxy) ethoxy)ethoxy)ethyl)piperazine-1-carboxylate (Int-7)

A solution of tert-butyl 4-(2-(2-(2-(2-aminoethoxy)ethoxy)ethoxy)ethyl)piperazine-1-carboxylate (Int-6) (300 mg, 0.83 mmol), 2-(2,6-dioxopiperidin-3-yl)-4-fluoroisoindoline-1,3-dione (275 mg, 1.0 mmol) and Et₃N (350 μL, 2.5 mmol) in dimethylacetamide (DMA) (2 mL) was heated at 100° C. for 4 hours. The mixture was purified by reverse phase HPLC using a gradient of 1%-70% MeCN in H₂O to give the desired product as a yellow solid (137 mg, 27% yield).

MS (ESI) m/z: 618.31 (M+H)⁺.

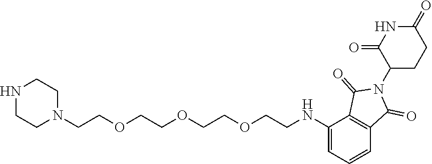 [SMILES: O=C1CCC(N2C(=O)c3cccc(NCCOCCOCCOCCN4CCNCC4)c3C2=O)C(=O)N1]

### 2-(2,6-dioxopiperidin-3-yl)-4-((2-(2-(2-(2-(piperazin-1-yl)ethoxy)ethoxy)ethoxy)ethyl) amino)isoindoline-1,3-dione (Int-8)

To a solution of tert-butyl 4-(2-(2-(2-(2-((2-(2,6-dioxopiperidin-3-yl)-1,3-dioxoisoindolin-4-yl)amino)ethoxy)ethoxy)ethoxy)ethyl)piperazine-1-carboxylate (Int-7) (137 mg, 0.222 mmol) in DCM (10 mL) was added TFA (1 mL), and the mixture was stirred for 1 hour. The solvent was removed under reduced pressure to give the TFA salt of the desired product as a yellow foam that was used without further purification (115 mg, 100% yield).

MS (ESI) m/z: 518.75 (M+H)⁺.

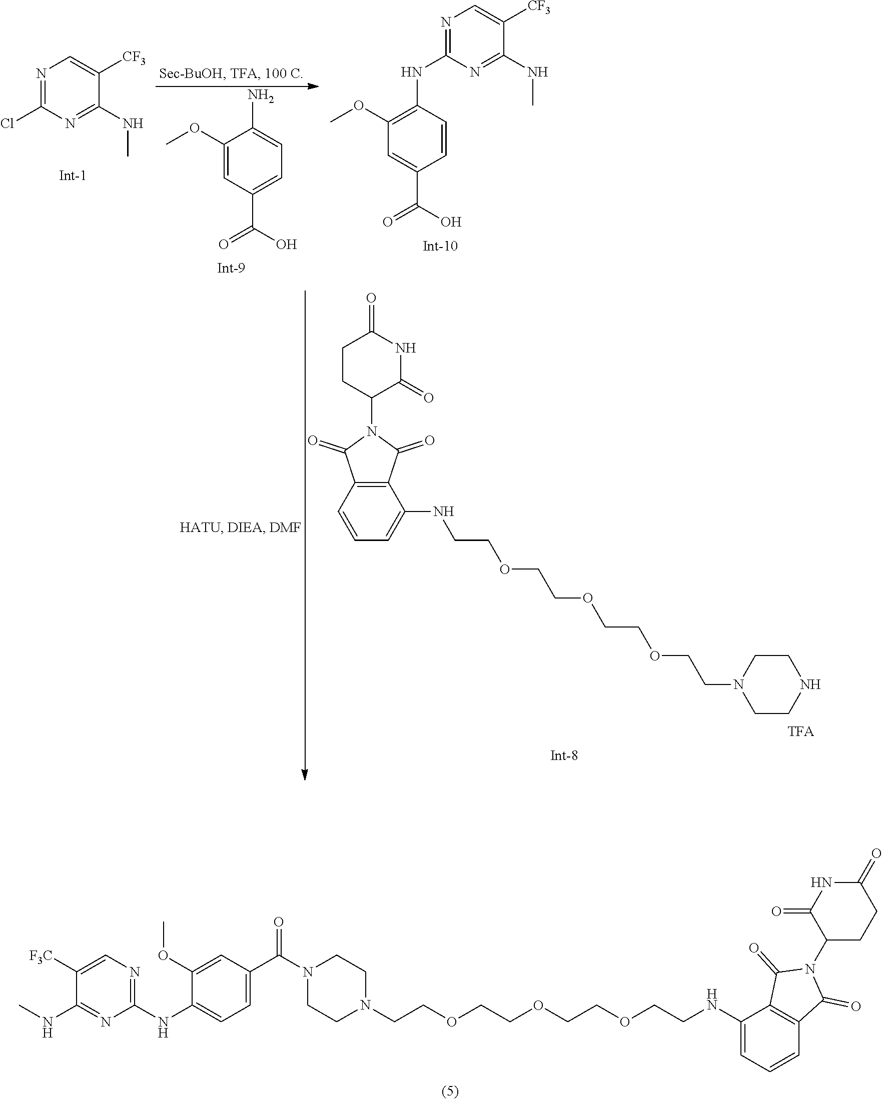

Intermediates Int-1 and Int-10 were prepared using the appropriate pyrimidine, aniline and boc-protected piperazine according to the procedures described in Choi, et al., ACS Med. Chem. Lett. 3(8):658-662 (2012).

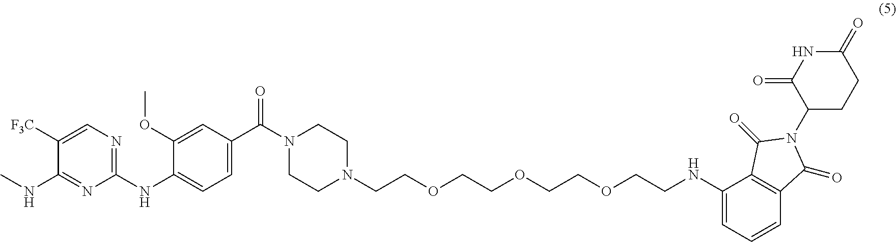 [SMILES: CNc1nc(Nc2ccc(C(=O)N3CCN(CCOCCOCCOCCNc4cccc5c4C(=O)N(C4CCC(=O)NC4=O)C5=O)CC3)cc2OC)ncc1C(F)(F)F]

To a solution of 3-methoxy-4-((4-(methylamino)-5-(trifluoromethyl)pyrimidin-2-yl)amino)benzoic acid (Int-10) (10 mg, 0.029 mmol) in DMF (2 mL) was added 2-(2,6-dioxopiperidin-3-yl)-4-((2-(2-(2-(2-(piperazin-1-yl)ethoxy)ethoxy)ethoxy)ethyl)amino) isoindoline-1,3-dione (Int-8) (15 mg, 0.029 mmol) and HATU (22 mg, 0.058 mmol), followed by DIEA (25 μL, 0.145 mmol). The mixture stirred for 30 minutes. The crude product was purified by reverse phase HPLC using a gradient of 1%-70% MeCN in H₂O to give the desired product as a yellow solid (7 mg, 37% yield).

MS (ESI) m/z: 842. 61 (M+H)⁺.

### Example 5: Synthesis of 4-((2-(2-(2-(2-(4-(4-((5-chloro-4-(methylamino)pyrimidin-2-yl)amino)-3-methoxybenzoyl)piperazin-1-yl)ethoxy)ethoxy)ethoxy)ethyl)amino)-2-(2,6-dioxopiperidin-3-yl)isoindoline-1,3-dione (4)

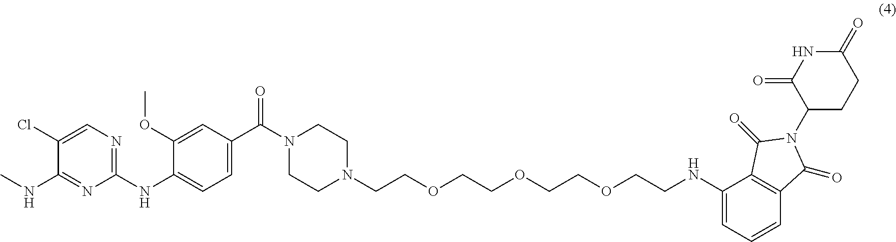 [SMILES: CNc1nc(Nc2ccc(C(=O)N3CCN(CCOCCOCCOCCNc4cccc5c4C(=O)N(C4CCC(=O)NC4=O)C5=O)CC3)cc2OC)ncc1Cl]

Compound 4 was prepared in an analogous manner to compound 5 in Example 4. The desired product was isolated as a yellow solid (9 mg, 56% yield).

MS (ESI) m/z: 809.61 (M+H)⁺.

### Example 6: Synthesis of 4-((14-(4-(4-((5-chloro-4-(methylamino)pyrimidin-2-yl)amino)-3-methoxybenzoyl)piperazin-1-yl)-3,6,9,12-tetraoxatetradecyl)amino)-2-(2,6-dioxopiperidin-3-yl)isoindoline-1,3-dione (6)

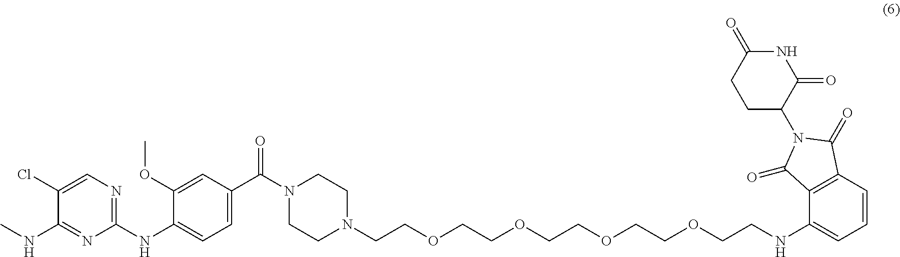 [SMILES: CNc1nc(Nc2ccc(C(=O)N3CCN(CCOCCOCCOCCOCCNc4cccc5c4C(=O)N(C4CCC(=O)NC4=O)C5=O)CC3)cc2OC)ncc1Cl]

Compound 6 was prepared in an analogous manner to compound 5 in Example 4. The desired product was isolated as a yellow solid (6 mg, 21% yield).

MS (ESI) m/z: 881.36 (M+H)⁺.

### Example 7: Synthesis of 2-(2,6-dioxopiperidin-3-yl)-4-((14-(4-(3-methoxy-4-((4-(methylamino)-5-(trifluoromethyl)pyrimidin-2-yl)amino)benzoyl)piperazin-1-yl)-3,6,9,12-tetraoxatetradecyl)amino)isoindoline-1,3-dione (7)

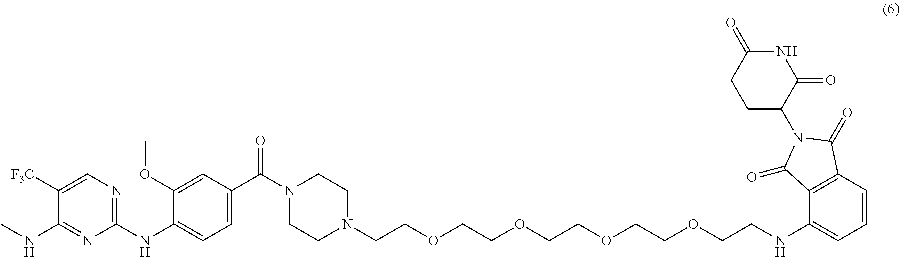 [SMILES: CNc1nc(Nc2ccc(C(=O)N3CCN(CCOCCOCCOCCOCCNc4cccc5c4C(=O)N(C4CCC(=O)NC4=O)C5=O)CC3)cc2OC)ncc1C(F)(F)F]

Compound 7 was prepared in an analogous manner to compound 5 in Example 4. The desired product was isolated as a yellow solid (8 mg, 30% yield).

¹H NMR (500 MHz, DMSO) δ 11.10 (br, 1H), 9.96 (br, 1H), 8.57 (br, 1H), 8.30 (d, J=9 Hz, 1H), 8.28 (s, 1H), 7.64 (s, 1H), 7.58 (m, 1H), 7.14-7.09 (m, 3H), 7.05 (d, J=5 Hz, 2H), 6.58 (br, 1H), 5.05 (dd, J=5 Hz, 6 Hz, 1H), 3.91 (s, 3H), 3.72-3.32 (m, 27H), 2.94 (d, 5 Hz, 3H), 2.62-2.55 (m, 3H), 2.09-1.99 (m, 1H).

MS (ESI) m/z: 914.45 (M+H)⁺.

### Example 8: Synthesis of 2-(2,6-dioxopiperidin-3-yl)-4-((2-(2-(3-(4-(6-(5-(1-methylcyclopropoxy)-1H-indazol-3-yl)pyrimidin-4-yl)piperazin-1-yl)-3-oxopropoxy)ethoxy)ethyl)amino)isoindoline-1,3-dione (8)

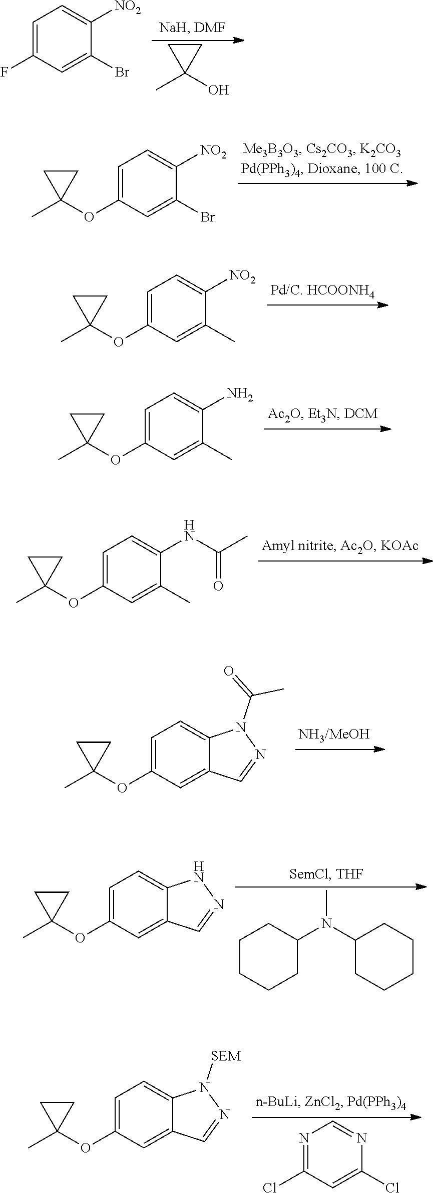

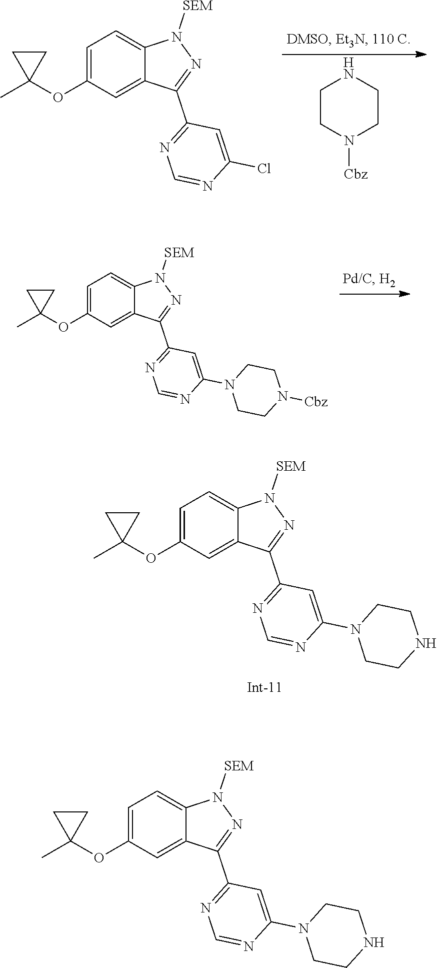 [SMILES: CC1(Oc2ccc3c(c2)c(-c2cc(Cl)ncn2)nn3COCC[Si](C)(C)C)CC1 ; O=C(OCc1ccccc1)N1CCNCC1 ; CC1(Oc2ccc3c(c2)c(-c2cc(N4CCN(C(=O)OCc5ccccc5)CC4)ncn2)nn3COCC[Si](C)(C)C)CC1 ; CC1(Oc2ccc3c(c2)c(-c2cc(N4CCNCC4)ncn2)nn3COCC[Si](C)(C)C)CC1 ; CC1(Oc2ccc3c(c2)c(-c2cc(N4CCNCC4)ncn2)nn3COCC[Si](C)(C)C)CC1]

### 5-(1-methylcyclopropoxy)-3-(6-(piperazin-1-yl)pyrimidin-4-yl)-1-((2-(trimethylsilyl)ethoxy) methyl)-1H-indazole (Int-11)

Intermediate Int-11 was prepared according to the procedure described in Scott et al., J. Med. Chem. 60(7):2983-2992 (2017).

MS (ESI) m/z 481.42 (M+H)⁺.

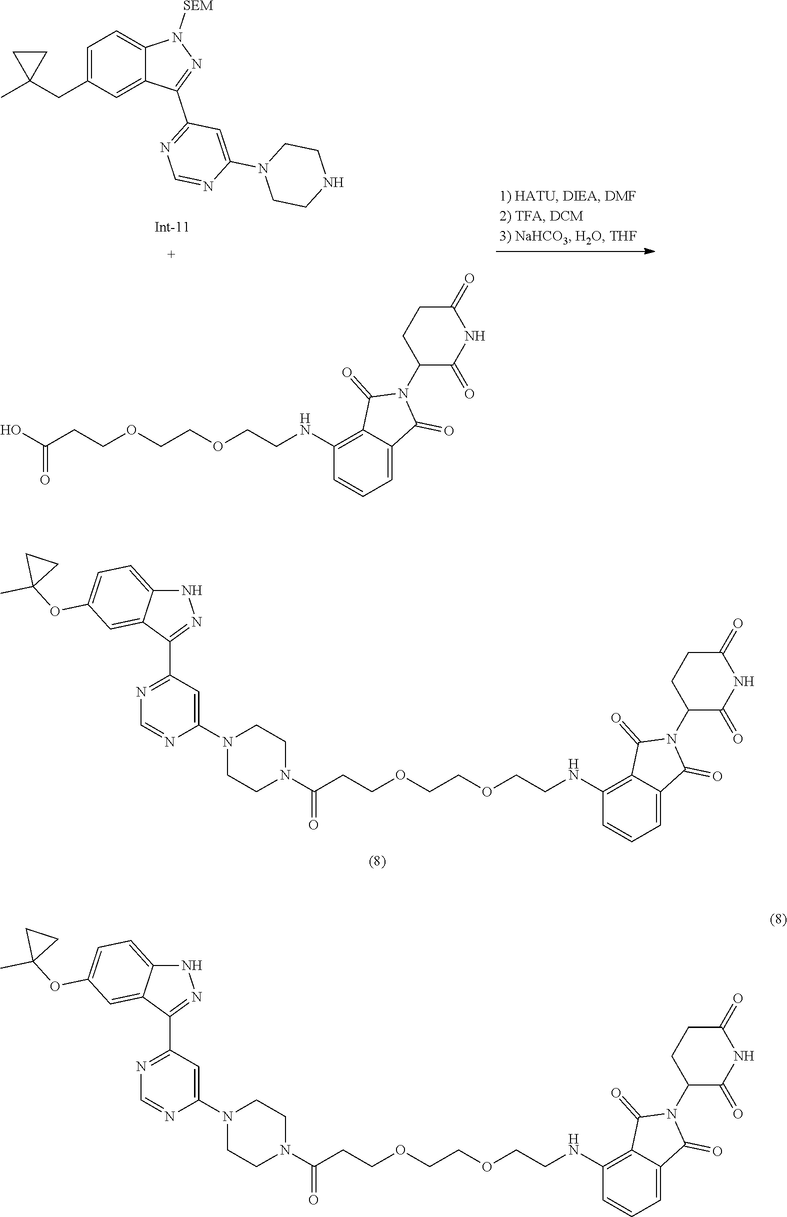 [SMILES: CC1(Cc2ccc3c(c2)c(-c2cc(N4CCNCC4)ncn2)nn3COCC[Si](C)(C)C)CC1 ; O=C(O)CCOCCOCCNc1cccc2c1C(=O)N(C1CCC(=O)NC1=O)C2=O ; CC1(Oc2ccc3[nH]nc(-c4cc(N5CCN(C(=O)CCOCCOCCNc6cccc7c6C(=O)N(C6CCC(=O)NC6=O)C7=O)CC5)ncn4)c3c2)CC1 ; CC1(Oc2ccc3[nH]nc(-c4cc(N5CCN(C(=O)CCOCCOCCNc6cccc7c6C(=O)N(C6CCC(=O)NC6=O)C7=O)CC5)ncn4)c3c2)CC1]

To a solution of 5-(1-methylcyclopropoxy)-3-(6-(piperazin-1-yl)pyrimidin-4-yl)-1-((2-(trimethylsilyl)ethoxy)methyl)-1H-indazole (Int-11) (20 mg, 0.042 mmol) and 3-(2-(2-((2-(2,6-dioxopiperidin-3-yl)-1,3-dioxoisoindolin-4-yl)amino)ethoxy)ethoxy)propanoic acid (20 mg, 0.046 mmol) was added HATU (32 mg, 0.084 mmol), followed by DIEA (40 μL, 0.21 mmol). The mixture was stirred for 30 minutes. The reaction was quenched with H₂O and extracted with EtOAc. The combined organic extracts were washed with brine, dried over MgSO₄, and condensed under reduced pressure to give a brown residue. To a solution of the brown product in DCM (10 mL) was added TFA (1 mL), and the resulting mixture was stirred for 1 hour. The solvent was removed under reduced pressure. The residue was redissolved in THF (10 mL) before adding saturated aqueous NaHCO₃(2 mL), and the mixture was stirred at room temperature for 1 hour. The reaction was quenched with H₂O and extracted with EtOAc. The combined organic extracts were washed with brine, dried over MgSO₄, and condensed under reduced pressure to give a brown residue. The crude product was purified by reverse phase HPLC using a gradient of 1%-80% MeCN in H₂O to give the desired product as a yellow oil (6 mg, 19% yield).

¹H NMR (500 MHz, DMSO) δ 13.81 (br, 1H), 11.09 (s, 1H), 8.71 (s, 1H), 7.60 (d, J=8 Hz, 1H), 7.56 (t, J=10 Hz, 1H), 7.39 (s, 1H), 7.19 (m, 1H), 7.12 (d, J=8 Hz, 1H), 7.04 (d, J=6 Hz, 1H), 6.59 (s, 1H), 5.04 (dd, J=5 Hz, 6 Hz, 1H), 3.84 (m, 4H), 3.89-3.42 (m, 15H), 2.92-2.84 (m, 1H), 2.65-2.58 (m, 3H), 2.07 (s, 1H), 2.03 (m, 1H), 1.55 (s, 3H), 0.98 (m, 2H), 0.79 (m, 2H).

MS (ESI) m/z: 766.37 (M+H)⁺.

### Example 9: Synthesis of 2-(2,6-dioxopiperidin-3-yl)-4-((15-(4-(6-(5-(1-methylcyclopropoxy)-1H-indazol-3-ylpyrimidin-4-yl)piperazin-1-yl)-15-oxo-3,6,9,12-tetraoxapentadecyl)amino)isoindoline-1,3-dione (9)

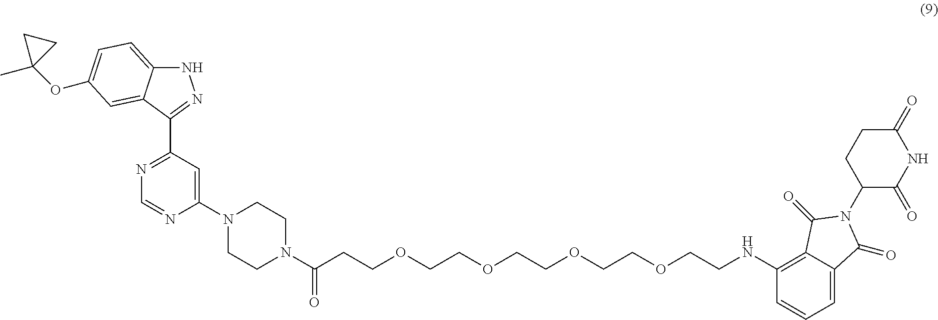 [SMILES: CC1(Oc2ccc3[nH]nc(-c4cc(N5CCN(C(=O)CCOCCOCCOCCOCCNc6cccc7c6C(=O)N(C6CCC(=O)NC6=O)C7=O)CC5)ncn4)c3c2)CC1]

Compound 9 was prepared in an analogous manner to compound 8 in Example 8.

MS (ESI) m/z: 854.62 (M+H)⁺.

### Example 10: Synthesis of 2-(2,6-dioxopiperidin-3-yl)-4-((2-(3-(4-(6-(5-(1-methylcyclopropoxy)-1H-indazol-3-yl)pyrimidin-4-yl)piperazin-1-yl)-3-oxopropoxy)ethyl)amino)isoindoline-1,3-dione (10)

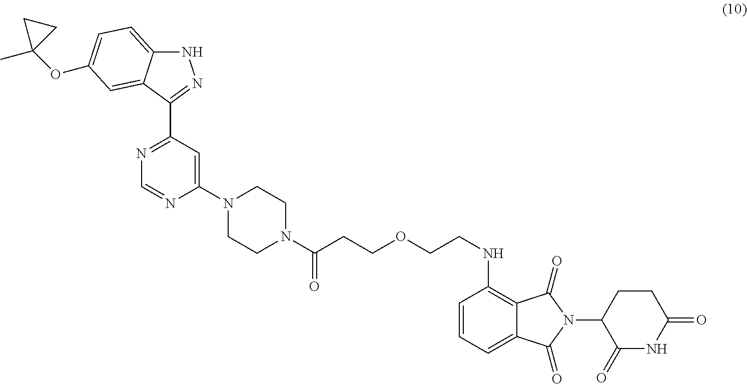 [SMILES: CC1(Oc2ccc3[nH]nc(-c4cc(N5CCN(C(=O)CCOCCNc6cccc7c6C(=O)N(C6CCC(=O)NC6=O)C7=O)CC5)ncn4)c3c2)CC1]

Compound 10 was prepared in an analogous manner to compound 8 in Example 8. The desired product was isolated as a yellow oil (4 mg, 13% yield).

¹H NMR (500 MHz, DMSO) δ 11.09 (s, 1H), 8.72 (s, 1H), 7.65 (d, J=8 Hz, 1H), 7.57 (t, J=10 Hz, 1H), 7.36 (s, 1H), 7.24 (m, 1H), 7.14 (d, J=8 Hz, 1H), 7.01 (d, J=6 Hz, 1H), 6.58 (s, 1H), 5.04 (dd, J=5 Hz, 6 Hz, 1H), 3.84 (m, 4H), 3.89-3.42 (m, 15H), 2.85 (m, 1H), 2.67 (m, 2H), 1.55 (s, 3H), 0.99 (m, 2H), 0.81 (m, 2H). MS (ESI) m/z: 722.48 (M+H)⁺.

### Example 11: Synthesis of 3-(7-((2-(3-(4-(6-(5-(1-methylcyclopropoxy)-1H-indazol-3-yl)pyrimidin-4-yl)piperazin-1-yl)-3-oxopropoxy)ethyl)amino)-1-oxoisoindolin-2-yl)piperidine-2,6-dione (11)

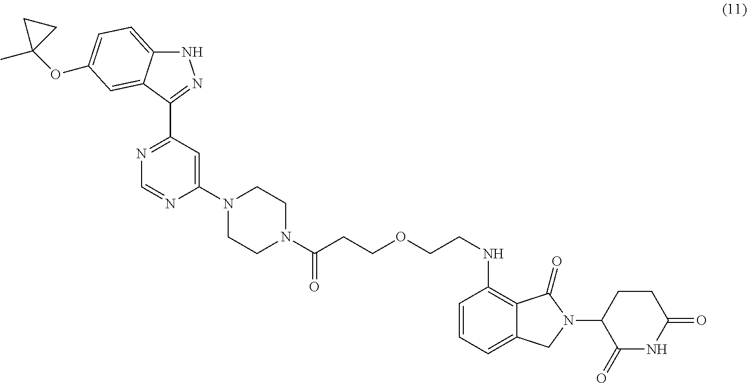 [SMILES: CC1(Oc2ccc3[nH]nc(-c4cc(N5CCN(C(=O)CCOCCNc6cccc7c6C(=O)N(C6CCC(=O)NC6=O)C7)CC5)ncn4)c3c2)CC1]

Compound 11 was prepared in an analogous manner to compound 8 in Example 8. The desired product was isolated as a brown solid (3 mg, 10% yield).

MS (ESI) m/z: 708.61 (M+H)⁺.

### Example 12: Synthesis of 3-(7-((2-(2-(3-(4-(6-(5-(1-methylcyclopropoxy)-1H-indazol-3-yl)pyrimidin-4-yl)piperazin-1-yl)-3-oxopropoxy)ethoxy)ethyl)amino)-1-oxoisoindolin-2-yl)piperidine-2,6-dione (12)

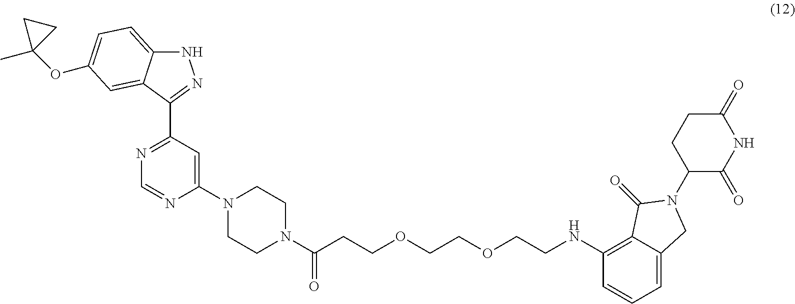 [SMILES: CC1(Oc2ccc3[nH]nc(-c4cc(N5CCN(C(=O)CCOCCOCCNc6cccc7c6C(=O)N(C6CCC(=O)NC6=O)C7)CC5)ncn4)c3c2)CC1]

Compound 12 was prepared in an analogous manner to compound 8 in Example 8. The desired product was isolated as a brown oil (1 mg, 3% yield).

¹H NMR (500 MHz, DMSO) δ 11.09 (s, 1H), 8.72 (s, 1H), 7.65 (d, J=8 Hz, 1H), 7.57 (t, J=10 Hz, 1H), 7.36 (s, 1H), 7.24 (m, 1H), 7.14 (d, J=8 Hz, 1H), 7.01 (d, J=6 Hz, 1H), 6.58 (s, 1H), 5.04 (dd, J=5 Hz, 6 Hz, 1H), 3.84 (m, 4H), 3.89-3.42 (m, 15H), 2.85 (m, 1H), 2.67 (m, 2H), 1.55 (s, 3H), 0.99 (m, 2H), 0.81 (m, 2H).

MS (ESI) m/z: 752.78 (M+H)⁺.

### Example 13: Synthesis of 3-(7-((15-(4-(6-(5-(1-methylcyclopropoxy)-1H-indazol-3-yl)pyrimidin-4-ylpiperazin-1-yl)-15-oxo-3,6,9,12-tetraoxapentadecyl)amino)-1-oxoisoindolin-2-ylpiperidine-2,6-dione (13)

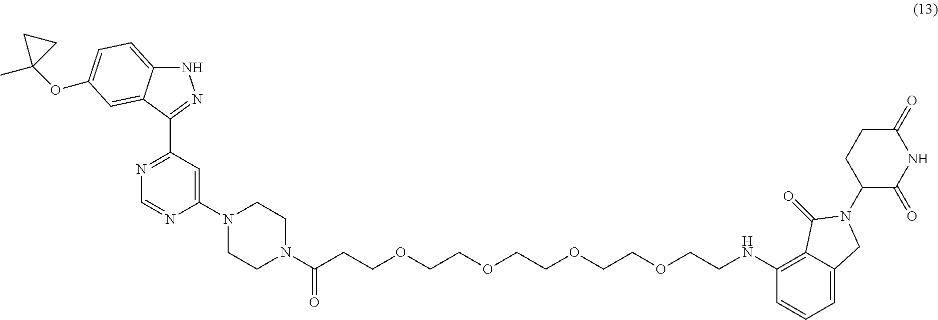 [SMILES: CC1(Oc2ccc3[nH]nc(-c4cc(N5CCN(C(=O)CCOCCOCCOCCOCCNc6cccc7c6C(=O)N(C6CCC(=O)NC6=O)C7)CC5)ncn4)c3c2)CC1]

Compound 13 was prepared in an analogous manner to compound 8 in Example 8. The desired product was isolated as a brown oil (2 mg, 6% yield).

MS (ESI) m/z: 840.14 (M+H)⁺.

### Example 14: Synthesis of (2R,4S)-1-((R)-2-(tert-butyl)-16-(4-(6-(5-(1-methylcyclopropoxy)-1H-indazol-3-yl)pyrimidin-4-yl)piperazin-1-yl)-4,16-dioxo-7,10,13-trioxa-3-azahexadecanoyl)-4-hydroxy-N—((R)-1-(4-(4-methylthiazol-5-yl)phenyl)ethyl)pyrrolidine-2-carboxamide (14)

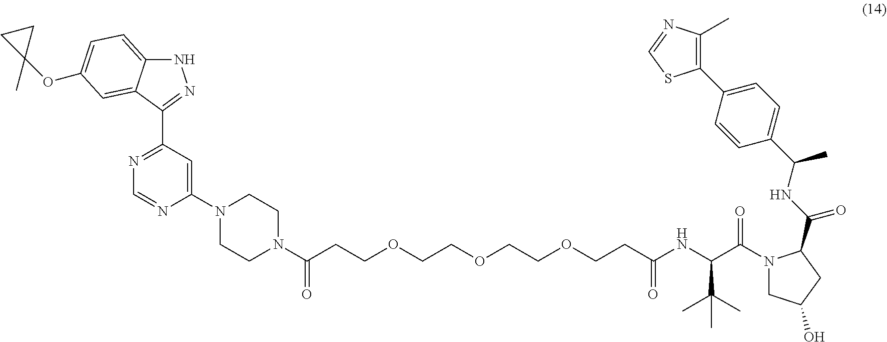 [SMILES: Cc1ncsc1-c1ccc([C@@H](C)NC(=O)[C@H]2C[C@H](O)CN2C(=O)[C@H](NC(=O)CCOCCOCCOCCC(=O)N2CCN(c3cc(-c4n[nH]c5ccc(OC6(C)CC6)cc45)ncn3)CC2)C(C)(C)C)cc1]

Compound 14 was prepared in an analogous manner to compound 8 in Example 8. The desired product was isolated as a brown oil (14 mg, 31% yield).

MS (ESI) m/z: 1010.65 (M+H)⁺.

### Example 15: Synthesis of (2S,4R)-1-((S)-2-(tert-butyl)-19-(4-(6-(5-(1-methylcyclopropoxy)-1H-indazol-3-yl)pyrimidin-4-ylpiperazin-1-yl)-4,19-dioxo-7,10,13,16-tetraoxa-3-azanonadecanoyl)-4-hydroxy-N—((S)-1-(4-(4-methylthiazol-5-yl)phenyl)ethyl)pyrrolidine-2-carboxamide (15)

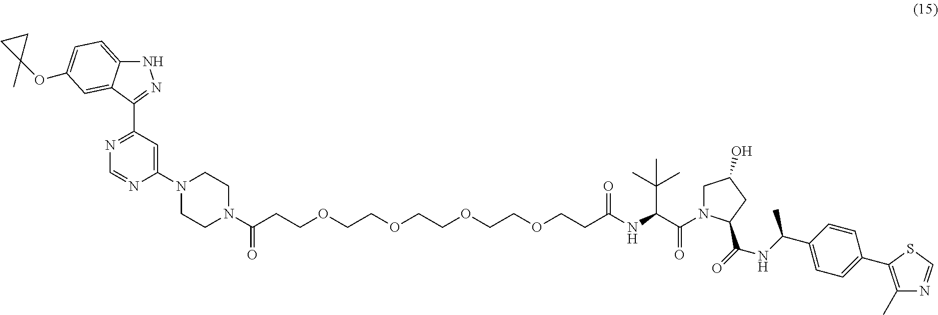 [SMILES: Cc1ncsc1-c1ccc([C@H](C)NC(=O)[C@@H]2C[C@@H](O)CN2C(=O)[C@@H](NC(=O)CCOCCOCCOCCOCCC(=O)N2CCN(c3cc(-c4n[nH]c5ccc(OC6(C)CC6)cc45)ncn3)CC2)C(C)(C)C)cc1]

Compound 15 was prepared in an analogous manner to compound 8 in Example 8. The desired product was isolated as a brown oil (11 mg, 24% yield).

MS (ESI) m/z: 1054.76 (M+H)⁺.

### Example 16: Synthesis of 4-((2-(2-(3-(4-(6-(5-(1-methylcyclopropoxy)-1H-indazol-3-yl)pyrimidin-4-ylpiperazin-1-yl)-3-oxopropoxy)ethoxy)ethyl)amino)-2-(2-oxopiperidin-3-yl)isoindoline-1,3-dione (16)

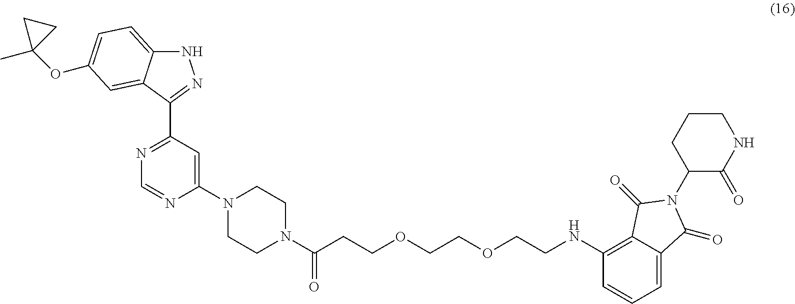 [SMILES: CC1(Oc2ccc3[nH]nc(-c4cc(N5CCN(C(=O)CCOCCOCCNc6cccc7c6C(=O)N(C6CCCNC6=O)C7=O)CC5)ncn4)c3c2)CC1]

Compound 16 was prepared in an analogous manner to compound 8 in Example 8. The desired product was isolated as a yellow oil (5 mg, 16% yield).

MS (ESI) m/z: 752.28 (M+H)⁺.

### Example 17: Cellular Degradation of LRRK2 with Inventive Compound 1

The materials and methods for the for this experiment are described below:

Cell lines used: Mouse embryonic fibroblast (MEF) WT, LRRK2 homozygous knock-ins in MEFs [R1441C; VPS35N(D620N); G2019S].

Tested concentration of LRRK2 degraders: 0 nM, 10 nM, 30 nM, 100 nM, 300 nM, 1000 nM. Additional concentrations tested for inventive compound 3: 2 uM, 5 uM and 10 uM.

Complete growth medium: DMEM supplemented with: 10% Fetal Bovine Serum; 1% pen/strep; 1% L-Glutamine; 1% MEM Non-essential Amino Acid Solution; 1% sodium pyruvate.

Commercial and in-house purified antibodies:
(a) Mouse anti-LRRK2/Dardarin antibody from Antibodies, Inc. (Cat #75-253).
(b) Rabbit monoclonal antibodies for total LRRK2 (UDD3) and pS935-LRRK2 (UDD2) were purified at the University of Dundee (as described in Dzamko et al., PLoS One 7(6): e39132 (2012).
(c) Loading controls: anti-α-tubulin (Cell Signaling Technology #5174); anti-GAPDH (Santa Cruz Biotechnology Cat. #sc-32233)
(d) (p)Rab10 antibodies: rabbit anti-RAB10 (phospho T73) antibody [MJF-R21] (ab230261); mouse MJFF-total Rab10 monoclonal antibody were generated by nanoTools (www.nanotools.de); rabbit Rab10 total was from Cell Signaling Technology (Rab10 (D36C4) XP® Rabbit mAb #8127)

Treatment: WT MEF, R1441C, VPS35N and G2019S mutants' cells were plated at equal density into 6-well plates in a final volume of 3 mL of complete growth medium/well. Degraders were reconstituted in DMSO and used at 1:1000 in cells i.e. 3 μl/3 ml. Treatment began when cells were >60% confluent, starting from a 48-hour time point, followed by a 24-hour time point, a 6-hour time point and finally a 1-hour time point.

Cell lysis: Media were aspirated, plates were placed on ice and cells were washed with Dulbecco's phosphate-buffered saline (DPBS). Fifty microliters of an ice-cold lysis buffer containing 50 mM Tris-HCl, pH 7.5, 1% (v/v) Triton X-100, 1 mM ethylene glycol-bis(μ-aminoethyl ether)-N,N,N′,N′-tetraacetic acid (EGTA), 1 mM sodium orthovanadate, 50 mM NaF, 0.1% (v/v) 2-mercaptoethanol, 10 mM 2-glycerophosphate, 5 mM sodium pyrophosphate, 0.1 μg/ml microcystin-LR (Enzo Life Sciences), 270 mM sucrose and complete EDTA-free protease inhibitor cocktail (Sigma-Aldrich Cat #11836170001) was added per well. Lysates were centrifuged at 20,817 g (14,000 rpm) for 15 min at 4° C. and supernatants were used to determine protein concentration using Bradford assay (Pierce™ Coomassie (Bradford) Protein Assay Kit, Thermo Scientific™ Cat #23200) and for Western blot analysis.

Western blot analysis: Cell lysates were mixed with 4× SDS-PAGE sample buffer [50 mM Tris-HCl, pH 6.8, 2% (w/v) SDS, 10% (v/v) glycerol, 0.02% (w/v) Bromophenol Blue and 1% (v/v) 2-mercaptoethanol] to a final total protein concentration of 1 μg/μl and heated at 95° C. for 5 minutes. Twenty micrograms of samples were loaded onto NuPAGE™ 4-12% Bis-Tris gradient gels (Life Technologies) along with 3 μl of BIO-RAD protein marker (Precision Plus Protein™ All Blue Prestained Protein Standards #1610373 kDa), gels were run in duplicates at 110V for 2 hours and 30 minutes with the NuPAGE™ MOPS SDS running buffer (Life Technologies, Cat #NP0001-02). After electrophoresis, the separated proteins were transferred onto the nitrocellulose membrane (GE Healthcare, Amersham Protran 0.45 μm NC) at 90 V for 90 minutes. Transferred membranes were briefly stained with Ponceau S stain and divided into 3 strips, as described earlier in Fan et al., Biochem. J. 475:23-44 (2018). Briefly, upper strip was cut from the top of the membrane to 75 kDa, middle strip cut was between 75 kDa—30 kDa and bottom strip cut was from 30 kDa—to the bottom of the membrane. Membrane strips were blocked at room temperature with 5% (w/v) dried skimmed milk dissolved in TBS-T [20 mM Tris-HCl, pH 7.5, 150 mM NaCl and 0.1% (v/v) Tween® 20] for 1 hour, washed four times with ten minutes intervals in TBS-T and incubated with primary antibodies diluted in 5% BSA (bovine serum albumin) in TBS-T overnight at 4° C. Primary antibodies were used as follow: upper strip from one of the membranes was incubated with 1 μg/ml of rabbit anti-LRRK2 pS935 UDD2 antibody combined with mouse anti-LRRK2 C-terminus total antibody, while the second upper strip was incubated with anti-LRRK2 N-terminus total antibody (UDD3) at a final concentration of 100 ng/ml; the middle strips were incubated with rabbit anti-α-tubulin (Cell Signaling Technology #5174) and mouse anti-GAPDH antibody (Santa Cruz Biotechnology #sc-32233) at a final concentration of 50 ng/ml. The bottom strips were blotted with rabbit MJFF-pRAB10 monoclonal antibody multiplexed with mouse MJFF-total Rab10 monoclonal antibody at a final concentration of 0.5 μg/ml for each of the antibody and with the total Rab10 (Rab10 (D36C4) XP® Rabbit mAb #8127 Cell Signaling Technology) at a final concentration of 1 μg/ml (Lis et al., Biochem. J. 475:1-22 (2018); Fan et al., Biochem. J. 475:23-44 (2018)). Membranes were washed as before and incubated at room temperature for 1 h with anti-rabbit and anti-mouse near-infrared fluorescent IRDye® antibodies (LI-COR® #925-68070, #925-32211) diluted (1:30 000 and 1:15 000, respectively) in TBS-T. Following incubation in secondary antibodies, membrane strips were washed and signal developed using the LI-COR® Odyssey® CLx Western Blot imaging system.

IC₅₀ experiments were performed using Invitrogen™'s Adapta™ assay.

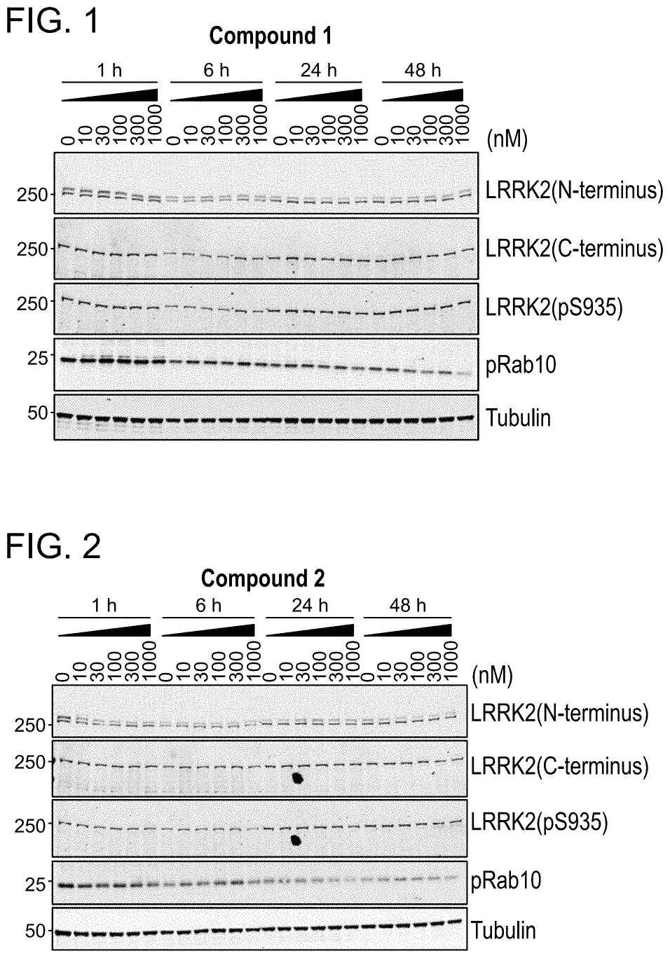

The results in FIG. 1 show that inventive compound 1 inhibited the phosphorylation of S935 and Rab10, but did not degrade LRRK2.

### Example 18: Cellular Degradation of LRRK2 with Inventive Compound 2

The experimental protocol is as in Example 17.

The results in FIG. 2 show that inventive compound 2 inhibited the phosphorylation of S935 and Rab10, but did not degrade LRRK2.

### Example 19: Cellular Degradation of LRRK2 with Inventive Compound 3

The experimental protocol is as in Example 17.

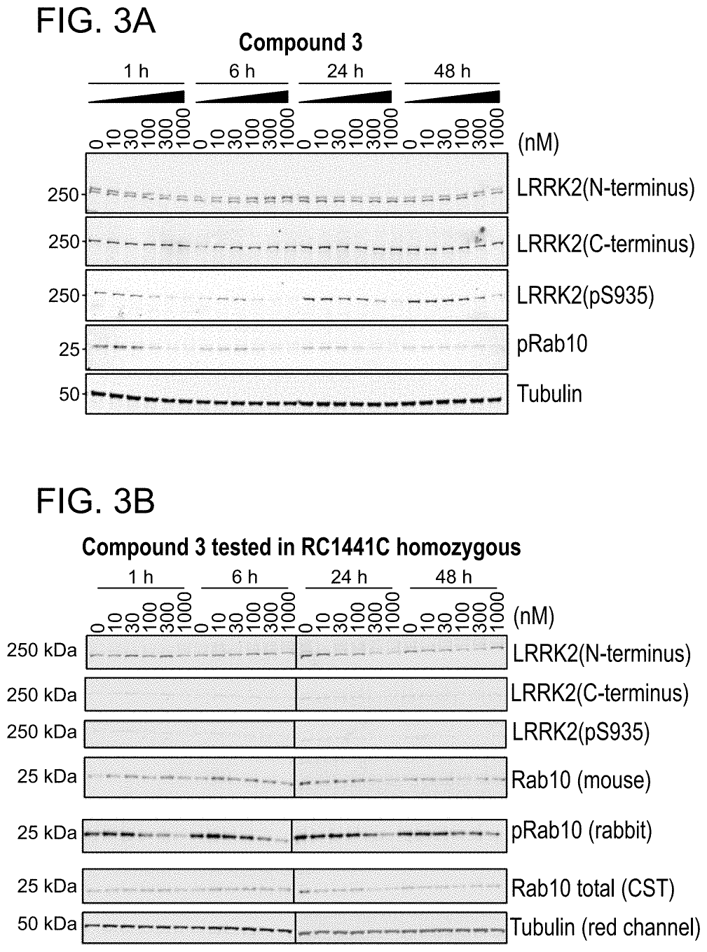

The results in FIG. 3A show that inventive compound 3 inhibited the phosphorylation of S935 and Rab10, but did not degrade LRRK2. The degradation of LRRK2 (C-terminus) by the inventive compound was observed in FIG. 3B.

TABLE 1
IC₅₀ of inventive compounds 1-3.

IC₅₀ (nM)

| Inventive Compound | LRRK2 wt | LRRK2 G2019S |
|---|---|---|
| 1 | 4.0 | 2.0 |
| 2 | 2.0 | 1.0 |
| 3 | 2.0 | 1.0 |

IC₅₀ values for the inventive compounds are reported in the table above.

### Example 20: Cellular Degradation of LRRK2 with Inventive Compound 4

The experimental protocol is as in Example 17.

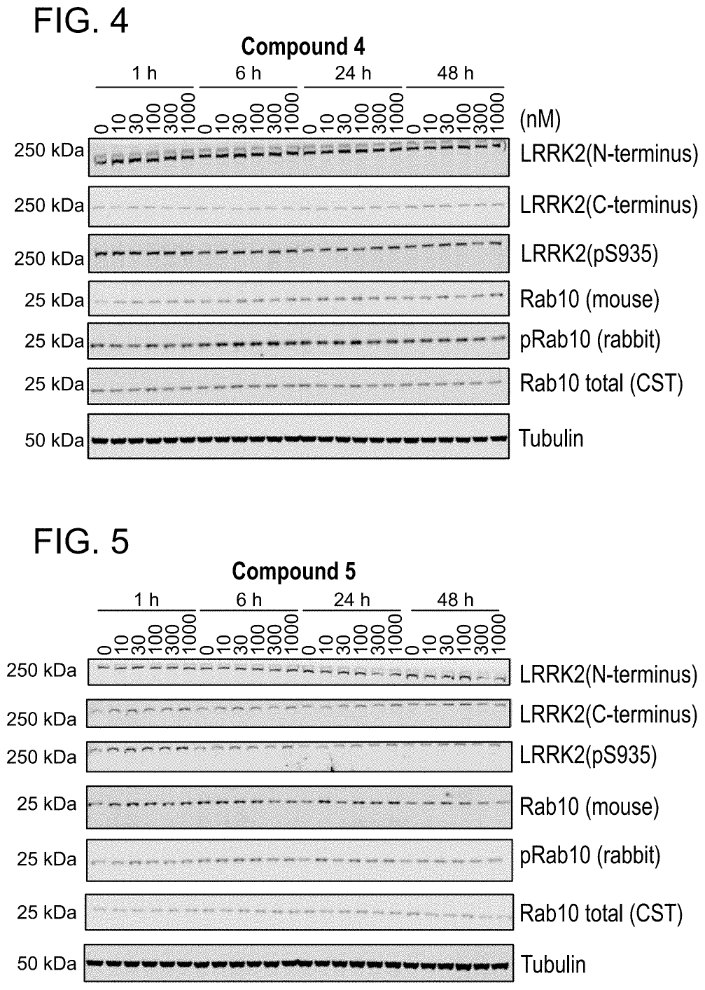

The results in FIG. 4 show that inventive compound 4 inhibited the phosphorylation of Rab10 and degraded LRRK2 (C-terminus). The degradation of LRRK2 (N-terminus) and the inhibition of the phosphorylation of S935 by the inventive compound were not observed.

### Example 21: Cellular Degradation of LRRK2 with Inventive Compound 5

The experimental protocol is as in Example 17.

The results in FIG. 5 show that inventive compound 5 inhibited the phosphorylation of S935 and Rab10, but did not degrade LRRK2.

### Example 22: Cellular Degradation of LRRK2 with Inventive Compound 6

The experimental protocol is as in Example 17.

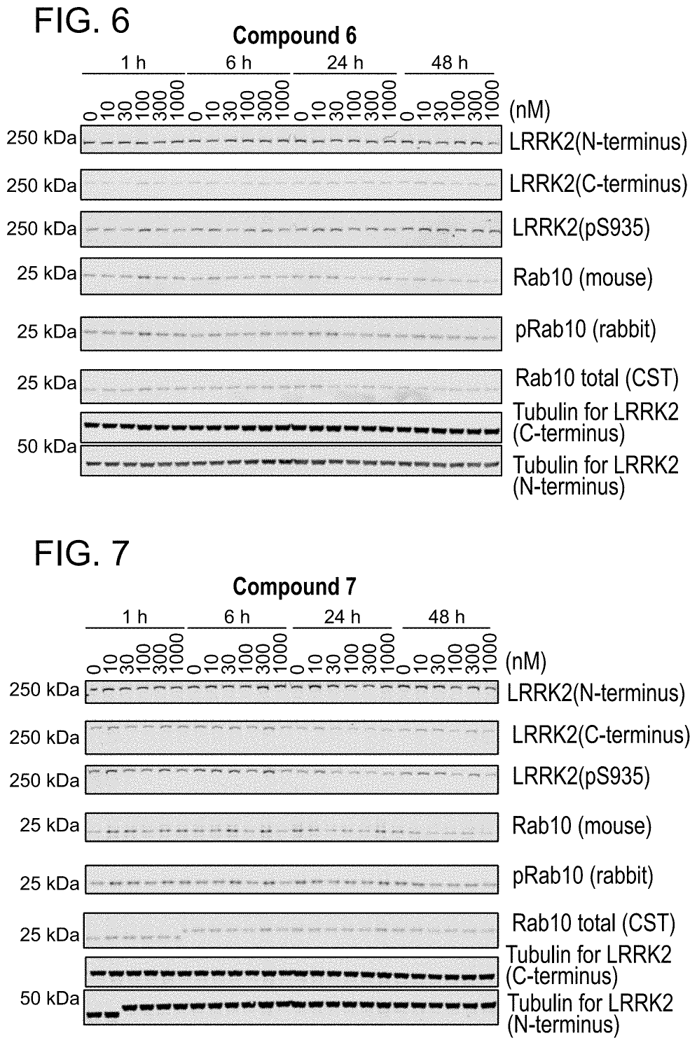

The results in FIG. 6 show that inventive compound 6 inhibited the phosphorylation of Rab10, and degraded LRRK2 (C-terminus). The degradation of LRRK2 (N-terminus) and the inhibition of the phosphorylation of S935 by the inventive compound were not observed.

### Example 23: Cellular Degradation of LRRK2 with Inventive Compound 7

The experimental protocol is as in Example 17.

The results in FIG. 7 show that inventive compound 7 inhibited the phosphorylation of Rab10 and S935, and degraded LRRK2 (C-terminus). The degradation of LRRK2 (N-terminus) by inventive compound 7 was not observed.

### Example 24: Intracellular CRBN Binding Experiment with Inventive Compounds and Positive Controls Lenalidomide and Pomalidomide

Compounds in Atto565-Lenalidomide displacement assay were dispensed in a 384-well microplate (Corning, 4514) using D300e Digital Dispenser (HP) and normalized to 1% DMSO into 10 nM Atto565-Lenalidomide, 100 nM DDB1ΔB-CRBN, 50 mM Tris pH 7.5, 200 mM NaCl, 0.1% Pluronic® F-68 solution (Sigma). The change in fluorescence polarization was monitored using a PHERAstar® FS microplate reader (BMG Labtech) for 30 cycles of 187s each. Data from four independent measurements (n=4) was plotted and IC₅₀ values estimated using variable slope equation in GraphPad Prism 7.

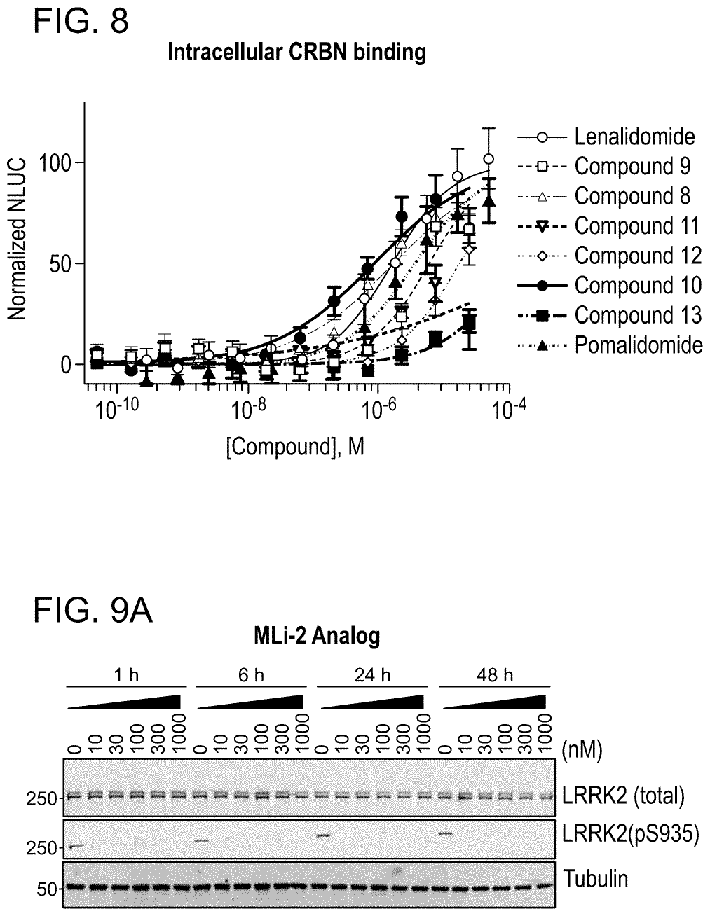

All of the inventive compounds in FIG. 8 were capable of cell penetration and bound CRBN with similar affinity as Pomalidomide and Lenalidomide.

### Example 25: Cellular Inhibition of LRRK2 with an Indazole

The experimental protocol is as in Example 17.

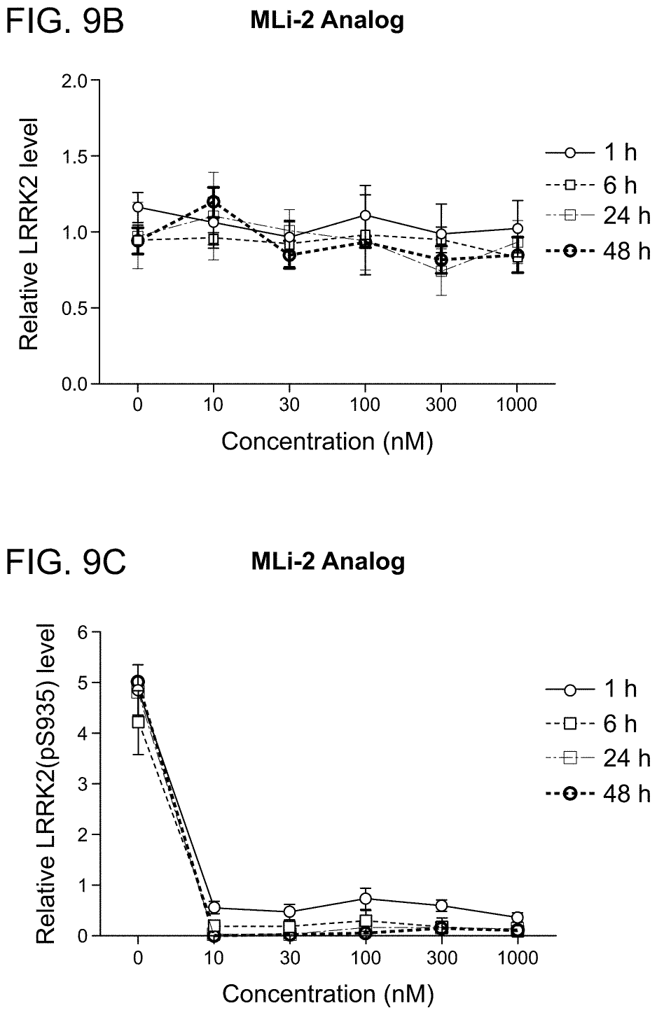

The results in FIG. 9A-FIG. 9C show that the indazole, which is an analog of a compound known as MLi-2 (see U.S. Patent Application Publication No. 2016/0009689 A1) inhibits the phosphorylation of S935, but did not decrease LRRK2 levels. The MLi-2 analog is illustrated in the structure below.

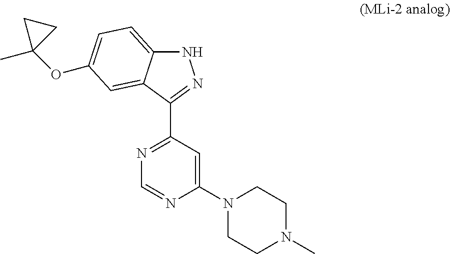

### Example 26: Cellular Degradation of LRRK2 with Inventive Compound 8

The experimental protocol is as in Example 17.

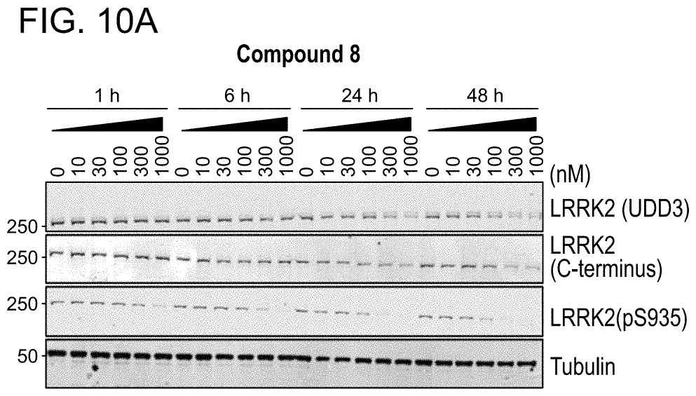

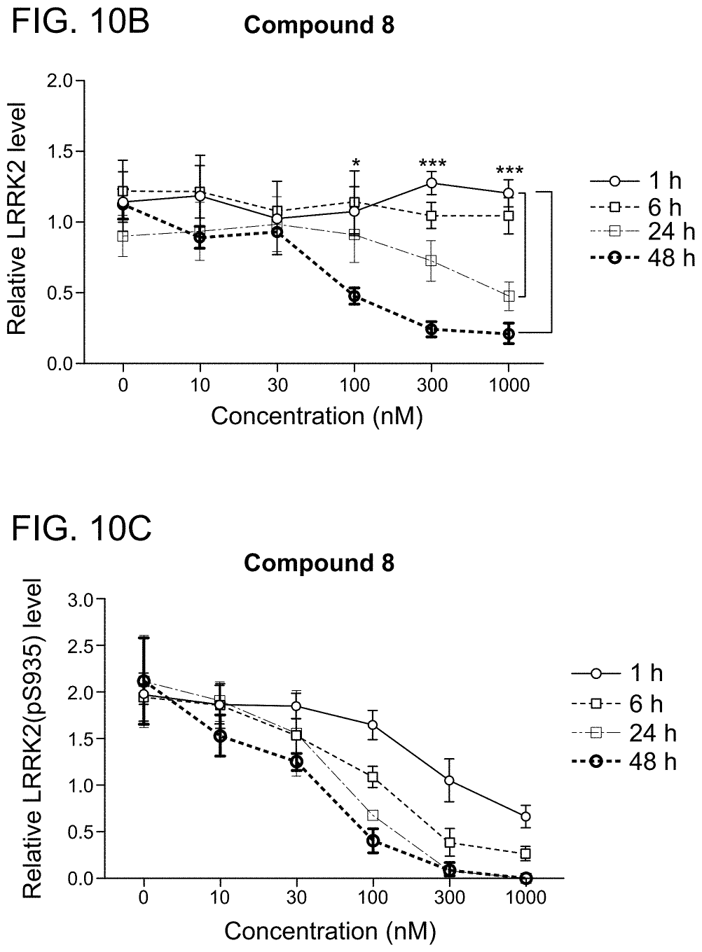

The results in FIG. 10A-FIG. 10C show that inventive compound 8 inhibited the phosphorylation of S935 as well as the MLi-2 analog, and also decreased the total level of LRRK2.

### Example 27: Cellular Degradation of LRRK2 with Inventive Compound 9

The experimental protocol is as in Example 17.

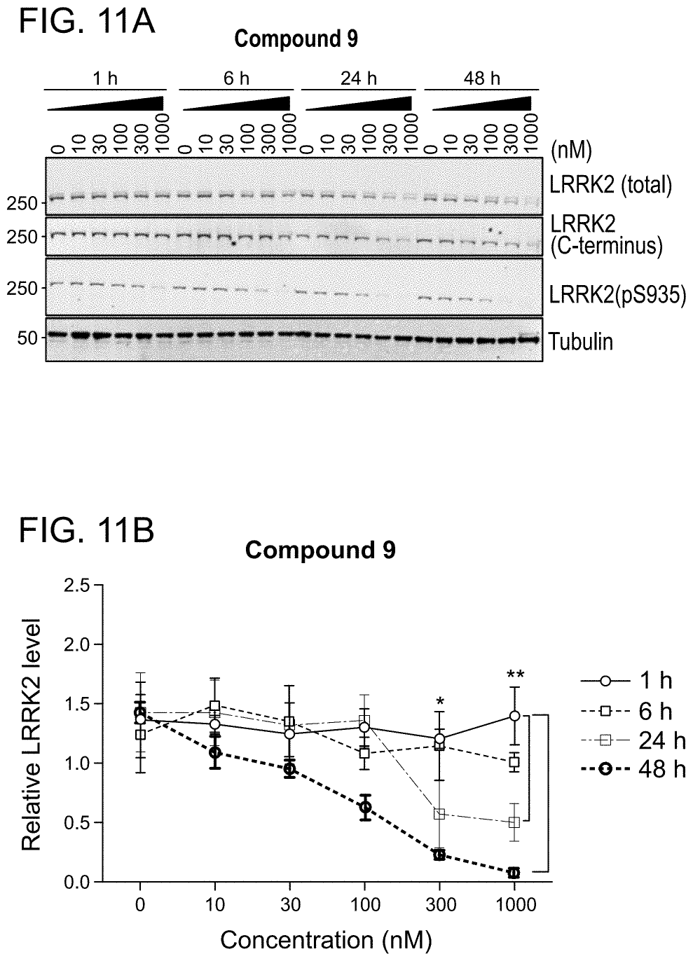

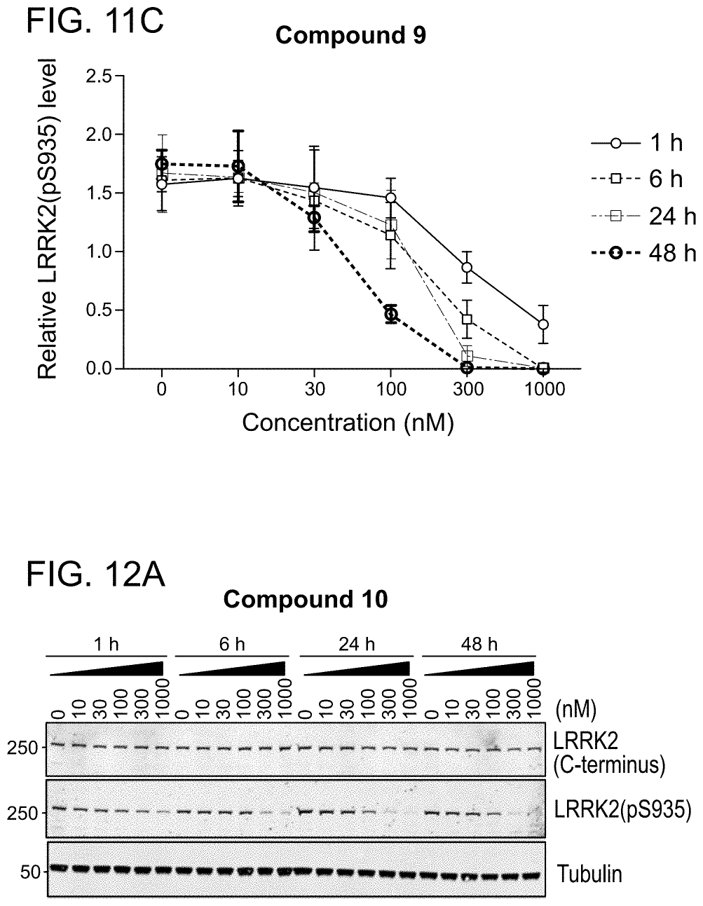

The results in FIG. 11A-FIG. 11C that inventive compound 9 inhibited the phosphorylation of S935 as well as the MLi-2 analog, and also decreased the total level of LRRK2.

### Example 28: Cellular Degradation of LRRK2 with Inventive Compound 10

The experimental protocol is as in Example 17.

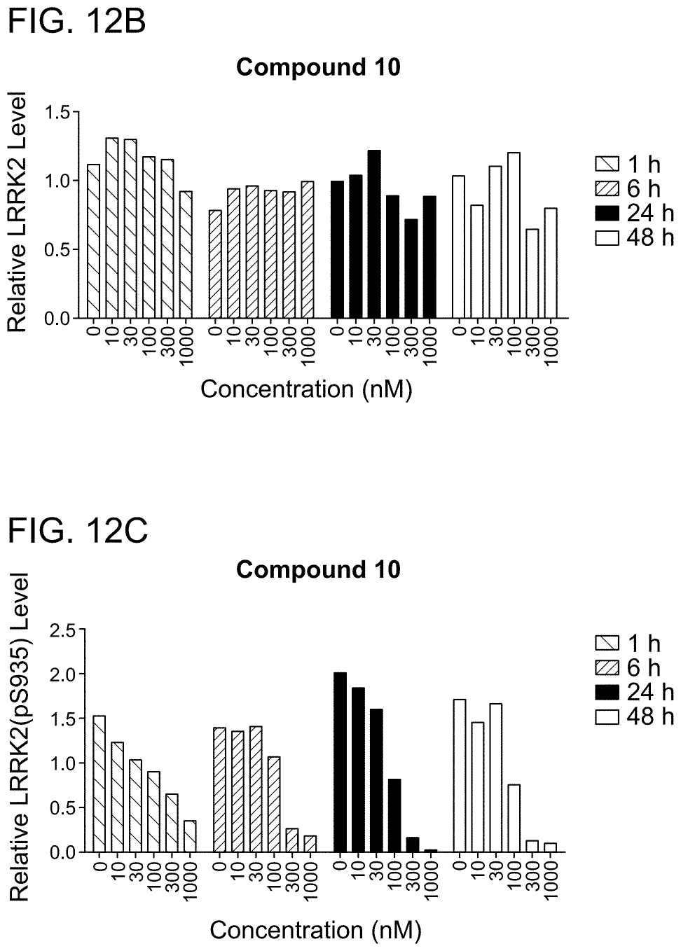

The results in FIG. 12A-FIG. 12C show that inventive compound 10 inhibited the phosphorylation of S935 as well as the MLi-2 analog. Less degradation of LRRK2 was observed with compound 10 compared to compound 9.

### Example 29: Cellular Degradation of LRRK2 with Inventive Compound 11

The experimental protocol is as in Example 17.

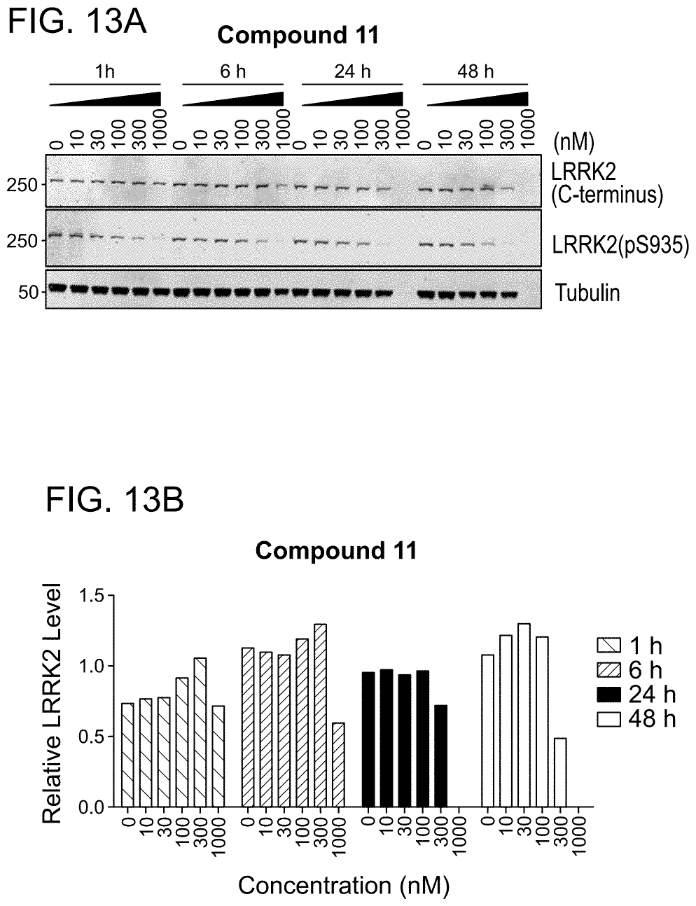

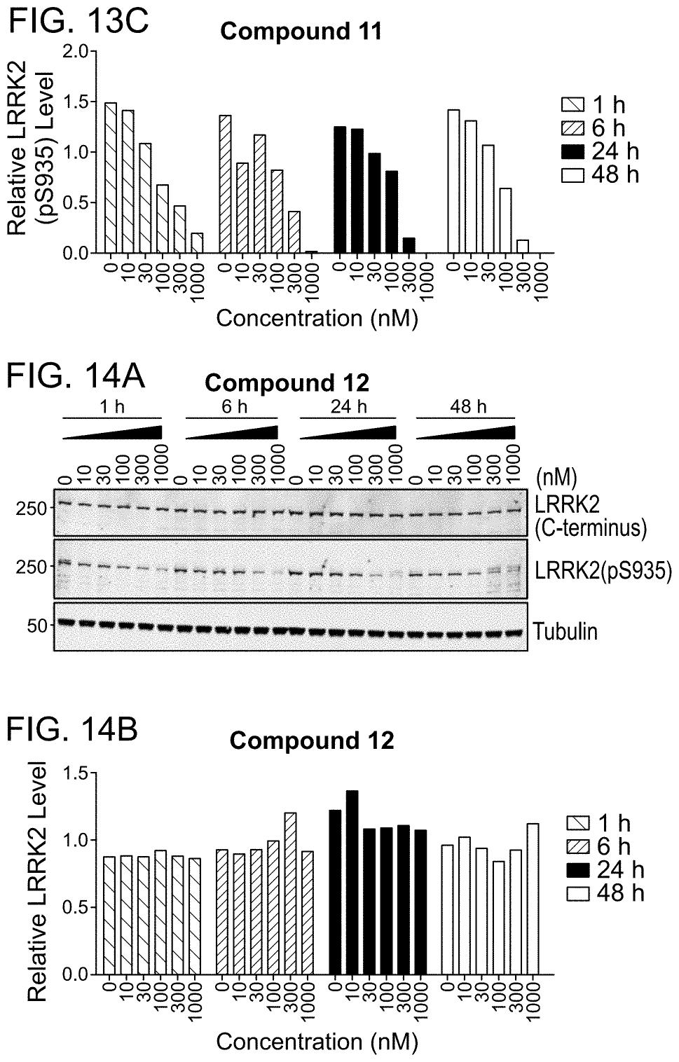

The results in FIG. 13A-FIG. 13C show that inventive compound 11 inhibited the phosphorylation of S935. Some degradation of LRRK2 was also observed.

### Example 30: Cellular Degradation of LRRK2 and LRRK2 pS935 with Inventive Compound 11

The experimental protocol is as in Example 17.

In FIG. 13A-FIG. 13C, the inventive compound 11 inhibits the phosphorylation of S935.

Some degradation of LRRK2 was also observed.

### Example 31: Cellular Degradation of LRRK2 with Inventive Compound 12

The experimental protocol is as in Example 17.

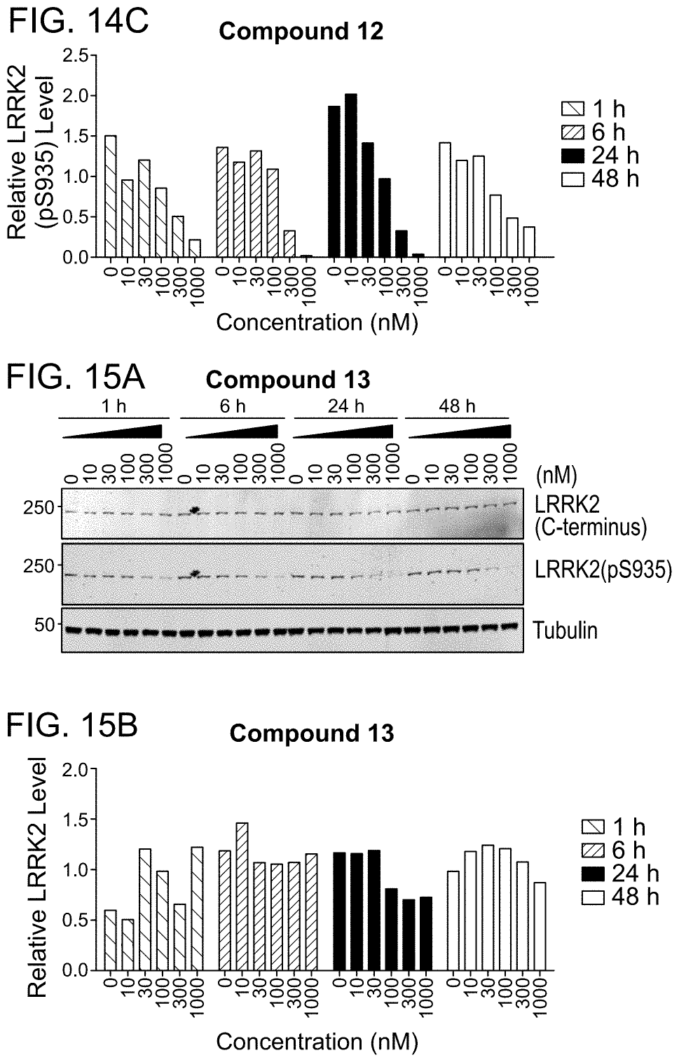

The results in FIG. 14A-FIG. 14C show that inventive compound 12 inhibited the phosphorylation of S935. Minor degradation of LRRK2 was also observed.

### Example 32: Cellular Degradation of LRRK2 with Inventive Compound 13

The experimental protocol is as in Example 17.

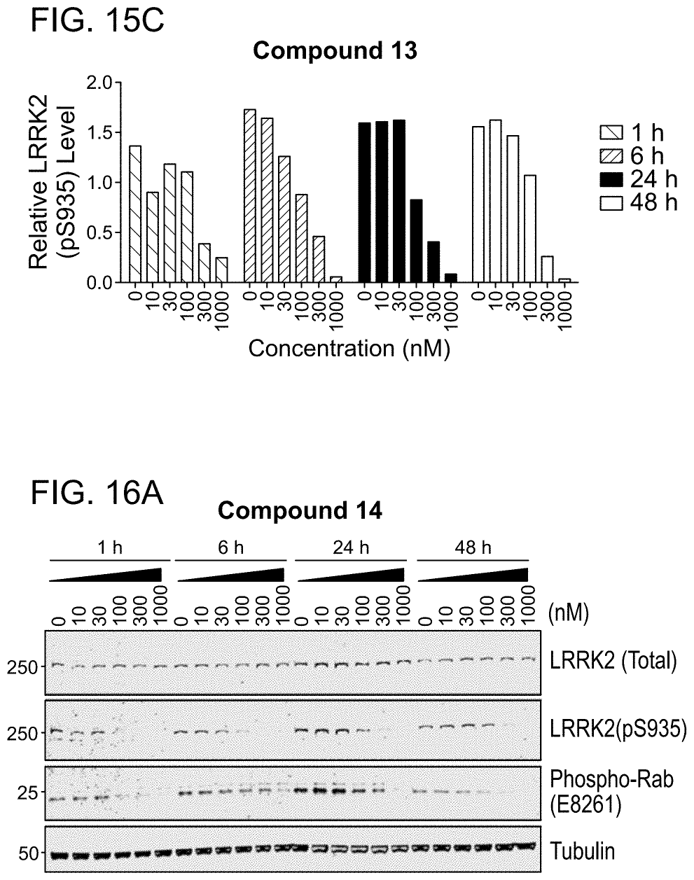

The results in FIG. 15A-FIG. 15C show that inventive compound 13 inhibited the phosphorylation of S935. Some degradation of LRRK2 was also observed.

TABLE 2
IC₅₀ of inventive compounds 8-13.

IC₅₀ (nM)

| Inventive Compound | LRRK2 wt | LRRK2 G2019S |
|---|---|---|
| 8 | 1.24 | 1.13 |
| 9 | 1.46 | 0.98 |
| 10 | 1.59 | 1.08 |
| 11 | 2.50 | 1.47 |
| 12 | 2.58 | 2.24 |
| 13 | 3.14 | 3.16 |

IC₅₀ values for the inventive compounds 8-13 are reported in the table above. The results show that the inventive compounds successfully inhibited WT LRRK2 and the phosphorylation of S935.

TABLE 3
LogP values for inventive compounds 8-11.

| Compound | 8 | 9 | 10 | 11 | 12 | 13 |
|---|---|---|---|---|---|---|
| CLogP | 3.56 | 3.20 | 3.73 | 3.50 | 3.33 | 2.98 |

Log P values for the inventive compounds 8-13 are set forth in the table above.

### Example 33: Cellular Degradation of LRRK2. LRRK2 pS935, and Phosoho-Rab (E826) with Inventive Compound 14

The experimental protocol is as in Example 17.

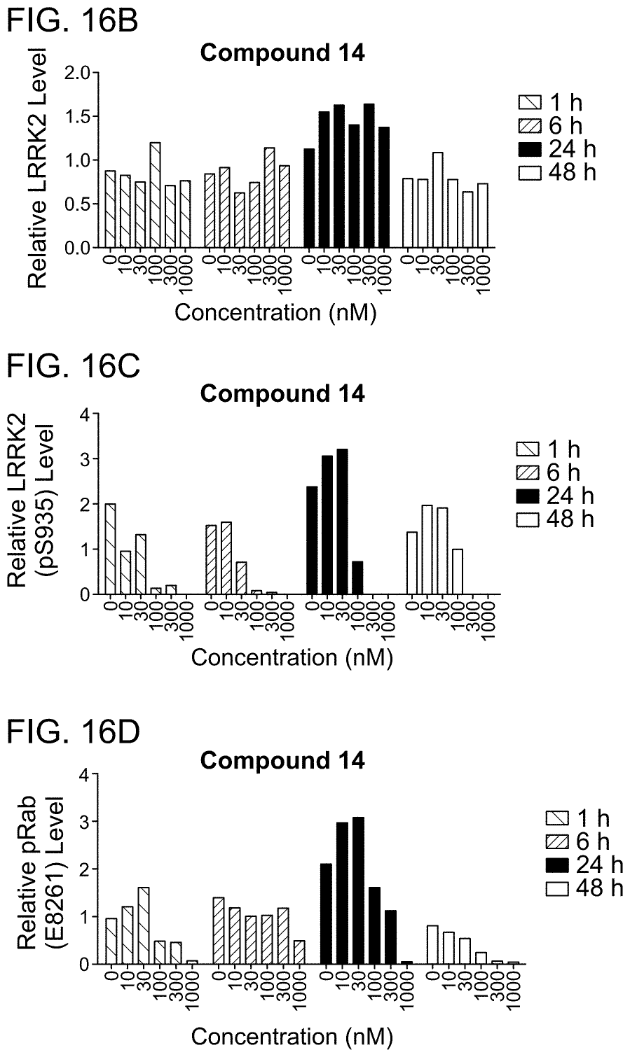

The results in FIG. 16A-FIG. 16D show that inventive compound 14 inhibited the phosphorylation of S935 and Rab(E826). No degradation of LRRK2 was also observed.

### Example 34: Cellular Degradation of LRRK2 with Inventive Compound 15

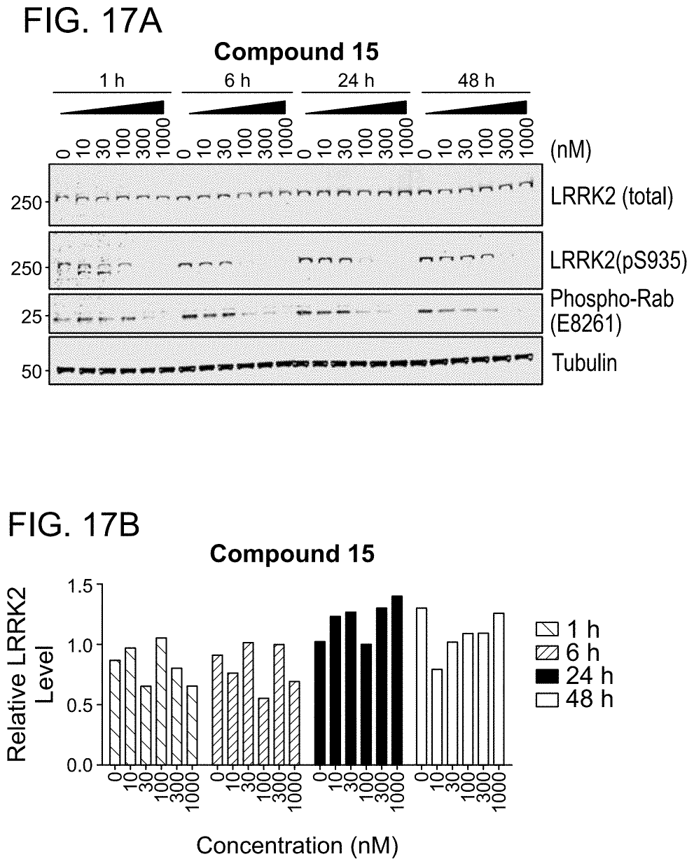

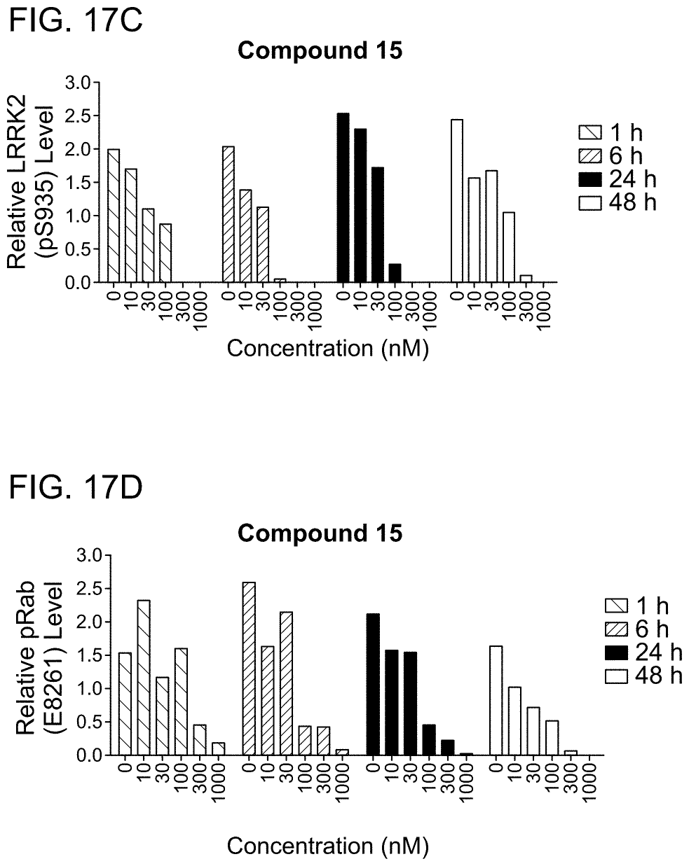

The results in FIG. 17A-FIG. 17D show that inventive compound 15 inhibited the phosphorylation of S935 and Rab(E826). No degradation of LRRK2 was also observed.

### Example 35: Cellular Degradation of LRRK2. LRRK2 pS935, and Phosoho-Rab (E826) with Inventive Compound 16 as a Negative Control

The experimental protocol is as in Example 17.

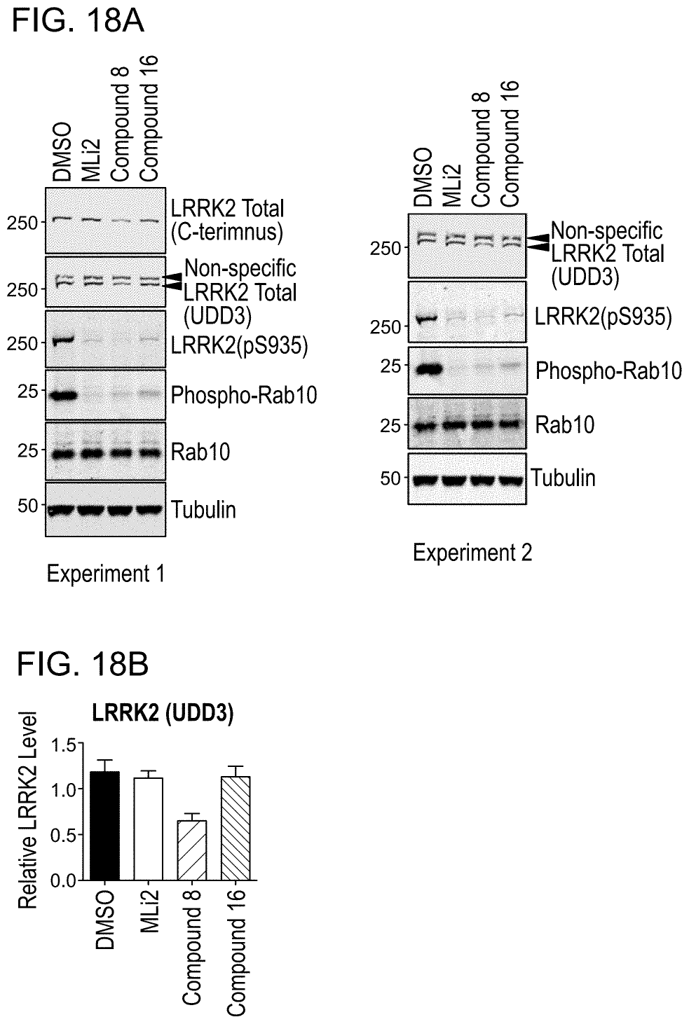

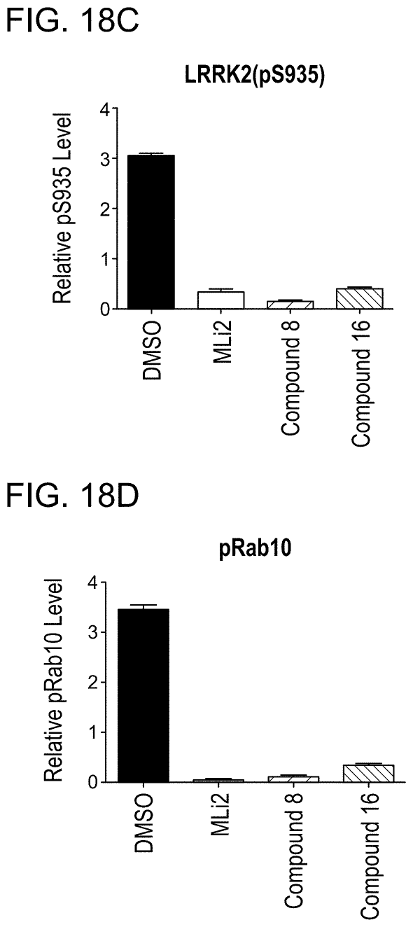

The results in FIG. 18A-FIG. 18D show that negative control 16 potently inhibited pS935 and pRAB10, but did not reduce the level of LRRK2, whereas positive control 8 showed similar inhibition of pS935 and pRAB10, and also degraded LRRK2.

All patent publications and non-patent publications are indicative of the level of skill of those skilled in the art to which this invention pertains. All these publications are herein incorporated by reference to the same extent as if each individual publication were specifically and individually indicated as being incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. bifunctional compound of formula (I), 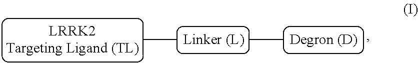
wherein the targeting ligand represents an aminopyrimidine or indazole that binds leucine-rich repeat kinase 2 (LRRK2), the degron represents a ligand that binds an E3 ubiquitin ligase, and the linker represents a moiety that connects covalently the degron and the targeting ligand, or a pharmaceutically acceptable salt or stereoisomer thereof.

2. The bifunctional compound of claim 1, wherein the LRRK2 targeting ligand is an aminopyrimidine.

3. The bifunctional compound of claim 2, wherein the aminopyrimidine has a structure represented by formula (TL1-a) or (TL1-b): 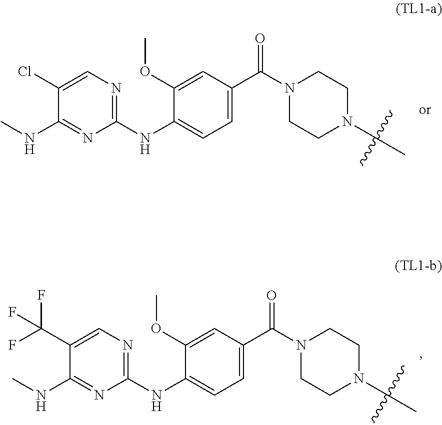 [SMILES: *N1CCN(C(=O)c2ccc(Nc3ncc(Cl)c(NC)n3)c(OC)c2)CC1 ; *N1CCN(C(=O)c2ccc(Nc3ncc(C(F)(F)F)c(NC)n3)c(OC)c2)CC1]
wherein the squiggle represents the point of attachment to

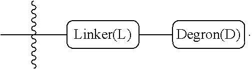

4. (canceled)

5. The bifunctional compound of claim 1, wherein the LRRK2 targeting ligand is an indazole.

6. bifunctional compound of claim 5, wherein the indazole has a structure represented by formula (TL2-a): 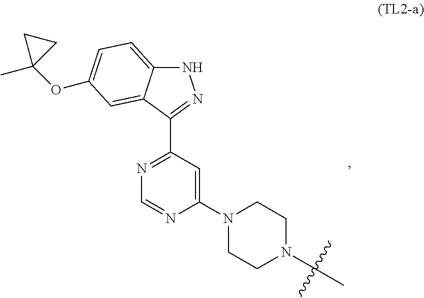 [SMILES: *N1CCN(c2cc(-c3n[nH]c4ccc(OC5(C)CC5)cc34)ncn2)CC1]
wherein the squiggle represents the point of attachment to

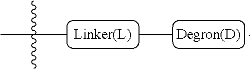

7. The bifunctional compound of claim 1, wherein the targeting ligand has a structure represented by formula (TL2-b): 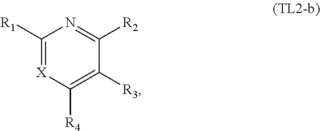
wherein:
X represents N, CR₅, or CR₆; wherein R₅ represents 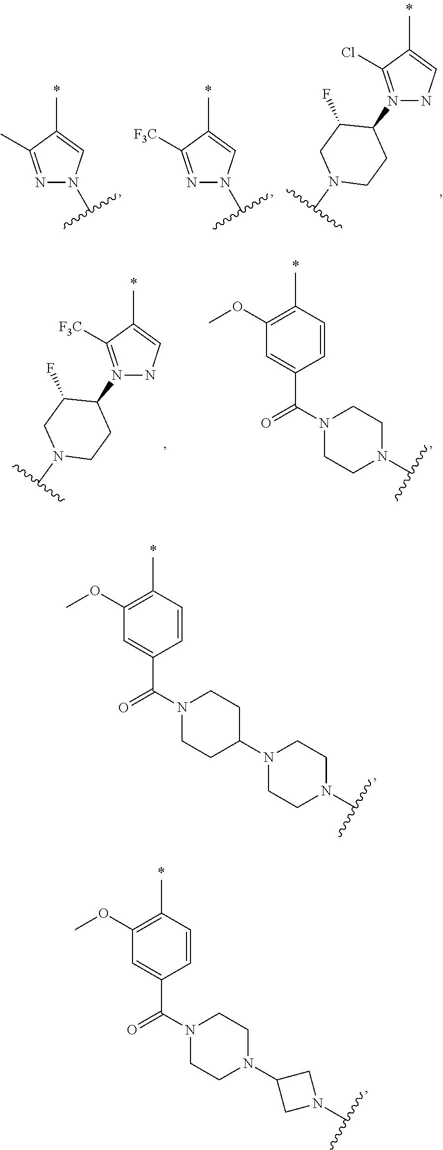 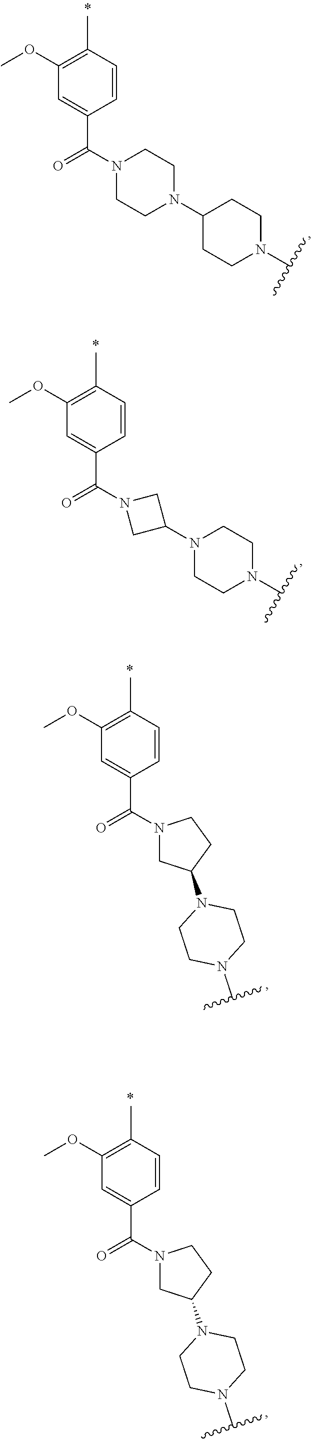 [SMILES: *N1CCC(N2CCN(C(=O)c3ccc(C)c(OC)c3)CC2)CC1 ; *N1CCN(C2CN(C(=O)c3ccc(C)c(OC)c3)C2)CC1 ; *N1CCN([C@@H]2CCN(C(=O)c3ccc(C)c(OC)c3)C2)CC1 ; *N1CCN([C@H]2CCN(C(=O)c3ccc(C)c(OC)c3)C2)CC1] 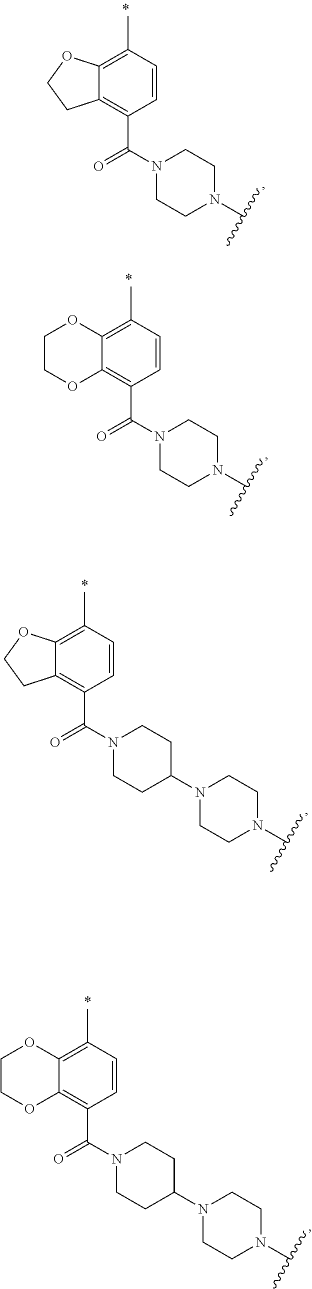 [SMILES: *N1CCN(C(=O)c2ccc(C)c3c2CCO3)CC1 ; *N1CCN(C(=O)c2ccc(C)c3c2OCCO3)CC1 ; *N1CCN(C2CCN(C(=O)c3ccc(C)c4c3CCO4)CC2)CC1 ; *N1CCN(C2CCN(C(=O)c3ccc(C)c4c3OCCO4)CC2)CC1] 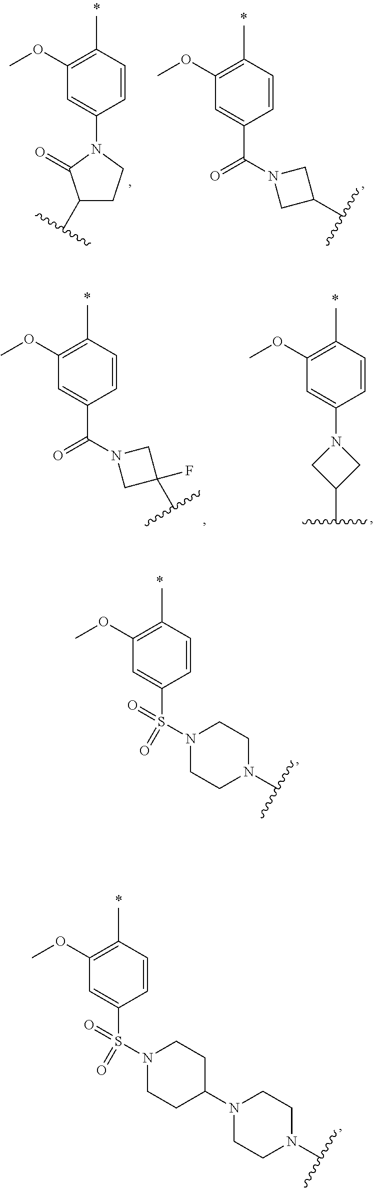 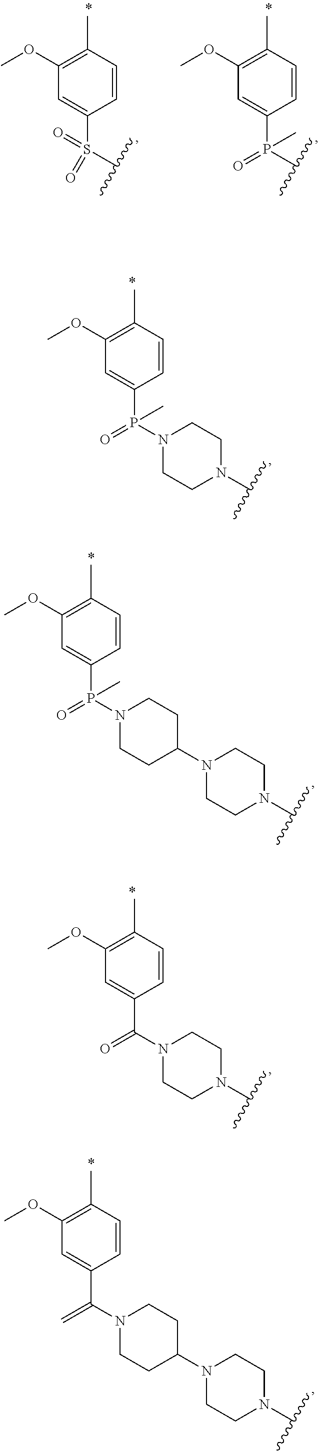 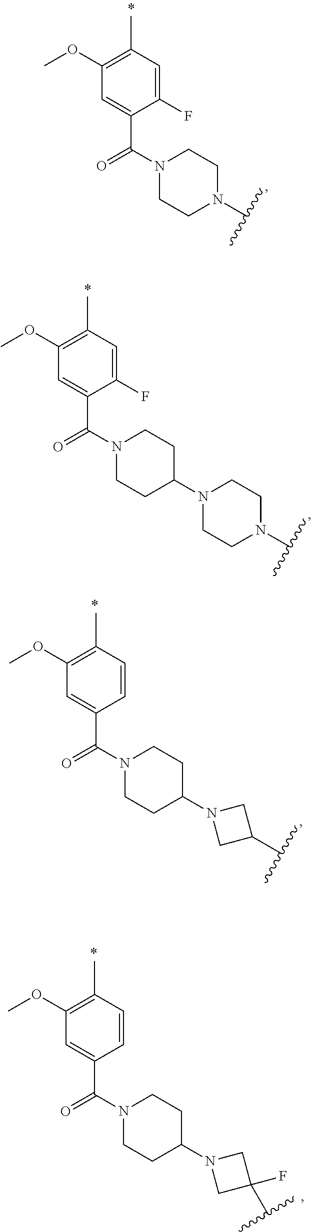 [SMILES: *N1CCN(C(=O)c2cc(OC)c(C)cc2F)CC1 ; *N1CCN(C2CCN(C(=O)c3cc(OC)c(C)cc3F)CC2)CC1 ; *C1CN(C2CCN(C(=O)c3ccc(C)c(OC)c3)CC2)C1 ; *C1(F)CN(C2CCN(C(=O)c3ccc(C)c(OC)c3)CC2)C1] 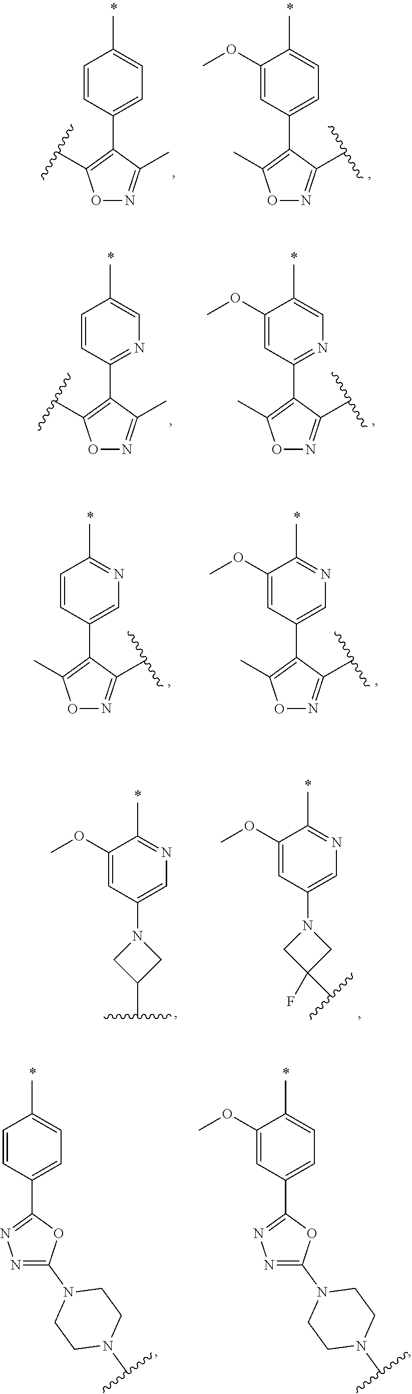 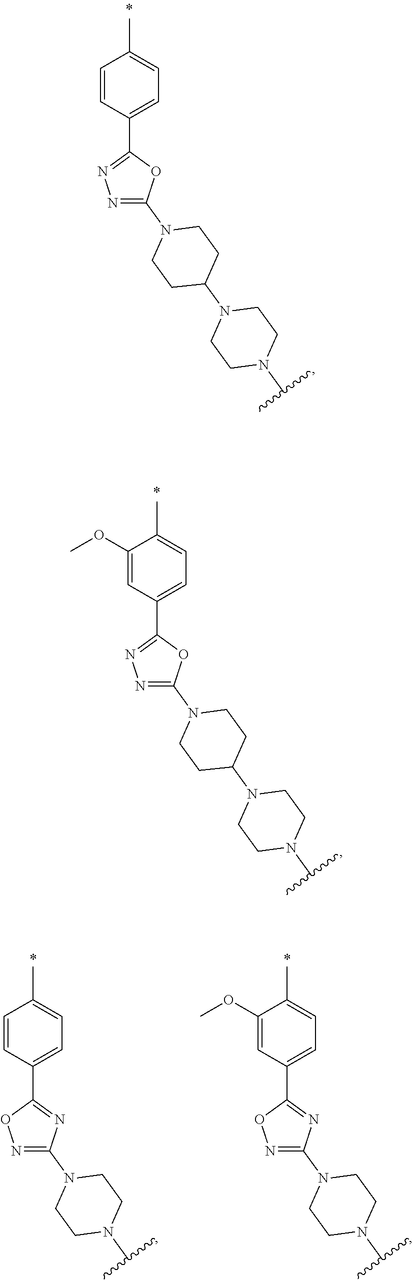 [SMILES: *N1CCN(C2CCN(c3nnc(-c4ccc(C)cc4)o3)CC2)CC1 ; *N1CCN(C2CCN(c3nnc(-c4ccc(C)c(OC)c4)o3)CC2)CC1 ; *N1CCN(c2noc(-c3ccc(C)cc3)n2)CC1 ; *N1CCN(c2noc(-c3ccc(C)c(OC)c3)n2)CC1] 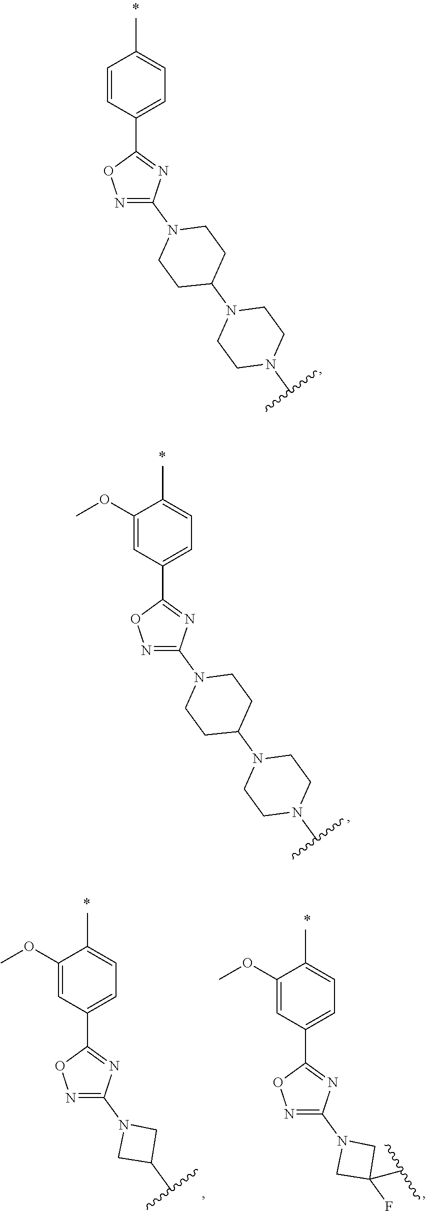 [SMILES: *N1CCN(C2CCN(c3noc(-c4ccc(C)cc4)n3)CC2)CC1 ; *N1CCN(C2CCN(c3noc(-c4ccc(C)c(OC)c4)n3)CC2)CC1 ; *C1CN(c2noc(-c3ccc(C)c(OC)c3)n2)C1 ; *C1(F)CN(c2noc(-c3ccc(C)c(OC)c3)n2)C1] 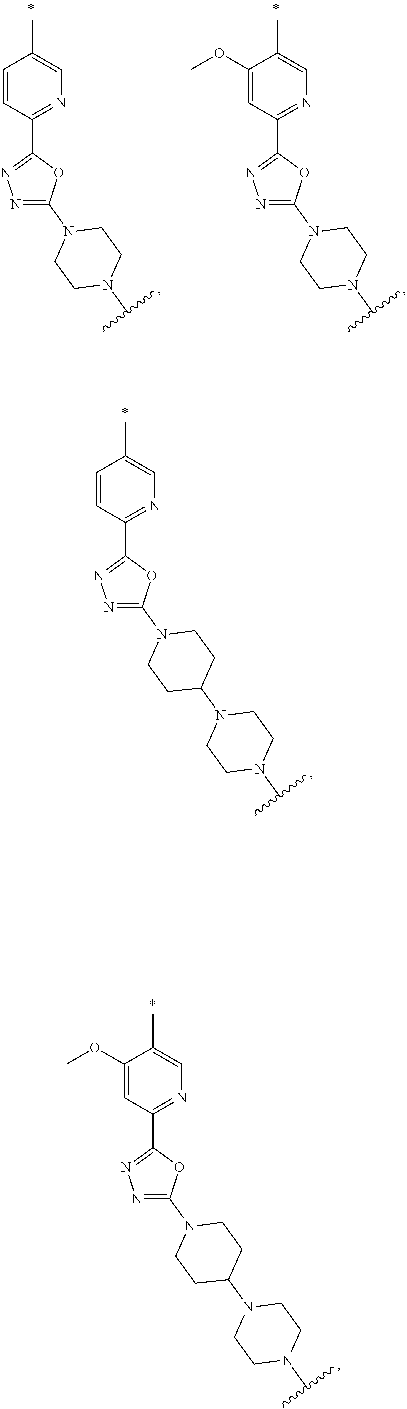 [SMILES: *N1CCN(c2nnc(-c3ccc(C)cn3)o2)CC1 ; *N1CCN(c2nnc(-c3cc(OC)c(C)cn3)o2)CC1 ; *N1CCN(C2CCN(c3nnc(-c4ccc(C)cn4)o3)CC2)CC1 ; *N1CCN(C2CCN(c3nnc(-c4cc(OC)c(C)cn4)o3)CC2)CC1] 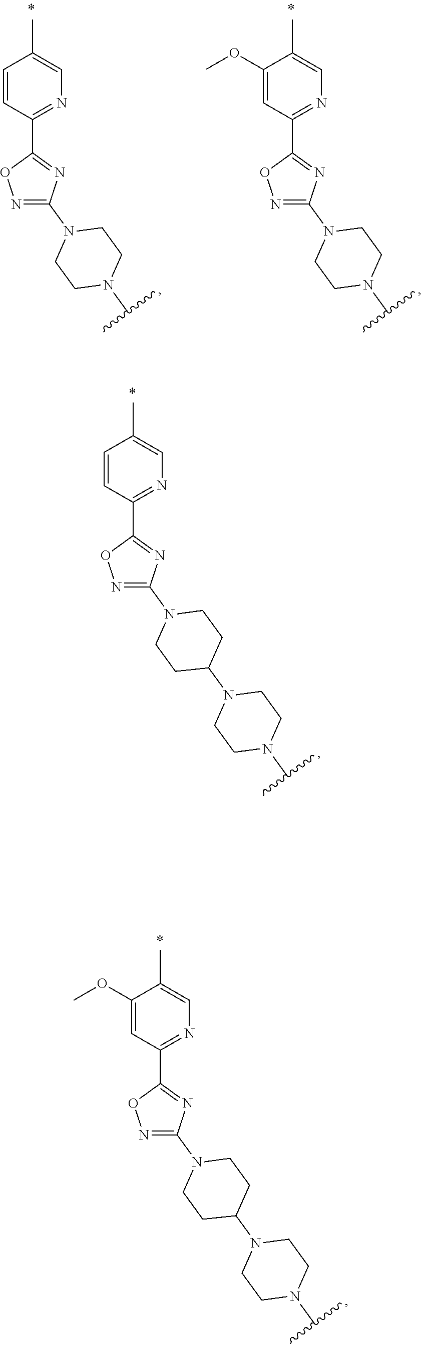 [SMILES: *N1CCN(c2noc(-c3ccc(C)cn3)n2)CC1 ; *N1CCN(c2noc(-c3cc(OC)c(C)cn3)n2)CC1 ; *N1CCN(C2CCN(c3noc(-c4ccc(C)cn4)n3)CC2)CC1 ; *N1CCN(C2CCN(c3noc(-c4cc(OC)c(C)cn4)n3)CC2)CC1] 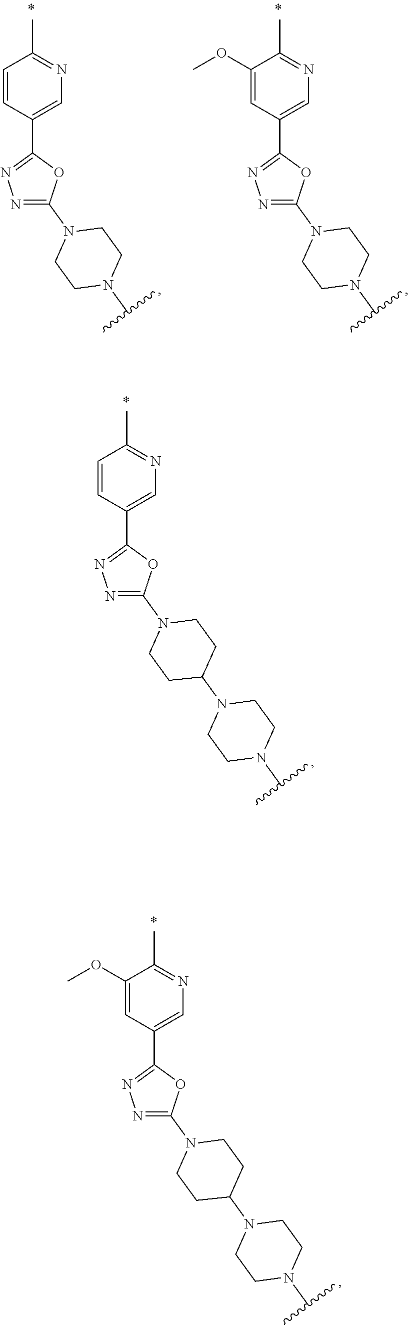 [SMILES: *N1CCN(c2nnc(-c3ccc(C)nc3)o2)CC1 ; *N1CCN(c2nnc(-c3cnc(C)c(OC)c3)o2)CC1 ; *N1CCN(C2CCN(c3nnc(-c4ccc(C)nc4)o3)CC2)CC1 ; *N1CCN(C2CCN(c3nnc(-c4cnc(C)c(OC)c4)o3)CC2)CC1] 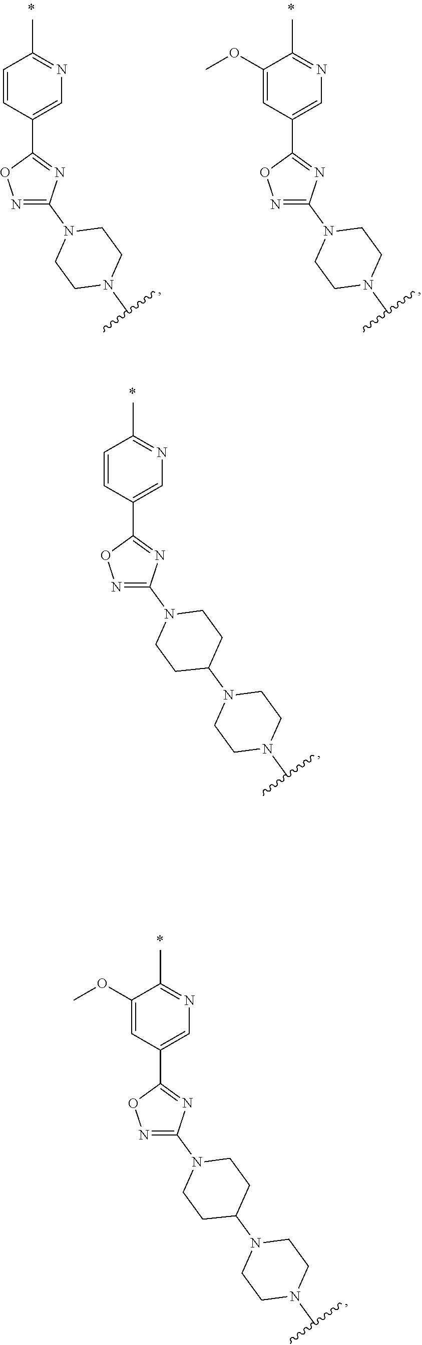 [SMILES: *N1CCN(c2noc(-c3ccc(C)nc3)n2)CC1 ; *N1CCN(c2noc(-c3cnc(C)c(OC)c3)n2)CC1 ; *N1CCN(C2CCN(c3noc(-c4ccc(C)nc4)n3)CC2)CC1 ; *N1CCN(C2CCN(c3noc(-c4cnc(C)c(OC)c4)n3)CC2)CC1] 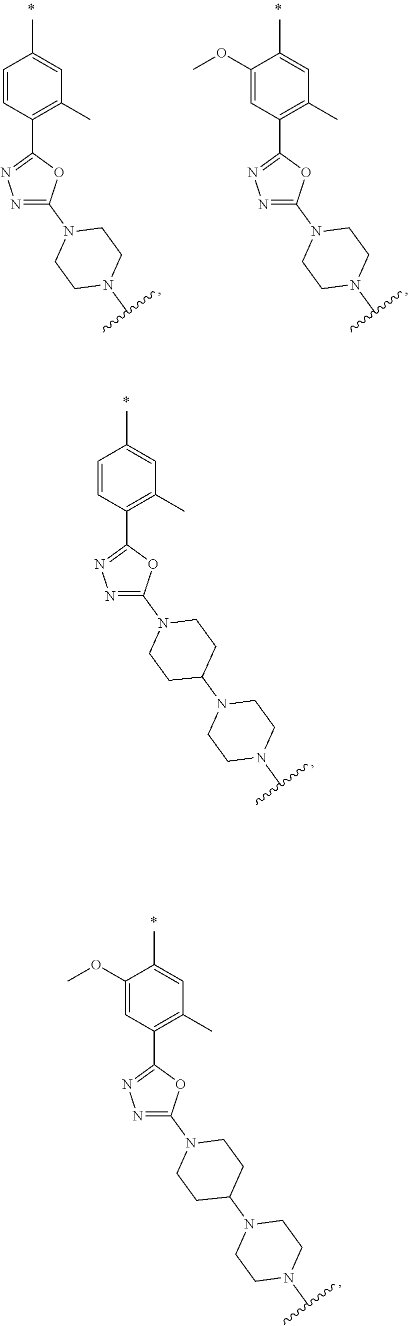 [SMILES: *N1CCN(c2nnc(-c3ccc(C)cc3C)o2)CC1 ; *N1CCN(c2nnc(-c3cc(OC)c(C)cc3C)o2)CC1 ; *N1CCN(C2CCN(c3nnc(-c4ccc(C)cc4C)o3)CC2)CC1 ; *N1CCN(C2CCN(c3nnc(-c4cc(OC)c(C)cc4C)o3)CC2)CC1] 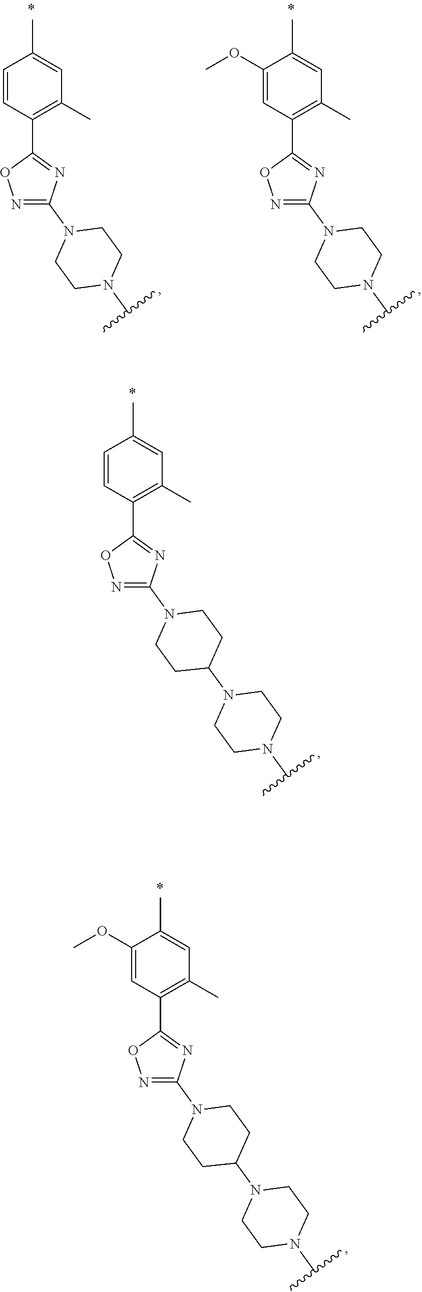 [SMILES: *N1CCN(c2noc(-c3ccc(C)cc3C)n2)CC1 ; *N1CCN(c2noc(-c3cc(OC)c(C)cc3C)n2)CC1 ; *N1CCN(C2CCN(c3noc(-c4ccc(C)cc4C)n3)CC2)CC1 ; *N1CCN(C2CCN(c3noc(-c4cc(OC)c(C)cc4C)n3)CC2)CC1] 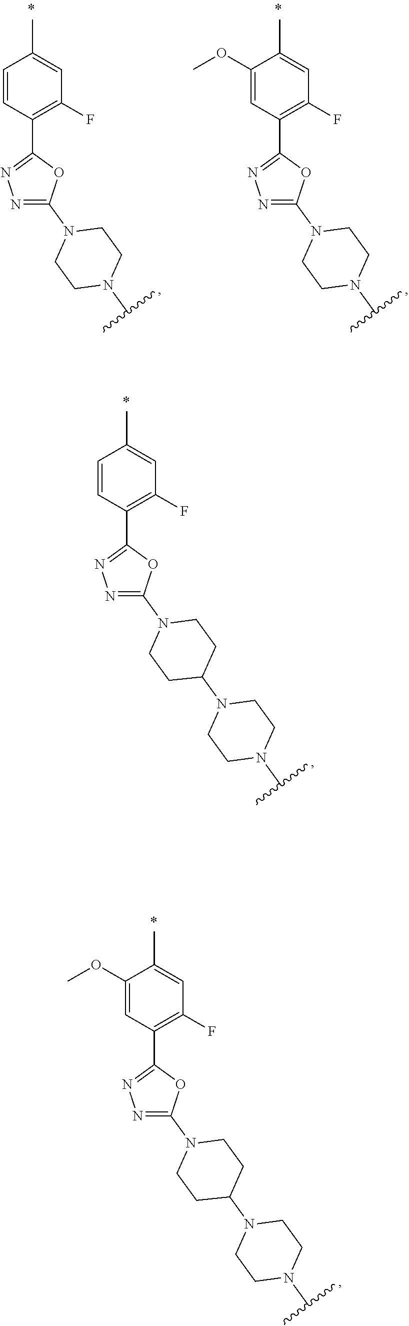 [SMILES: *N1CCN(c2nnc(-c3ccc(C)cc3F)o2)CC1 ; *N1CCN(c2nnc(-c3cc(OC)c(C)cc3F)o2)CC1 ; *N1CCN(C2CCN(c3nnc(-c4ccc(C)cc4F)o3)CC2)CC1 ; *N1CCN(C2CCN(c3nnc(-c4cc(OC)c(C)cc4F)o3)CC2)CC1] 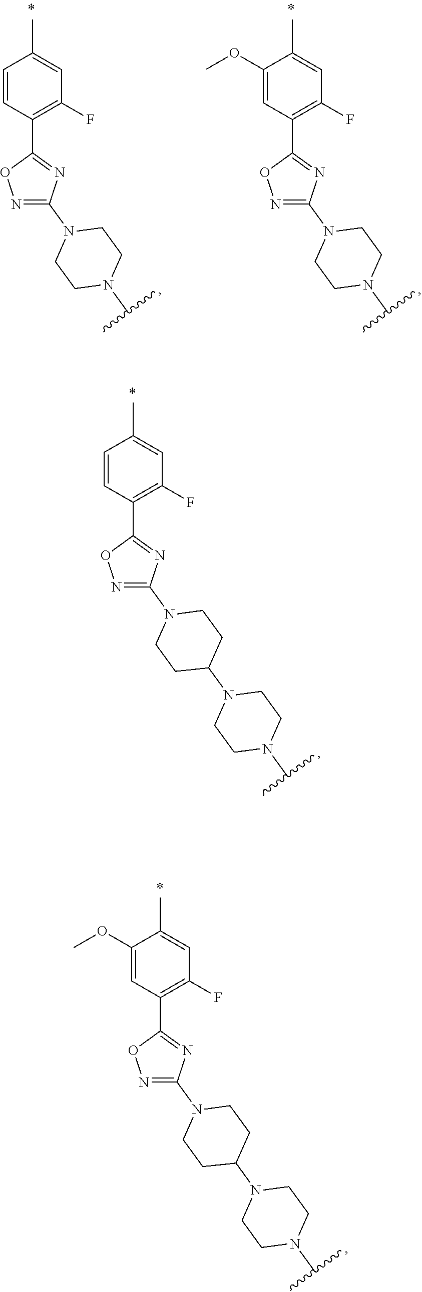 [SMILES: *N1CCN(c2noc(-c3ccc(C)cc3F)n2)CC1 ; *N1CCN(c2noc(-c3cc(OC)c(C)cc3F)n2)CC1 ; *N1CCN(C2CCN(c3noc(-c4ccc(C)cc4F)n3)CC2)CC1 ; *N1CCN(C2CCN(c3noc(-c4cc(OC)c(C)cc4F)n3)CC2)CC1] 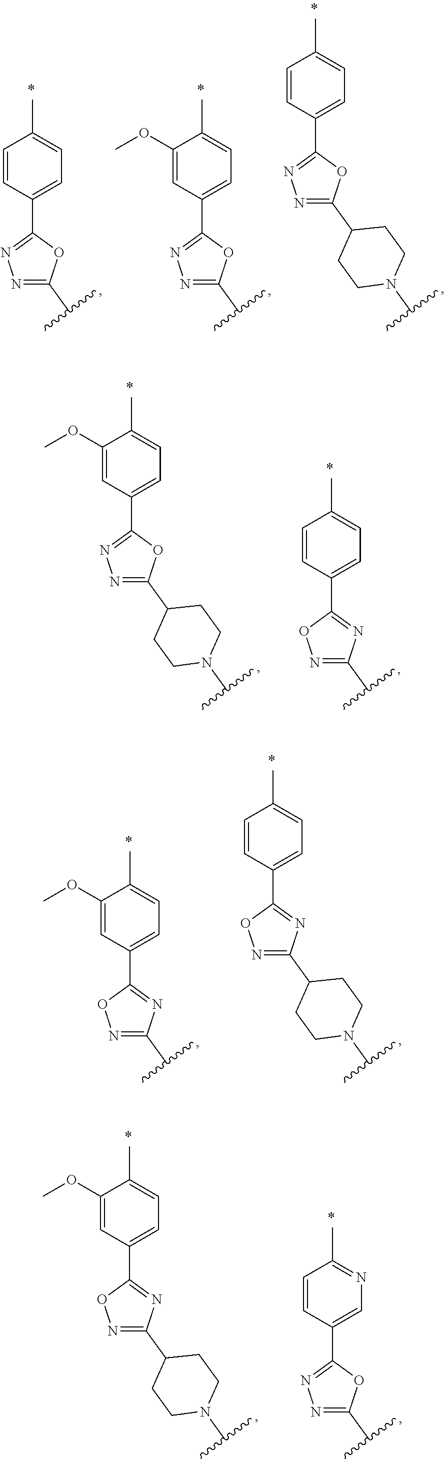 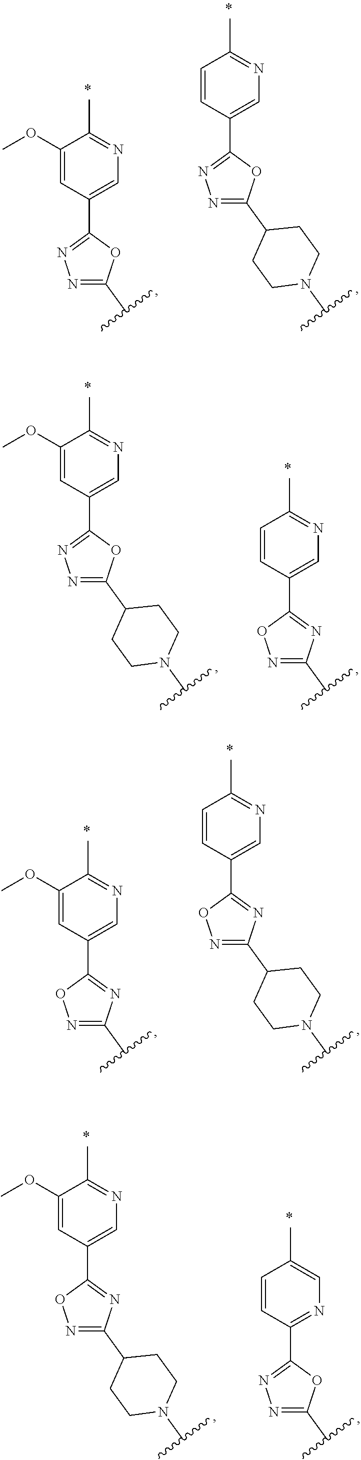 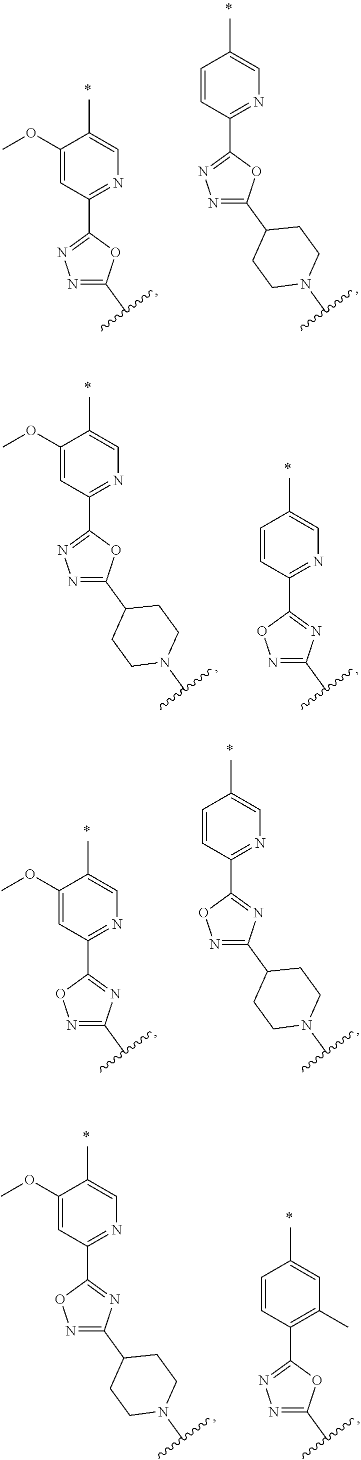 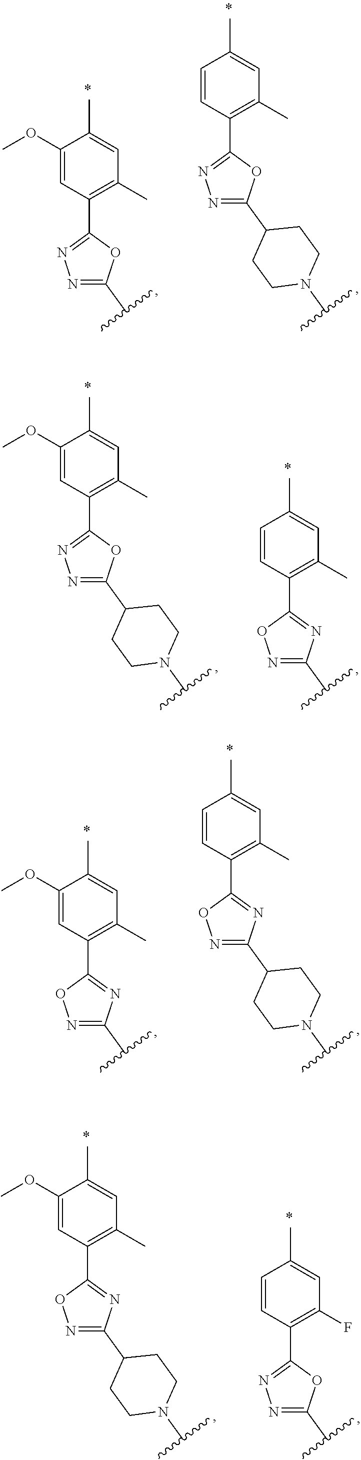 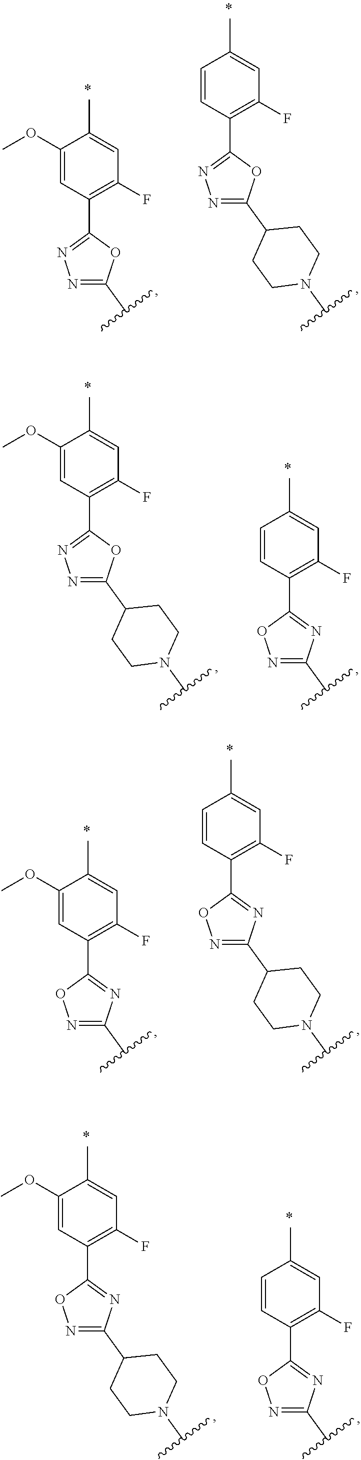 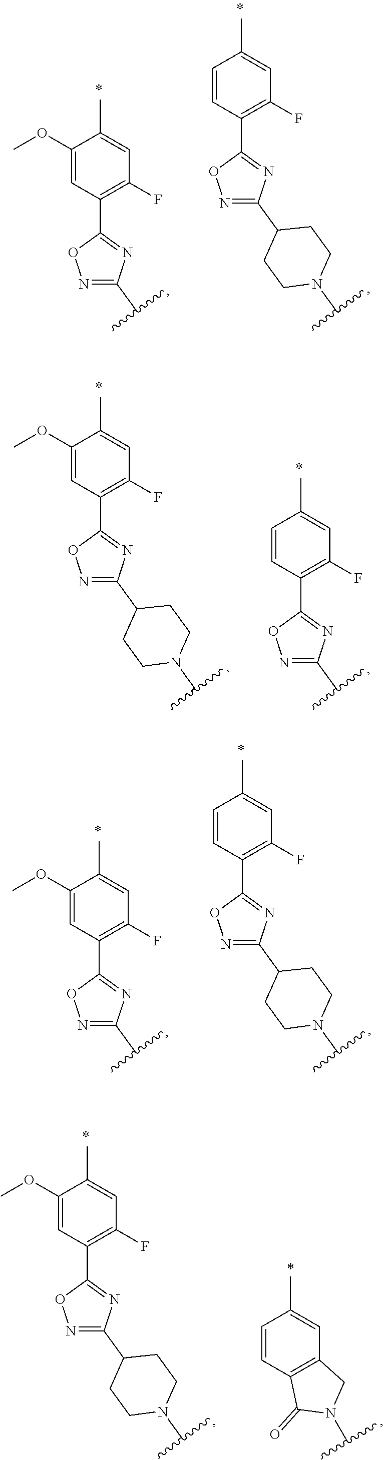 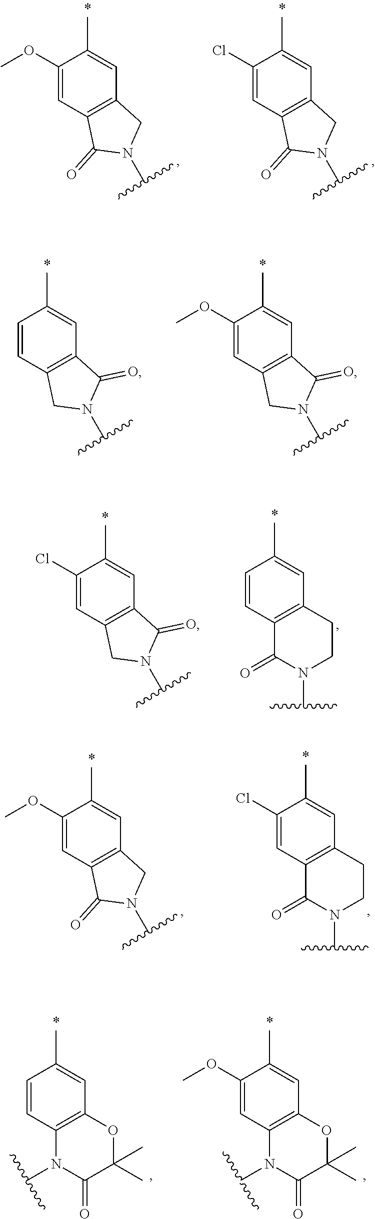 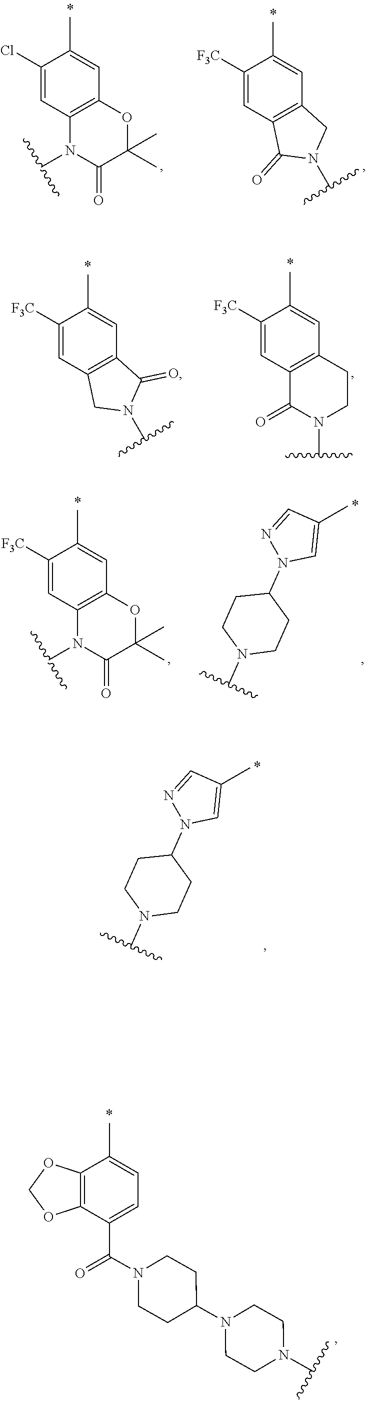 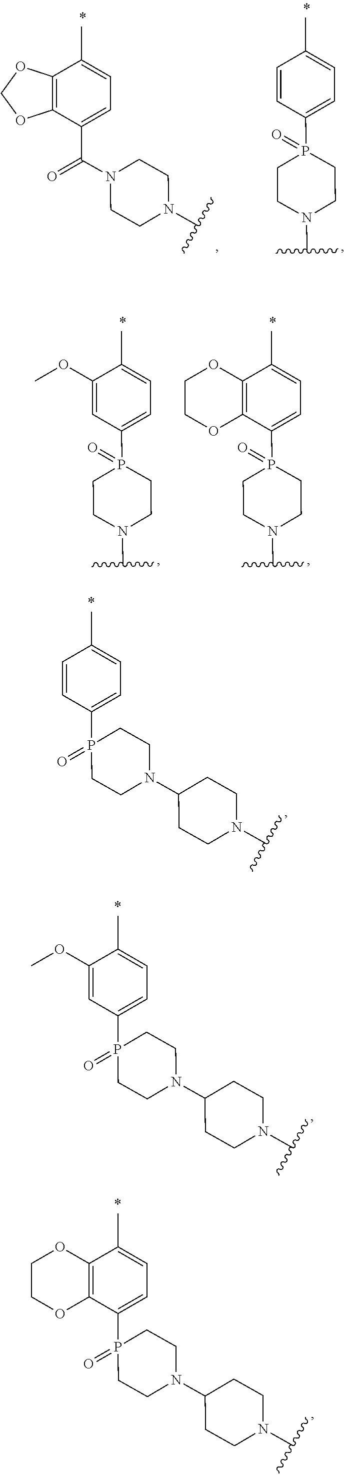 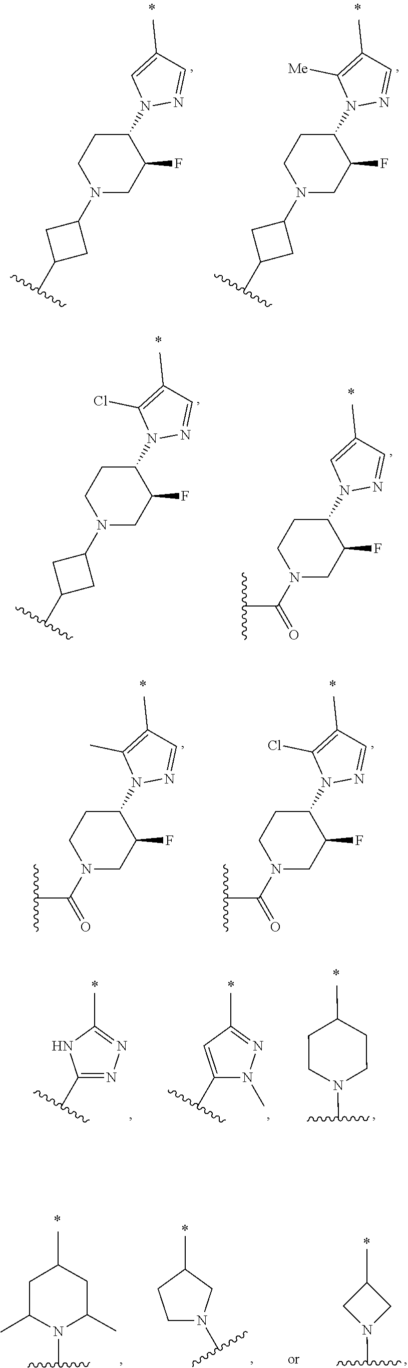
or represents H, wherein the asterisk (*) represents the point of attachment to the heterocyclic ring and the squiggle represents the point of attachment to 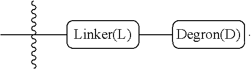
R₆ represents H, halo or CF₃;
R₁ represents 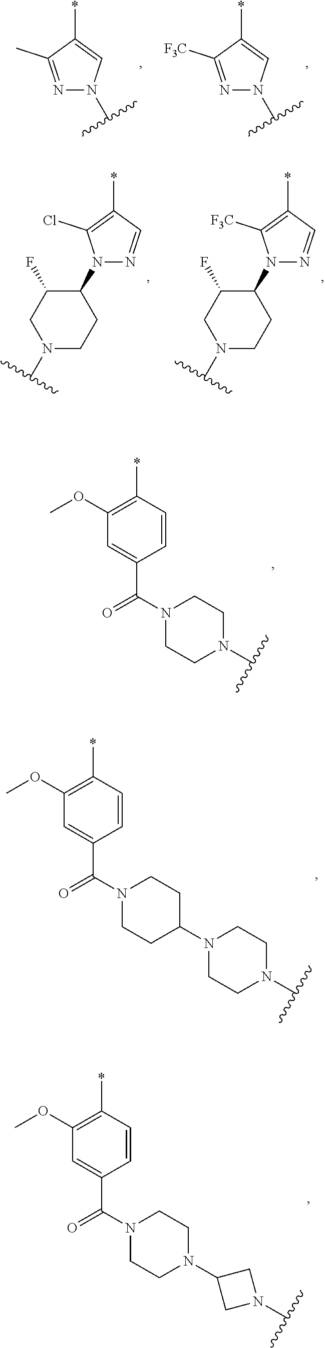 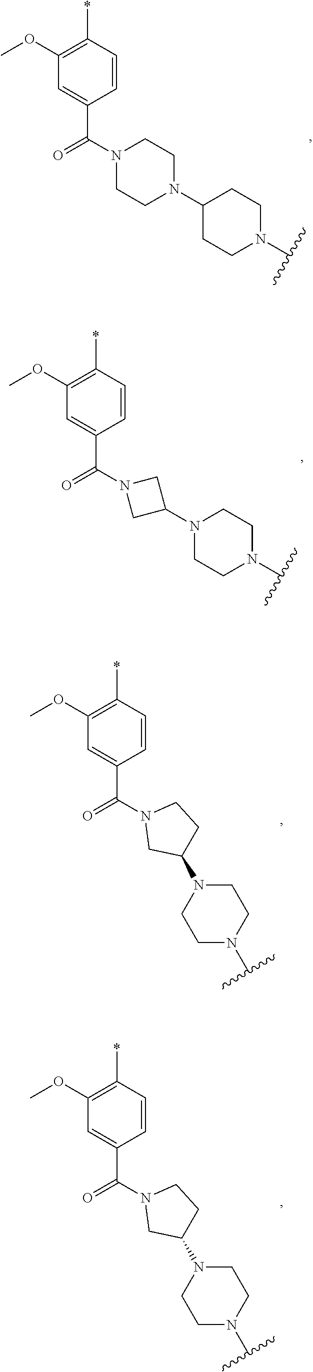 [SMILES: *c1ccc(C(=O)N2CCN(C3CCN(*)CC3)CC2)cc1OC ; *c1ccc(C(=O)N2CC(N3CCN(*)CC3)C2)cc1OC ; *c1ccc(C(=O)N2CC[C@@H](N3CCN(*)CC3)C2)cc1OC ; *c1ccc(C(=O)N2CC[C@H](N3CCN(*)CC3)C2)cc1OC] 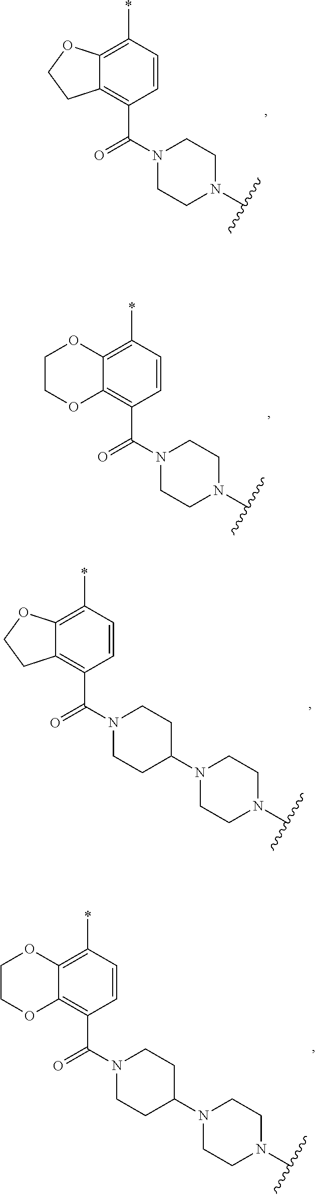 [SMILES: *c1ccc(C(=O)N2CCN(*)CC2)c2c1OCC2 ; *c1ccc(C(=O)N2CCN(*)CC2)c2c1OCCO2 ; *c1ccc(C(=O)N2CCC(N3CCN(*)CC3)CC2)c2c1OCC2 ; *c1ccc(C(=O)N2CCC(N3CCN(*)CC3)CC2)c2c1OCCO2] 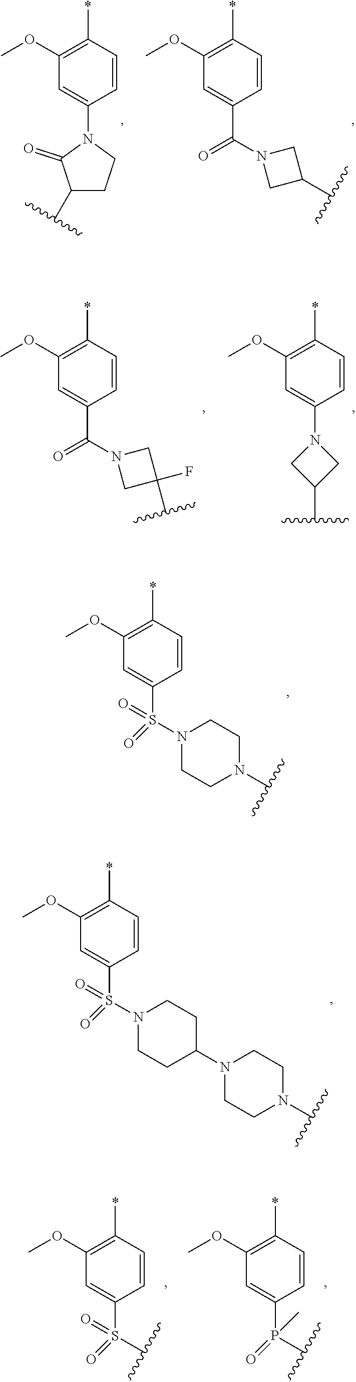 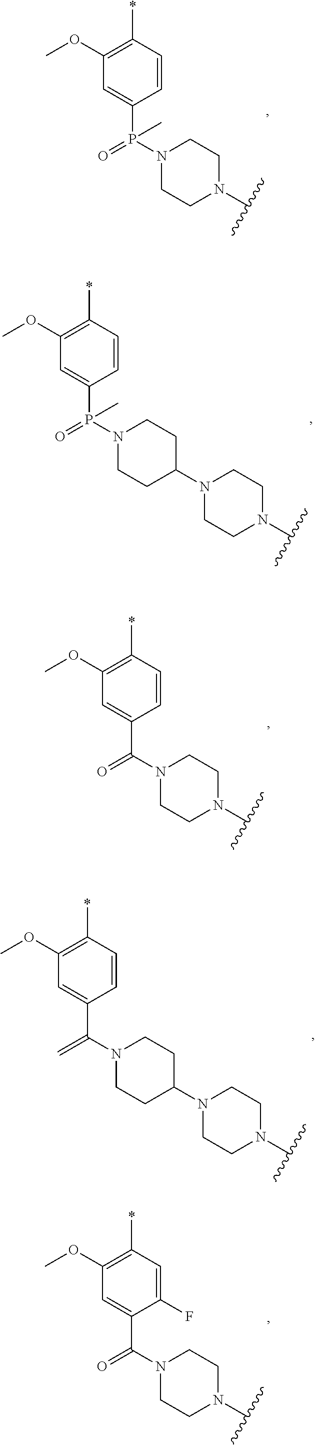 [SMILES: *c1ccc(P(C)(=O)N2CCN(*)CC2)cc1OC ; *c1ccc(P(C)(=O)N2CCC(N3CCN(*)CC3)CC2)cc1OC ; *c1ccc(C(=O)N2CCN(*)CC2)cc1OC ; *c1ccc(C(=C)N2CCC(N3CCN(*)CC3)CC2)cc1OC ; *c1cc(F)c(C(=O)N2CCN(*)CC2)cc1OC] 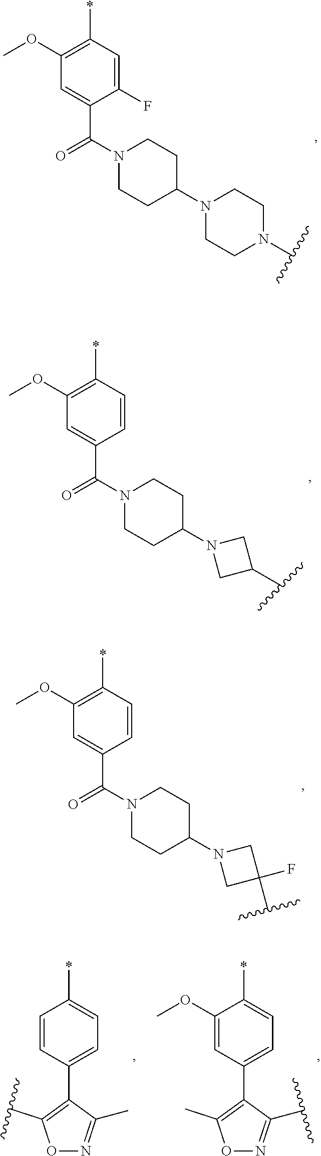 [SMILES: *c1cc(F)c(C(=O)N2CCC(N3CCN(*)CC3)CC2)cc1OC ; *c1ccc(C(=O)N2CCC(N3CC(*)C3)CC2)cc1OC ; *c1ccc(C(=O)N2CCC(N3CC(*)(F)C3)CC2)cc1OC ; *c1ccc(-c2c(C)noc2*)cc1 ; *c1ccc(-c2c(*)noc2C)cc1OC] 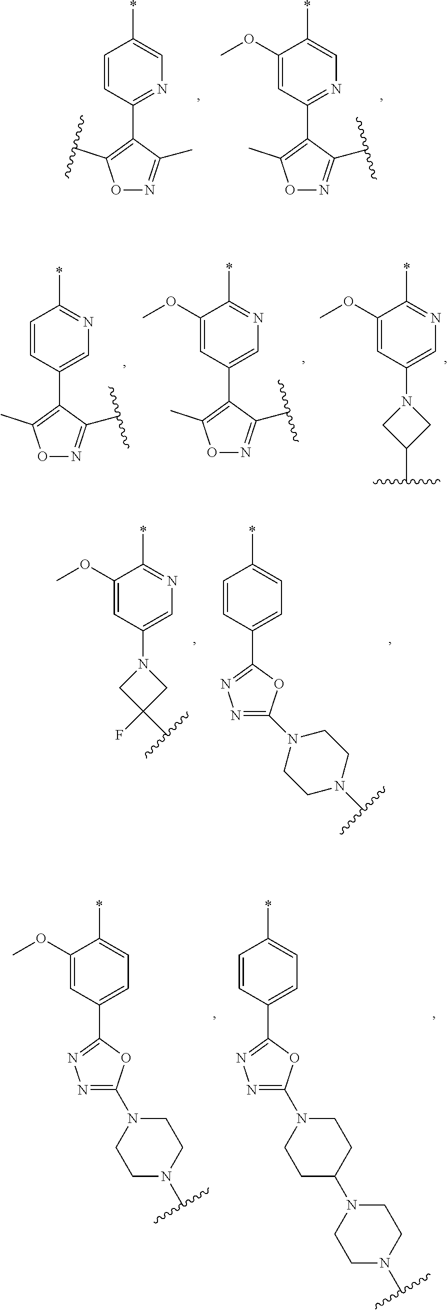 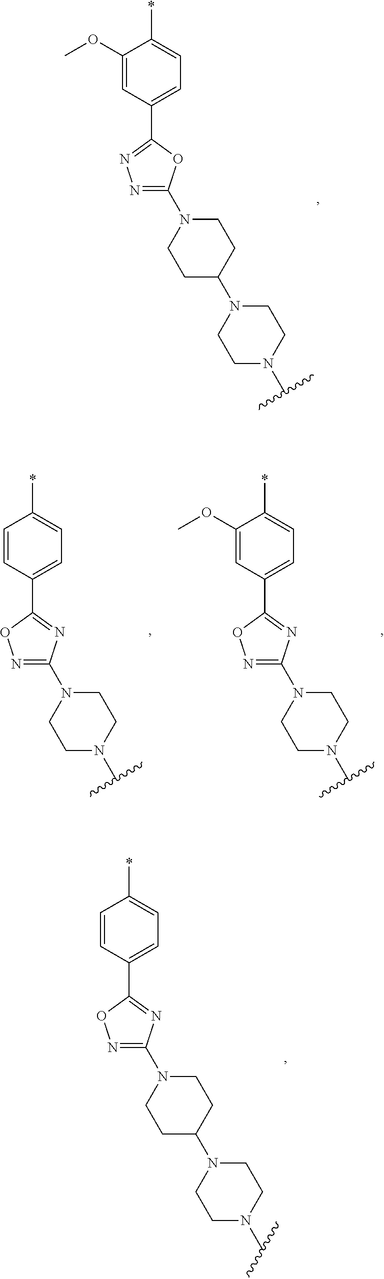 [SMILES: *c1ccc(-c2nnc(N3CCC(N4CCN(*)CC4)CC3)o2)cc1OC ; *c1ccc(-c2nc(N3CCN(*)CC3)no2)cc1 ; *c1ccc(-c2nc(N3CCN(*)CC3)no2)cc1OC ; *c1ccc(-c2nc(N3CCC(N4CCN(*)CC4)CC3)no2)cc1] 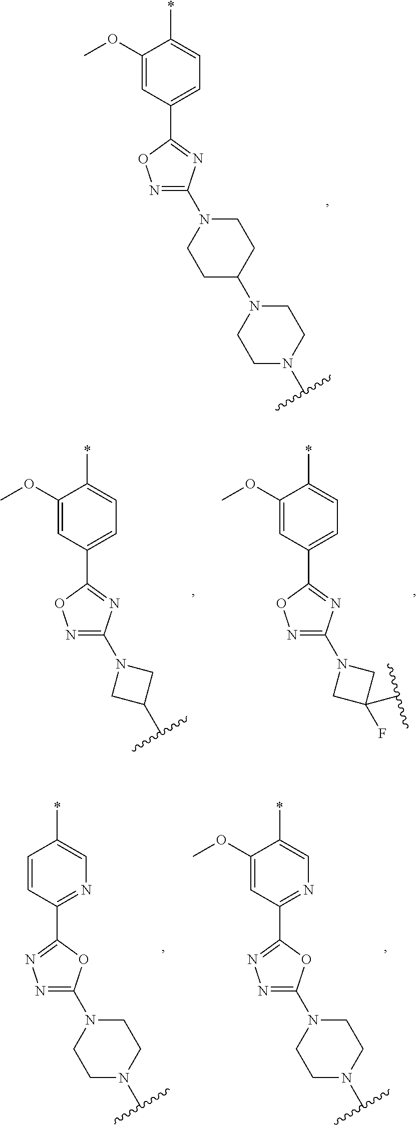 [SMILES: *c1ccc(-c2nc(N3CCC(N4CCN(*)CC4)CC3)no2)cc1OC ; *c1ccc(-c2nc(N3CC(*)C3)no2)cc1OC ; *c1ccc(-c2nc(N3CC(*)(F)C3)no2)cc1OC ; *c1ccc(-c2nnc(N3CCN(*)CC3)o2)nc1 ; *c1cnc(-c2nnc(N3CCN(*)CC3)o2)cc1OC] 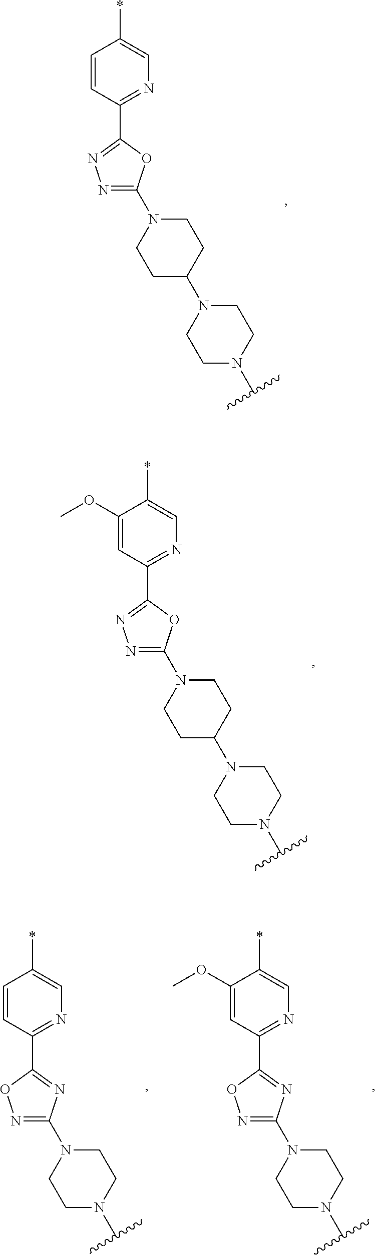 [SMILES: *c1ccc(-c2nnc(N3CCC(N4CCN(*)CC4)CC3)o2)nc1 ; *c1cnc(-c2nnc(N3CCC(N4CCN(*)CC4)CC3)o2)cc1OC ; *c1ccc(-c2nc(N3CCN(*)CC3)no2)nc1 ; *c1cnc(-c2nc(N3CCN(*)CC3)no2)cc1OC] 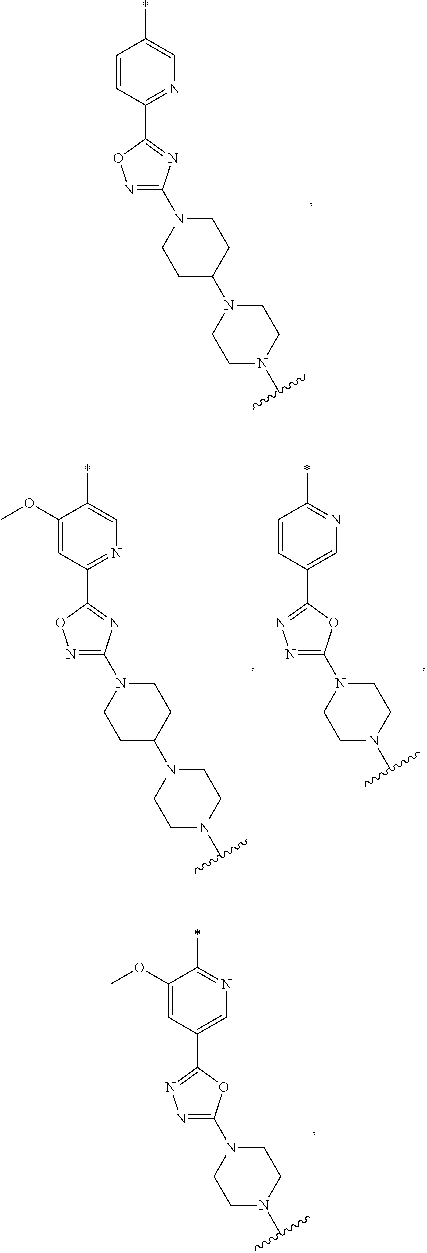 [SMILES: *c1ccc(-c2nc(N3CCC(N4CCN(*)CC4)CC3)no2)nc1 ; *c1cnc(-c2nc(N3CCC(N4CCN(*)CC4)CC3)no2)cc1OC ; *c1ccc(-c2nnc(N3CCN(*)CC3)o2)cn1 ; *c1ncc(-c2nnc(N3CCN(*)CC3)o2)cc1OC] 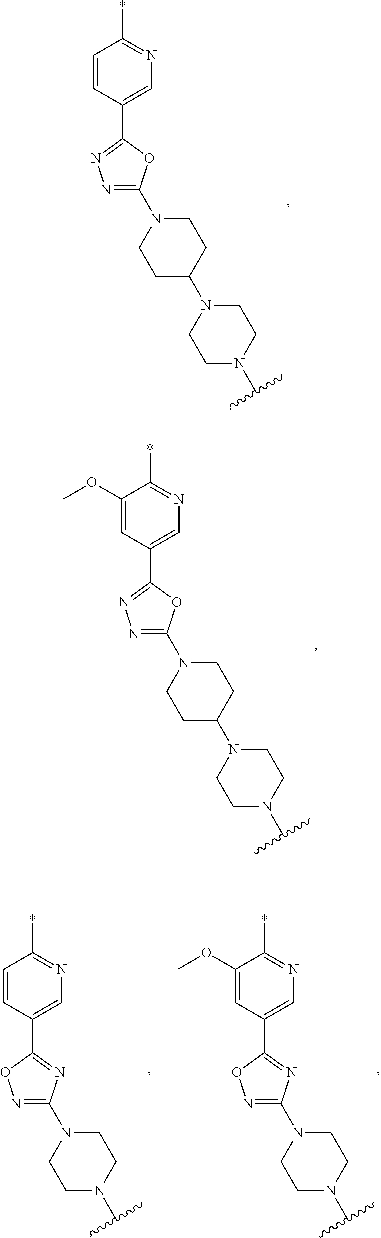 [SMILES: *c1ccc(-c2nnc(N3CCC(N4CCN(*)CC4)CC3)o2)cn1 ; *c1ncc(-c2nnc(N3CCC(N4CCN(*)CC4)CC3)o2)cc1OC ; *c1ccc(-c2nc(N3CCN(*)CC3)no2)cn1 ; *c1ncc(-c2nc(N3CCN(*)CC3)no2)cc1OC] 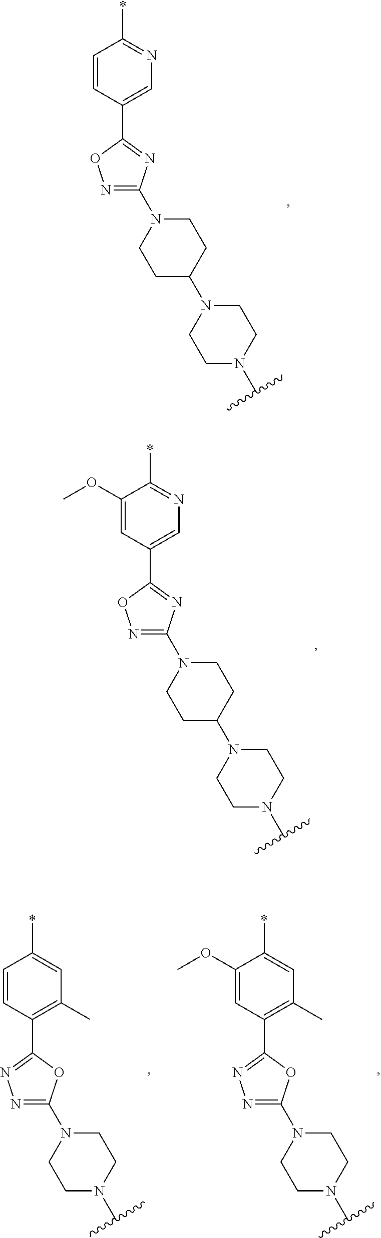 [SMILES: *c1ccc(-c2nc(N3CCC(N4CCN(*)CC4)CC3)no2)cn1 ; *c1ncc(-c2nc(N3CCC(N4CCN(*)CC4)CC3)no2)cc1OC ; *c1ccc(-c2nnc(N3CCN(*)CC3)o2)c(C)c1 ; *c1cc(C)c(-c2nnc(N3CCN(*)CC3)o2)cc1OC] 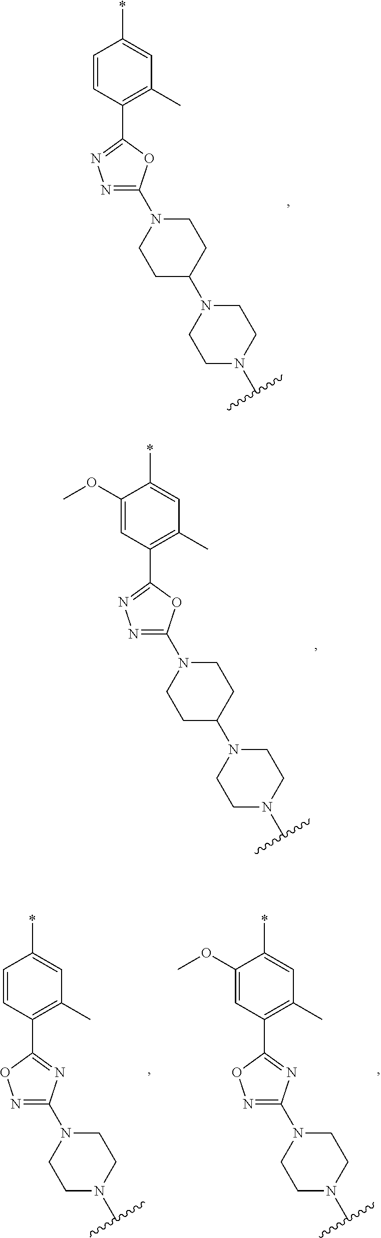 [SMILES: *c1ccc(-c2nnc(N3CCC(N4CCN(*)CC4)CC3)o2)c(C)c1 ; *c1cc(C)c(-c2nnc(N3CCC(N4CCN(*)CC4)CC3)o2)cc1OC ; *c1ccc(-c2nc(N3CCN(*)CC3)no2)c(C)c1 ; *c1cc(C)c(-c2nc(N3CCN(*)CC3)no2)cc1OC] 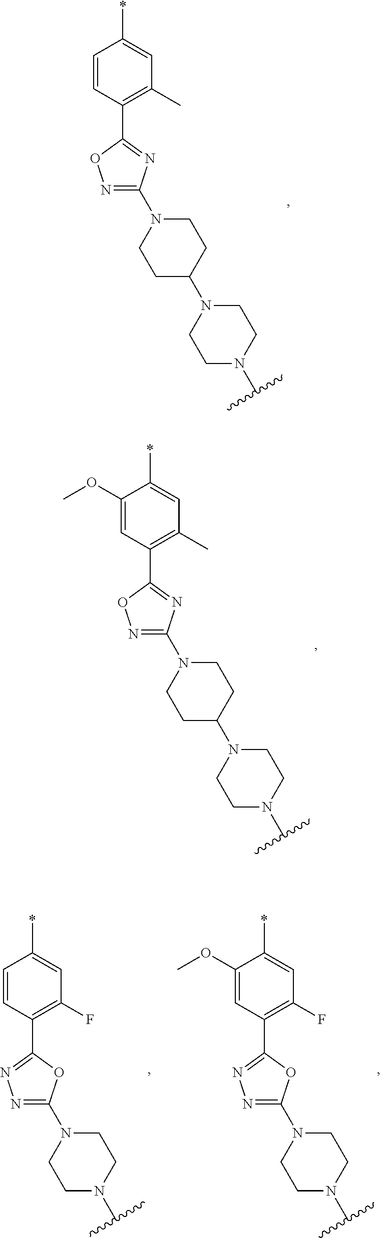 [SMILES: *c1ccc(-c2nc(N3CCC(N4CCN(*)CC4)CC3)no2)c(C)c1 ; *c1cc(C)c(-c2nc(N3CCC(N4CCN(*)CC4)CC3)no2)cc1OC ; *c1ccc(-c2nnc(N3CCN(*)CC3)o2)c(F)c1 ; *c1cc(F)c(-c2nnc(N3CCN(*)CC3)o2)cc1OC] 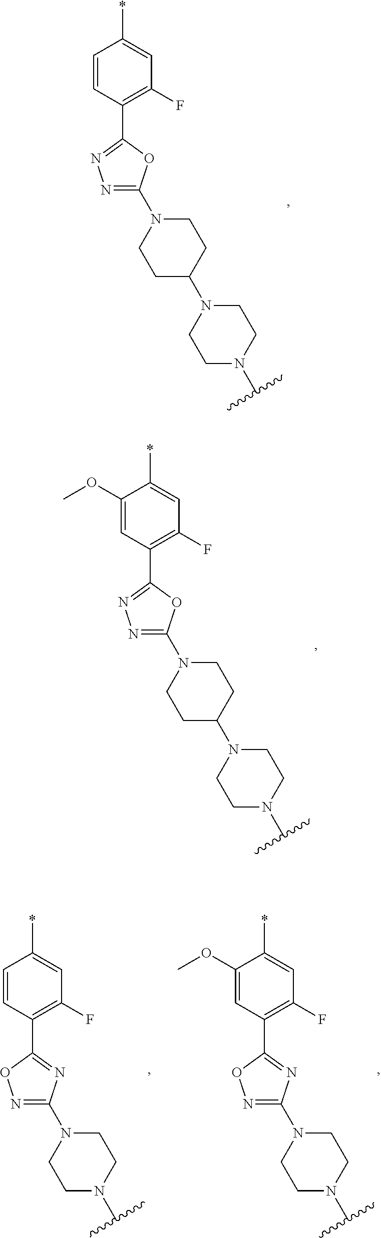 [SMILES: *c1ccc(-c2nnc(N3CCC(N4CCN(*)CC4)CC3)o2)c(F)c1 ; *c1cc(F)c(-c2nnc(N3CCC(N4CCN(*)CC4)CC3)o2)cc1OC ; *c1ccc(-c2nc(N3CCN(*)CC3)no2)c(F)c1 ; *c1cc(F)c(-c2nc(N3CCN(*)CC3)no2)cc1OC] 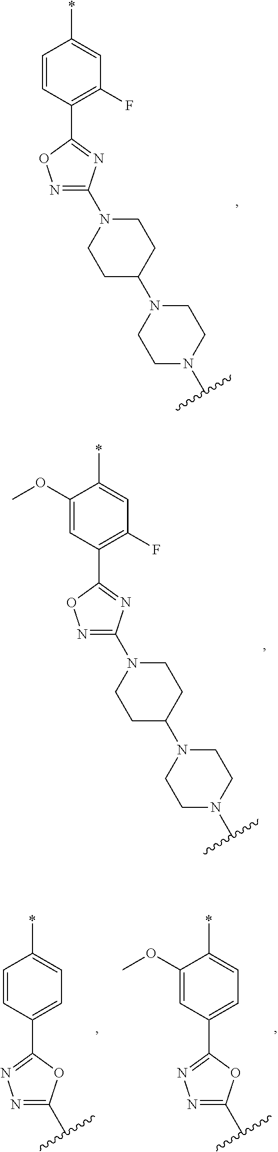 [SMILES: *c1ccc(-c2nc(N3CCC(N4CCN(*)CC4)CC3)no2)c(F)c1 ; *c1cc(F)c(-c2nc(N3CCC(N4CCN(*)CC4)CC3)no2)cc1OC ; *c1ccc(-c2nnc(*)o2)cc1 ; *c1nnc(-c2ccc(*)c(OC)c2)o1] 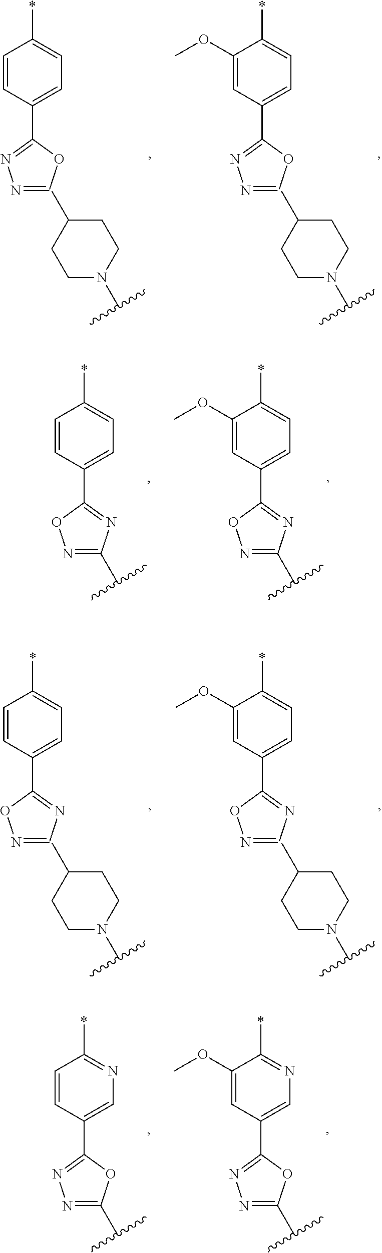 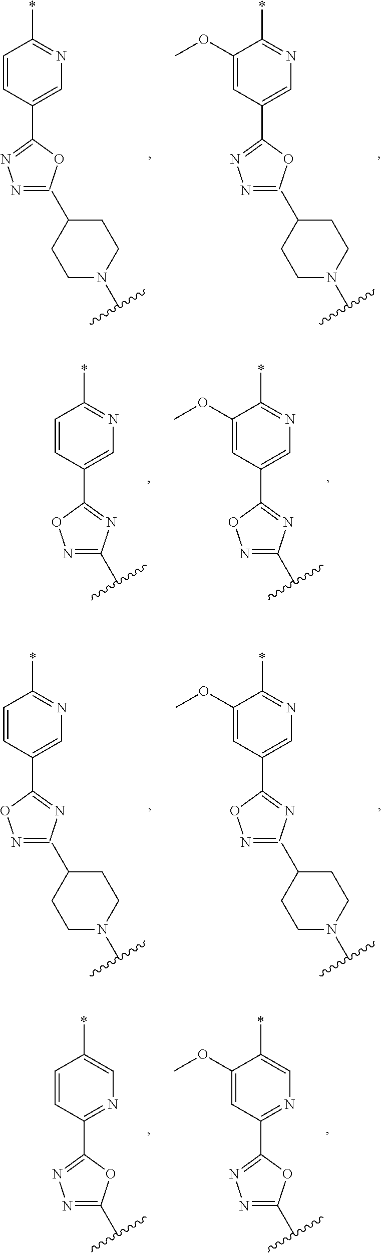 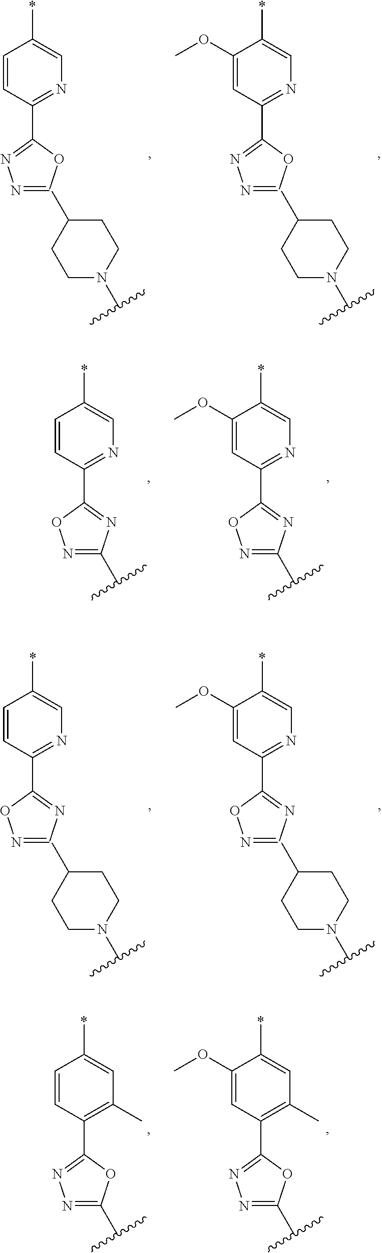 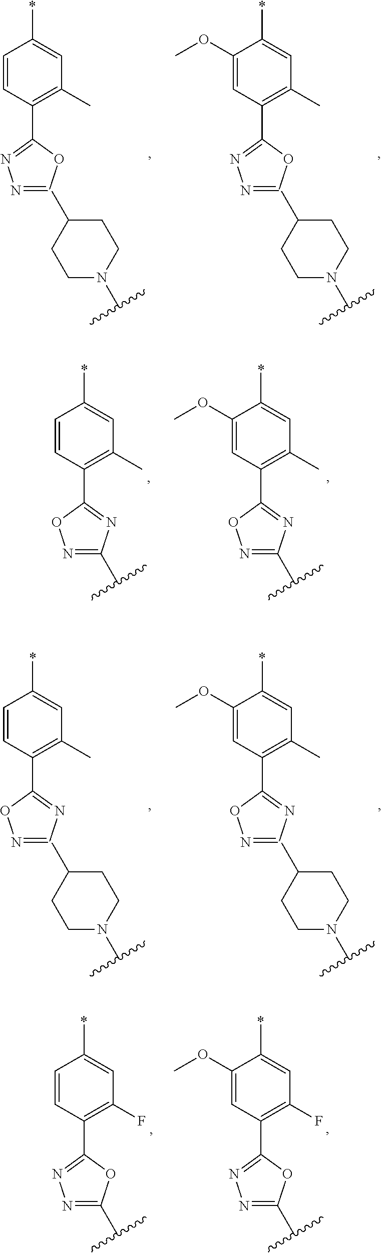 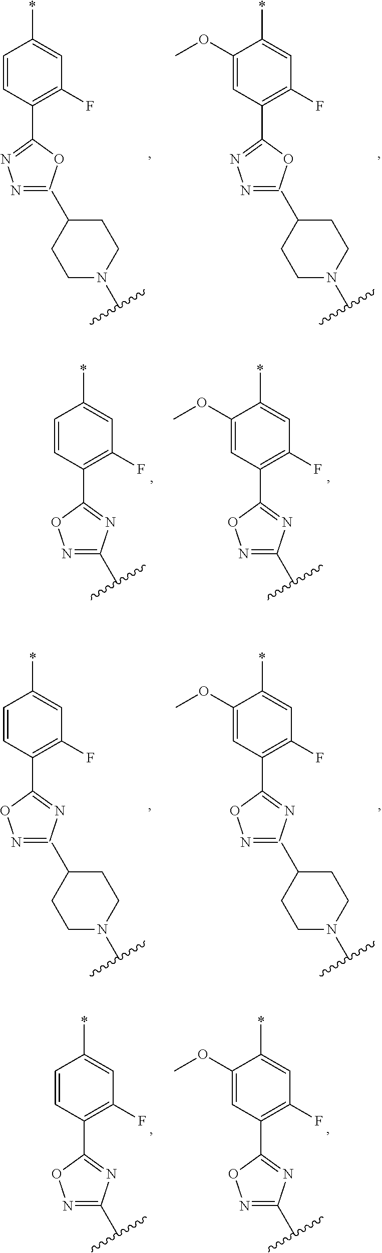 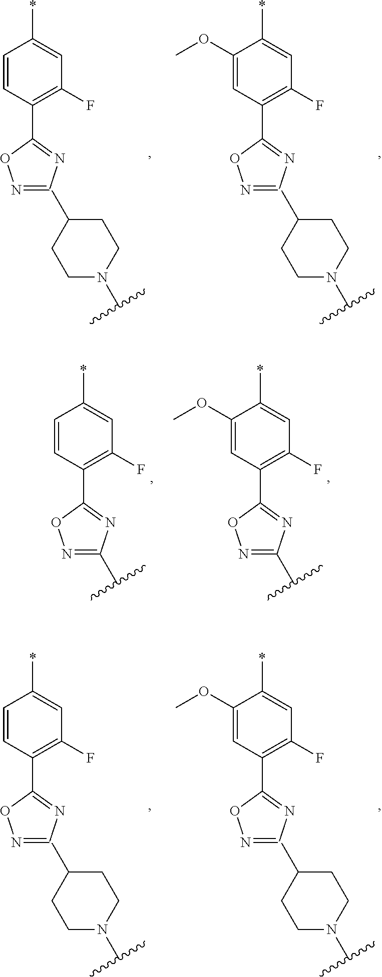 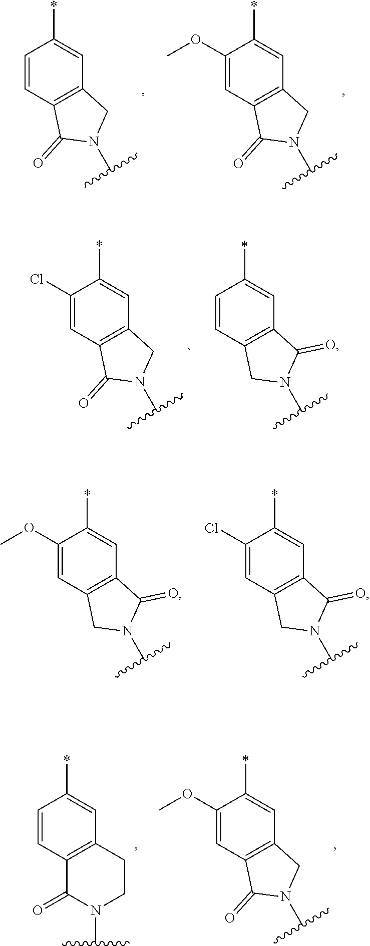 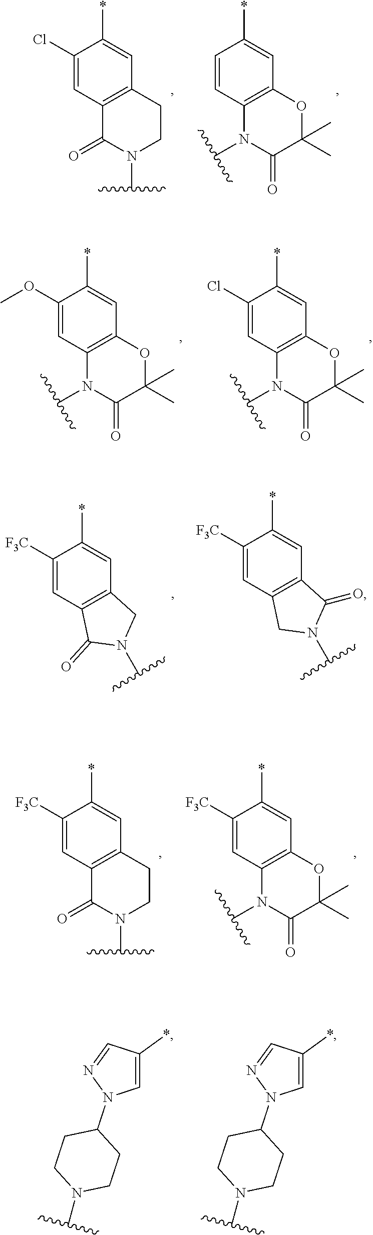 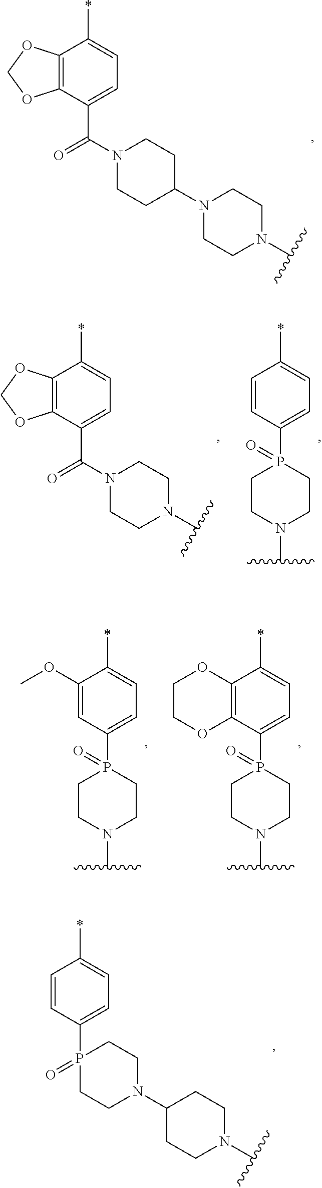 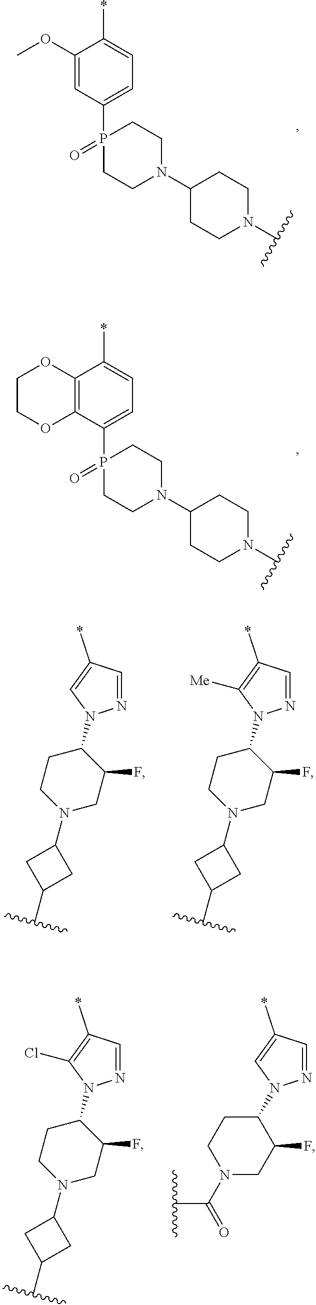 [SMILES: *c1ccc(P2(=O)CCN(C3CCN(*)CC3)CC2)cc1OC ; *c1ccc(P2(=O)CCN(C3CCN(*)CC3)CC2)c2c1OCCO2 ; *c1cnn([C@H]2CCN(C3CC(*)C3)C[C@@H]2F)c1 ; *c1cnn([C@H]2CCN(C3CC(*)C3)C[C@@H]2F)c1C ; *c1cnn([C@H]2CCN(C3CC(*)C3)C[C@@H]2F)c1Cl ; *C(=O)N1CC[C@H](n2cc(*)cn2)[C@@H](F)C1] 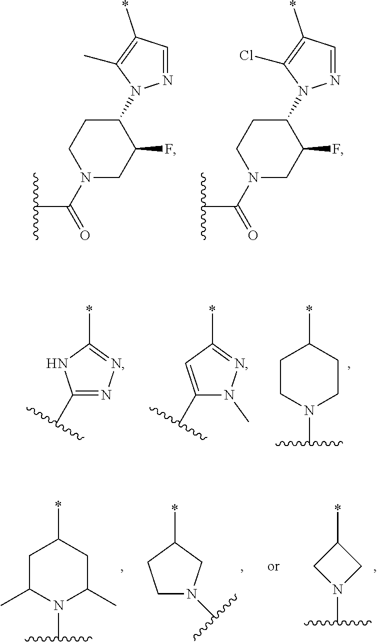
or represents H;
R₂ represents 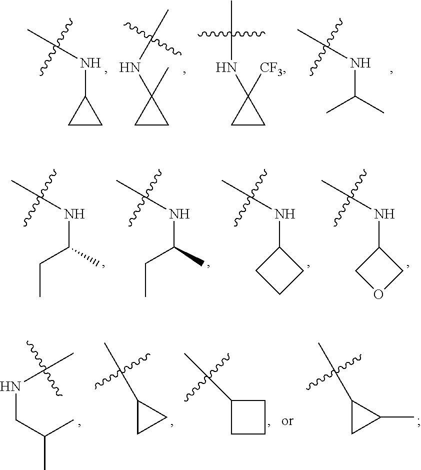
R₃ represents H, halo, or CF₃, or wherein R₃ represents CR₆, R₂ represents NH and together with the atoms to which they are bound form a pyrrolyl group substituted with R₆;
and R₄ represents H, 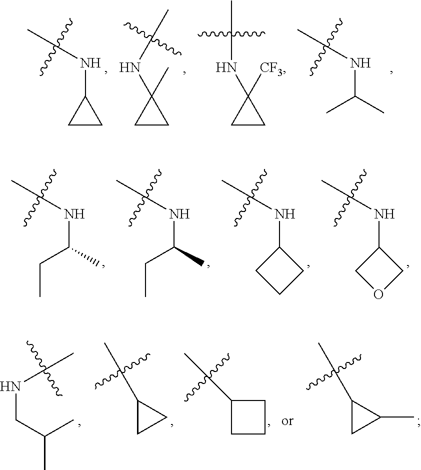
provided that one of R₁ and R₅ provides an attachment point for

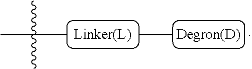

8. The bifunctional compound of claim 7, wherein X represents N, R₄ is H, and the targeting ligand has a structure represented by formula (TL2-b1): 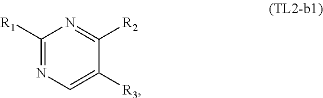
wherein
R₂ represents 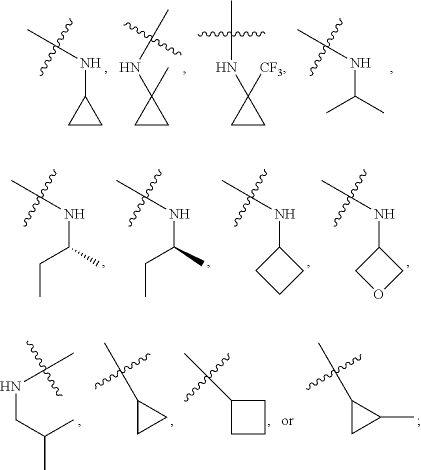
and
R₃ represents H, halo, or CF₃.

9. The bifunctional compound of claim 7, wherein X represents N and R2 represents NH, R₃ represents CR₆, and R₂ and R₃ together with the atoms to which they are bound form a pyrrolyl group substituted with R₆, and the targeting ligand has a structure represented by formula (TL2-b2):

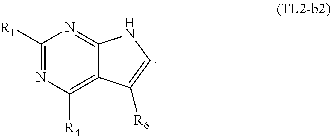

10. The bifunctional compound of claim 7, wherein X represents CR₅, wherein R₅ is H and R₂ represents NH, R₃ represents CR₆, and R₂ and R₃ together with the atoms to which they are bound form a pyrrolyl group substituted with R₆, and the targeting ligand has a structure represented by formula (TL2:b3):

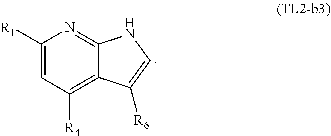

11. The bifunctional compound of claim 7, wherein R₁ is absent (which also means R₁ represents H), and R₂ represents NH, R₃ represents CR₆, and R₂ and R₃ together with the atoms to which they are bound form a pyrrolyl group substituted with R₆, and the targeting ligand has a structure represented by formula (TL2:b4):

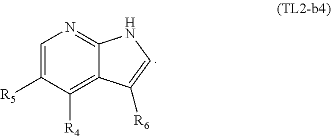

12. The bifunctional compound of claim 7, wherein X represents CR₆, R₁ is absent (which also means R₁ represents H), and R₂ represents NH, R₃ represents CR₅, and R₂ and R₃ together with the atoms to which they are bound form a pyrrolyl group substituted with R₅, the targeting ligand has a structure represented by formula (TL2-b5):

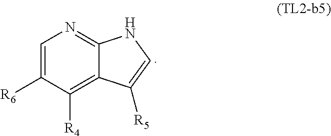

13. The bifunctional compound of claim 1, wherein the linker is represented by any one of structures:

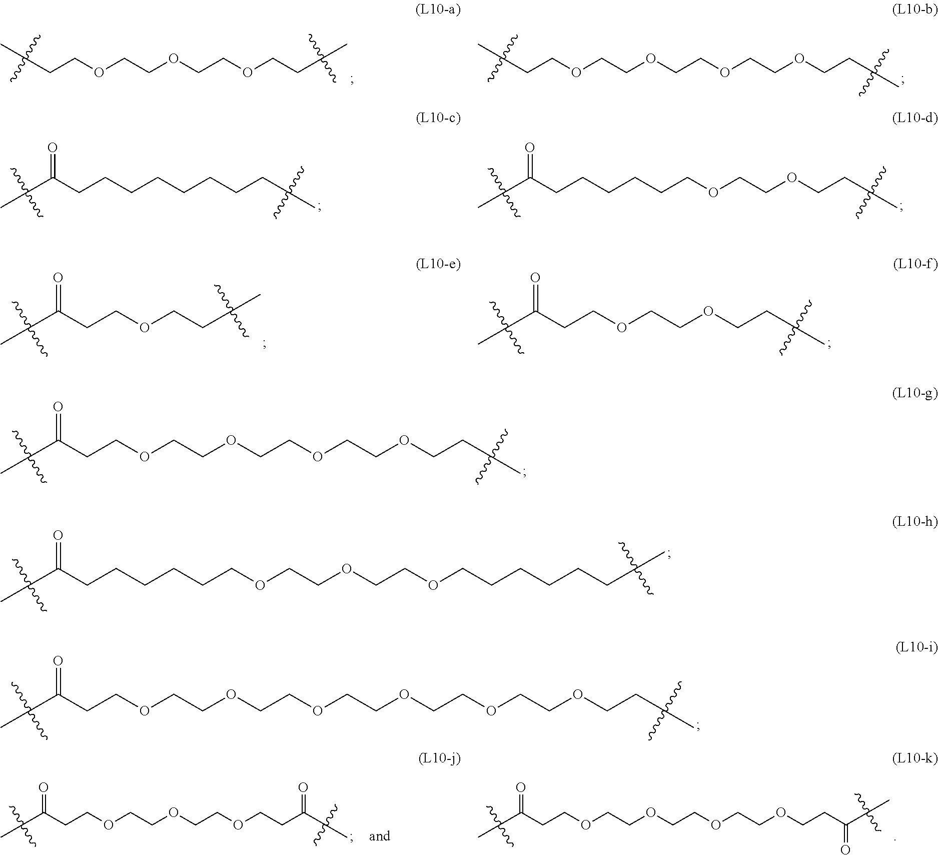

14. The bifunctional compound of claim 1, wherein the degron binds cereblon (CRBRN).

15. The bifunctional compound of claim 14, wherein the degron that binds cereblon is represented by any one of formulae: 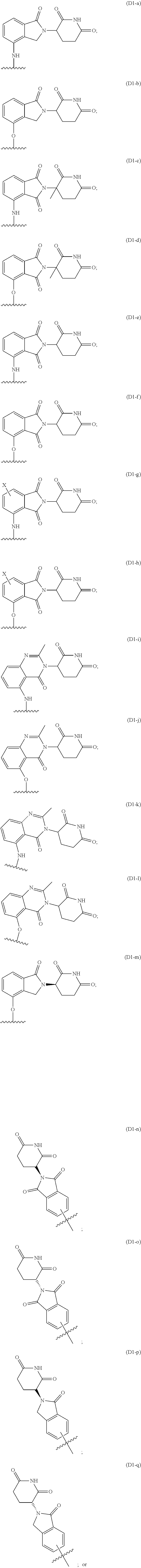 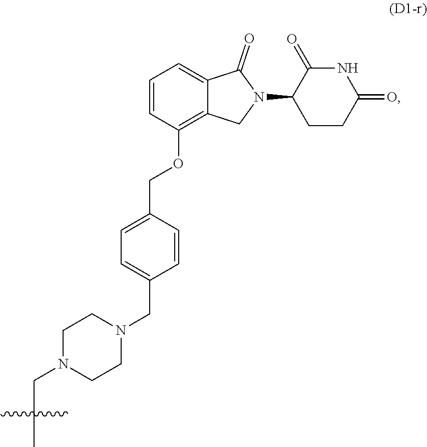
wherein X is alkyl, halo, CN, CF₃, OCHF₂ or OCF₃.

16. The bifunctional compound of claim 1, wherein the degron binds VHL.

17. The bifunctional compound of claim 16, wherein the degron has a structure represented by any one of structures: 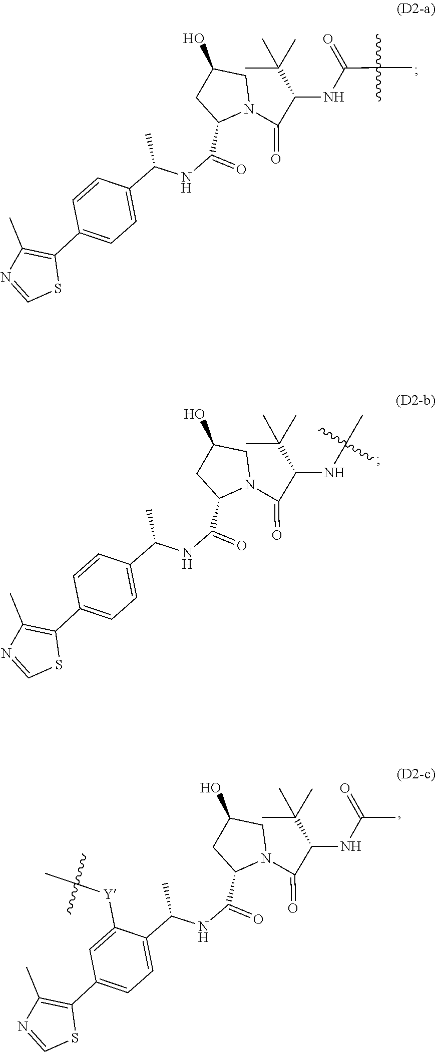
wherein Y′ is a bond, N, O or C; 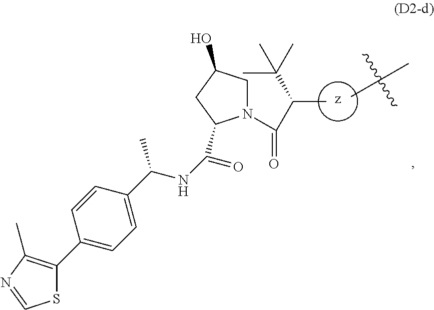
wherein Z is a C₅-C₆ carbocyclic or C₅-C₆ heterocyclic group, and

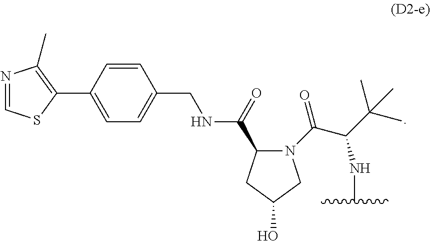 [SMILES: *N[C@H](C(=O)N1C[C@H](O)C[C@H]1C(=O)NCc1ccc(-c2scnc2C)cc1)C(C)(C)C]

18. The bifunctional compound of claim 1, wherein the degron binds an inhibitor of apoptosis protein.

19. The bifunctional compound of claim 18, wherein the degron has a structure represented by any one of structures:

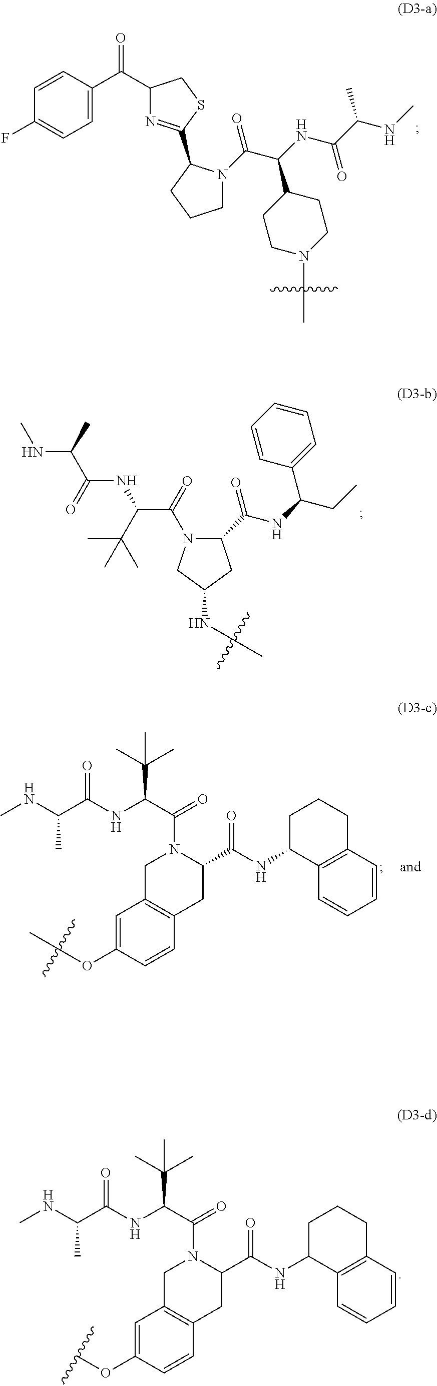

20. The bifunctional compound of claim 1, wherein the degron binds murine double minute 2.

21. The bifunctional compound of claim 20, wherein the degron has a structure represented by any one of structures:

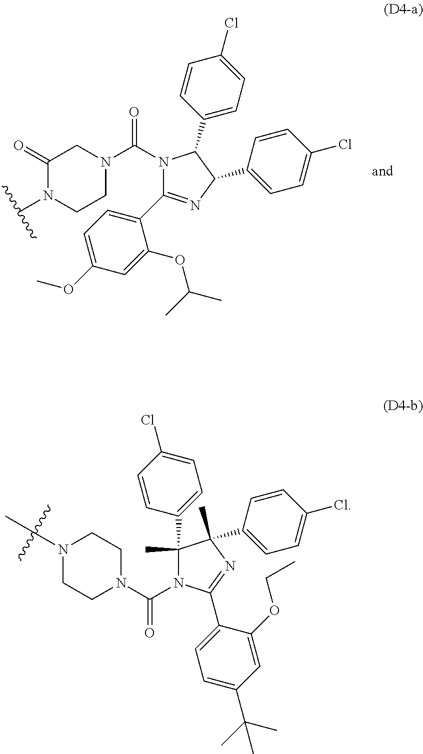 [SMILES: *N1CCN(C(=O)N2C(c3ccc(OC)cc3OC(C)C)=N[C@@H](c3ccc(Cl)cc3)[C@H]2c2ccc(Cl)cc2)CC1=O ; *N1CCN(C(=O)N2C(c3ccc(C(C)(C)C)cc3OCC)=N[C@@](C)(c3ccc(Cl)cc3)[C@@]2(C)c2ccc(Cl)cc2)CC1]

22. The bifunctional compound of claim 1, which is selected from the group consisting of: 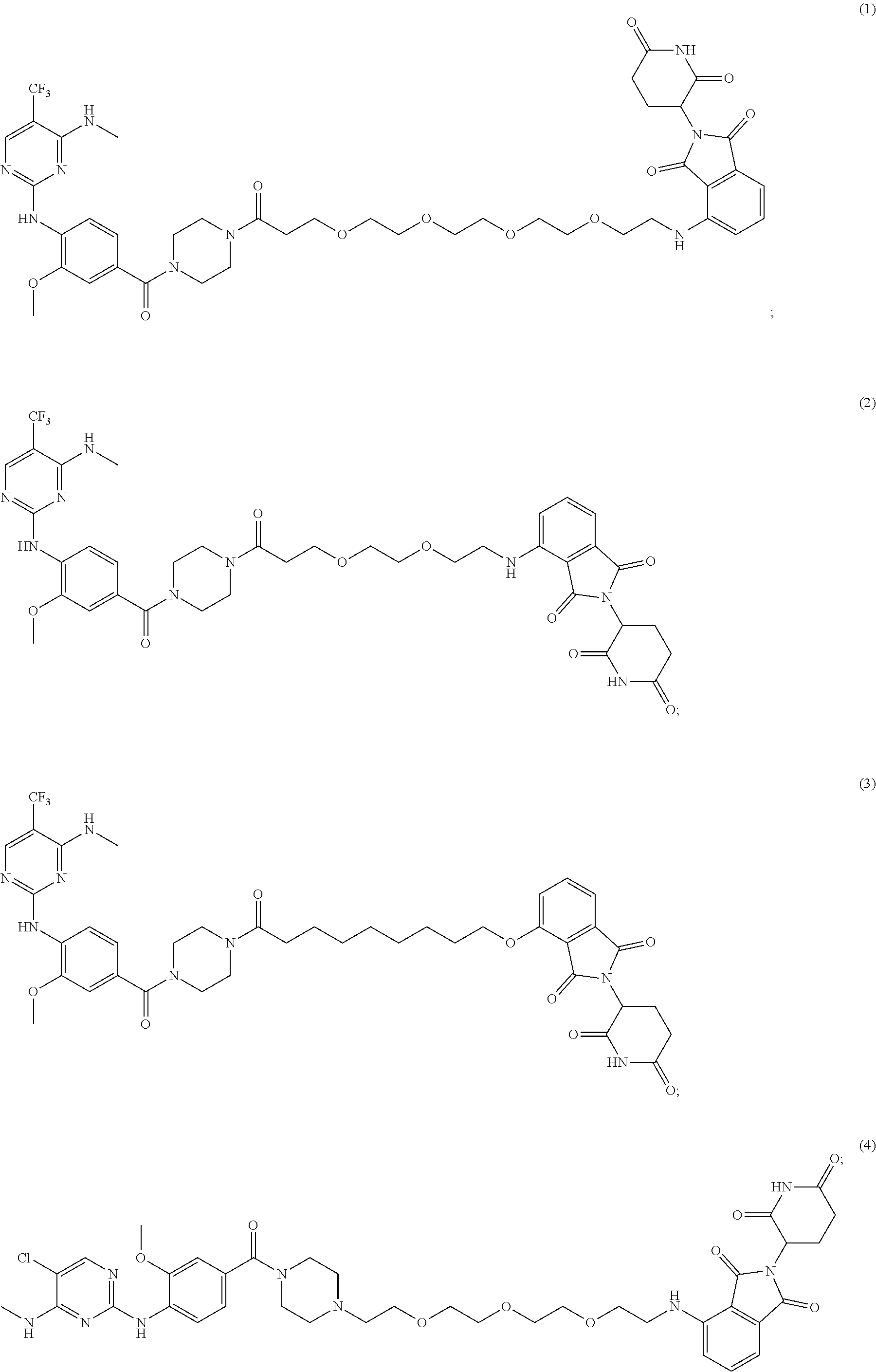 [SMILES: CNc1nc(Nc2ccc(C(=O)N3CCN(C(=O)CCOCCOCCOCCOCCNc4cccc5c4C(=O)N(C4CCC(=O)NC4=O)C5=O)CC3)cc2OC)ncc1C(F)(F)F ; CNc1nc(Nc2ccc(C(=O)N3CCN(C(=O)CCOCCOCCNc4cccc5c4C(=O)N(C4CCC(=O)NC4=O)C5=O)CC3)cc2OC)ncc1C(F)(F)F ; CNc1nc(Nc2ccc(C(=O)N3CCN(C(=O)CCCCCCCCOc4cccc5c4C(=O)N(C4CCC(=O)NC4=O)C5=O)CC3)cc2OC)ncc1C(F)(F)F ; CNc1nc(Nc2ccc(C(=O)N3CCN(CCOCCOCCOCCNc4cccc5c4C(=O)N(C4CCC(=O)NC4=O)C5=O)CC3)cc2OC)ncc1Cl] 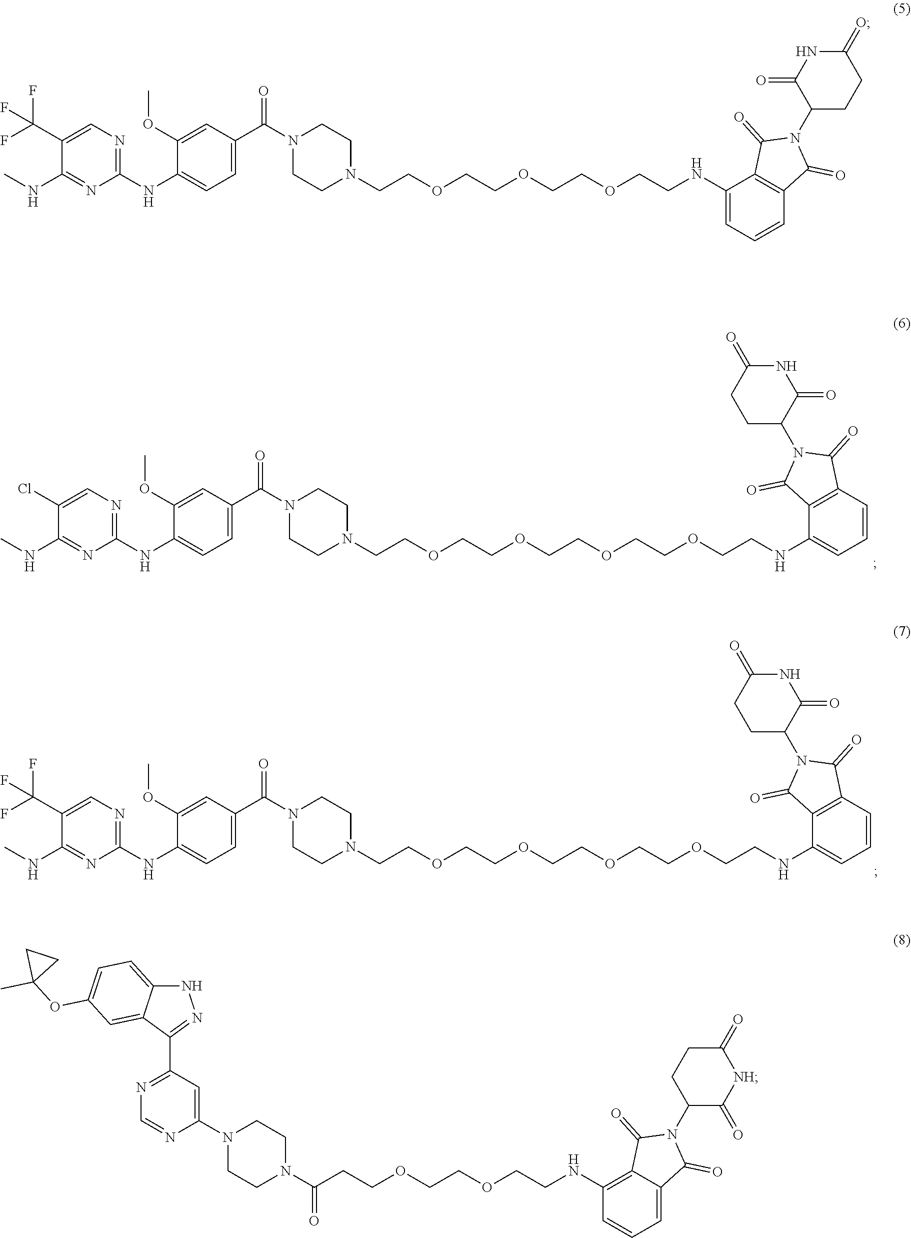 [SMILES: CNc1nc(Nc2ccc(C(=O)N3CCN(CCOCCOCCOCCNc4cccc5c4C(=O)N(C4CCC(=O)NC4=O)C5=O)CC3)cc2OC)ncc1C(F)(F)F ; CNc1nc(Nc2ccc(C(=O)N3CCN(CCOCCOCCOCCOCCNc4cccc5c4C(=O)N(C4CCC(=O)NC4=O)C5=O)CC3)cc2OC)ncc1Cl ; CNc1nc(Nc2ccc(C(=O)N3CCN(CCOCCOCCOCCOCCNc4cccc5c4C(=O)N(C4CCC(=O)NC4=O)C5=O)CC3)cc2OC)ncc1C(F)(F)F ; CC1(Oc2ccc3[nH]nc(-c4cc(N5CCN(C(=O)CCOCCOCCNc6cccc7c6C(=O)N(C6CCC(=O)NC6=O)C7=O)CC5)ncn4)c3c2)CC1] 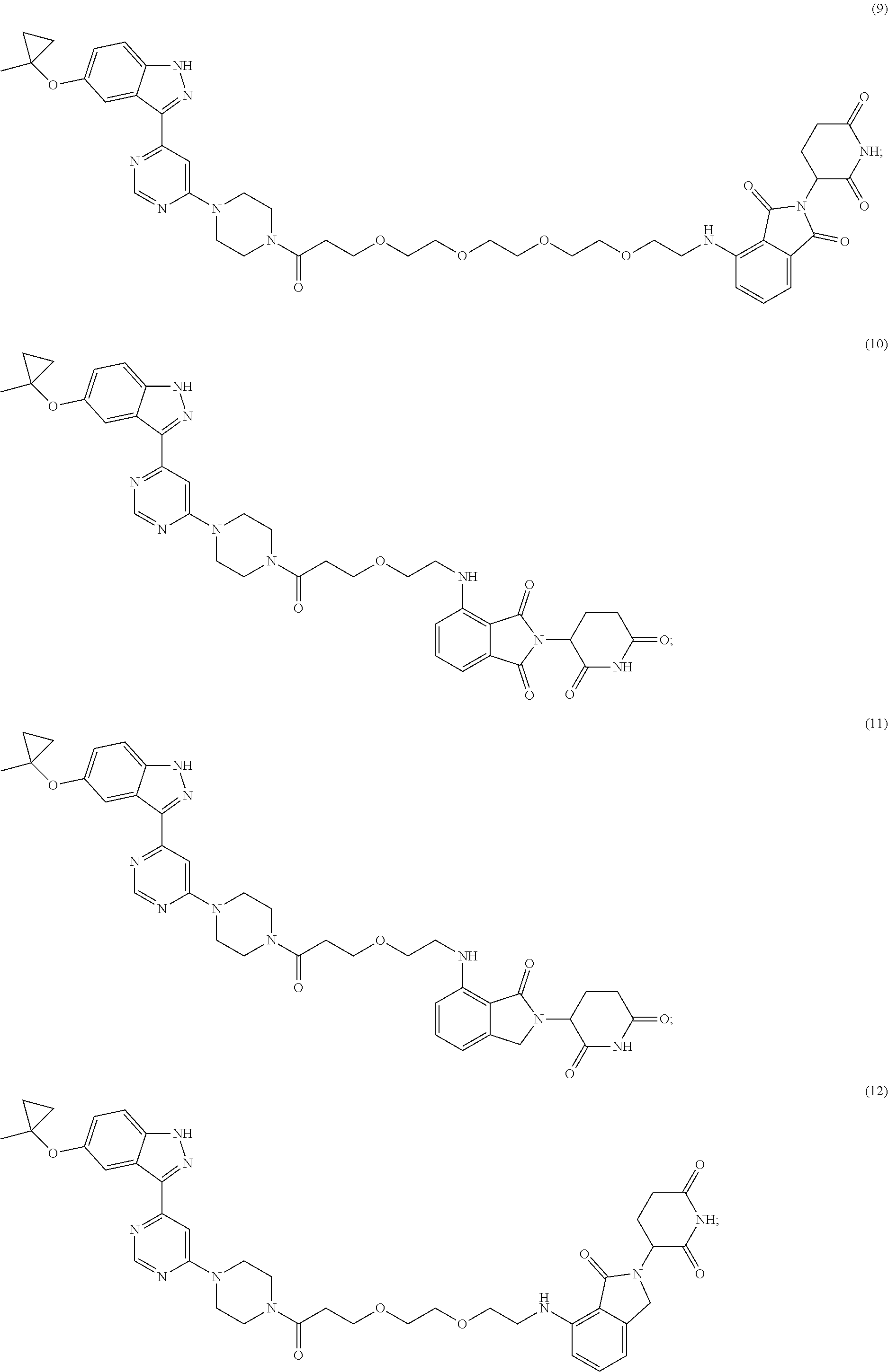 [SMILES: CC1(Oc2ccc3[nH]nc(-c4cc(N5CCN(C(=O)CCOCCOCCOCCOCCNc6cccc7c6C(=O)N(C6CCC(=O)NC6=O)C7=O)CC5)ncn4)c3c2)CC1 ; CC1(Oc2ccc3[nH]nc(-c4cc(N5CCN(C(=O)CCOCCNc6cccc7c6C(=O)N(C6CCC(=O)NC6=O)C7=O)CC5)ncn4)c3c2)CC1 ; CC1(Oc2ccc3[nH]nc(-c4cc(N5CCN(C(=O)CCOCCNc6cccc7c6C(=O)N(C6CCC(=O)NC6=O)C7)CC5)ncn4)c3c2)CC1 ; CC1(Oc2ccc3[nH]nc(-c4cc(N5CCN(C(=O)CCOCCOCCNc6cccc7c6C(=O)N(C6CCC(=O)NC6=O)C7)CC5)ncn4)c3c2)CC1] 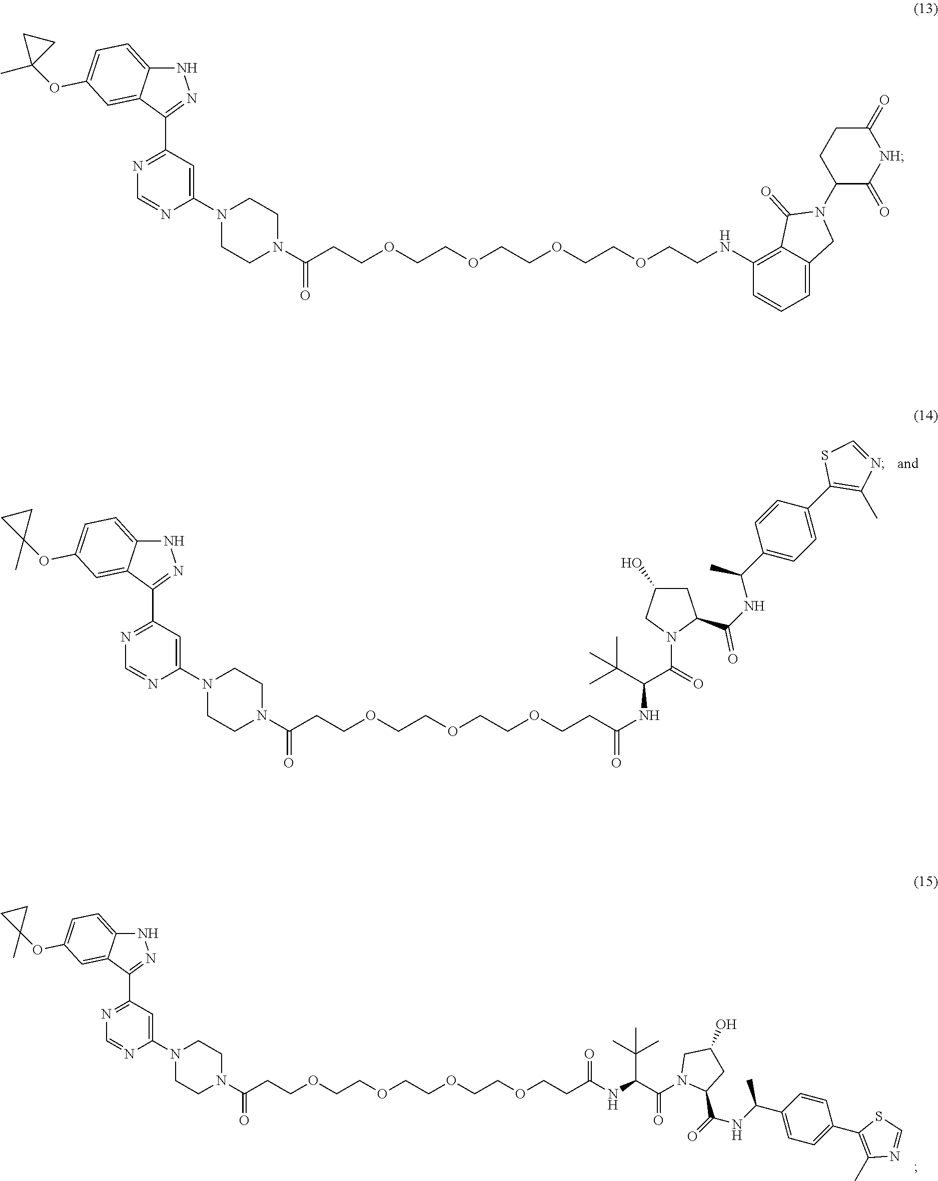 [SMILES: CC1(Oc2ccc3[nH]nc(-c4cc(N5CCN(C(=O)CCOCCOCCOCCOCCNc6cccc7c6C(=O)N(C6CCC(=O)NC6=O)C7)CC5)ncn4)c3c2)CC1 ; Cc1ncsc1-c1ccc([C@H](C)NC(=O)[C@@H]2C[C@@H](O)CN2C(=O)[C@@H](NC(=O)CCOCCOCCOCCC(=O)N2CCN(c3cc(-c4n[nH]c5ccc(OC6(C)CC6)cc45)ncn3)CC2)C(C)(C)C)cc1 ; Cc1ncsc1-c1ccc([C@H](C)NC(=O)[C@@H]2C[C@@H](O)CN2C(=O)[C@@H](NC(=O)CCOCCOCCOCCOCCC(=O)N2CCN(c3cc(-c4n[nH]c5ccc(OC6(C)CC6)cc45)ncn3)CC2)C(C)(C)C)cc1]
and pharmaceutically acceptable salts and stereoisomers thereof.

23. A pharmaceutical composition containing a therapeutically effective amount of the bifunctional compound of claim 1, or a pharmaceutically acceptable salt or stereoisomer thereof, and pharmaceutically acceptable carrier.

24. A method of treating a disease or disorder mediated by aberrant LRRK2 activity, comprising administering a therapeutically effective amount of the bifunctional compound of claim 1, or a pharmaceutically acceptable salt or stereoisomer thereof, to a subject in need thereof.

25. The method of claim 24, wherein the disease or disorder is Parkinson's disease or brain cancer.

26. The method of claim 25, wherein the brain cancer is a glioma or glioblastoma.
